SEQUENCE LISTING (276 sequences)

SEQ ID NO: 1 - protein, 158 aa, Artificial Sequence
  N-terminal Signal Sequence
  misc_feature (23)..(23): Xaa can be any naturally occurring amino acid
  misc_feature (99)..(99): Xaa can be any naturally occurring amino acid
MPFSNKIVNGDFETGTLIPWSSXNVAITNLQSHTGFFSALLFGNTANSLLFQAVPVTPGDSFEFFLSIAKLGNLVSPQVNIALLYLDVAATPVGIGLNXTIPVGHLPDNTANIWTTIYETTSVVPATATQALLIIHKIPSLSTADIVVDDIVLLQTSG

SEQ ID NO: 2 - protein, 186 aa, Artificial Sequence
  N-terminal Signal Sequence
  misc_feature (112)..(112): Xaa can be any naturally occurring amino acid
MVRTLSKIEDSTYTTLAPINPIMEVVIYLPFSNKVVNGDFETGTLFPWSFTNVSITNLQSHTGFFSALLFGDTANSLLFQAIPVTPGDSFELFLSIAKLGNLVSPQVNIALXYLDVATIPVGIGLNLTIPVGHLPDNTANIWTTIYETTSVVPATATQALLIINKIPALSTADIVVDDIGILQTSG

SEQ ID NO: 3 - protein, 29 aa, Artificial Sequence
  N-terminal Signal Sequence
MLPLIFPPQGCPAIFPGPTLDPPDPPDPP

SEQ ID NO: 4 - protein, 56 aa, Artificial Sequence
  N-terminal Signal Sequence
MMILTIKIFRKERAKINDKRANAFNKKILPLIFPPQGCPAIFPGPTLDPPDPPDPP

SEQ ID NO: 5 - protein, 49 aa, Artificial Sequence
  N-terminal Signal Sequence
MKELHDIKRIWDEVANTDLEEFTRKNPIYMKSSNGISSSSSTFCWTFIR

SEQ ID NO: 6 - protein, 52 aa, Artificial Sequence
  N-terminal Signal Sequence
MKELHDIKRIWDEVANTDLEEFTRKNPIYMKSSNGISSSSSTFCWTFIRGKT

SEQ ID NO: 7 - protein, 187 aa, Artificial Sequence
  N-terminal Signal Sequence
  misc_feature (170)..(170): Xaa can be any naturally occurring amino acid
MVLALSKIGESTYNASVPINLIMEVVIYLPFSNIVENGNFESGTLFPWSFTNAAITDLQSHSGFFSALLFGGTANSLLFQTVPVTPGDSFELFLSIAKSGNLISPQVNIVLIYLDAANIPVGIGLNLTIPVGHLPDNTANIWTTIYETTAVVPATAIEALLIISKVPALXSTADVILDDIELLQTSG

SEQ ID NO: 8 - protein, 164 aa, Artificial Sequence
  N-terminal Signal Sequence
MKAVIYLPFGNKIVNGNFETGSLVPWNFSNVTINNLQSHTGSFSALFFGNTTNSVLYQTVPVTSGEIFEFFLSIGKIGNLPSPQVNIALIYLNAASTPENIGINIILPVGKLPNNLNNNWSTIYEISSVVPAAATQAMVIIHKIPAPSTADIVVDDIVLVQTGA

SEQ ID NO: 9 - protein, 158 aa, Artificial Sequence
  N-terminal Signal Sequence
MPFGNKIVNGNFETGSLIPWSSSNVAISNLQSHTGSYSALLFGNAANSFLFQAVPVTVGDSFEFFLSIAKIGNLPSPQVDIALIYLNAASTPISNGMSIILPTGHLPDNLNNNWSTIYEISSVVPAMATHAMVIIHKIPSPSTSDIVVDDIVLVQTGA

SEQ ID NO: 10 - protein, 154 aa, Artificial Sequence
  N-terminal Signal Sequence
NKVVNGDFETGTLFPWSFTNVSITNLQSHTGFFSALFFGDTANSLLFQAIPVTPGDSFELFLSIAKLGNLVSPQVNIALLYLDVATIPVGIGLNLTIPVGHLPDNTANIWTTIYETTSVVPATATQALLIINKISALSTADIVVDDIGILQTSG

SEQ ID NO: 11 - protein, 110 aa, Artificial Sequence
  N-terminal Signal Sequence
  misc_feature (93)..(93): Xaa can be any naturally occurring amino acid
MFQAVPVTVGDSFEFFLSIAKIGNLPSPEVNIALIYLNVASTPISIGMSIILPIGHLPDNLNNNWSTIYEISSVVPATATHAMVIIHKIPSPXSTSDIVVDDIVLVQTGA

SEQ ID NO: 12 - protein, 164 aa, Artificial Sequence
  N-terminal Signal Sequence
MKAVIYLPLGNKIVNGNFETGSLVPWNFSNVAIYNLQSHTGSFSALFFGNTTNSVLYQTVPVTSGEIFEFFLSIGKIGNLPSPQVNIALIYLNAASTPENIGINIILPVGKLPNNLNNNWSTIYEISSVVPAAATQAMVIIHKIPAPSTADIVVDDIVLVQTGA

SEQ ID NO: 13 - protein, 158 aa, Artificial Sequence
  N-terminal Signal Sequence
MPFSNKIVNGDFETGTLIPWSFVNVAITNLQSHTGFFSAQLFGTTANSLLFQAVPVTPGDSFEFFLSIAKLGNLLSPQVNIALLYLDVAATPVGIGLNITIPIGHLPDNAANIWTTIYETTTVVPATATQALLIINKIPTLTTADIVVDDIALLQVSG

SEQ ID NO: 14 - protein, 95 aa, Artificial Sequence
  N-terminal Signal Sequence
MFLSIAKSGNVISPQVNIVLIYLDVANIPVGIGLNLTIPVGHLPDNTANIWTTIYETTSVVPATAIEALLIINKVPALSTADVILDDIGLLQTSG

SEQ ID NO: 15 - protein, 142 aa, Artificial Sequence
  N-terminal Signal Sequence
LLPWSFTNAAITDLQSHSGFFSALLFGGTANSLLFQTVPVTPGDSFELFLSIAKSGNLISPQVNIVLIYLDAANIPVGIGLNLTIPVGHLPDNTANIWTTIYETTAVVPATAIEALLIISKVPALSTADVILDDIELLQTSG

SEQ ID NO: 16 - protein, 158 aa, Artificial Sequence
  N-terminal Signal Sequence
MPFSNKIVNGDFETGTLIPWSFTNVAITNLQSHSGFFSARLFGTTVNSLLFQSVPVTPGDSFEFFLSIAKLGTLLSPQVNIALLYLDVTATPVGIGLNITIPIGHLPDNTANIWTTIYETTTVVPATATQALLIINKIPTLTTADIVVDDIELLQVSG

SEQ ID NO: 17 - protein, 161 aa, Artificial Sequence
  N-terminal Signal Sequence
MSFINKIVNGDFETGTLIPWSFVNVAINPLQSHTGFFSARLFGNTANSLLFQAVPVTPGDSFEFFLSIAKIGNLTSPQVNIVLFYLDALANPISLGLNITIPSGHLPDNVDNNWTTIYETTSAVPATATQALLIINKIPALFTTADIVVDDIALLQTGAGV

SEQ ID NO: 18 - protein, 158 aa, Artificial Sequence
  N-terminal Signal Sequence
MPFGNKIVNGNFETGSLIPWSSSNVVISNLQSHTGSYSALLFGNEANSLMFQAVPVTVGDSFEFFLSIAKIGNLPSPQVNIALIYLNVASTPISIGMSIILPIGHLPDNLNNNWSTIYEISSVVPATATHAMVIIHKIPSPSTSDIVVDDIVLVQTGA

SEQ ID NO: 19 - protein, 158 aa, Artificial Sequence
  N-terminal Signal Sequence
MPVKNRAVNGDFETGSLVPWNSINVTISNQQSHSGTFSALLFGTSANSLLFQTVPVITGDSFEFFLSIAKIGNLPSPQVNIAVIYVNATSTPLSIGMSVILPVNHLPNNLNNNWLTIYNTTSVVPVTATQAIIIIHKIPAPSTADIVVDDIALIQTGG

SEQ ID NO: 20 - protein, 158 aa, Artificial Sequence
  N-terminal Signal Sequence
MPFSNKIVNGNFETGALIPWSSINVAITNLQSHTGFFSARLFGNTTNSLLFQAVPVTPGDSFEFLLSIAKLGNLVSPQVNIALLYLDVTATPVGIGLNFTIPVGHLPDNTANIWTTIYETTSVVPATATQALLIINKIAALSTADIVVDDIGILQTSG

SEQ ID NO: 21 - protein, 158 aa, Artificial Sequence
  N-terminal Signal Sequence
MPFSNKIVNGNFETGALIPWSSINVAITNLQSHTGFFSARLFGNTTNSLLFQAVPVTPGDSFEFLLSIAKLGNLVSPQVNIALLYLDVTATPVGIGLNFTIPVGHLPDNTANIWTTIYETTSVVPATAIQALLIIEKVAALTTADVVVDDIQLLQTSG

SEQ ID NO: 22 - protein, 109 aa, Artificial Sequence
  N-terminal Signal Sequence
MFQAVPVTVGDSFEFFLSIAKIGNLPSPEVNIALIYLNVASTPISIGMSIILPIGHLPDNLNNNWSTIYEISSVVPATATHAMVIIHKIPSPSTSDIVVDDIVLVQTGA

SEQ ID NO: 23 - protein, 158 aa, Artificial Sequence
  N-terminal Signal Sequence
MPFGNKIVNGNFETGSLIPWSSSNVVISNLQSHTGSYSALLFGNAANSLMFQAVPVTVGDSFEFFLSIAKIGNLPSPQVNIALIYLNVASTPISIGMSIILPIGHLPDNLNNNWSTIYEISSVVPATATHAMVIIHKIPSPSTSDIVVDDIVLVQTGA

SEQ ID NO: 24 - protein, 158 aa, Artificial Sequence
  N-terminal Signal Sequence
MPFSNKIVNGNFETGALIPWSSINVAITNLQSHTGFFSARLFGNTTNSLLFQAVPVTLGDSFEFLLSIAKLGNLVSPQVNIALLYLDVTATPVGIGLNFTIPVGHLPDNTANIWTTIYETTSVVPATAIQALLIIEKVAALTTADVVVDDIQLLQTSG

SEQ ID NO: 25 - protein, 186 aa, Artificial Sequence
  N-terminal Signal Sequence
MVRTLSKIEDSTYTTLAPINPIMEVVIYLPFSNKVVNGDFETGTLFPWSFTNVSITNLQSHTGFFSALLFGDTANSLLFQAIPVTPGDSFELFLSIAKLGNLVSPQVNIALLYLDVATIPVGIGLNLTIPVGHLPDNTANIWTTIYETTSVVPATATQALLIINKISALSTADIVVDDIGILQTSG

SEQ ID NO: 26 - protein, 158 aa, Artificial Sequence
  N-terminal Signal Sequence
MPFGNKIVNGNFETGSLIPWSSSNVAISNLQSHTGSYSALLFGNAANSFLFQAVPVTVGDSFEFFLSIAKIGNLPSPQVDIALIYLNAASTPISIGMSIILPTGHLPDNLNNNWSTIYEISSVVPAMATHAMVIIHKIPSPSTSDIVVDDIVLVQTGA

SEQ ID NO: 27 - protein, 186 aa, Artificial Sequence
  N-terminal Signal Sequence
MVRTLSKIEDSTYTTLAPINPIMEVVIYLPFSNKVVNGDFETGTLFPWSFTNVSITNLQSHTGFFSAILFGDTANSLLFQAIPVTPGDSFELFLSIAKLGNLVSPQVNIALLYLDVATIPVGIGLNLTIPVGHLPDNTANIWTTIYETTSVVPANATQALLIINKISALSTADIVVDDIGILQTSG

SEQ ID NO: 28 - protein, 149 aa, Artificial Sequence
  N-terminal Signal Sequence
GDFETGTLFPWSFTNVSITNLQSHTGFFSAILFGDTANSLLFQAIPVTPGDSFELFLSIAKLGNLVSPQVNIALLYLDVATIPVGIGLNLTIPVGHLPDNTANIWTTIYETTSVVPANATQALLIINKISALSTADIVVDDIGILQTSG

SEQ ID NO: 29 - protein, 158 aa, Artificial Sequence
  N-terminal Signal Sequence
MPFGNKIVNGNFETGSLIPWSSSNVAISNLQSHTGSYSALLFGNTANSLMFQAVPVTVGDSFEFFLSIAKIGNLPSAQVNIALIYLNVASTPISIGMSIILPIGHLPDNLNNNWSTIYEISSVVPAMATHAMVIIHKIPSPSTSDIVVDDIVLVQTGA

SEQ ID NO: 30 - protein, 158 aa, Artificial Sequence
  N-terminal Signal Sequence
MPFSNKIVNGDFETGTLIPWSSINVAITNLQSHTGFFSARLFGNTANSLLFQAVPVTPGDSFEFLLSIAKLGNLVSPQVNIALLYLDVTATPVGIGLNLTIPVGHLPDNTADIWTTIYETTSVVPATAIQALLIIEKVPGLTTADVVVDDIELLQTSG

SEQ ID NO: 31 - protein, 158 aa, Artificial Sequence
  N-terminal Signal Sequence
MPFGNKIVNGNFETGSLIPWSSSNVAISNLQSHTGSYSALLFGNTANSLMFQAVPVTVGDSFEFFLSIAKIGNLPSAQVNIALIYLNVASTPISIGMSIILPIGHLPDNLNNNWSTIYEISSVVPAMATHAMVIIHKIPSPSTSDIVVDDIVFVQTGA

SEQ ID NO: 32 - protein, 75 aa, Artificial Sequence
  N-terminal Signal Sequence
VYLNVVATPIGIGMSTILPIDHLPDNTNKNWTTIYETTSVVPATAIRALVIIHKIPSPSTADIVVDDIVLLQTGL

SEQ ID NO: 33 - protein, 158 aa, Artificial Sequence
  N-terminal Signal Sequence
MPFSNKIVNGDFETGTLIPWSSINVAITNLQSHTGFFSARLFGNIANSLLFQAVPVTPGDSFEFLLSIAKLGNLVSPQVNIALLYLDVTATPVGIGLNLTIPVGHLPDNTANIWTTIYETTSVVPATAIQALLIIEKVASLTTADVVVDDIELLQTSG

SEQ ID NO: 34 - protein, 158 aa, Artificial Sequence
  N-terminal Signal Sequence
MPFGNKIVNGNFETGSLIPWSSSNVVISNLQSHTGSYSALLFGNAANSLMFQAVPVTVGDSFEFFLSIAKIGNLPSPQVNIALIYLNVASTPISIGMSIILPIDHLPDNLNNNWSTIYEISSVVPATATHAMVIIHKIPSPSTSDIVVDDIVLVQTGA

SEQ ID NO: 35 - protein, 158 aa, Artificial Sequence
  N-terminal Signal Sequence
MPFSNKIVNGDFETGTLIPWSSINVAITNLQSHTGFFSARLFGNIANSLLFQAVPVTPGDSFEFLLSIAKLGNLVSPQVNIALLYLDVTATPVGIGLNFTIPVGHLPDNTANIWTTIYETTSVVPATAIQALLIIEKVASLTTADVVVDDIELLQTSG

SEQ ID NO: 36 - protein, 97 aa, Artificial Sequence
  N-terminal Signal Sequence
FELFLSIAKLGNLVSPQVNIALLYLDVATIPVGIGLNLTIPVGHLPDNTANIWTTIYETTSVVPATATQALLIINKIAALSTADIVVDDIGILQTSE

SEQ ID NO: 37 - protein, 158 aa, Artificial Sequence
  N-terminal Signal Sequence
MPFGNKIVNGNFETGSLIPWSSSNVVISNLQSHTGSYSALLFGNATNSLMFQAVPVTVGDSFEFFLSIAKIGNLPSPQVNIALIYLNVASTPISIGMSIILPIGHLPDNLNNNWSTIYEISSVVPATATHAMVIIHKIPSPSTSDIVVDDIVLVQTGA

SEQ ID NO: 38 - protein, 158 aa, Artificial Sequence
  N-terminal Signal Sequence
MPFSNKIVNGDFETGTLIPWSSINVAITNLQSHTGFFSARLFGNIANSLLFQAVPVTPGDSFEFLLSIAKLGNLVSPQVNIALLYLDVTATPVGIGLNFTIPVGHLPDNTANIWTTIYETTSVVPATATQALLIINKIAALSTADIVVDDIGILQTSE

SEQ ID NO: 39 - DNA, 135 bp, Artificial Sequence
  N-terminal Signal Sequence
atgcgctttagctatagccgcaactttgataactttaaatgcgatagcaaatgcaactatccgatgaaatataaatgcaacgaatttggctgcaacggctatccgtatgatctgagcggcattcaggatccgatt

SEQ ID NO: 40 - DNA, 114 bp, Artificial Sequence
  N-terminal Signal Sequence
atgcattttggcaactttaacctggatcgcgtgtgcagcaaatgcaacagctttccgtgctgctgcaaaaccggcccgattccgagctgcccgatttggccggtgaaaccgacc

SEQ ID NO: 41 - protein, 45 aa, Artificial Sequence
  N-terminal Signal Sequence
MRFSYSRNFDNFKCDSKCNYPMKYKCNEFGCNGYPYDLSGIQDPI

SEQ ID NO: 42 - protein, 38 aa, Artificial Sequence
  N-terminal Signal Sequence
MHFGNFNLDRVCSKCNSFPCCCKTGPIPSCPIWPVKPT

SEQ ID NO: 43 - protein, 44 aa, Artificial Sequence
  N-terminal Signal Sequence
MRFSYNRNFDNFKCDAKCNYPMKYKCNEFGCNGYPYVYGIEDPI

SEQ ID NO: 44 - protein, 45 aa, Artificial Sequence
  N-terminal Signal Sequence
MRFSYSRNFDNFKCDSKCNYPMKYKCNEFGCNGYPYDFSGIQDPI

SEQ ID NO: 45 - protein, 45 aa, Artificial Sequence
  N-terminal Signal Sequence
MRFSYSRNFDNFKCDSKCNYPMKYKCNEFGCNGYPYDFSGIQDPM

SEQ ID NO: 46 - protein, 45 aa, Artificial Sequence
  N-terminal Signal Sequence
MRFSYSRNFDYFKCDSKCNYPMKYKCYEFGCNGYPYDFSGIQDPI

SEQ ID NO: 47 - protein, 45 aa, Artificial Sequence
  N-terminal Signal Sequence
MRFSYSRNSDNFKCDSKCNYPMKYKCNEFGCNGYPYDFSGIQDPI

SEQ ID NO: 48 - protein, 45 aa, Artificial Sequence
  N-terminal Signal Sequence
MRFSYSRNFDNFKCDSKSNYPMKYKCNEFGCNGYPYDFSGIQDPI

SEQ ID NO: 49 - protein, 45 aa, Artificial Sequence
  N-terminal Signal Sequence
MRFSYSRNFDNFKCDSKCNYPMKYKCNEFGCNGYPYDFLGIQDPI

SEQ ID NO: 50 - protein, 45 aa, Artificial Sequence
  N-terminal Signal Sequence
MRFSYSRNFDNFKCDSKCNYPMKYKCNEFGCNGYPYDLLGIQDPI

SEQ ID NO: 51 - protein, 45 aa, Artificial Sequence
  N-terminal Signal Sequence
MRFSYSRNFDNFKCDSKCNYPLKFKCNEFGCNGYPYDFSGIQDPI

SEQ ID NO: 52 - protein, 45 aa, Artificial Sequence
  N-terminal Signal Sequence
MRFSYSRNFDNFKCDSKCNYPMKYKCNEFGCNGYPYNLSGIQDPI

SEQ ID NO: 53 - protein, 45 aa, Artificial Sequence
  N-terminal Signal Sequence
MRFSYSRNFDNFKCDSKCNYPKKYKCNEFGCNGYPYDLSGIQDPI

SEQ ID NO: 54 - protein, 45 aa, Artificial Sequence
  N-terminal Signal Sequence
MRFSYSRNFDNFKCDSKCNYPLKYKCNEFGCNGYPYDLSGIQDPI

SEQ ID NO: 55 - protein, 45 aa, Artificial Sequence
  N-terminal Signal Sequence
MRLSYSRNFDNFKCDSKCNYPMKYKCNEFGCNGYPYDFSGIQDPI

SEQ ID NO: 56 - protein, 45 aa, Artificial Sequence
  N-terminal Signal Sequence
LRFSYSRNFDNFKCDSKCNYPMKYKCNEFGCNGYPYDLSGIQDPI

SEQ ID NO: 57 - protein, 45 aa, Artificial Sequence
  N-terminal Signal Sequence
MRFRYSRNFDNFKCDSKCNYPMKYKCNEFGCNGYPYDFSGIQDPI

SEQ ID NO: 58 - protein, 45 aa, Artificial Sequence
  N-terminal Signal Sequence
MRVSYSRNFDNFKCDSKCNYPMKYKCNEFGCNGYPYDFSGIQDPI

SEQ ID NO: 59 - protein, 45 aa, Artificial Sequence
  N-terminal Signal Sequence
MRFSYSRYFDNFKCDSKCNYPMKYKCNEFGCNGYPYDFSGIQDPI

SEQ ID NO: 60 - protein, 45 aa, Artificial Sequence
  N-terminal Signal Sequence
MRISYSRNFDNFKCDSKCNYPMKYKCNEFGCNGYPYDFSGIQDPM

SEQ ID NO: 61 - protein, 45 aa, Artificial Sequence
  N-terminal Signal Sequence
MRFSYSRNFDNFKCDSKCNYPMKYKCNEFGCNGYPYDLSGIQEPM

SEQ ID NO: 62 - protein, 45 aa, Artificial Sequence
  N-terminal Signal Sequence
MRFSYSRNFDNFKCDSKCNYPMKYKCNEFGCNGYPYDLSGIQDLI

SEQ ID NO: 63 - protein, 45 aa, Artificial Sequence
  N-terminal Signal Sequence
MRFSYSRNFDNFKCDSKCNYPMKYKCNEFGCNGYPYDLSGIQVPI

SEQ ID NO: 64 - protein, 45 aa, Artificial Sequence
  N-terminal Signal Sequence
MRFSYSRNFDNFKCDSKFNYPMKYKCNEFGCNGYPYDLSGIQDPI

SEQ ID NO: 65 - protein, 45 aa, Artificial Sequence
  N-terminal Signal Sequence
MRFSYSRNFDYFKCDSKCNYPMKYKCNEFGCNGYPYDLSGIQDPI

SEQ ID NO: 66 - protein, 45 aa, Artificial Sequence
  N-terminal Signal Sequence
MRFSYSRNFDNFKCDSKCNYPMKNKCNEFGCNGYPYDLSGIQDPI

SEQ ID NO: 67 - protein, 45 aa, Artificial Sequence
  N-terminal Signal Sequence
MRFSYSRNFDNFKCDSKCNYPMKYKCNEFGCNGYPYDLSAIQDPI

SEQ ID NO: 68 - protein, 45 aa, Artificial Sequence
  N-terminal Signal Sequence
MRFSYSRNFDNFKCDSKCNYPMKYKCNEFGCNGYPYDLSGFQDPI

SEQ ID NO: 69 - protein, 45 aa, Artificial Sequence
  N-terminal Signal Sequence
MRFSYSRNFDNFKCDSKCNYPMKYKCNEFGCNGYPYDLSGFQDPT

SEQ ID NO: 70 - protein, 45 aa, Artificial Sequence
  N-terminal Signal Sequence
MRFSYSRNFDNYKCDSKCNYPMKYKCNEFGCNGYPYDLSGIQDPI

SEQ ID NO: 71 - protein, 45 aa, Artificial Sequence
  N-terminal Signal Sequence
MRFSYCRNFDNFKCDSKCNYPMKYKCNEFGCNGYPYDLSGIQDPI

SEQ ID NO: 72 - protein, 45 aa, Artificial Sequence
  N-terminal Signal Sequence
MRFSYSRNLDNFKCDSKCNYPMKYKCNEFGCNGYPYDLSGIQDLI

SEQ ID NO: 73 - protein, 45 aa, Artificial Sequence
  N-terminal Signal Sequence
MRFSYSRNFDNFKCDSKCNYPMKYKCNEFGCIGYPYNLSGIQDPI

SEQ ID NO: 74 - protein, 45 aa, Artificial Sequence
  N-terminal Signal Sequence
MRFSYSRNFDNFKCDSKCNYPMKYKCNEFGCNGYAYNLSGIQDPI

SEQ ID NO: 75 - protein, 45 aa, Artificial Sequence
  N-terminal Signal Sequence
MRFSYSRNLDNFKCDSKCNYPMKYKCNEFGCNGYPYNLSGIQDPI

SEQ ID NO: 76 - protein, 45 aa, Artificial Sequence
  N-terminal Signal Sequence
LRFSYSRNFDNFKCDSKCNYPMKYKCNEFGCNGYPYNLSGIQDPI

SEQ ID NO: 77 - protein, 45 aa, Artificial Sequence
  N-terminal Signal Sequence
MRFSYIRNFDNFKCDSKCNYPMKYKCNEFGCNGYPYNLSGIQDPI

SEQ ID NO: 78 - protein, 45 aa, Artificial Sequence
  N-terminal Signal Sequence
MRVSDSRNFDNFKCDSKCNYPMKYKCNEFGCNGYPYDFSGIQDPI

SEQ ID NO: 79 - protein, 45 aa, Artificial Sequence
  N-terminal Signal Sequence
MRFSYSRNFDNFKCYSKCNYPMKYKCNEFGCNGYPYDLSGFQDPI

SEQ ID NO: 80 - protein, 45 aa, Artificial Sequence
  N-terminal Signal Sequence
MRFSYSRNFDNFKCDSKCNYPMKYKCNEFWCNGYPYDLSGFQDPI

SEQ ID NO: 81 - protein, 45 aa, Artificial Sequence
  N-terminal Signal Sequence
MRFSNSRNFDNFKCDSKCNYPMKYKCNEFGCNGYPYDLSGFQDPI

SEQ ID NO: 82 - protein, 45 aa, Artificial Sequence
  N-terminal Signal Sequence
MRFSYSRNLDNFKCDSKCNYPMKYKCNEFGCNGYPYDLSGFQDPI

SEQ ID NO: 83 - protein, 43 aa, Artificial Sequence
  N-terminal Signal Sequence
FSYSRNFDNFKCDSKCNYPMKYKCNEFGCNGYAYNLSGIQDPI

SEQ ID NO: 84 - protein, 45 aa, Artificial Sequence
  N-terminal Signal Sequence
MRFSYSRNFDNFKCDSKCNYPMKYKCNEYGCNGYAYNLSGIQDPI

SEQ ID NO: 85 - protein, 45 aa, Artificial Sequence
  N-terminal Signal Sequence
MRLSYSRNLDNFKCDSKCNYPMKYKCNEFGCNGYPYNLSGIQDPI

SEQ ID NO: 86 - protein, 45 aa, Artificial Sequence
  N-terminal Signal Sequence
MRFSYSRNFDNFKCDSKCNYPMKYKCNEFGCNGYPFDFSGFQDPI

SEQ ID NO: 87 - protein, 45 aa, Artificial Sequence
  N-terminal Signal Sequence
MRFSYSRNFDNFKSDTKCNYPMKYKCNEFGCNGYPYDLSGIQDPI

SEQ ID NO: 88 - protein, 44 aa, Artificial Sequence
  N-terminal Signal Sequence
MRFSYNRNFDNFKCDAKCNYPMKYKCNEFGCNGYPYVYGIQDPI

SEQ ID NO: 89 - protein, 44 aa, Artificial Sequence
  N-terminal Signal Sequence
MRFSFNRNFDNFKCDAKCNYPMKYKCNEFGCNGYPYVYGIEDPI

SEQ ID NO: 90 - protein, 44 aa, Artificial Sequence
  N-terminal Signal Sequence
MRFSYNRNFDNFKCDAKCNYPMKYKCNEFGCNGYPYVYGIEDQI

SEQ ID NO: 91 - protein, 44 aa, Artificial Sequence
  N-terminal Signal Sequence
MRFSYIRNFDNFKCDAKCNYPLKYKCNEFGCNGYPYVYGIQEPI

SEQ ID NO: 92 - protein, 44 aa, Artificial Sequence
  N-terminal Signal Sequence
MRFSYNLNFDNFKCDAKCNYPLKYKCNEFGCNGYPYVYGIQEPI

SEQ ID NO: 93 - protein, 44 aa, Artificial Sequence
  N-terminal Signal Sequence
MRFCYYRNFDNFKCDANCYYPMKYKCNEFGCNGYPYVYGIEDPI

SEQ ID NO: 94 - protein, 45 aa, Artificial Sequence
  N-terminal Signal Sequence
MRFSYSRNFDNFKCDSKCNYHMKYKCNEFGCNGYPYVLSDIQDPI

SEQ ID NO: 95 - protein, 41 aa, Artificial Sequence
  N-terminal Signal Sequence
YSRNFDKIKCDSKCNYHMKYKCNEFGCNGYPYDFSGIQDPI

SEQ ID NO: 96 - protein, 45 aa, Artificial Sequence
  N-terminal Signal Sequence
MRFNYNRNNDNFKCDAKCNYPMKFKCNEFGCNGYPYGAYEIQDPV

SEQ ID NO: 97 - protein, 45 aa, Artificial Sequence
  N-terminal Signal Sequence
MRFNYNRNNDNFKCDTKCNYPMKFKCNEFGCNGYPYGAYEIQDPV

SEQ ID NO: 98 - protein, 37 aa, Artificial Sequence
  N-terminal Signal Sequence
MHFGKFKERVCSKCNTFPCCCSPCPPQSCPIWPVKPT

SEQ ID NO: 99 - protein, 37 aa, Artificial Sequence
  N-terminal Signal Sequence
MHFGKFKERVCSKCNTFPCCCSPYPPQSCPIWPVKPT

SEQ ID NO: 100 - protein, 37 aa, Artificial Sequence
  N-terminal Signal Sequence
MHFGNFIDRVCSKCHSFPCCCKTGPIPSCPIWPVKPT

SEQ ID NO: 101 - protein, 37 aa, Artificial Sequence
  N-terminal Signal Sequence
MHFGNFIDRVCSKCHSFPCYCKTGPIPSCPIWPVKPT

SEQ ID NO: 102 - protein, 37 aa, Artificial Sequence
  N-terminal Signal Sequence
MHFGNFIDRVCSKCHSFPCCCKTGPIPSCPIWPVNPT

SEQ ID NO: 103 - protein, 37 aa, Artificial Sequence
  N-terminal Signal Sequence
MHFGNFIDRVCSKCHSFPCCCKAGPIPSCPIWPVKPT

SEQ ID NO: 104 - protein, 37 aa, Artificial Sequence
  N-terminal Signal Sequence
MHFGNFIDRVCSKCHSFPCCCKTGPIPNCPIWPVKPT

SEQ ID NO: 105 - protein, 37 aa, Artificial Sequence
  N-terminal Signal Sequence
MHYGNFIDRVCSKCHSFPCCCKTGPIPSCPIWPVKPT

SEQ ID NO: 106 - protein, 37 aa, Artificial Sequence
  N-terminal Signal Sequence
MHFGNFIVRVCSKCHSFPCCCKTGPIQSCPIWPVKPT

SEQ ID NO: 107 - protein, 37 aa, Artificial Sequence
  N-terminal Signal Sequence
MHFGIIIDRVCSKCHSFPCCCKTGPIPSCPIWPVKPT

SEQ ID NO: 108 - protein, 37 aa, Artificial Sequence
  N-terminal Signal Sequence
MHFGKFIDRVCTKCHSYPCCCKTGPIPSCPIWPVKPT

SEQ ID NO: 109 - protein, 37 aa, Artificial Sequence
  N-terminal Signal Sequence
MHFGKFLDRVCNKCHSFPCCCKTGPIPGCPIWPVKPT

SEQ ID NO: 110 - protein, 37 aa, Artificial Sequence
  N-terminal Signal Sequence
MHFGKFLDRVCSKCHSFPCCCKTGPIPGCPIWPVKPT

SEQ ID NO: 111 - protein, 37 aa, Artificial Sequence
  N-terminal Signal Sequence
MHFGKFLDRVCSKCNSFPCCCKTGPSPSCPIWPVKPT

SEQ ID NO: 112 - protein, 37 aa, Artificial Sequence
  N-terminal Signal Sequence
MNFGKFLDRVCSKCNSFPCCCKTGPSPSCPIWPVKPT

SEQ ID NO: 113 - protein, 37 aa, Artificial Sequence
  N-terminal Signal Sequence
MLFGKFLDRVCSKCNSFPCCCKTGPSPSCPIWPVKPT

SEQ ID NO: 114 - protein, 37 aa, Artificial Sequence
  N-terminal Signal Sequence
MHFGKFLDRVCSKCNSFPCCCKTGPSPSCPNWPVKPT

SEQ ID NO: 115 - protein, 37 aa, Artificial Sequence
  N-terminal Signal Sequence
MHFGNYLKRVCSKCNSFPCCCRTGPIPSCPVWPVKPT

SEQ ID NO: 116 - protein, 37 aa, Artificial Sequence
  N-terminal Signal Sequence
MHFGNYLKRVCSKCNYFPCCCRTGPIPSCPVWPVKPT

SEQ ID NO: 117 - protein, 37 aa, Artificial Sequence
  N-terminal Signal Sequence
MHFGNYLKRVCSKCNSFPCCCRPGPIPSCPVWPVKPT

SEQ ID NO: 118 - protein, 37 aa, Artificial Sequence
  N-terminal Signal Sequence
MHFGNYLKQVCSKCNSFPCCCRTGPIPSCPVWPVKPT

SEQ ID NO: 119 - protein, 37 aa, Artificial Sequence
  N-terminal Signal Sequence
MHFGNYLKRVCSKCNSFPCCCRTGPIQSCPVWPVKPT

SEQ ID NO: 120 - protein, 37 aa, Artificial Sequence
  N-terminal Signal Sequence
MHFGNYLKQVCNKCNSFPCCCRTGPIPSCPVWPVKPT

SEQ ID NO: 121 - protein, 37 aa, Artificial Sequence
  N-terminal Signal Sequence
MHFGNYLKQVCSKCNSFPCCCRNGPIPSCPVWPVKPT

SEQ ID NO: 122 - protein, 37 aa, Artificial Sequence
  N-terminal Signal Sequence
MHFGNYLKQVCSKCNSFPCCCRTGPIPSCPIWPVKPT

SEQ ID NO: 123 - protein, 37 aa, Artificial Sequence
  N-terminal Signal Sequence
LHFGNYLKQVCSKCNSFPCCCRTGPIPSCPIWPVKPT

SEQ ID NO: 124 - protein, 37 aa, Artificial Sequence
  N-terminal Signal Sequence
MLFGNYLKQVCSKCNSFPCCCRTGPIPSCPIWPVKPT

SEQ ID NO: 125 - protein, 37 aa, Artificial Sequence
  N-terminal Signal Sequence
MHFGNYLKQVSSKCNSFPCCCRNGPIPSCPVWPVKPT

SEQ ID NO: 126 - protein, 37 aa, Artificial Sequence
  N-terminal Signal Sequence
MHFGRFNERVCSKCNTFPCCCNPCAAQSCPIWPVKPT

SEQ ID NO: 127 - protein, 37 aa, Artificial Sequence
  N-terminal Signal Sequence
MHFGRFKERVCSKCNTFPCCCNPCAAQSCPIWPVKPT

SEQ ID NO: 128 - protein, 37 aa, Artificial Sequence
  N-terminal Signal Sequence
MHFGRFNERVCSKCNSFPCCCNPCAAQSCPIWPVKPT

SEQ ID NO: 129 - protein, 37 aa, Artificial Sequence
  N-terminal Signal Sequence
MHFGKFNERVCSKCNTFPCCCNPCAAQSCPIWPVKPS

SEQ ID NO: 130 - protein, 35 aa, Artificial Sequence
  N-terminal Signal Sequence
MHFGRFNERVCSKCNTFPCCCCAAQSCPIWPVKPT

SEQ ID NO: 131 - protein, 37 aa, Artificial Sequence
  N-terminal Signal Sequence
MHFGKFKDSVCSKCNTFPCCCNPCAAQSCPIWPVKPT

SEQ ID NO: 132 - protein, 34 aa, Artificial Sequence
  N-terminal Signal Sequence
MHFGRFNERVCSKCNTFPCCCCAAQSCPIWPVKP

SEQ ID NO: 133 - protein, 37 aa, Artificial Sequence
  N-terminal Signal Sequence
MHFGKFKERVCSKCNSFPCCCSPCPPQSCPIWPVKPT

SEQ ID NO: 134 - protein, 37 aa, Artificial Sequence
  N-terminal Signal Sequence
MHFGKFKDRVCSKCNTFPCCCSPCPPQSCPIWPVKPT

SEQ ID NO: 135 - protein, 37 aa, Artificial Sequence
  N-terminal Signal Sequence
MHFGRFNERVCSKCNTFPCCCSPCDVQSCPIWPVKPT

SEQ ID NO: 136 - protein, 37 aa, Artificial Sequence
  N-terminal Signal Sequence
MQFGRFNERVCSKCTTFPCCCSPCDVQSCPIWPVKPT

SEQ ID NO: 137 - protein, 35 aa, Artificial Sequence
  N-terminal Signal Sequence
MHFGKFNDRICSKCNTFPCCCNPCAPSCPVWPVKP

SEQ ID NO: 138 - protein, 37 aa, Artificial Sequence
  N-terminal Signal Sequence
MHFGNFIDRVCSKCNTFPCCCRGGPVPGCPIWPVKPT

SEQ ID NO: 139 - protein, 37 aa, Artificial Sequence
  N-terminal Signal Sequence
MHFGNFIDRVCSKCKTFPCCCRGGPVPGCPIWPVKPT

SEQ ID NO: 140 - protein, 37 aa, Artificial Sequence
  N-terminal Signal Sequence
MHFGNFIDRVCSKCNTFPCCCRGGPVPGCPIWPVKPP

SEQ ID NO: 141 - protein, 37 aa, Artificial Sequence
  N-terminal Signal Sequence
MHFGNFIDRVCSKCNTFPCCCRGGPVPGCHIWPVKPT

SEQ ID NO: 142 - protein, 37 aa, Artificial Sequence
  N-terminal Signal Sequence
MHFGNFIDRVCSKCKTFPCCCRGGPVPCCPIWPVKPT

SEQ ID NO: 143 - protein, 37 aa, Artificial Sequence
  N-terminal Signal Sequence
MHFGNFIDRVCSKCKTIPCCCRGGPVPGCPIWPVKPT

SEQ ID NO: 144 - protein, 37 aa, Artificial Sequence
  N-terminal Signal Sequence
MHFGNFIDRVCSKCNTFPCCCKGGPVPGCHIWPVKPT

SEQ ID NO: 145 - protein, 37 aa, Artificial Sequence
  N-terminal Signal Sequence
MHFGNFIDRVCSKCKTFPCCCGSGPVPGCPIWPVKPT

SEQ ID NO: 146 - protein, 37 aa, Artificial Sequence
  N-terminal Signal Sequence
MHFGNFIDRVCSKCKTSPCCCGSGPVPGCPIWPVKPT

SEQ ID NO: 147 - protein, 37 aa, Artificial Sequence
  N-terminal Signal Sequence
MHFGNFIDRVCTKCKIFPRCCRGGPVPGCPIWPVKPT

SEQ ID NO: 148 - protein, 36 aa, Artificial Sequence
  N-terminal Signal Sequence
MHFGKFTDRVCSKCNTFPCCCNTCPAPNCPIWPVKP

SEQ ID NO: 149 - protein, 36 aa, Artificial Sequence
  N-terminal Signal Sequence
MNFGNFNNRICSKCNTFPCCCIPCAPSCPVWPVKPG

SEQ ID NO: 150 - protein, 37 aa, Artificial Sequence
  N-terminal Signal Sequence
MYFGNFLDRICSKCNTFPCCCLSGPILRSPIWPVRPT

SEQ ID NO: 151 - protein, 41 aa, Artificial Sequence
  N-terminal Signal Sequence
MHFGNFLDRVCNKCNTFPCCCLSGPIHRSPIWPVPPAKATR

SEQ ID NO: 152 - protein, 35 aa, Artificial Sequence
  N-terminal Signal Sequence
MQFGKYNDRICRKCNTFPCCCNPSAPSCPGWPVKP

SEQ ID NO: 153 - protein, 38 aa, Artificial Sequence
  N-terminal Signal Sequence
MKFQDFNINKICNNCHSYPCCCHNGPVQRCPVWPVKPT

SEQ ID NO: 154 - protein, 38 aa, Artificial Sequence
  N-terminal Signal Sequence
MKFQDFNINKICNNCHSYPCCCHNGPVHRCPVWPVKPT

SEQ ID NO: 155 - protein, 38 aa, Artificial Sequence
  N-terminal Signal Sequence
MKFQDFNINKICNNCNSYPCCCHNGPVQRCPVWPVKPT

SEQ ID NO: 156 - protein, 38 aa, Artificial Sequence
  N-terminal Signal Sequence
MKFQDFNINKICNNCHSYPCCCHNGPVLRCPVWPVKPT

SEQ ID NO: 157 - protein, 38 aa, Artificial Sequence
  N-terminal Signal Sequence
MKFLDFNINKICNNCHSYPCCCHNGPVHRCPVWPVKPT

SEQ ID NO: 158 - protein, 38 aa, Artificial Sequence
  N-terminal Signal Sequence
MKFQDFNINKICNNCHSYPCCCHKGPVHRCPVWPVKPT

SEQ ID NO: 159 - protein, 38 aa, Artificial Sequence
  N-terminal Signal Sequence
MKFQDFNINKICNICHSYPFCCHNGPVQRCPVWPVKPT

SEQ ID NO: 160 - protein, 37 aa, Artificial Sequence
  N-terminal Signal Sequence
MKFQDFNINMICNNCHSYPCCCHNGPVHRCPVWPVKP

SEQ ID NO: 161 - protein, 38 aa, Artificial Sequence
  N-terminal Signal Sequence
MKFHDFNLNNICGKCLSHPCCCNNGPAQRCPIWPVKPT

SEQ ID NO: 162 - protein, 38 aa, Artificial Sequence
  N-terminal Signal Sequence
MKFHDFNLNNICGKCLSHPCCCNNVPAQRCPIWPVKPT

SEQ ID NO: 163 - protein, 38 aa, Artificial Sequence
  N-terminal Signal Sequence
MKFHDFNLNNICGKCLSHPCCCNNGPVQRCPIWPVKPT

SEQ ID NO: 164 - protein, 37 aa, Artificial Sequence
  N-terminal Signal Sequence
MDFANFKNRICSRCNSFPCNCKRNFPQNCPIWPVKPT

SEQ ID NO: 165 - protein, 37 aa, Artificial Sequence
  N-terminal Signal Sequence
MDFANFKNRICSRCNSFPCNCKRNSPQNCPIWPVKPT

SEQ ID NO: 166 - protein, 37 aa, Artificial Sequence
  N-terminal Signal Sequence
MDFANFKNRICSRCNTFPCNCKRNFPQNCPIWPVKPT

SEQ ID NO: 167 - protein, 37 aa, Artificial Sequence
  N-terminal Signal Sequence
MDFANFKNRICSRCNSFPCNCKGNFPQNCPIWPVKPT

SEQ ID NO: 168 - protein, 37 aa, Artificial Sequence
  N-terminal Signal Sequence
MDFAYFKNRICSRCNSFPCNCKRNFPQNCPIWPVKPT

SEQ ID NO: 169 - protein, 37 aa, Artificial Sequence
  N-terminal Signal Sequence
MDFANFKNRICSRCNSFPCNCIRNIPQNCPIWPVKPT

SEQ ID NO: 170 - protein, 37 aa, Artificial Sequence
  N-terminal Signal Sequence
MDLANFKNRICSRCNSFPCNCKRNFPQNCPIWPVKPT

SEQ ID NO: 171 - protein, 37 aa, Artificial Sequence
  N-terminal Signal Sequence
MDFANFKNRICSRCNSFPCNCKSNYPQNCSIWPVKPT

SEQ ID NO: 172 - protein, 38 aa, Artificial Sequence
  N-terminal Signal Sequence
MNFEDYFLNNVCSKCKTFPCACIRIPEHHCCIWPVKPT

SEQ ID NO: 173 - protein, 38 aa, Artificial Sequence
  N-terminal Signal Sequence
MNFEDYFLNNVCSKCKTFPCACIRIPGHHCSIWPVKPT

SEQ ID NO: 174 - protein, 38 aa, Artificial Sequence
  N-terminal Signal Sequence
MNFEDYFLNNVHVLCKTFPCACIRIPEHHCCIWPVKPT

SEQ ID NO: 175 - protein, 38 aa, Artificial Sequence
  N-terminal Signal Sequence
MNFEDYFLNNVCSKCRTFPCGCRRIPEHHSCVWPVKPT

SEQ ID NO: 176 - protein, 38 aa, Artificial Sequence
  N-terminal Signal Sequence
MNFEDYFLKNVCSKCRTFPCGCTRIPEQHCCVWPVKPT

SEQ ID NO: 177 - protein, 38 aa, Artificial Sequence
  N-terminal Signal Sequence
MNFHDFILNNICKRCNSYPCRCINRPEQNCSIWPVKPT

SEQ ID NO: 178 - DNA, 150 bp, Artificial Sequence
  N-terminal Signal Sequence
atggattcaaataaaacgaggtgtgctgtttgtaataaatatcaatgttcttgtaactccaaaaaaagaagtcctaaaacagttattagagatttagattatatagatccatgtgaacctctttatataatagatcattgtcataataga

SEQ ID NO: 179 - protein, 50 aa, Artificial Sequence
  N-terminal Signal Sequence
  misc_feature (9)..(9): Xaa can be any naturally occurring amino acid
MDSNKTRCXVCNKYQCSCNHKKRSPKTVIRDLDYIDPCEPLYIIDHCHNR

SEQ ID NO: 180 - DNA, 159 bp, Artificial Sequence
  N-terminal Signal Sequence
atggactcaaataaaacgaggtgtcctatttgtaataattatcaatgttcttgtaacgctaaaaaaagaaggcctacaagatgtattagatatttagattatatagatcaatgtgaacaatgtgaacctatttctataatagatcattgtcataataga

SEQ ID NO: 181 - protein, 53 aa, Artificial Sequence
  N-terminal Signal Sequence
MDSNKTRCPICNNYQCSCNAKKRRPTRCIRYLDYIDQCEQCEPISIIDHCHNR

SEQ ID NO: 182 - DNA, 330 bp, Artificial Sequence
  N-terminal Signal Sequence
atggactcaaataaaacgaggtgtcctatttgtaataattatcaatgttcttgtaacgctaaaaaaagaaggcctacaagatgtattagatatttagattatatagatcaatgtgaacaatgtgaacctatttctataatagatcattgtcataatagaggtaaaacaggaccgaccggagcaactggtgcaaccggaccaactggagcaactggagcaacaggaccaacaggaccaactggagcaacaggacctacaggagcaacaggaccaacaggtgcaactggggcgacaggaatcaccgggccgactggagcaacgggagcaaca

SEQ ID NO: 183 - protein, 110 aa, Artificial Sequence
  N-terminal Signal Sequence
MDSNKTRCPICNNYQCSCNAKKRRPTRCIRYLDYIDQCEQCEPISIIDHCHNRGKTGPTGATGATGPTGATGATGPTGPTGATGPTGATGPTGATGATGITGPTGATGAT

SEQ ID NO: 184 - DNA, 150 bp, Artificial Sequence
  N-terminal Signal Sequence
atggaatcaaataaaacgaggtgtattgtttgtaataaatatcaatgttcttgtaactccaaaaaaagaagtcctaaaacagttattagagatttagattatatagatccatgtgaacctctttatataatagatcattgtcataataga

SEQ ID NO: 185 - protein, 50 aa, Artificial Sequence
  N-terminal Signal Sequence
MESNKTRCIVCNKYQCSCNSKKRSPKTVIRDLDYIDPCEPLYIIDHCHNR

SEQ ID NO: 186 - DNA, 378 bp, Artificial Sequence
  N-terminal Signal Sequence
atggaatcaaataaaacgaggtgtattgtttgtaataaatatcaatgttcttgtaactccaaaaaaagaagtcctaaaacagttattagagatttagattatatagatccatgtgaacctctttatataatagatcattgtcataatagaggtaaaacaggaccaacaggaccaacgggaccaaccggagtgactggagcaaccggaccaacaggagaaacaggaccaacgggagcaaccggagcaactggagcaacaggagcaacaggtccaatgggagtaactggctcgaccggagcaactggtgtaacgggagcaacaggagcaacagggtcaaccggagcaacaggtgaaaccggagcgactggtgaaaccgga

SEQ ID NO: 187 - protein, 126 aa, Artificial Sequence
  N-terminal Signal Sequence
MESNKTRCIVCNKYQCSCNSKKRSPKTVIRDLDYIDPCEPLYIIDHCHNRGKTGPTGPTGPTGVTGATGPTGETGPTGATGATGATGATGPMGVTGSTGATGVTGATGATGSTGATGETGATGETG

SEQ ID NO: 188 - DNA, 150 bp, Artificial Sequence
  N-terminal Signal Sequence
atgtattcaaataaaaagaggtgtgctgtttgtaataaatatcaatgttcttgtaactccaaaaaaagaagtcctaaaacagttattagagatttagattatatagatccatgtgaacctctttatataatagatcattgtcataataga

SEQ ID NO: 189 - protein, 50 aa, Artificial Sequence
  N-terminal Signal Sequence
MYSNKKRCAVCNKYQCSCNSKKRSPKTVIRDLDYIDPCEPLYIIDHCHNR

SEQ ID NO: 190 - DNA, 474 bp, Artificial Sequence
  N-terminal Signal Sequence
atgtattcaaataaaaagaggtgtgctgtttgtaataaatatcaatgttcttgtaactccaaaaaaagaagtcctaaaacagttattagagatttagattatatagatccatgtgaacctctttatataatagatcattgtcataatagaggtaaaacaggaccaacaggagcaacaggtcatacaggaacaaccggaacaacaggagagactggaccaacaggagcaacaggagcaacaggaataacgggagcaacgggagaaacaggagcaacaggaccaacgggaacaactggtgtaaccggagaaaccggagcaacaggtccaactggagaaactggtgaaacaggaccaacgggagcaactgggaaccggagcaacaggagaaacaggaacaactggagcaacaggaaaaacgggaacaacaggagaaacaggagaaacaggagcaacaggagaaacaggaaaaacagg

SEQ ID NO: 191 - protein, 158 aa, Artificial Sequence
  N-terminal Signal Sequence
MYSNKKRCAVCNKYQCSCNSKKRSPKTVIRDLDYIDPCEPLYIIDHCHNRGKTGPTGATGHTGTTGTTGETGPTGATGATGITGATGETGATGPTGTTGVTGETGATGPTGETGETGPTGATGNRSNRRNRNNWSNRKNGNNRRNRRNRSNRRNRKNR

SEQ ID NO: 192 - DNA, 150 bp, Artificial Sequence; identical to SEQ ID NO: 188
  N-terminal Signal Sequence

SEQ ID NO: 193 - protein, 50 aa, Artificial Sequence; identical to SEQ ID NO: 189
  N-terminal Signal Sequence

SEQ ID NO: 194 - DNA, 330 bp, Artificial Sequence
  N-terminal Signal Sequence
atgtattcaaataaaaagaggtgtgctgtttgtaataaatatcaatgttcttgtaactccaaaaaaagaagtcctaaaacagttattagagatttagattatatagatccatgtgaacctctttatataatagatcattgtcataatagaggtaaaacaggaccaacaggagcaacaggtcctacaggatcaacgggaccaacaggagtgactggaccaacaggagcaacaggaacaacaggatcaacaggaacaacaggagcaacaggagcaacaggagaaacaggagcaacaggaccaacaggaccaacaggagaaacaggaacaaca

SEQ ID NO: 195 - protein, 110 aa, Artificial Sequence
  N-terminal Signal Sequence
MYSNKKRCAVCNKYQCSCNSKKRSPKTVIRDLDYIDPCEPLYIIDHCHNRGKTGPTGATGPTGSTGPTGVTGPTGATGTTGSTGTTGATGATGETGATGPTGPTGETGTT

SEQ ID NO: 196 - DNA, 150 bp, Artificial Sequence
  N-terminal Signal Sequence
atggattcaaataaaaagaggtgtgctgtttgtaataaatatcaatgttcttgtaactccaaaaaaagaagtcctaaaacagttattagagatttagattatatagatccatgtgaaccgctttatataatagatcattgtcataataga

SEQ ID NO: 197 - protein, 50 aa, Artificial Sequence
  N-terminal Signal Sequence
MDSNKKRCAVCNKYQCSCNSKKRSPKTVIRDLDYIDPCEPLYIIDHCHNR

SEQ ID NO: 198 - DNA, 2484 bp, Artificial Sequence
  N-terminal Signal Sequence
atggattcaaataaaaagaggtgtgctgtttgtaataaatatcaatgttcttgtaactccaaaaaaagaagtcctaaaacagttattagagatttagattatatagatccatgtgaaccgctttatataatagatcattgtcataatagaggtaaaacaggaccaacaggagcaacaggtcctacaggatcaatgggaccaaccggagtgactggatcaacaggagcaaccggaccaacagggtcaaccggagtaactggggaaacaggatcaaccgggacaacgggaccaagcggatcaactggaccaactggtgcaacagggtcaaccggagaaaccggagcaacagggtcaaccggagcaactggagaaacaggtccaacaggaccaacgggaacaactggtgtaaccggagaaaccggagcaacaggtccaactggagcaactggagcaactggtgaaacaggttcaacaggaccaacgggagcaactggagaaactggtgaaacaggttcaacaggaccaacgggagcaacgggatcgactggagcaacgggaccaacgggagcaacgggatccactggagcaacgggatcgactggagcaacgggatcgactggagcaacaggaccaacaggagcaacagggtcaaccggagaaacaggaccaactggtgaaacaggttcaacgggagcaactgggtcgaccggagcaacaggtgaaacaggaccaacgggaacaactggagtaaccggagaaaccggagcaacaggtccaacaggagcaactggagcaactggagaaactggtgaaacaggttcaacaggaccaacgggagcaactggagaaactggtgaaacaggttcaacaggaccaacgggagcaacgggatcgactggagcaacgggaccaactggagcaacgggatcgactggagcaacgggatcgactggagcaacgggatcgactggagcaacaggaccaacaggagcaacagggtcaaccggagaaacaggaccaactggtgaaacaggttcaacgggagcaactgggtcgaccggagcaacaggtgaaacaggaccaacgggagcaactgggtcgaccggagcaactggtgaaacaggtgcaacaggaccaacaggaccaacgggaacaactggtgtaaccggagcaacaggtccaactggagcaactggagaaacaggaccaacgggagcgactggagaagcaggaccaacagggtcaaccggagcgactggaccaaccggggcaacgggaatcacgggagcaacaggaccaacaggatcaactggtgcaacagggtcaaccggagaaactggatcaactggagaaaccggaccaacaggagcaactggtgtaaccggagcaacaggtccaactggagaaacaggttcaacaggaccaatgggagcaaccggagaaactggttcgaccggatcaactggtgtaacgggagcaacaggagcaacgggagaaaccggagcaacaggatcaacgggagcaactggcacgaccggagcaacaggagaaacaggaccaacaggaccaactgggtccaccggagcaacaggagaaacaggtccaacaggaccaacgggagcaactggagtaaccggagcaacaggagcaacgggagcaactggagaaacaggttcaacaggaccaatgggagcaaccggagaaactggttcgaccggatcaactggtgtaacgggagcaacaggagcgactggagtaacaggagcaactggtgaaacaggatcaacgggagcaactgggtcgaccggagcaacaggtgaaaccggaccaacaggtccaacaggtccaacaggaccaacgggaacaactggtgtaaccggagtaactggagaaaccggaccaacaggagcgactggtgaaacaggttcaacaggaccaacgggaacaactggtgtaaccggagaaaccggagcaacaggaccaactggtgcaacagggtcaaccggagaaaccggagcaactggagaaacaggtcgaacaggaccaacgggaacaactggtgtaaccggagaaaccggagcaactggagaaacaggaccaacaggagcaacgggatcgactggagcaacaggaccaacgggagcaactggggcgaccggagcaacaggtgaaacaggaccaacgggagcgactggagaaacaggagcaacgggagcaactgggtcaaccggagcaacaggtccaacaggaccaacgggaacaactggagtaaccggagcaacaggtccaactggagcaactggagaaacaggaccaacgggagcaactggagaagcaggaccaacagggtcaaccggatcgactggaccaaccggggcaacgggaatcacgggagcaacaggaccaacaggagcaactggtgcaacagggtcaaccggagaaactggatcaactggagaaaccggaccaacaggatcaactggt

SEQ ID NO: 199 - protein, 828 aa, Artificial Sequence
  N-terminal Signal Sequence
MDSNKKRCAVCNKYQCSCNSKKRSPKTVIRDLDYIDPCEPLYIIDHCHNRGKTGPTGATGPTGSMGPTGVTGSTGATGPTGSTGVTGETGSTGTTGPSGSTGPTGATGSTGETGATGSTGATGETGPTGPTGTTGVTGETGATGPTGATGATGETGSTGPTGATGETGETGSTGPTGATGSTGATGPTGATGSTGATGSTGATGSTGATGPTGATGSTGETGPTGETGSTGATGSTGATGETGPTGTTGVTGETGATGPTGATGATGETGETGSTGPTGATGETGETGSTGPTGATGSTGATGPTGATGSTGATGSTGATGSTGATGPTGATGSTGETGPTGETGSTGATGSTGATGETGPTGATGSTGATGETGATGPTGPTGTTGVTGATGPTGATGETGPTGATGEAGPTGSTGATGPTGATGITGATGPTGSTGATGSTGETGSTGETGPTGATGVTGATGPTGETGSTGPMGATGETGSTGSTGVTGATGATGETGATGSTGATGTTGATGETGPTGPTGSTGATGETGPTGPTGATGVTGATGATGATGETGSTGPMGATGETGSTGSTGVTGATGATGVTGATGETGSTGATGSTGATGETGPTGPTGPTGPTGTTGVTGVTGETGPTGATGETGSTGPTGTTGVTGETGATGPTGATGSTGETGATGETGRTGPTGTTGVTGETGATGETGPTGATGSTGATGPTGATGATGATGETGPTGATGETGATGATGSTGATGPTGPTGTTGVTGATGPTGATGETGPTGATGEAGPTGSTGSTGPTGATGITGATGPTGATGATGSTGETGSTGETGPTGSTG

SEQ ID NO: 200 - DNA, 150 bp, Artificial Sequence
  N-terminal Signal Sequence
atggattcaaataaaacgaggtgtgctgtttgtaataaatatcaatgttcttgtaactccaaaaaaagaagtcctaaaacagttattagagatttagattatatagatccatgtgaacctctttatataataaatcattgtcataataga

SEQ ID NO: 201 - protein, 50 aa, Artificial Sequence
  N-terminal Signal Sequence
MDSNKTRCAVCNKYQCSCNSKKRSPKTVIRDLDYIDPCEPLYIINHCHNR

SEQ ID NO: 202 - DNA, 369 bp, Artificial Sequence
  N-terminal Signal Sequence
atggattcaaataaaacgaggtgtgctgtttgtaataaatatcaatgttcttgtaactccaaaaaaagaagtcctaaaacagttattagagatttagattatatagatccatgtgaacctctttatataataaatcattgtcataatagaggtaaaactggaccaactggaccaaccggatcgaccggaccaacaggaccaactggtgaaaccggaccaactggagcaacaggaccaacaggagctactggagcaactggagcaactggtgcaactggagcaacgggatcaactggagcaacaggatcaactggggcagccggagtaactggtgaaacaggactaacagggtcaaccggagcgacagga

SEQ ID NO: 203 - protein, 123 aa, Artificial Sequence
  N-terminal Signal Sequence
MDSNKTRCAVCNKYQCSCNSKKRSPKTVIRDLDYIDPCEPLYIINHCHNRGKTGPTGPTGSTGPTGPTGETGPTGATGPTGATGATGATGATGATGSTGATGSTGAAGVTGETGLTGSTGATG

SEQ ID NO: 204 - DNA, 150 bp, Artificial Sequence
  N-terminal Signal Sequence
atggattcaaataaaacgaggtgtgctgtttgtaataaatatcaatgttcttgtaataccaaaaaaagaagtcctaaaacagttattagagatttagattatatagatccatgtgaacctctttatataatagatcattgtcataataga

SEQ ID NO: 205 - protein, 50 aa, Artificial Sequence
  N-terminal Signal Sequence
MDSNKTRCAVCNKYQCSCNTKKRSPKTVIRDLDYIDPCEPLYIIDHCHNR

SEQ ID NO: 206 - DNA, 399 bp, Artificial Sequence
  N-terminal Signal Sequence
atggattcaaataaaacgaggtgtgctgtttgtaataaatatcaatgttcttgtaataccaaaaaaagaagtcctaaaacagttattagagatttagattatatagatccatgtgaacctctttatataatagatcattgtcataatagaggtaaaacaggaccaacaggaccaacaggagcaactggaccaaccggaccaactggagcaacaggggcaaccggagcaacaggtccaacaggatcaactggcgcaacaggagcaactggacaaacgggagcaactggagcaacgggaccaacaggagcaacaggagcaactggagaaacaggagcaacaggagcaacaggagcaacaggagcaacaggaccaacgggagcaactggagaaacaggagca

SEQ ID NO: 207 - protein, 133 aa, Artificial Sequence
  N-terminal Signal Sequence
MDSNKTRCAVCNKYQCSCNTKKRSPKTVIRDLDYIDPCEPLYIIDHCHNRGKTGPTGPTGATGPTGPTGATGATGATGPTGSTGATGATGQTGATGATGPTGATGATGETGATGATGATGATGPTGATGETGA

SEQ ID NO: 208 - DNA, 150 bp, Artificial Sequence; identical to SEQ ID NO: 178
  N-terminal Signal Sequence

SEQ ID NO: 209 - protein, 50 aa, Artificial Sequence
  N-terminal Signal Sequence
MDSNKTRCAVCNKYQCSCNSKKRSPKTVIRDLDYIDPCEPLYIIDHCHNR

SEQ ID NO: 210 - DNA, 399 bp, Artificial Sequence
  N-terminal Signal Sequence
atggattcaaataaaacgaggtgtgctgtttgtaataaatatcaatgttcttgtaactccaaaaaaagaagtcctaaaacagttattagagatttagattatatagatccatgtgaacctctttatataatagatcattgtcataatagaggtaaaacaggaccaacaggagcaacaggtccaacgggaccaaccggagcaacgggatcaactggtccaacaggatcaactggagcaactggagcaacagggtcaaccggagcgactggagaaacaggaccaactggggtaacgggaatcacgggaccgactggtgtaactggagcaacagggttcacaggactgactggagcaactggagcgacaggaaatacaggtccaacaggaccaaccggagca

SEQ ID NO: 211 - protein, 133 aa, Artificial Sequence
  N-terminal Signal Sequence
MDSNKTRCAVCNKYQCSCNSKKRSPKTVIRDLDYIDPCEPLYIIDHCHNRGKTGPTGATGPTGPTGATGSTGPTGSTGATGATGSTGATGETGPTGVTGITGPTGVTGATGFTGLTGATGATGNTGPTGPTGA

SEQ ID NO: 212 - DNA, 150 bp, Artificial Sequence
  N-terminal Signal Sequence
atggattcaaataaaacgaggtgtactgtttgtaataaatatcaatgttcatgtaaccacaaaaaaagaagtcctaaaacagttattagagatttagattatatagatccatgtgaaccgctttatataatagatcattgtcataataga

SEQ ID NO: 213 - protein, 50 aa, Artificial Sequence
  N-terminal Signal Sequence
MDSNKTRCTVCNKYQCSCNHKKRSPKTVIRDLDYIDPCEPLYIIDHCHNR

SEQ ID NO: 214 - DNA, 285 bp, Artificial Sequence
  N-terminal Signal Sequence
atggattcaaataaaacgaggtgtactgtttgtaataaatatcaatgttcatgtaaccacaaaaaaagaagtcctaaaacagttattagagatttagattatatagatccatgtgaaccgctttatataatagatcattgtcataatagaggtaaaacaggtccaacggggccaaccggagcaactggagcaacaggtgcaacgggatcaactgggccgacaggagtaactggtgcaaactcatatacaaaacaaacactgccaacgacgtcaacagagaagaaa

SEQ ID NO: 215 - protein, 95 aa, Artificial Sequence
  N-terminal Signal Sequence
MDSNKTRCTVCNKYQCSCNHKKRSPKTVIRDLDYIDPCEPLYIIDHCHNRGKTGPTGPTGATGATGATGSTGPTGVTGANSYTKQTLPTTSTEKK

SEQ ID NO: 216 - DNA, 150 bp, Artificial Sequence
  N-terminal Signal Sequence
atggattcaaataaaacgaggtgtactgtttgtaataaatatcaatgttcatgtaaccccaaaaaaagaagtcctaaaacagttattagagatttagattatataaatccatgtgaaccgctttatataatagatcattgtcataataga

SEQ ID NO: 217 - protein, 50 aa, Artificial Sequence
  N-terminal Signal Sequence
MDSNKTRCTVCNKYQCSCNPKKRSPKTVIRDLDYINPCEPLYIIDHCHNR

SEQ ID NO: 218 - DNA, 1266 bp, Artificial Sequence
  N-terminal Signal Sequence
atggattcaaataaaacgaggtgtactgtttgtaataaatatcaatgttcatgtaaccccaaaaaaagaagtcctaaaacagttattagagatttagattatataaatccatgtgaaccgctttatataatagatcattgtcataatagaggtaaaacaggtccaacggggccaaccggagcaaccggagcaacggggccaaccggaccaactggagcaacagggccaaccggagcgactggagcaaccggaccaacgggatcaacaggtcctacaggatcaaccggagcaacaggaccgactggggctacaggagtaactggagcaacgggagcaactggagcaacgggagtaactggggcaacaggaccaacgggaccaacagggttaaccggagcgactggagcaacaggaacgactggggctacaggagcaactggggcaacgggagtaactggagcaacaggaccgactggggcaacgggagtaactggagcaacgggagtaactggggcaacaggaccaactggagcaacaggatcgactggagaaactggaacaacaggagtaactggagcaacgggagtaactggagcaacaggtccaaccgggtcaatcggatcaacgggagcaactggggcaaccggaccaactggagcaaccggggcaacaggagcaaccggaccaacgggagaaaccggaccaaccggaccaacaggcccaacgggaatcaccgggccaaccggagcaactggttcaggaggcgtaatcccatttgcatcaggaggcacagtaacaataaattcactattaactggggtacctaataccggaggtattattgcatttggcaatagtactactagtgtagggattggtaatgctacagtaaatcttcaaaatattaataatgaagcgtttgtcgttccacatgctggagcaattacaaatgttgctgcattctttaatattacagttgctcttaatatagctgttttaggtacagctactattacggcacaagtatggaaatcacaaaatgcatcatctaacgtatttgcacctttagcatcgactcttataagctttacaccattagctggtgccatagctgcatttactacattaaatggtgctcttgcaaatttaaatgaaccagtaaatgttggagatcgtttaattatggtattctctgtggctactacagggttaagtgtaataactgctattacaggtacagcaagtgcgggtattacaattagttga

SEQ ID NO: 219 - protein, 421 aa, Artificial Sequence
  N-terminal Signal Sequence
MDSNKTRCTVCNKYQCSCNPKKRSPKTVIRDLDYINPCEPLYIIDHCHNRGKTGPTGPTGATGATGPTGPTGATGPTGATGATGPTGSTGPTGSTGATGPTGATGVTGATGATGATGVTGATGPTGPTGLTGATGATGTTGATGATGATGVTGATGPTGATGVTGATGVTGATGPTGATGSTGETGTTGVTGATGVTGATGPTGSIGSTGATGATGPTGATGATGATGPTGETGPTGPTGPTGITGPTGATGSGGVIPFASGGTVTINSLLTGVPNTGGIIAFGNSTTSVGIGNATVNLQNINNEAFVVPHAGAITNVAAFFNITVALNIAVLGTATITAQVWKSQNASSNVFAPLASTLISFTPLAGAIAAFTTLNGALANLNEPVNVGDRLIMVFSVATTGLSVITAITGTASAGITIS

SEQ ID NO: 220 - protein, 395 aa, Artificial Sequence
  N-terminal Signal Sequence
GVTGATGATGPTGATGATGATGPTGVTGATGAAGGDTGATGATGQTGETGVAGATGATGQTGETGVAGATGATGQTGETGVAGATGATGQTGETGVAGATGATGQTGETGVAGATGATGQTGETGVAGATGATGQTGETGVAGATGATGQTGETGVAGATGATGQTGETGVAGATGATGQTGETGKAGATGATGQTGETGVTGATGATGQTGETGEAGATGAAGQTGETGVTGATGATGQTGETGVAGATGATGQTGETGVAGATGATGATGQTGETGVVGATGATGQTGETGVVGATGATGQTGETGVAGATGATGATGQTGETGVAGATGATGQTGETGVAGATGATGQTGETGVAGATGATGQTGETGVAGATGATGQTGEAGVAGVTGATG

SEQ ID NO: 221 - DNA, 165 bp, Artificial Sequence
  N-terminal Signal Sequence
tgaaaagatggggaattctccatcttttttcttttgtctggagtggtgagatatacgacatacaacgaatgagcataacatgaatagtttataaaatcgcaaaaaatttttcggaaggagagagcgaaaattaacaataagcgggctaacgcattcaataagaag

SEQ ID NO: 222 - protein, 510 aa, Artificial Sequence
  N-terminal Signal Sequence
GVTGATGATGATGAQGPPGAPGPQGVAGAMGPRGEIGPQGVAGATGATGPQGEMGLQGVAGVTGASGPQGEIGPQGVAGVTGAPGPQGEIGPQGVAGATGVPGPQGEIGPQGVAGETGATGPQGEMGLQGVAGVTGATGPQGEMGPQGVAGVTGATGPQGEIGPQGVAGVTGAPGPQGEIGPQGVAGVTGAPGPQGEMGLQGVAGVTGATGPQGIIGATGLRGLAGITGATGERGLAGITGATGVQGVAGSTGATGSQGITGATGVQGVTGSTGATGSQGITGATGVQGVTGSTGATGSQGITGATGVQGVTGSTGATGSQGITGATGVQGVAGSTGATGSQGITGATGVQGVTGSTGATGARGTTGVTGTTGPSGSGLMEVFLSTDQSVGNNDFLGTGNSSASFVRSSIVIPENATIRSLTLNIRDHALSAGQTASAQIFVSTNCGFTSVATGIIATVTGPNSSTTPNCCAKVIANYPVSSCTLLSVQVSTTGGAFSNGVSATVLFNTV

SEQ ID NO: 223 - DNA, 156 bp, Artificial Sequence
  N-terminal Signal Sequence
tgttgaaacatattattgagcattgggttttatatgacctaatgttctttttattttaagatacatatttaaatcattctaatattctaggtgcattaattatctcaataagtagacttttgaatatgataaaaagggacaggaggtgaagtcgca

SEQ ID NO: 224 - protein, 778 aa, Artificial Sequence
  N-terminal Signal Sequence
GKTGPTGATGPTGSMGPTGVTGSTGATGPTGSTGVTGETGSTGTTGPSGSTGPTGATGSTGETGATGSTGATGETGPTGPTGTTGVTGETGATGPTGATGATGETGSTGPTGATGETGETGSTGPTGATGSTGATGPTGATGSTGATGSTGATGSTGATGPTGATGSTGETGPTGETGSTGATGSTGATGETGPTGTTGVTGETGATGPTGATGATGETGETGSTGPTGATGETGETGSTGPTGATGSTGATGPTGATGSTGATGSTGATGSTGATGPTGATGSTGETGPTGETGSTGATGSTGATGETGPTGATGSTGATGETGATGPTGPTGTTGVTGATGPTGATGETGPTGATGEAGPTGSTGATGPTGATGITGATGPTGSTGATGSTGETGSTGETGPTGATGVTGATGPTGETGSTGPMGATGETGSTGSTGVTGATGATGETGATGSTGATGTTGATGETGPTGPTGSTGATGETGPTGPTGATGVTGATGATGATGETGSTGPMGATGETGSTGSTGVTGATGATGVTGATGETGSTGATGSTGATGETGPTGPTGPTGPTGTTGVTGVTGETGPTGATGETGSTGPTGTTGVTGETGATGPTGATGSTGETGATGETGRTGPTGTTGVTGETGATGETGPTGATGSTGATGPTGATGATGATGETGPTGATGETGATGATGSTGATGPTGPTGTTGVTGATGPTGATGETGPTGATGEAGPTGSTGSTGPTGATGITGATGPTGATGATGSTGETGSTGETGPTGSTG

SEQ ID NO: 225 - DNA, 371 bp, Artificial Sequence
  N-terminal Signal Sequence
atattttagaaagtgaaaaatacagataaaagcctttgctgattcatttttctggatttatttaaaagaacgattggagagtattcttcagccgtatcttttttttgttgctaaaaatattaataattttacttgaggattttaatagattaagaaatttttaattctgatacctatattgtattacaggaatattttagaaataagcatatcccattttcattttacagtttaatagcatgctagagaaatacataattatcagactaccatgaatgagatgattatccgatttttttctgtcacattacaattgtccaatatcaatctatttcacgagaatagtatgtatatgatgggaggtgataata

SEQ ID NO: 226 - DNA, 357 bp, Paenibacillus sp. NRRL B-50972
atggtagtattatctactggacctattgcaaacgatcctgttctaggagtcagacccacccaactggtcacagtaaaaatagataaccgagattctgtaaattcttctatcgttttgatcgagggttttattttaaacggtagcagaacattatatgtacaacaattagtggtagtgggaccaaatgcggttataacgaggaatttctttgcaaatgtagacgcatttgaattcgtttttaccactagcggaccagcagagaatgaaactcaaatttctgtttggggtaaagatgcattggggcaattagtacctgcccatcggttagtatctgacgaacttttaggaaccgatcga

SEQ ID NO: 227 - protein, 119 aa, Paenibacillus sp. NRRL B-50972
MVVLSTGPIANDPVLGVRPTQLVTVKIDNRDSVNSSIVLIEGFILNGSRTLYVQQLVVVGPNAVITRNFFANVDAFEFVFTTSGPAENETQISVWGKDALGQLVPAHRLVSDELLGTDR

SEQ ID NO: 228 - DNA, 231 bp, Paenibacillus sp. NRRL B-50972
atgcctgccttggatgaatggagtagtatacaacaaatcgatatggaggtgtttgtattgggtcgtcccgaattgaaacgaaagaaaggccgtaaaaaagacgtttttatccgctcttggtttagtaaaaaacgtccgaagagaaaatgccattcgaaacgaaagtgcttttgcaaggaaatcgtcgtcagaaagcaaatcgtccgtgtaaatatacctcaaaatgtttta

SEQ ID NO: 229 - protein, 77 aa, Paenibacillus sp. NRRL B-50972
MPALDEWSSIQQIDMEVFVLGRPELKRKKGRKKDVFIRSWFSKKRPKRKCHSKRKCFCKEIVVRKQIVRVNIPQNVL

SEQ ID NO: 230 - DNA, 438 bp, Paenibacillus sp. NRRL B-50972
atgaaacacagaaaaccgttcaggttcagtggtgcttcaaaaaaagacgaggactgcaaaccacctaaaattagcagagaaacggaagaacttctcaaactgattaaggaattagtcgccatcatcccgctcgttttcgcaaacccgtctgtggctaatgtaacttcattgcaacagattttacagcgattattagctctcgcaaataaattgagacttagaggctcggctaagacagatttattagcggcgttggaactggctatcgtggcgtcggaagccactcttttctccccgatcggtgttggaacgacactgcaacaactgctggaagtcttattgtctattattttgcaggaaccccttgatcctgctcttaaagacagtttgatcagtgcaatcagaaatgccgaaacggctatcagtattgcgttgggt

SEQ ID NO: 231 - protein, 146 aa, Paenibacillus sp. NRRL B-50972
MKHRKPFRFSGASKKDEDCKPPKISRETEELLKLIKELVAIIPLVFANPSVANVTSLQQILQRLLALANKLRLRGSAKTDLLAALELAIVASEATLFSPIGVGTTLQQLLEVLLSIILQEPLDPALKDSLISAIRNAETAISIALG

SEQ ID NO: 232 - DNA, 360 bp, Paenibacillus sp. NRRL B-50972
atggcggttatatcaactggacccatagaaaataattatgtcagtggtattcggcctactcatcgagttaccgtgaaaattgataatcgtgatactgtgaattcttctacggtattgattcagggtttttatctaaatggtacaagaacgttatatgtgcttgattttataactgtaaattcaaatgaagtgattacaaaagattattatgctgattttaattcatttgagtttgtttttaccactgaaagtgttacagaaaatgagattcaagtttcagtctggggtaaaaattcaatggggcagttagtgacagctcaccgtgttgtatcttccgaattgcttgtagcaaaaggcgcg

SEQ ID NO: 233 - protein, 120 aa, Paenibacillus sp. NRRL B-50972
MAVISTGPIENNYVSGIRPTHRVTVKIDNRDTVNSSTVLIQGFYLNGTRTLYVLDFITVNSNEVITKDYYADFNSFEFVFTTESVTENEIQVSVWGKNSMGQLVTAHRVVSSELLVAKGA

SEQ ID NO: 234 - DNA, 330 bp, Paenibacillus sp. NRRL B-50972
ttgggaaatttattgttgcgtaaaagatatcgcttgacccaggtggcaaggaaaaaaaagaaggaaagagatcaaaagatgggagcgttccgttttatgcccatttatcgtacaggaacgagctgcattcgtaacaaaaagggaaataaacgtatttatagacagggtagaagaagagagagaatatgcgcttatagacatcatttgcacgctgagcgggtgccctcaggtttatcaaataaaaaaatctgttttatgaaattcaaaggtcaacgaagactgcgaggcggcgaacaggagcctcaaggcaattcaggaggagcagttcaa

SEQ ID NO: 235 - protein, 110 aa, Paenibacillus sp. NRRL B-50972
LGNLLLRKRYRLTQVARKKKKERDQKMGAFRFMPIYRTGTSCIRNKKGNKRIYRQGRRRERICAYRHHLHAERVPSGLSNKKICFMKFKGQRRLRGGEQEPQGNSGGAVQ

SEQ ID NO: 236 - protein, 110 aa, Paenibacillus sp. NRRL B-50972
MGNLLLRKRYRLTQVARKKKKERDQKMGAFRFMPIYRTGTSCIRNKKGNKRIYRQGRRRERICAYRHHLHAERVPSGLSNKKICFMKFKGQRRLRGGEQEPQGNSGGAVQ

SEQ ID NO: 237 - DNA, 200 bp, Paenibacillus sp. NRRL B-50972
gaaacgggagtggtgaaatcattgatgctcagcgcattgttgcggatgagcaactagattcttgaaacacaacatatgtacagagatagaaccacaatcgtaacaaatggttgagacataaaatagagggaacaggatcttgagaaagatctcattgttcacaaaaaagcttgattttactagaaaggagggagtatcca

SEQ ID NO: 238 - DNA, 200 bp, Paenibacillus sp. NRRL B-50972
ctatatacatgcgcaaaaaacggcttcaaactgcttcataattacggcacgtttcttctggcgccttcggctgttccttggtgtgaaccaaggtaacagccgggggcgctatttttatataactagatgaatgtacctgtacaaagacccatttttatccaaaattagatcattgcctatcaaccacaggacagatgtcc

SEQ ID NO: 239 - DNA, 200 bp, Paenibacillus sp. NRRL B-50972
agcgttacaagttggaagcccggtttggaaatacagaaaatcgatattaaagcttatgtacaagcatccaataataattcttgtgtggtgattcacccttttcgcttcagtaaatatattgttaatatctgcgaaacggggcgatgatccacctgtcacctctacagtagggagaaatgtgaaggaggagatatttgaac

SEQ ID NO: 240 - DNA, 200 bp, Paenibacillus sp. NRRL B-50972
agcggtattttttgtgccccacaaaaaaggctcccttatcaaaaggggtttttatcacataggaaatgtccacacgtatatatagatgttacatattatataaatcgtgaacattcgaatctcaatactagttatagaagaggtggcattagtgataggattatagcttcgttactttagacaaaaggagaatccaatat

SEQ ID NO: 241 - DNA, 200 bp, Paenibacillus sp. NRRL B-50972
atttatttttttgaaaaattacaggggattcagtcccactttcagtaaattcagaaagaaaaataatgtaacggcgaaatggaagtgagcattaaaaatttatttttttggaaaaaaatttaaggaggtcatctgtccaatcaggttcgtttagattccataagataatgaaactgtacttaattatggaggtgtcagta

SEQ ID NO: 242 - DNA, 3582 bp, Paenibacillus sp. NRRL B-50972
atggtagtattatctactggacctattgcaaacgatcctgttctaggagtcagacccacccaactggtcacagtaaaaatagataaccgagattctgtaaattcttctatcgttttgatcgagggttttattttaaacggtagcagaacattatatgtacaacaattagtggtagtgggaccaaatgcggttataacgaggaatttctttgcaaatgtagacgcatttgaattcgtttttaccactagcggaccagcagagaatgaaactcaaatttctgtttggggtaaagatgcattggggcaattagtacctgcccatcggttagtatctgacgaacttttaggaaccgatcgaggaatccaaggacctcaaggagttcagggagcccaaggcgaccaaggtgaccaaggacctcagggtgttcaaggacctcaaggagttcagggagcccaaggagaccaaggagttcaaggcgtacaaggagaccaaggacctcaaggagtccaaggcgaccaaggtgaccaaggacctcaaggagttcaaggagcgcaaggtgaccaaggccctcaaggagttcagggagcccaaggtgaccaaggacctcaaggcgttcagggagcgcaaggtgaccaaggacctcaaggtgatcaaggacctcagggagttcaaggagaccaaggcgatcaaggaccacagggagttcaaggcgtacaaggtgatcaaggacctcagggtgttcaaggagaccaaggcgaccaaggacctcagggtgttcaaggcgtacaaggtgaccaaggacctcagggtgttcaaggcgtacaaggtgaccaaggacctcagggagttcaaggagaccaaggcgatcaaggaccacagggagttcaaggcgtacaaggtgatcaaggacctcagggtgttcaaggagaccaaggcgaccaaggacctcagggtgttcaaggcgtacaaggtgaccaaggacctcagggtgttcaaggcgtacaaggtgaccaaggacctcagggtgttcaaggcgtacaaggtgaccaaggacctcaaggagttcagggagcccaaggtgaccaaggaccacagggagttcaaggcgaccaaggacctcaaggacctcaaggagttcaaggtgaccaaggacctcagggcgttcaaggatcccaaggtgatcaaggacctcaaggagttcaaggcgtacaaggacctcaaggagttcaaggcgtacaaggcgaccaaggacctcaaggtgttcagggagcccaaggcgaccaaggccctcaaggagttcaaggagtccaaggtgaccaaggaccacagggagttcaaggaccgcaaggtgaccaaggaccacagggagttcagggagtccaaggcgaccaaggacctcaaggagtccaaggcgaccaaggtgaccaaggacctcaaggagttcaaggagcgcaaggtgaccaaggccctcaaggagttcagggagcccaaggtgaccaaggacctcaaggcgttcagggagcgcaaggtgaccaaggacctcaaggtgatcaaggacctcagggagttcaaggagaccaaggcgatcaaggaccacagggagttcaaggcgtacaaggtgatcaaggacctcagggtgttcaaggagaccaaggcgaccaaggacctcagggtgttcaaggcgtacaaggtgaccaaggacctcagggtgttcaaggcgtacaaggtgaccaaggacctcagggtgttcaaggcgtacaaggtgaccaaggacctcaaggagttcagggagcccaaggtgaccaaggaccacagggagttcaaggcgaccaaggacctcaaggacctcaaggagttcaaggtgaccaaggacctcagggcgttcaaggatcccaaggtgatcaaggacctcaaggagttcaaggcgtacaaggacctcaaggagttcaaggcgtacaaggcgaccaaggacctcaaggtgttcagggagcccaaggcgaccaaggccctcaaggagttcaaggagtccaaggtgaccaaggaccacagggagttcaaggaccgcaaggtgaccaaggaccacagggagttcagggagtccaaggcgaccaaggacctcaaggtgaccaaggacctcaaggtgaccaaggacctcaaggtgttcaaggtgaccaaggacctcaaggagttcagggagcccaaggcgaccaaggacctcaaggagttcaaggaccgcaaggtgaccaaggacctcaaggagttcaaggcgtacaaggtgatcaaggacctcaaggagttcaaggcgtacaaggtgaccaaggaccacagggtgttcaaggcgtacaaggtgaccaaggacctcaaggtgttcaaggagtccaaggtgatcaaggacctcaaggagttcagggagcccaaggcgaccaaggacctcagggagttcagggagcccaaggcgaccaaggacctcagggagttcagggagcccaaggtgaccaaggacctcagggcgttcaaggagtacaaggtgaccaaggatctcaaggagttcaaggaccgcaaggtgaccaaggacctcaaggagttcaaggcgtacaaggtgaccaaggacctcaaggagttcaaggagtccaaggtgaccaaggacctcaaggtgttcagggagcccaaggtggccaaggacctcagggcgttcaaggcgaccaaggtgaccaaggaccacagggtgttcaaggatctcaaggtgaccaaggaccacaaggcgttcaaggagcccaaggcgaccaaggaccacagggtgttcaaggcgtacaaggtgaccaaggccctcaaggagttcaaggagttcaaggtgaccaaggaccacagggagttcaaggtgttcaaggaccgcaaggtgaccaaggaccacagggtgttcaaggagcccaaggcgaccaaggaccacagggtgttcaaggagtgcaaggtgaccaaggaccgcaaggcgaccaaggtgaccaaggacctcaaggtgttcagggagtccaaggcgaccaaggacctcaaggtgttcagggagtccaaggcgaccaaggacctcaaggtgttcaaggtgaccaaggaccacagggagttcagggagcccaaggtgaccaaggacctcagggagttcaaggtgaccaaggtgaccaaggacctcaaggagttcaaggtgtacaaggtgaccaaggacctcaaggtgttcagggagcccaaggtgaccaaggacctcagggcgtacaaggcgaccaaggtgaccaaggaccacagggtgttcaaggcgtacaaggtgatcaaggacctcaaggagttcaaggcgtacaaggtgaccaaggaccacagggtgttcaaggcgtacaaggtgaccaaggaccacagggtgttcaaggcgaccaaggtgaccaaggacctcaaggcgtacaaggcgatcaaggacctcagggtgttcaaggacctcagggtgttcaaggacctcagggtgttcaaggacctcaaggcgaccaaggagctcaaggtgttcaaggaccacaaggtgaccaaggaccgcaaggcattctgtaa

SEQ ID NO: 243 - protein, 1193 aa, Paenibacillus sp. NRRL B-50972
MVVLSTGPIANDPVLGVRPTQLVTVKIDNRDSVNSSIVLIEGFILNGSRTLYVQQLVVVGPNAVITRNFFANVDAFEFVFTTSGPAENETQISVWGKDALGQLVPAHRLVSDELLGTDRGIQGPQGVQGAQGDQGDQGPQGVQGPQGVQGAQGDQGVQGVQGDQGPQGVQGDQGDQGPQGVQGAQGDQGPQGVQGAQGDQGPQGVQGAQGDQGPQGDQGPQGVQGDQGDQGPQGVQGVQGDQGPQGVQGDQGDQGPQGVQGVQGDQGPQGVQGVQGDQGPQGVQGDQGDQGPQGVQGVQGDQGPQGVQGDQGDQGPQGVQGVQGDQGPQGVQGVQGDQGPQGVQGVQGDQGPQGVQGAQGDQGPQGVQGDQGPQGPQGVQGDQGPQGVQGSQGDQGPQGVQGVQGPQGVQGVQGDQGPQGVQGAQGDQGPQGVQGVQGDQGPQGVQGPQGDQGPQGVQGVQGDQGPQGVQGDQGDQGPQGVQGAQGDQGPQGVQGAQGDQGPQGVQGAQGDQGPQGDQGPQGVQGDQGDQGPQGVQGVQGDQGPQGVQGDQGDQGPQGVQGVQGDQGPQGVQGVQGDQGPQGVQGVQGDQGPQGVQGAQGDQGPQGVQGDQGPQGPQGVQGDQGPQGVQGSQGDQGPQGVQGVQGPQGVQGVQGDQGPQGVQGAQGDQGPQGVQGVQGDQGPQGVQGPQGDQGPQGVQGVQGDQGPQGDQGPQGDQGPQGVQGDQGPQGVQGAQGDQGPQGVQGPQGDQGPQGVQGVQGDQGPQGVQGVQGDQGPQGVQGVQGDQGPQGVQGVQGDQGPQGVQGAQGDQGPQGVQGAQGDQGPQGVQGAQGDQGPQGVQGVQGDQGSQGVQGPQGDQGPQGVQGVQGDQGPQGVQGVQGDQGPQGVQGAQGGQGPQGVQGDQGDQGPQGVQGSQGDQGPQGVQGAQGDQGPQGVQGVQGDQGPQGVQGVQGDQGPQGVQGVQGPQGDQGPQGVQGAQGDQGPQGVQGVQGDQGPQGDQGDQGPQGVQGVQGDQGPQGVQGVQGDQGPQGVQGDQGPQGVQGAQGDQGPQGVQGDQGDQGPQGVQGVQGDQGPQGVQGAQGDQGPQGVQGDQGDQGPQGVQGVQGDQGPQGVQGVQGDQGPQGVQGVQGDQGPQGVQGDQGDQGPQGVQGDQGPQGVQGPQGVQGPQGVQGPQGDQGAQGVQGPQGDQGPQGIL

SEQ ID NO: 244 - protein, 572 aa, Paenibacillus sp. NRRL B-50972
MVVLSTGPIANDPVLGVRPTQLVTVKIDNRDSVNSSIVLIEGFILNGSRTLYVQQLVVVGPNAVITRNFFANVDAFEFVFTTSGPAENETQISVWGKDALGQLVPAHRLVSDELLGTDRGIQGPQGVQGAQGDQGDQGPQGVQGPQGVQGAQGDQGVQGVQGDQGPQGVQGDQGDQGPQGVQGAQGDQGPQGVQGAQGDQGPQGVQGAQGDQGPQGDQGPQGVQGDQGDQGPQGVQGVQGDQGPQGVQGDQGDQGPQGVQGVQGDQGPQGVQGVQGDQGPQGVQGVQGDQGPQGVQGAQGDQGPQGVQGDQGPQGLQGVQGDQGPQGVQGSQGDQGPQGVQGVQGPQGGQGVQGDQGPQGVQGAQGDQGPQGVQGDQGDQGPQGVQGAQGDQGPQGVQGDQGDQGPQGVQGDQGTKELKEYKVTKELKEFKEPKVTKDLRAFKATKVTKDHRVFKEFKVTKDLKEFKEYKVTKDHRVFKAYKVTKDLKVFKATKVTKDLKAYKAIKDLRVFKDLRVFKDLRVFKDLKATKELKVFKDHKVTKDRKAFCKLKVKVYLDDSKVIITFGSFFVLS

SEQ ID NO: 245 - protein, 450 aa, Paenibacillus sp. NRRL B-50972
MVVLSTGPIANDPVLGVRPTQLVTVKIDNRDSVNSSIVLIEGFILNGSRTLYVQQLVVVGPNAVITRNFFANVDAFEFVFTTSGPAENETQISVWGKDALGQLVPAHRLVSDELLGTDRGIQGPQGVQGAQGDQGDQGPQGVQGPQGVQGAQGDQGVQGVQGDQGPQGVQGDQGDQGPQGVQGAQGDQGPQGVQGAQGDQGPQGVQGAQGDQGPQGDQGPQGVQGDQGDQGPQGVQGVQGDQGPQGVQGDQGDQGPQGVQGVQGDQGPQGVQGVQGDQGPQGVQGVQGDQGPQGVQGAQGDQGPQGVQGDQGPQGPQGVQGDQGPQGVQGAQGDQGPQGVQGVQGPQGVQGVQGDQGPQGVQGAQGDQGPQGVQGVQGDQGPQGVQGAQGDQGPQGVQGVQGDQGPQGVQGDQGDQGPQGVQGAQGDQGPQGVQGAQGDQGPQGVQGAQG

SEQ ID NO: 246 - DNA, 1032 bp, Paenibacillus sp. NRRL B-50972
atgcctgccttggatgaatggagtagtatacaacaaatcgatatggaggtgtttgtattgggtcgtcccgaattgaaacgaaagaaaggccgtaaaaaagacgtttttatccgctcttggtttagtaaaaaacgtccgaagagaaaatgccattcgaaacgaaagtgcttttgcaaggaaatcgtcgtcagaaagcaaatcgtccgtgtaaatatacctcaaaatgttttaggtataacaggcgcaactggagctataggtgtagcaggtaacgtaggtgcagcgggcactgtgggtgctgctggagccgtcggaactgcgggaaatgtcggggctgccggtaatgtgggtactgcgggcaccgttgggactgccggaaatgtaggcgcagcgggggctgtgggcactgcgggcgctgttggagctgcgggtgcggtaggaccagtaggtcccgtaggtcctgcgggcattccaggggcagtcggtccagcaggtcctgcgggcgttgcaggggcggtcggtcctgtaggtcctgcgggtgcggtaggtgccactggggctacgggtaccgcaggagcgacggggtccaccggggctacgggagctacaggaaccgcaggtggaatagctcagtttggttatatctacaacttaggatcccgagtcgttccaatagaagcggatgtcattttcgatacgaacggtatacttacacctggaattacccacgctcccggcactacgcagattgcagttaccgatgcggggaactatgaagttaacttttcagtatcgggtgtagagccaggccaatttgccatatttatcaatggcactctggcagcaggaaccatatacggctcaggagctggtacgcagcaaaacacagggcaggccatcctcgctctagcatccggtgatgttcttaccctgcgaaatcatagctctgccgctgcggttaccctgcaaaccttggctggaggtacccaagccaacgtaaacgcttctgtcgttatcaaaaaattaagttag

SEQ ID NO: 247 - protein, 343 aa, Paenibacillus sp. NRRL B-50972
MPALDEWSSIQQIDMEVFVLGRPELKRKKGRKKDVFIRSWFSKKRPKRKCHSKRKCFCKEIVVRKQIVRVNIPQNVLGITGATGAIGVAGNVGAAGTVGAAGAVGTAGNVGAAGNVGTAGTVGTAGNVGAAGAVGTAGAVGAAGAVGPVGPVGPAGIPGAVGPAGPAGVAGAVGPVGPAGAVGATGATGTAGATGSTGATGATGTAGGIAQFGYIYNLGSRVVPIEADVIFDTNGILTPGITHAPGTTQIAVTDAGNYEVNFSVSGVEPGQFAIFINGTLAAGTIYGSGAGTQQNTGQAILALASGDVLTLRNHSSAAAVTLQTLAGGTQANVNASVVIKKLS

SEQ ID NO: 248 - DNA, 1497 bp, Paenibacillus sp. NRRL B-50972
atgaaacacagaaaaccgttcaggttcagtggtgcttcaaaaaaagacgaggactgcaaaccacctaaaattagcagagaaacggaagaacttctcaaactgattaaggaattagtcgccatcatcccgctcgttttcgcaaacccgtctgtggctaatgtaacttcattgcaacagattttacagcgattattagctctcgcaaataaattgagacttagaggctcggctaagacagatttattagcggcgttggaactggctatcgtggcgtcggaagccactcttttctccccgatcggtgttggaacgacactgcaacaactgctggaagtcttattgtctattattttgcaggaaccccttgatcctgctcttaaagacagtttgatcagtgcaatcagaaatgccgaaacggctatcagtattgcgttgggtggcacggcaggaacccccggtccacaagggcccgctgggcctgctggtccgggcggtgctccaggacctgtcggtggaccagggccggtgggtgcggcaggaccagcaggtccagttggacctgctggtcctgtcggacctgtcggggctgccggacctgttggagccgccggacctgttggagccgccggacctatcggcgccgctgggccagtaggcgccgccggggctgctggagccaccggggctacaggagctacaggcgcggcaggacctgccgggggggctaccggggccacgggcgccgttggagccacaggcgctacgggcgcagcgggggtcgctggggctacaggaactacgggcacggcgggcgctgtcggagctaccggggccacgggcacggcgggggccattggagctaccggggccacaggcacggcgggggccgtcggagctaccggggccacaggcacggcgggcgctgtcggagctaccggggccacgggtacagcaggggttactggagccaccggttcgggggcaatcattccatttgcttcgggtggaccagcaattttgacaaccattgtcggcgggctggttggaaccacaagtttgatcggctttggaagctcagcaacaggcattagccttgtgggtggaaccattgacctgacaggcacacttgcagggccactgattaactttgctttttctgtaccacgggatggcgtaattacatccatcgctggatattttagtacaacagctgcgctaactctcgttggatcaaccgcgacgattactgcccagttgtttagttcgactacacctgataacacctttacagcggtccctggggctaccgttacattagctccaccactgactggcatcattgccttgggtaccatttccaatggcatcactaccggattggctataccagtaaccgcgcagactcgtctgctccttgtcttctctgcaacagctacgggactctccctcgtaaacaccatcgtgggttatgcgagcgcaggcattaccatcacctga

SEQ ID NO: 249 - protein, 498 aa, Paenibacillus sp. NRRL B-50972
MKHRKPFRFSGASKKDEDCKPPKISRETEELLKLIKELVAIIPLVFANPSVANVTSLQQILQRLLALANKLRLRGSAKTDLLAALELAIVASEATLFSPIGVGTTLQQLLEVLLSIILQEPLDPALKDSLISAIRNAETAISIALGGTAGTPGPQGPAGPAGPGGAPGPVGGPGPVGAAGPAGPVGPAGPVGPVGAAGPVGAAGPVGAAGPIGAAGPVGAAGAAGATGATGATGAAGPAGGATGATGAVGATGATGAAGVAGATGTTGTAGAVGATGATGTAGAIGATGATGTAGAVGATGATGTAGAVGATGATGTAGVTGATGSGAIIPFASGGPAILTTIVGGLVGTTSLIGFGSSATGISLVGGTIDLTGTLAGPLINFAFSVPRDGVITSIAGYFSTTAALTLVGSTATITAQLFSSTTPDNTFTAVPGATVTLAPPLTGIIALGTISNGITTGLAIPVTAQTRLLLVFSATATGLSLVNTIVGYASAGITIT

SEQ ID NO: 250 - DNA, 3744 bp, Paenibacillus sp. NRRL B-50972
atggcggttatatcaactggacccatagaaaataattatgtcagtggtattcggcctactcatcgagttaccgtgaaaattgataatcgtgatactgtgaattcttctacggtattgattcagggtttttatctaaatggtacaagaacgttatatgtgcttgattttataactgtaaattcaaatgaagtgattacaaaagattattatgctgattttaattcatttgagtttgtttttaccactgaaagtgttacagaaaatgagattcaagtttcagtctggggtaaaaattcaatggggcagttagtgacagctcaccgtgttgtatcttccgaattgcttgtagcaaaaggcgcgggaccgacagggctaacgggagccactggcgctaccggagctactggcgtcacgggtgttaccggagtcactggcgctaccggaactacgggcgttatgggtgataccggagtcactggagttaccggagttactggcgttaccggggctatcggagtcactggcgctatcggagtcacgggggctaccggagccacaggagttacgggggccactggagttaccggggctattggagttactggcgctatcggagtcactggcgctaccggagctactggcgttactggggctactggcgctactggagtcacaggagttaccggggctactggcgttaccggagttaccggagttactggcatcaccggggctatcggagctactggcgttaccggagctactggcgtcacgggtattaccggagtcactggcgttaccggggctactggcgttactggagttactggcatcacaggcgttaccggagttactggtgttactggtgttactggagctactggcgttaccggggctactggcgctaccggagccactggcgttactggagttactggcgttactggcgctactggagctactggtgttaccggggctaccggggctaccggtgtcacgggtgataccggtgtcactggcgctaccggggctaccggagtttctggcgctactggggctactggtgtcacgggtgataccggagttaccggagctactggcgctacaggtgctaccggagttactggcggaacaggtgcaaccggagttactggagttactggcgttaccggggctatcggagtcactggcgctactggagctactggagctgctggaatcacgggtgttaccggagttactggcatcaccggtgctaccggggctacgggcgctaccggagttactggcatcacaggagtcactggcgctaccggagttactggcgtaacaggtgcaaccggagttactggagttaccggggctatcggagttactggtgtcaccggagctactggcgtcacgggtgttaccggagtcactggcgctaccggagctactggcgttacgggtgttaccggagttaccggagttactggcgttaccggagctactggcgttaccggagttactggagttactggagttattggagttactggagttactggagttactggagttactggagttaccggagttaccggagttactggagttaccggggctatcggagtcactggcgctatcggagtcacgggggctaccggggtcactggcgctaccggagctactggcgtaacaggggctactggagttaccggggctatcggagtcactggcgctactggagctgctggaatcacgggtgttaccggagtcactggtgttactggagttaccggagctactggcatcacgggtgataccggagtcactggcgctaccggagctactggcgttacgggtgttaccggagtcactggggctaccggagctactggcgtcacgggtgataccggagttactggagtcactggcgctaccggagttactggcgtaacaggtgcagccggagttactggcatcacgggggctaccggagttactggagttaccggggctattggagtcactggcgctatcggagtcacgggggctaccggagccacaggagttacgggtattaccggagctactggcgctactggagccacaggtgctaccggagttactggagttactggcgctaccggagctactggcgctactggcgtcacgggttctactggggtcactggcgctactggcgttaccggagctactggcgtcacgggttctactggggtcactggcgctactggcgttaccggagctactggcgtcacgggtattaccggagtcactggcgttaccggagttactggtgctactggagctactggcgttaccggggctaccggagtcactggggctaccggagctactggcgtcacgggtattaccggagtcactggggctaccggagctactggcgtcacgggtgttaccggagtcaccggagtcactggagttactggagttactggcgctaccggagctactggcgttaccggagctactggcgctactggcgtcacgggtgataccggagtcactggggctaccggagttaccggagtcactggcgctactggggctactggtgtcacgggtgttaccggagtcactggcgctaccggggctactggtgtcacgggtgttaccggggctaccggagctactggcgacacgggtgttaccggagtcactggagtcactggagttaccggagtttctggcgctaccggagttaccggagtttctggcgctaccggagttaccggagctactggcgttaccggggctggggctaccggagctactggcgctactggagtcacaggtgttaccggagtcactggcgctaccggagctactggcgctactggagtcacgggtgttaccggagtcactggcgctaccggggctactggtgtcacgggtgttaccggggctaccggagctactggcgacacgggtgttaccggagtcactggagtcactggagttaccggagtttctggcgctaccggagttaccggagctactggcgttaccggggctggggctaccggagctactggcgctactggagtcacaggtgttaccggagtcactggcgctaccggagctactggcgctactggagtcacgggtgttaccggagtcactggcgctaccggggctactggcgctactggagtcacgggtgttactggcgttacgggtgttaccggagtttctggcatcaccggtgctaccggggctattggacctactggtgccacaggtgttggtataacaggttcaacaggttcaaccggccccactggcccacctcctacgtttatagacgcatactttaacggtaatattcaacctcagacaattgcttcgggatcaaacattttaaatattactccaaaccaatctactgcacttacttataacgcagtaacaagtgttttcacaatacaaaatgcggggttgtataacattagtgttgtaataaatcttgcaactgccacactaccagaagcaacaattgggttatcactaaataattctacagcatatctcgctcctgctgtaaccacggcaacaagtggtcaattggttttagttcaaattgaggctcttgctgtcggagatacaattcaatttagaaatatatctgggtttcctattaccattgctaattcaccagtaatagctaacagctcaggtcatgtagctatttcgagattctcagctttttcataa

SEQ ID NO: 251 - protein, 1247 aa, Paenibacillus sp. NRRL B-50972
MAVISTGPIENNYVSGIRPTHRVTVKIDNRDTVNSSTVLIQGFYLNGTRTLYVLDFITVNSNEVITKDYYADFNSFEFVFTTESVTENEIQVSVWGKNSMGQLVTAHRVVSSELLVAKGAGPTGLTGATGATGATGVTGVTGVTGATGTTGVMGDTGVTGVTGVTGVTGAIGVTGAIGVTGATGATGVTGATGVTGAIGVTGAIGVTGATGATGVTGATGATGVTGVTGATGVTGVTGVTGITGAIGATGVTGATGVTGITGVTGVTGATGVTGVTGITGVTGVTGVTGVTGATGVTGATGATGATGVTGVTGVTGATGATGVTGATGATGVTGDTGVTGATGATGVSGATGATGVTGDTGVTGATGATGATGVTGGTGATGVTGVTGVTGAIGVTGATGATGAAGITGVTGVTGITGATGATGATGVTGITGVTGATGVTGVTGATGVTGVTGAIGVTGVTGATGVTGVTGVTGATGATGVTGVTGVTGVTGVTGATGVTGVTGVTGVIGVTGVTGVTGVTGVTGVTGVTGVTGAIGVTGAIGVTGATGVTGATGATGVTGATGVTGAIGVTGATGAAGITGVTGVTGVTGVTGATGITGDTGVTGATGATGVTGVTGVTGATGATGVTGDTGVTGVTGATGVTGVTGAAGVTGITGATGVTGVTGAIGVTGAIGVTGATGATGVTGITGATGATGATGATGVTGVTGATGATGATGVTGSTGVTGATGVTGATGVTGSTGVTGATGVTGATGVTGITGVTGVTGVTGATGATGVTGATGVTGATGATGVTGITGVTGATGATGVTGVTGVTGVTGVTGVTGATGATGVTGATGATGVTGDTGVTGATGVTGVTGATGATGVTGVTGVTGATGATGVTGVTGATGATGDTGVTGVTGVTGVTGVSGATGVTGVSGATGVTGATGVTGAGATGATGATGVTGVTGVTGATGATGATGVTGVTGVTGATGATGVTGVTGATGATGDTGVTGVTGVTGVTGVSGATGVTGATGVTGAGATGATGATGVTGVTGVTGATGATGATGVTGVTGVTGATGATGATGVTGVTGVTGVTGVSGITGATGAIGPTGATGVGITGSTGSTGPTGPPPTFIDAYFNGNIQPQTIASGSNILNITPNQSTALTYNAVTSVFTIQNAGLYNISVVINLATATLPEATIGLSLNNSTAYLAPAVTTATSGQLVLVQIEALAVGDTIQFRNISGFPITIANSPVIANSSGHVAISRFSAFS

SEQ ID NO: 252 - DNA, 849 bp, Paenibacillus sp. NRRL B-50972
ttgggaaatttattgttgcgtaaaagatatcgcttgacccaggtggcaaggaaaaaaaagaaggaaagagatcaaaagatgggagcgttccgttttatgcccatttatcgtacaggaacgagctgcattcgtaacaaaaagggaaataaacgtatttatagacagggtagaagaagagagagaatatgcgcttatagacatcatttgcacgctgagcgggtgccctcaggtttatcaaataaaaaaatctgttttatgaaattcaaaggtcaacgaagactgcgaggcggcgaacaggagcctcaaggcaattcaggaggagcagttcaaggggtgcatggattaagggggaccgatggtaatgctgggcatcaaggcatacaaggtccggctgggccacagggcattccgggtagtgccggaccccagggccaggcgggcgccataggcccccaaggtgaacagggtcttcagggggttccagggattcaaggcttgcaaggagaggctgggccacagggagagcagggaccaccgcttaatttggatgggatcacggttgtgcctgaggtacagcgatatttctattttgccgattcagatctgacgggtacggttgaaatccctatttcccagtttacgaatgatgatggacagttggcaagtcagcttccagaattgggtgcgaacagctacacggatttgtatattaatggggtactgcaggaaagcaggttgtaccagataagtagtaccacattgactgttgaattggaagaagctcttgtaattgcgggtacgccgtttattttcgaggtttttcaatttacattaagaatggcgaactga

SEQ ID NO: 253 - protein, 282 aa, Paenibacillus sp. NRRL B-50972
MGNLLLRKRYRLTQVARKKKKERDQKMGAFRFMPIYRTGTSCIRNKKGNKRIYRQGRRRERICAYRHHLHAERVPSGLSNKKICFMKFKGQRRLRGGEQEPQGNSGGAVQGVHGLRGTDGNAGHQGIQGPAGPQGIPGSAGPQGQAGAIGPQGEQGLQGVPGIQGLQGEAGPQGEQGPPLNLDGITVVPEVQRYFYFADSDLTGTVEIPISQFTNDDGQLASQLPELGANSYTDLYINGVLQESRLYQISSTTLTVELEEALVIAGTPFIFEVFQFTLRMAN

SEQ ID NO: 254 - protein, 60 aa, Paenibacillus sp. NRRL B-50972
  misc_feature (2)..(2): Xaa can be any naturally occurring amino acid
  misc_feature (4)..(4): Xaa can be any naturally occurring amino acid
  misc_feature (10)..(10): Xaa can be any naturally occurring amino acid
  misc_feature (12)..(13): Xaa can be any naturally occurring amino acid
  misc_feature (15)..(15): Xaa can be any naturally occurring amino acid
  misc_feature (17)..(17): Xaa can be any naturally occurring amino acid
  misc_feature (21)..(22): Xaa can be any naturally occurring amino acid
  misc_feature (32)..(32): Xaa can be any naturally occurring amino acid
  misc_feature (37)..(37): Xaa can be any naturally occurring amino acid
  misc_feature (41)..(41): Xaa can be any naturally occurring amino acid
  misc_feature (44)..(44): Xaa can be any naturally occurring amino acid
  misc_feature (48)..(48): Xaa can be any naturally occurring amino acid
  misc_feature (54)..(58): Xaa can be any naturally occurring amino acid
  misc_feature (60)..(60): Xaa can be any naturally occurring amino acid
MXVXSTGPIXNXXVXGXRPTXXVTVKIDNRDXVNSSXVLIXGFXLNGXRTLYVXXXXXVX

SEQ ID NO: 255 - protein, 120 aa, Paenibacillus sp. NRRL B-50972
  misc_feature (2)..(2): Xaa can be any naturally occurring amino acid
  misc_feature (4)..(4): Xaa can be any naturally occurring amino acid
  misc_feature (10)..(10): Xaa can be any naturally occurring amino acid
  misc_feature (12)..(13): Xaa can be any naturally occurring amino acid
  misc_feature (15)..(15): Xaa can be any naturally occurring amino acid
  misc_feature (17)..(17): Xaa can be any naturally occurring amino acid
  misc_feature (21)..(22): Xaa can be any naturally occurring amino acid
  misc_feature (32)..(32): Xaa can be any naturally occurring amino acid
  misc_feature (37)..(37): Xaa can be any naturally occurring amino acid
  misc_feature (41)..(41): Xaa can be any naturally occurring amino acid
  misc_feature (44)..(44): Xaa can be any naturally occurring amino acid
  misc_feature (48)..(48): Xaa can be any naturally occurring amino acid
  misc_feature (54)..(58): Xaa can be any naturally occurring amino acid
  misc_feature (60)..(61): Xaa can be any naturally occurring amino acid
  misc_feature (63)..(63): Xaa can be any naturally occurring amino acid
  misc_feature (67)..(70): Xaa can be any naturally occurring amino acid
  misc_feature (72)..(75): Xaa can be any naturally occurring amino acid
  misc_feature (83)..(86): Xaa can be any naturally occurring amino acid
  misc_feature (90)..(90): Xaa can be any naturally occurring amino acid
  misc_feature (92)..(92): Xaa can be any naturally occurring amino acid
  misc_feature (98)..(100): Xaa can be any naturally occurring amino acid
  misc_feature (105)..(105): Xaa can be any naturally occurring amino acid
  misc_feature (109)..(109): Xaa can be any naturally occurring amino acid
  misc_feature (112)..(112): Xaa can be any naturally occurring amino acid
  misc_feature (117)..(120): Xaa can be any naturally occurring amino acid
MXVXSTGPIXNXXVXGXRPTXXVTVKIDNRDXVNSSXVLIXGFXLNGXRTLYVXXXXXVXXNXVITXXXXAXXXXFEFVFTTXXXXENEXQXSVWGKXXXGQLVXAHRXVSXELLVXXXX

SEQ ID NO: 256 - DNA, 300 bp, Paenibacillus sp. NRRL B-50972
atggtagtattatctactggacctattgcaaacgatcctgttctaggagtcagacccacccaactggtcacagtaaaaatagataaccgagattctgtaaattcttctatcgttttgatcgagggttttattttaaacggtagcagaacattatatgtacaacaattagtggtagtgggaccaaatgcggttataacgaggaatttctttgcaaatgtagacgcatttgaattcgtttttaccactagcggaccagcagagaatgaaactcaaatttctgtttggggtaaagatgcattg

SEQ ID NO: 257 - protein, 100 aa, Paenibacillus sp. NRRL B-50972
MVVLSTGPIANDPVLGVRPTQLVTVKIDNRDSVNSSIVLIEGFILNGSRTLYVQQLVVVGPNAVITRNFFANVDAFEFVFTTSGPAENETQISVWGKDAL

SEQ ID NO: 258 - DNA, 240 bp, Paenibacillus sp. NRRL B-50972
atggtagtattatctactggacctattgcaaacgatcctgttctaggagtcagacccacccaactggtcacagtaaaaatagataaccgagattctgtaaattcttctatcgttttgatcgagggttttattttaaacggtagcagaacattatatgtacaacaattagtggtagtgggaccaaatgcggttataacgaggaatttctttgcaaatgtagacgcatttgaattcgttttt

SEQ ID NO: 259 - protein, 80 aa, Paenibacillus sp. NRRL B-50972
MVVLSTGPIANDPVLGVRPTQLVTVKIDNRDSVNSSIVLIEGFILNGSRTLYVQQLVVVGPNAVITRNFFANVDAFEFVF

SEQ ID NO: 260 - DNA, 120 bp, Paenibacillus sp. NRRL B-50972
atggtagtattatctactggacctattgcaaacgatcctgttctaggagtcagacccacccaactggtcacagtaaaaatagataaccgagattctgtaaattcttctatcgttttgatc

SEQ ID NO: 261 - protein, 40 aa, Paenibacillus sp. NRRL B-50972
MVVLSTGPIANDPVLGVRPTQLVTVKIDNRDSVNSSIVLI

SEQ ID NO: 262 - DNA, 66 bp, Paenibacillus sp. NRRL B-50972
atgtttaccactagcggaccagcagagaatgaaactcaaatttctgtttggggtaaagatgcattg

SEQ ID NO: 263 - protein, 22 aa, Paenibacillus sp. NRRL B-50972
MFTTSGPAENETQISVWGKDAL

SEQ ID NO: 264 - DNA, 36 bp, Paenibacillus sp. NRRL B-50972
atgactcaaatttctgtttggggtaaagatgcattg

SEQ ID NO: 265 - protein, 12 aa, Paenibacillus sp. NRRL B-50972
MTQISVWGKDAL

SEQ ID NO: 266 - DNA, 21 bp, Paenibacillus sp. NRRL B-50972
atgactcaaatttctgtttgg

SEQ ID NO: 267 - protein, 7 aa, Paenibacillus sp. NRRL B-50972
MTQISVW

SEQ ID NO: 268 - DNA, 27 bp, Paenibacillus sp. NRRL B-50972
atgcaaatttctgtttggggtaaagat

SEQ ID NO: 269 - protein, 9 aa, Paenibacillus sp. NRRL B-50972
MQISVWGKD

SEQ ID NO: 270 - DNA, 21 bp, Paenibacillus sp. NRRL B-50972
atgtggggtaaagatgcattg

SEQ ID NO: 271 - protein, 7 aa, Paenibacillus sp. NRRL B-50972
MWGKDAL

SEQ ID NO: 272 - protein, 9 aa, Artificial Sequence
  Truncated N-terminal targeting sequence derived from Paenibacillus
MQISVWGKB

SEQ ID NO: 273 - DNA, 2370 bp, Bacillus thuringiensis
atgaacaaaaacaacactaaattatctactcgtgctcttccatctttcattgactacttcaacggtatttacggtttcgctacaggtatcaaagacattatgaacatgatcttcaaaactgatactggtggtgacttaactcttgatgaaattcttaagaaccaacaattacttaacgacatctctggtaaacttgatggtgttaacggttctcttaacgacttaatcgcacaaggtaaccttaacactgaactaagcaaagaaatcctaaaaattgctaatgaacaaaaccaagttcttaatgatgttaacaacaaactagacgctattaacactatgttacgtgtataccttccaaaaatcacttcaatgctttctgatgttatgaaacaaaactacgctttatctttacaaatcgaatacttatctaagcaattacaggaaatctctgataaattagacatcatcaacgttaacgtattaattaacagcactcttacagaaattacgccagcttaccaacgtatcaaatacgttaatgaaaaattcgaagaacttacattcgctactgaaacttcttctaaagttaaaaaagacggttctcctgctgatattttagatgaattaactgaattaactgaacttgctaaatctgtaactaaaaacgatgttgacggtttcgaattctaccttaacactttccacgatgtaatggtaggcaacaaccttttcggtcgctctgctcttaaaactgctagtgaattaatcactaaagaaaacgttaaaacttctggtagcgaagtaggtaacgtttacaatttcttaatcgtactaacagctcttcaagcacaagcattccttactcttactacttgccgtaaattacttggtttagctgatattgattacacttctatcatgaacgaacatcttaacaaagaaaaagaagaattccgtgttaatatccttccgacattatctaacactttttctaacccaaattacgctaaggttaaaggtagcgatgaagatgcaaaaatgatcgtagaagctaaaccaggtcacgcattaatcggtttcgagatctcaaacgacagcatcacagttttaaaagtttatgaagctaaacttaaacaaaactaccaagtagataaagacagcctgtctgaagttatctacggtgacatggataaattattatgtccagatcaaagtgaacaaatctactacactaataacatcgtattcccaaacgaatatgtaatcactaaaatcgatttcacaaagaaaatgaaaactcttcgttacgaggtaactgctaacttttacgattcatctactggtgaaattgatcttaacaagaaaaaagttgaatcttctgaagctgaataccgtacattatctgctaacgatgatggtgtttatatgcctttaggtgtaatttctgaaacattcttaactcctatcaacggtttcggtcttcaagctgatgaaaactctcgcttaatcactttaacatgcaaatcttaccttcgcgaacttttattagctacagatttatctaacaaagaaactaaacttattgttcctccatctggattcattagcaatatcgttgagaacggtagcattgaagaagataaccttgaaccttggaaagctaacaacaaaaacgcatacgttgatcatacaggtggtgttaacggtacaaaagctctttacgtacacaaagatggtggtattagccaattcatcggcgacaaattaaagccaaaaactgaatacgttatccaatacactgtaaaaggtaaaccatcaattcatttaaaagatgaaaatactggttacattcattacgaagacactaacaacaaccttgaagattaccaaacaatcaacaaacgttttacaacaggaactgacttaaaaggtgtttacttaattttaaaatctcaaaacggtgacgaggcatggggcgacaacttcatcattctagaaatttctccttctgaaaaattactttctcctgaattaatcaatactaacaactggacttctacaggttctactaacatttctggaaacactttaacactttaccaaggtggtcgtggtatcttaaaacaaaacttacaattagattcattctctacataccgtgtttatttctctgtatctggtgacgctaacgtacgtatccgtaactctcgtgaagtattatttgaaaaacgttacatgtcaggagctaaagatgtatctgaaatgttcactactaaattcgaaaaagataatttctacattgaattatctcaaggtaacaacctttacggtggtccaatcgttcacttctacgatgtttctatcaaataa

SEQ ID NO: 274 - protein, 789 aa, Bacillus thuringiensis
MNKNNTKLSTRALPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAQAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 275 - protein, 60 aa, Artificial Sequence; identical to SEQ ID NO: 254
  Consensus sequence
  misc_feature (2)..(2): Xaa can be any naturally occurring amino acid
  misc_feature (4)..(4): Xaa can be any naturally occurring amino acid
  misc_feature (10)..(10): Xaa can be any naturally occurring amino acid
  misc_feature (12)..(13): Xaa can be any naturally occurring amino acid
  misc_feature (15)..(15): Xaa can be any naturally occurring amino acid
  misc_feature (17)..(17): Xaa can be any naturally occurring amino acid
  misc_feature (21)..(22): Xaa can be any naturally occurring amino acid
  misc_feature (32)..(32): Xaa can be any naturally occurring amino acid
  misc_feature (37)..(37): Xaa can be any naturally occurring amino acid
  misc_feature (41)..(41): Xaa can be any naturally occurring amino acid
  misc_feature (44)..(44): Xaa can be any naturally occurring amino acid
  misc_feature (48)..(48): Xaa can be any naturally occurring amino acid
  misc_feature (54)..(58): Xaa can be any naturally occurring amino acid
  misc_feature (60)..(60): Xaa can be any naturally occurring amino acid

SEQ ID NO: 276 - protein, 120 aa, Artificial Sequence; identical to SEQ ID NO: 255
  Consensus Sequence
  misc_feature (2)..(2): Xaa can be any naturally occurring amino acid
  misc_feature (4)..(4): Xaa can be any naturally occurring amino acid
  misc_feature (10)..(10): Xaa can be any naturally occurring amino acid
  misc_feature (12)..(13): Xaa can be any naturally occurring amino acid
  misc_feature (15)..(15): Xaa can be any naturally occurring amino acid
  misc_feature (17)..(17): Xaa can be any naturally occurring amino acid
  misc_feature (21)..(22): Xaa can be any naturally occurring amino acid
  misc_feature (32)..(32): Xaa can be any naturally occurring amino acid
  misc_feature (37)..(37): Xaa can be any naturally occurring amino acid
  misc_feature (41)..(41): Xaa can be any naturally occurring amino acid
  misc_feature (44)..(44): Xaa can be any naturally occurring amino acid
  misc_feature (48)..(48): Xaa can be any naturally occurring amino acid
  misc_feature (54)..(58): Xaa can be any naturally occurring amino acid
  misc_feature (60)..(61): Xaa can be any naturally occurring amino acid
  misc_feature (63)..(63): Xaa can be any naturally occurring amino acid
  misc_feature (67)..(70): Xaa can be any naturally occurring amino acid
  misc_feature (72)..(75): Xaa can be any naturally occurring amino acid
  misc_feature (83)..(86): Xaa can be any naturally occurring amino acid
  misc_feature (90)..(90): Xaa can be any naturally occurring amino acid
  misc_feature (92)..(92): Xaa can be any naturally occurring amino acid
  misc_feature (98)..(100): Xaa can be any naturally occurring amino acid
  misc_feature (105)..(105): Xaa can be any naturally occurring amino acid
  misc_feature (109)..(109): Xaa can be any naturally occurring amino acid
  misc_feature (112)..(112): Xaa can be any naturally occurring amino acid
  misc_feature (117)..(120): Xaa can be any naturally occurring amino acid

(19) United States
(12) Patent Application Publication
(10) Pub. No.: US 2024/0228550 A1
(43) Pub. Date: Jul. 11, 2024

(54) INTERGENERIC ENDOSPORE DISPLAY PLATFORMS, PRODUCTS AND METHODS

(71) Applicant: Ginkgo Bioworks, Inc, Boston, MA (US)

(72) Inventors: Damian Curtis, Davis, CA (US); Ryan McCann, Davis, CA (US); Kyle Tipton, San Jose, CA (US)

(21) Appl. No.: 18/563,054

(22) PCT Filed: May 21, 2022
(86) PCT No.: PCT/US22/30432

§ 371 Date: Nov. 21, 2023

Related U.S. Application Data
(60) Provisional application No. 63/191,656, filed on May 21, 2021.

(51) Int. Cl. C07K 14/195; A01N 63/20; A01N 63/25; C12N 15/62; C12N 15/74

(52) U.S. Cl. CPC C07K 14/195; A01N 63/20; A01N 63/25; C12N 15/625; C12N 15/74; C07K 2319/02

(57) ABSTRACT

Signal sequences useful for targeting proteins and peptides to the surface of endospores produced by multiple bacterial genera (e.g., Brevibacillus, Lysinibacillus, Viridibacillus, and/or Paenibacillus family members) and methods of using the same are provided. The display of heterologous molecules, such as peptides, polypeptides and other recombinant constructs, on the exosporium of Brevibacillus, Lysinibacillus, Viridibacillus, and/or Paenibacillus family members, using particular N-terminal targeting sequences and derivatives of the same, and likewise are provided.

### CROSS REFERENCE TO RELATED APPLICATIONS

This application claims the benefit of, and priority to, U.S. Provisional Patent Application No. 63/191,656, filed May 21, 2021, the entire disclosure of which is incorporated herein by reference.

### REFERENCE TO SEQUENCE LISTING SUBMITTED ELECTRONICALLY

The official copy of the sequence listing is submitted electronically via EFS-Web as an ASCII-formatted sequence listing with a file named “BCS209001WO_ST25.txt” created on May 21, 2022, and having a size of 259 kilobytes, and is filed concurrently with the specification. The sequence listing contained in this ASCII-formatted document is part of the specification and is herein incorporated by reference in its entirety.

### TECHNICAL FIELD

The disclosure provides products useful for various applications, such as delivering heterologous molecules of interest to plants. In particular, the disclosure describes endospore display methods and associated targeting sequences. For example, the disclosure provides intergeneric N-terminal signal sequences useful for various applications, such as targeting heterologous proteins, peptides, and other recombinant constructs to the exosporium of at least two different bacterial genera selected from Brevibacillus, Lysinibacillus, Viridibacillus, and/or Paenibacillus, as well as endospores and methods of using the same. Such intergeneric N-terminal signal sequences may be used as targeting signals for fusion proteins expressed in various Brevibacillus, Lysinibacillus, Viridibacillus, and/or Paenibacillus species as disclosed herein, resulting in endospores with a molecule of interest localized on the exosporium surface. These endospores may be administered to a host, such as a plant host. The resulting platform is amenable to high through-put screening of proteins of interest for beneficial traits, such as agricultural traits (resistance to herbicides, promotion of plant growth and/or health, protection from insects, protection from fungal, bacterial or viral phytopathogens, etc.) The disclosure provides other related products and methods.

### BACKGROUND OF THE DISCLOSURE

Modern agricultural techniques rely heavily on compositions that promote or enhance plant health and growth in order to improve the yield and quality of crops. Such compositions generally include organic or inorganic fertilizers, nutrients and other chemical compounds that promote proper plant growth and development. However, it is well established that long-term or overuse of many of these compositions may result in negative side effects, such as soil acidification or destabilization of the nutrient balance in the soil. Moreover, overuse may result in the enrichment of harmful end-products in crops grown for human consumption.

Modern farms also typically rely on the use of a wide variety of chemicals (e.g., insecticides, herbicides, bactericides, nematicides, and fungicides) to control pests and ensure a high yield of commercially-grown crops. Many of these chemical compounds exhibit broad activity and may be potentially harmful to humans and animals in high concentrations. In addition, some chemical compounds exhibit off-target effects. Moreover, at least some of these synthetic compounds are non-biodegradable. In recent years, there has been increasing pressure from consumers for agricultural products that have been raised and harvested with reduced or no exposure to synthetic insecticides or fungicides. A further problem arising with the use of synthetic insecticides or fungicides is that the repeated and/or exclusive use often leads to selection of resistant pests. Normally, resistant pests are also cross-resistant against other active ingredients having the same mode of action. As a result, pest control compositions and compounds are difficult and expensive to develop (e.g., due to safety concerns and the rapid development of resistance).

Genetic engineering methods have also been used to promote plant growth and/or health without reliance on synthetic chemicals. For example, crops can be modified to introduce or modify genes related to plant growth and/or health, and/or to introduce genes that encode natural or synthetic pest control agents. Transgenes may be introduced into a target plant using a viral vector. In recent years, there has been some success reported using bacteria for delivery of recombinant proteins to plants. However, to date, such success is largely limited to members of the Bacillaceae family and more specifically, Bacillus subtilis, which is the most well-characterized, Gram-positive bacteria and the primary bacterial model for sporulation research. The focus on B. subtilis as a delivery and expression platform is further due to the fact that the B. subtilis genome and biological pathways related to protein synthesis and secretion are well understood. However, due to the high degree of genetic diversity among bacteria, research findings based on B. subtilis studies are often not directly translatable to members inside and outside the Bacillaceae family. For example, B. subtilis endospores lack the exosporium layer that B. cereus family members produce.

Accordingly, while certain methods of delivering heterologous genetic materials are known, there is a need in the art for developing new delivery and expression platforms for such genetic materials.

### BRIEF SUMMARY OF EXEMPLARY EMBODIMENTS OF THE DISCLOSURE

The disclosure describes methods, compositions and genetic constructs that address the needs identified above by, for example, providing, among other things, a new platform for delivering recombinant enzymes and other molecules of interest (e.g., a peptide or protein) to an environment (e.g., a plant or field) using spore-forming members of the Brevibacillus, Lysinibacillus, Viridibacillus, and Paenibacillus genera. The genetic constructs described herein are further advantageous, in some aspects, because they maintain exosporium-targeting functionality when expressed in multiple genera of bacteria. In view of this intergeneric compatibility, a single genetic construct can be designed for use with multiple bacterial hosts (e.g., members of the Brevibacillus, Lysinibacillus, Viridibacillus, and Paenibacillus genera), reducing research and development costs, as well as the time required to bring new products (e.g., seed treatments) to market that incorporate such genetic constructs.

In one aspect, the disclosure provides a recombinant exosporium-producing Brevibacillus, Lysinibacillus, Viridibacillus, or Paenibacillus cell that expresses a fusion protein comprising: (i) at least one heterologous protein or peptide that confers or modifies a plant trait or attribute (e.g., an enzyme involved in the production or activation of a plant growth stimulating compound, an enzyme that degrades or modifies a bacterial, fungal, or plant nutrient source; or an enzyme, protein, or peptide that protects a plant from a pathogen or a pest); and (ii) an N-terminal targeting sequence (i.e., a signal peptide) that localizes the fusion protein to an exosporium of an endospore produced by the respective Brevibacillus, Lysinibacillus, Viridibacillus, or Paenibacillus cell; wherein the N-terminal targeting sequence also localizes the fusion protein to an exosporium of an endospore produced by a second cell (e.g., a Brevibacillus, Lysinibacillus, Viridibacillus, or Paenibacillus cell), that is a member a bacterial genus different from that of the recombinant exosporium-producing Brevibacillus, Lysinibacillus, Viridibacillus, or Paenibacillus cell. For example, in some aspects the N-terminal targeting sequence may be capable of localizing the fusion protein to the exosporium when expressed in members of the genus Brevibacillus and members of the genus Lysinibacillus, or in members of the genus Paenibacillus and members of the genus Viridibacillus. In some aspects, the N-terminal targeting sequence may be capable of localizing the fusion protein to the exosporium when expressed in members of any three, or all four, of these genera (i.e., when expressed in members of Brevibacillus, Lysinibacillus, Viridibacillus, and/or Paenibacillus). In still further aspects, the N-terminal targeting sequence may be capable of localizing the fusion protein to the exosporium when expressed in two or more specific members of these genera (e.g., any of the species described herein).

Compositions comprising the recombinant cells or fusion proteins described herein may further include additional components (e.g., that promote plant growth and/or health). Moreover, particular embodiments of the methods disclosed herein provide for an efficient high-throughput screening of heterologous proteins and peptide that confer or otherwise modify plant traits or attributes. When reference is made in this disclosure to N-terminal signal peptides (or targeting sequences), it is understood that such sequences must retain exosporium-targeting functionality in two or more of Brevibacillus, Lysinibacillus, Viridibacillus, and/or Paenibacillus (i.e., allowing for intergeneric use of such sequences). Exemplary N-terminal targeting sequences compatible with Brevibacillus are shown in Table 1 and FIGS. 1-3, exemplary N-terminal targeting sequences compatible with Lysinibacillus are shown in Table 2 and FIGS. 4-5, exemplary N-terminal targeting sequences compatible with Viridibacillus are shown in Table 3 and FIG. 6, and exemplary N-terminal targeting sequences compatible with Paenibacillus are shown in Table 4 and FIG. 7. Variants and fragments of these sequences that retain N-terminal targeting functionality in each of these respective bacterial genera are also disclosed herein. As demonstrated by experimental data disclosed herein (see, e.g., Table 5), the N-terminal targeting sequences described herein retain exosporium-targeting functionality in multiple genera, allowing for the use of such sequences in fusion protein constructs intended for expression in multiple genera (e.g., in Brevibacillus, Lysinibacillus, Viridibacillus, and/or Paenibacillus). In some aspects described herein, such N-terminal targeting sequences may be used in constructs that can be expressed in two or more particular species of these genera.

In another aspect, the disclosure provides a nucleic acid molecule encoding a fusion protein, comprising (a) a first polynucleotide sequence encoding an N-terminal signal peptide, operably linked to (b) a second polynucleotide sequence encoding a polypeptide heterologous to the N-terminal signal peptide, wherein the first polynucleotide sequence comprises: (i) a polynucleotide sequence having at least 60%, 70%, 80, 90, or 95% sequence identity with any of the polynucleotide sequences disclosed in Tables 1-4; or (ii) a polynucleotide sequence comprising a fragment of at least 30, 45, or 60 consecutive nucleotides of any of the polynucleotide sequences disclosed in Tables 1-4; and wherein the N-terminal signal peptide is capable of targeting the fusion protein to an exosporium when expressed in at least two different genera (e.g., allowing for intergeneric use in Brevibacillus, Lysinibacillus, Viridibacillus, and/or Paenibacillus cells or endospores). In some aspects, these fusion proteins may be localized to the exosporium when expressed in members of any three, or all four, of these genera. In still further aspects, such fusion proteins may be localized to the exosporium when expressed in two or more particular species of these genera, as described in further detail herein.

In selected aspects, the polypeptide heterologous to the N-terminal signal peptide comprises: (a) at least one of a plant growth or immune stimulating protein; (b) an enzyme; (c) a protein; (d) a polypeptide heterologous to Brevibacillus, Lysinibacillus, Viridibacillus, and/or Paenibacillus; or (e) a therapeutic protein. In selected aspects, the nucleic acid molecule further comprises a third polynucleotide sequence, encoding: (a) a polypeptide comprising one or more protease cleavage sites, wherein the polypeptide is positioned between the N-terminal signal peptide and the polypeptide heterologous to the N-terminal signal peptide; (b) a polypeptide comprising a selectable marker; (c) a polypeptide comprising a visualization marker; (d) a polypeptide comprising a protein recognition/purification domain; or (e) a polypeptide comprising a flexible linker element, which connects the N-terminal signal peptide and the polypeptide heterologous to the N-terminal signal peptide.

In selected aspects, the Brevibacillus endospore is an endospore formed by a Brevibacillus species, comprising: B. agri, B. aydinogluensis, B. borstelensis, B. brevis, B. centrosporus, B. choshinensis, B. fluminis, B. formosus, B. fulvus, B. ginsengisoli, B. invocatus, B. laterosporus, B. levickii, B. limnophilus, B. massiliensis, B. nitrificans, B. panacihumi, B. parabrevis, B. reuszeri, or B. thermoruber; or an endospore formed by a bacterium that possesses a 16S rRNA gene that shares at least 97%, 98% or 99% identity with a 16S rRNA gene of a Brevibacillus species.

In selected aspects, the Lysinibacillus endospore is an endospore formed by a Lysinibacillus species, comprising: Lysinibacillus sphaericus, Lysinibacillus boronitolerans, Lysinibacillus fusiformis, Lysinibacillus acetophenoni, Lysinibacillus alkaliphilus, Lysinibacillus chungkukjangi, Lysinibacillus composti, Lysinibacillus contaminans, Lysinibacillus cresolivorans, Lysinibacillus macroides, Lysinibacillus manganicus, Lysinibacillus mangiferihumi, Lysinibacillus massiliensis, Lysinibacillus meyeri, Lysinibacillus odysseyi, Lysinibacillus pakistanensis, Lysinibacillus parviboronicapiens, Lysinibacillus sinduriensis, Lysinibacillus tabacifolii, Lysinibacillus varians, Lysinibacillus xylanilyticus, or Lysinibacillus halotolerans; or an endospore formed by a bacterium that possesses a 16S rRNA gene that shares at least 97%, 98% or 99% identity with a 16S rRNA gene of a Lysinibacillus species.

In select aspects, the Viridibacillus endospore is an endospore formed by a Viridibacillus species, comprising: Viridibacillus arvi, Viridibacillus arenosi, or Viridibacillus neidei; or an endospore formed by a bacterium that possesses a 16S rRNA gene that shares at least 97%, 98% or 99% identity with a 16S rRNA gene of a Viridibacillus species.

In select aspects, the Paenibacillus endospore is an endospore formed by a Paenibacillus species, comprising: Paenibacillus sp. NRRL B-50972, Paenibacillus terrae, Paenibacillus polymyxa, or Paenibacillus peoriae; or an endospore formed by a bacterium that possesses a 16S rRNA gene that shares at least 97%, 98% or 99% identity with a 16S rRNA gene of a Paenibacillus species.

In selected aspects, the nucleic acid molecule is operatively linked to a promoter element that is heterologous to at least one of the second polynucleotide sequence and Brevibacillus, Lysinibacillus, Viridibacillus, and/or Paenibacillus.

In selected aspects, the polypeptide heterologous to the N-terminal signal peptide comprises: (a) at least one of a plant growth or immune stimulating protein; (b) an enzyme; (c) a polypeptide heterologous to Brevibacillus, Lysinibacillus, Viridibacillus, and/or Paenibacillus; (d) a therapeutic protein (e.g., an antibiotic or anti-inflammatory protein); or (e) a protein that provides an agriculturally-significant property, included, but not limited to: insecticidal activity, fungicidal activity, plant growth, health or immune-stimulating activity, and/or improved environmental resistance. Other agriculturally-significant properties include improved crop characteristics including: emergence, crop yields, protein content, oil content, starch content, more developed root system, improved root growth, improved root size maintenance, improved root effectiveness, improved stress tolerance (e.g., against drought, heat, salt, UV, water, cold), reduced ethylene (reduced production and/or inhibition of reception), tillering increase, increase in plant height, bigger leaf blade, less dead basal leaves, stronger tillers, greener leaf color, pigment content, photosynthetic activity, less input needed (such as fertilizers or water), less seeds needed, more productive tillers, earlier flowering, early grain maturity, less plant verse (lodging), increased shoot growth, enhanced plant vigor, increased plant stand and early and better germination.

In selected aspects, the fusion protein further comprises: (a) a polypeptide containing one or more protease cleavage sites, positioned between the N-terminal signal peptide and the polypeptide heterologous to the N-terminal signal peptide; (b) a polypeptide comprising a selectable marker (e.g., a protein that confers resistance to an antibiotic); (c) a polypeptide comprising a visualization element (e.g., a fluorescent tag such as GFP); (d) a polypeptide comprising at least one protein recognition/purification domain (e.g., a HIS-tag); or (e) a polypeptide comprising a flexible linker element, connecting the signal peptide and the polypeptide heterologous to the N-terminal signal peptide.

In an alternative aspect, the disclosure provides a recombinant Brevibacillus, Lysinibacillus, Viridibacillus, or Paenibacillus cell comprising a bacterial chromosome comprising a nucleic acid molecule of any one of the aspects disclosed herein.

In an alternative aspect, the disclosure provides a vector comprising a nucleic acid molecule of any one of the aspects disclosed herein, wherein the vector comprises a plasmid, an artificial chromosome, or a viral vector.

In selected aspects, the vector further comprising at least one of the following: (a) an origin of replication that provides stable maintenance in at least two of a Brevibacillus, a Lysinibacillus, a Viridibacillus, and/or a Paenibacillus cell; (b) an origin of replication that provides selectively non-stable maintenance in at least two of a Brevibacillus, a Lysinibacillus, a Viridibacillus, and/or a Paenibacillus cell; (c) a temperature-sensitive origin of replication that provides selectively non-stable maintenance in at least two of a Brevibacillus, a Lysinibacillus, a Viridibacillus, and/or a Paenibacillus cell; (d) a polynucleotide encoding a selection marker, operably linked to an expression control sequence; or (e) a polynucleotide encoding a plant growth stimulating protein, operably linked to an expression control sequence;

In alternative aspects, the disclosure provides a recombinant Brevibacillus, Lysinibacillus, Viridibacillus, or Paenibacillus cell transformed with a vector comprising the nucleic acid molecule of any one of the aspects disclosed herein.

In selected aspects, the Brevibacillus cell is a Brevibacillus species, comprising: B. agri, B. aydinogluensis, B. borstelensis, B. brevis, B. centrosporus, B. choshinensis, B. fluminis, B. formosus, B. fulvus, B. ginsengisoli, B. invocatus, B. laterosporus, B. levickii, B. limnophilus, B. massiliensis, B. nitrificans, B. panacihumi, B. parabrevis, B. reuszeri, or B. thermoruber.

In selected aspects, the Lysinibacillus cell is a Lysinibacillus species, comprising: Lysinibacillus sphaericus, Lysinibacillus boronitolerans, Lysinibacillus fusiformis, Lysinibacillus acetophenoni, Lysinibacillus alkaliphilus, Lysinibacillus chungkukjangi, Lysinibacillus composti, Lysinibacillus contaminans, Lysinibacillus cresolivorans, Lysinibacillus macroides, Lysinibacillus manganicus, Lysinibacillus mangiferihumi, Lysinibacillus massiliensis, Lysinibacillus meyeri, Lysinibacillus odysseyi, Lysinibacillus pakistanensis, Lysinibacillus parviboronicapiens, Lysinibacillus sinduriensis, Lysinibacillus tabacifolii, Lysinibacillus varians, Lysinibacillus xylanilyticus, or Lysinibacillus halotolerans.

In selected aspects, the Viridibacillus cell is a Viridibacillus species, comprising: Viridibacillus arvi, Viridibacillus arenosi, or Viridibacillus neidei.

In selected aspects, the Paenibacillus cell is a Paenibacillus species, comprising: Paenibacillus sp. NRRL B-50972, Paenibacillus terrae, Paenibacillus polymyxa, or Paenibacillus peoriae.

In alternative aspects, the disclosure provides a composition comprising: a) one or more recombinant exosporium-producing Brevibacillus, Lysinibacillus, Viridibacillus, and/or Paenibacillus cells that express at least one fusion protein according to any of the aspects disclosed herein, wherein the polypeptide heterologous to the N-terminal signal peptide comprises a plant growth or immune stimulating protein; and b) at least one biological control agent; optionally, in a synergistically effective amount.

In alternative aspects, the disclosure provides a seed treated with at least one of the nucleic acids, fusion proteins, bacterial cells or compositions of any one of the aspects disclosed herein.

In alternative aspects, the disclosure provides a method of treating a plant, a seed, a plant part, or the soil surrounding the plant to enhance plant growth and/or promote plant health comprising the step of simultaneously or sequentially applying: a) recombinant exosporium-producing Brevibacillus, Lysinibacillus, Viridibacillus, and/or Paenibacillus endospores that express the fusion protein of any of the aspects disclosed herein, wherein the polypeptide heterologous to the N-terminal signal peptide comprises a plant growth or immune stimulating protein; and b) at least one biological control agent; optionally, in a synergistically effective amount.

In alternative aspects, the disclosure provides a method of screening a host plant treated with recombinant Brevibacillus, Lysinibacillus, Viridibacillus, and/or Paenibacillus endospores, comprising the following steps: a) applying a composition comprising Brevibacillus, Lysinibacillus, Viridibacillus, and/or Paenibacillus endospores modified to express (or produced by a cell expressing) a fusion protein according to any of the aspects disclosed herein, to a seed, a seedling, or a vegetative plant capable of being permanently or transiently colonized by a Brevibacillus, Lysinibacillus, Viridibacillus, and/or Paenibacillus, to produce a treated seed, seedling, or vegetative plant; b) screening the treated seed, seedling, or vegetative plant by detecting and optionally measuring a trait, component, or attribute of the treated seed, seedling, or vegetative plant.

In selected aspects, the screening step comprises one or more of the following: (a) at least one in vitro assay comprising detecting and optionally quantifying the presence, level, change in level, activity, or localization of one or more compounds contained in an extract prepared from a cell or tissue sample obtained from the treated seed, seedling, or vegetative plant; and/or (b) at least one in vivo assay comprising detecting and optionally quantifying a trait, component, or attribute of the treated seed, seedling, or vegetative plant.

In alternative aspects, the disclosure provides a method of screening heterologous proteins or peptides expressed in a Brevibacillus, Lysinibacillus, Viridibacillus, or Paenibacillus cell for agriculturally-significant properties, comprising: (a) modifying a Brevibacillus, Lysinibacillus, Viridibacillus, or Paenibacillus cell to express a fusion protein according to any of the aspects disclosed herein to produce a recombinant Brevibacillus, Lysinibacillus, Viridibacillus, or Paenibacillus cell; and (b) screening the Brevibacillus, Lysinibacillus, Viridibacillus, or Paenibacillus cell by detecting and optionally quantifying a level or activity of a compound produced by the recombinant Brevibacillus, Lysinibacillus, Viridibacillus, or Paenibacillus cell.

In alternative aspects, the disclosure provides a method of treating a plant, a plant seed, a human, or an animal, comprising: administering to the plant, plant seed, human, or animal a composition comprising an exosporium isolated from an endospore produced by a recombinant Brevibacillus, Lysinibacillus, Viridibacillus, or Paenibacillus cell; wherein the recombinant Brevibacillus, Lysinibacillus, Viridibacillus, or Paenibacillus cell expresses the fusion protein of any one of the aspects disclosed herein. In selected aspects, the animal may include animals raised as livestock, such as cattle.

In selected aspects, the composition has been heat-inactivated or sterilized such that no viable Brevibacillus, Lysinibacillus, Viridibacillus, and/or Paenibacillus cells remain.

In alternative aspects, the disclosure provides a composition comprising an isolated and/or purified fusion protein according to any one of the aspects disclosed herein.

In alternative aspects, the disclosure provides a composition comprising an isolated and/or purified exosporium produced by recombinant Brevibacillus, Lysinibacillus, Viridibacillus, and/or Paenibacillus endospore(s), which has been modified to express a fusion protein according to any of the aspects disclosed herein.

In alternative aspects, the disclosure provides a composition comprising an exosporium produced by recombinant Brevibacillus, Lysinibacillus, Viridibacillus, and/or Paenibacillus endospore(s), wherein the recombinant endospore(s) have been modified to express (or were produced by one or more cells expressing) a fusion protein according to any of the aspects disclosed herein.

In selected aspects, the exosporium produced by a recombinant Brevibacillus, Lysinibacillus, Viridibacillus, or Paenibacillus endospore comprises: (a) a basal layer of an exosporium; (b) a hair-like layer of an exosporium; (c) a mixture of both (a) and (b); (d) a fraction or extract of a crude exosporium obtained from a Brevibacillus, Lysinibacillus, Viridibacillus, or Paenibacillus endospore; and/or (e) a fraction or extract of a crude exosporium obtained from a Brevibacillus, Lysinibacillus, Viridibacillus, or Paenibacillus endospore that is enriched in an amount or concentration of the fusion protein compared to a same amount of the crude exosporium.

In alternative aspects, the disclosure provides a method of delivering a protein of interest to a plant, seed or field, comprising: applying a composition comprising an exosporium obtained from recombinant Brevibacillus, Lysinibacillus, Viridibacillus, and/or Paenibacillus endospore(s) to a plant, seed, or field; wherein the recombinant Brevibacillus, Lysinibacillus, Viridibacillus, and/or Paenibacillus endospore(s) have been modified to express (or were produced by one or more cells expressing) a fusion protein according to any of the aspects disclosed herein.

In selected aspects, the composition is applied to a field: (a) pre- or post-planting; (b) pre- or post-emergence; (c) as a powder, suspension or solution; or (d) wherein the composition further comprises one or more additional compounds that stimulate plant growth.

### BRIEF DESCRIPTION OF THE DRAWINGS

FIG. 1 shows a first sequence alignment of the amino acid sequences of the N-terminal portion of various Brevibacillus proteins that possess variants of an N-terminal signal peptide according to an exemplary aspect of the disclosure. A consensus sequence is shown below the alignment.

FIG. 2 shows a second sequence alignment of the amino acid sequences of the N-terminal portion of various Brevibacillus proteins that possess variants of an N-terminal signal peptide according to an exemplary aspect of the disclosure. A consensus sequence is shown below the alignment.

FIG. 3 shows a third sequence alignment of the amino acid sequences of the N-terminal portion of various Brevibacillus proteins that possess variants of an N-terminal signal peptide according to an exemplary aspect of the disclosure. A consensus sequence is shown below the alignment.

FIG. 4 shows a sequence alignment of the amino acid sequences of the N-terminal portion of various Lysinibacillus proteins that possess variants of an N-terminal signal peptide according to an exemplary aspect of the disclosure. A consensus sequence (SEQ ID NO: 41) is shown below the alignment.

FIG. 5 shows a sequence alignment of the amino acid sequences of the N-terminal portion of various Lysinibacillus proteins that possess variants of an N-terminal signal peptide according to another exemplary aspect of the disclosure. A consensus sequence is shown below the alignment.

FIG. 6 shows a sequence alignment of the amino acid sequences of the N-terminal portion of various proteins identified in Viridibacillus sp. strains (e.g., SEQ ID NOs: 181, 185, 189, 193, 197, 201, 205, 209, 213, and 217) that possess variants of an N-terminal signal peptide according to an exemplary aspect of the disclosure. A consensus sequence (i.e., SEQ ID NO: 179) is shown below the alignment. Several groups of Viridibacillus sp. strains expressed proteins having an identical N-terminal signal peptide.

FIG. 7 shows a sequence alignment of the amino acid sequences of exemplary Paenibacillus N-terminal signal peptides according to an aspect of the disclosure. A consensus sequence is shown below the alignment.

FIG. 8A shows a brightfield (left) and epifluorescent (right) micrographs (1000× magnification) of a Brevibacillus sp. NRRL B-67865 endospore expressing an exemplary N-terminal targeting sequence according to the disclosure, specifically a tdTomato fusion protein construct which is localized to the endospore surface as illustrated by this figure. The fluorescence produced by tdTomato in the right panel corresponds with the image of the spores observed with brightfield microscopy in the left panel indicating correct localization of the tdTomato to the endospore surface.

FIG. 8B shows a flow cytometry histogram of Brevibacillus sp. NRRL B-67865 endospores expressing the same exemplary N-terminal targeting sequence used to transform the endospores shown in FIG. 8A. Wild-type Brevibacillus sp. NRRL B-67865 endospores with no observable tdTomato fluorescence are shown for comparison (dotted line trace, open area). 50,000 events are recorded for each population shown on the figure.

FIG. 9A shows a brightfield (left) and epifluorescent (right) micrographs (1000× magnification) of a Lysinibacillus sp. NRRL B-67864 endospore expressing an exemplary N-terminal targeting sequence according to the disclosure, specifically a tdTomato fusion protein construct which is localized to the endospore surface as illustrated by this figure. The fluorescence produced by tdTomato in the right panel corresponds with the image of the spores observed with brightfield microscopy in the left panel indicating correct localization of the tdTomato to the endospore surface.

FIG. 9B shows a flow cytometry histogram of Lysinibacillus sp. NRRL B-67864 endospores expressing the same exemplary N-terminal targeting sequence used to transform the endospores shown in FIG. 9A. Wild-type Lysinibacillus sp. NRRL B-67864 endospores with no observable tdTomato fluorescence are shown for comparison (dotted line trace, open area). 50,000 events are recorded for each population shown on the figure.

FIG. 10A shows brightfield (left) and epifluorescent (right) micrographs (1000× magnification) of a Viridibacillus sp. NRRL B-67869 endospore expressing an exemplary N-terminal targeting sequence according to the disclosure, specifically a tdTomato fusion protein construct which is localized to the endospore surface as illustrated by this figure. The fluorescence produced by tdTomato in the right panel corresponds with the image of the spores observed with brightfield microscopy in the left panel indicating correct localization of the tdTomato to the endospore surface.

FIG. 10B shows a flow cytometry histogram of Viridibacillus sp. NRRL B-67869 endospores expressing the same exemplary N-terminal targeting sequence used to transform the endospores shown in FIG. 10A. Wild-type Viridibacillus sp. NRRL B-67869 endospores with no observable tdTomato fluorescence are shown for comparison (dotted line trace, open area). 50,000 events are recorded for each population shown on the figure.

FIG. 11 depicts a transmission electron micrograph of a Paenibacillus sp. NRRL B-50972 endospore. Hair-like structures comprised of collagen-like protein are shown extending from the endospore surface and one such structure is denoted by an arrow.

FIG. 12A depicts phase contrast (left) and epifluorescent (right) micrographs (1000× magnification) of a Paenibacillus sp. NRRL B-50972 endospore expressing an exemplary N-terminal targeting sequence according to the disclosure, specifically a GFP fusion protein construct which is localized to the endospore surface as shown by this figure. The fluorescence produced by the GFP protein in the right panel corresponds with the image of the cell observed with phase contrast microscopy in the left panel indicating correct localization of the GFP to the endospore surface.

FIG. 12B depicts a flow cytometry histogram of Paenibacillus sp. NRRL B-50972 endospores expressing the same exemplary N-terminal targeting sequence used to transform the endospores shown in FIG. 12A, specifically a GFP fusion protein construct, which is localized to the endospore surface (shaded area). Wild-type Paenibacillus sp. NRRL B-50972 endospores with no observable GFP fluorescence are shown for comparison (open, dotted line area). 10,000 events are shown for each spore population on this figure.

### DETAILED DESCRIPTION

The disclosure provides genetic constructs capable of targeting a fusion protein to an exosporium when expressed in different genera (e.g., in at least two of Brevibacillus, Lysinibacillus, Viridibacillus, and/or Paenibacillus), as well as compositions and methods that use these intergeneric constructs to deliver heterologous molecules of interest (e.g., peptides or proteins) to various environments, such as plants. For example, following treatment with the recombinant Brevibacillus, Lysinibacillus, Viridibacillus, and/or Paenibacillus endospores described herein, treated plants may be screened to detect changes attributable to the heterologous protein delivered via the Brevibacillus, Lysinibacillus, Viridibacillus, and/or Paenibacillus endospores. Such changes may include alterations in the host plant's growth rate or yield; enhanced plant health (e.g., resistance to environmental stress, disease or pests); and the display of enhanced, modified or otherwise new attributes, compared to host plants grown under the same conditions absent treatment with the recombinant Brevibacillus, Lysinibacillus, Viridibacillus, and/or Paenibacillus endospores. The use of a targeting sequence that efficiently targets the heterologous protein to the exosporium also provides a platform for high-throughput screening for useful heterologous proteins that, for example, are capable of enhancing, modifying, and/or conferring new plant traits or attributes.

Brevibacillus, Lysinibacillus, Viridibacillus, and Paenibacillus bacteria produce endospores that contain an exosporium layer. This structure is absent in B. subtilis, which produces endospores that terminate in an outer spore coat. The canonical spore formation process (elucidated based on studies using B. subtilis) involves asymmetric cell division of a vegetative cell to form a mother cell and a forespore, which develop as two distinct compartments separated by an intervening septum. Eventually, the peptidoglycan in the septum is degraded and the forespore is engulfed by the mother cell, forming a cell within a cell. Intercellular communication between the mother cell and forespore coordinates cell-specific gene expression in each cell, resulting in the production of endospore-specific compounds, formation of a cortex layer around the forespore and deposition of the coat.

In some Bacillus species, e.g., B. subtilis, B. licheniformis, and B. pumilus, this coat will go on to become the outermost layer of the endospore. In many species of the B. cereus group, the forespore is further enclosed by a loose-fitting and balloon-like exosporium composed of a paracrystalline basal layer surrounded by a hair-like nap layer. In these species, the exosporium is separated from the coat by an interstitial connecting region known as the interspace. In either case, after the formation of a terminal coat layer or exosporium, the forespore undergoes a final dehydration and maturation into a complete endospore. The mother cell is subsequently degraded via programmed cell death, resulting in a release of the endospore into the environment. The endospore will then typically remain in a dormant state until more favorable conditions or particular stimuli trigger germination and a return to the vegetative state.

As the outermost surface between the spore and the environment, the coat layer (or exosporium, in exosporium-forming species) serves many critical functions. In particular, this layer acts as a semipermeable barrier to environmental insults and mediates interactions with the soil, and thus plays an important role in maintaining the viability of the spore and in the sensing of conditions that trigger germination of the endospore. The coat layer is also a target of clinical research as it contains cell surface molecules in pathogenic strains of bacteria that contribute to host immune cell recognition. Methods of displaying heterologous proteins on the spore coat of B. subtilis have been developed using fusion protein constructs containing a B. subtilis spore coat protein such as CotC fused to a protein of interest. However, B. subtilis lacks an exosporium and thus studies using this species fail to provide guidance as to how fusion proteins may be targeted to the exosporium produced by other bacterial genera, such as Brevibacillus, Lysinibacillus, Viridibacillus, or Paenibacillus.

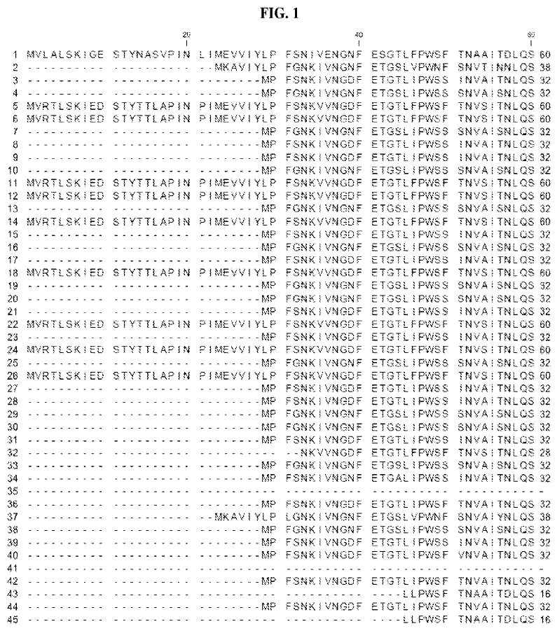

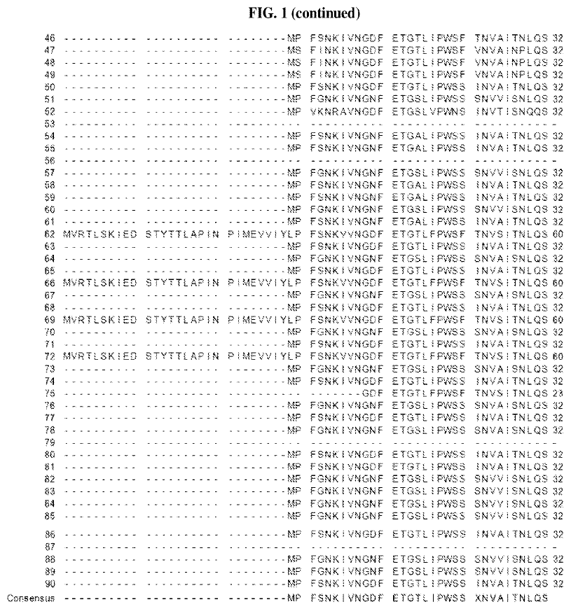

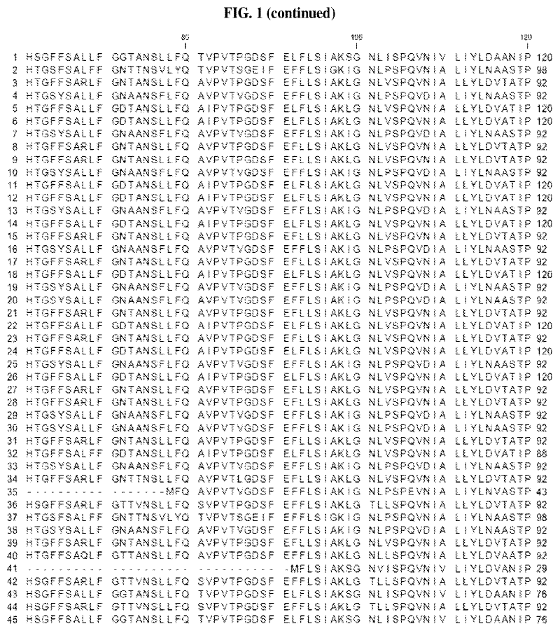

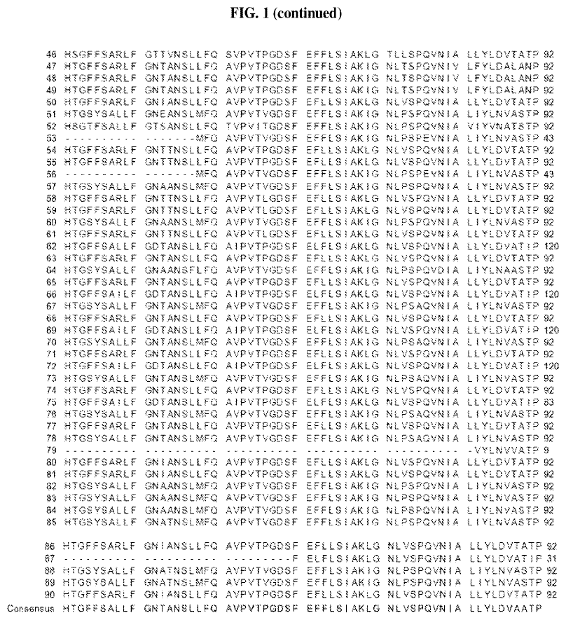

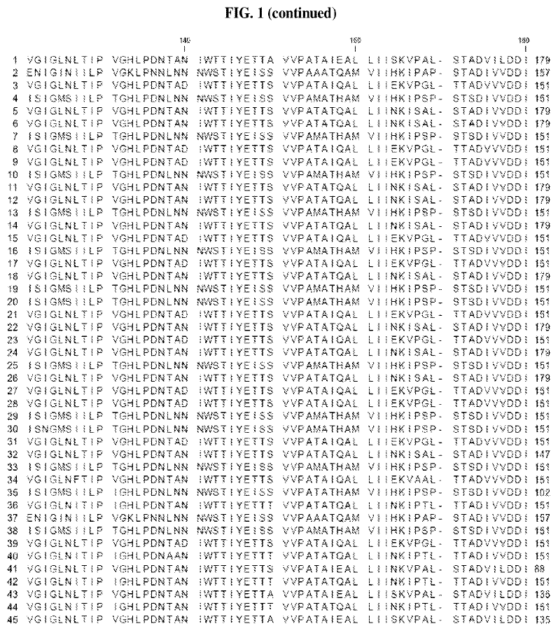

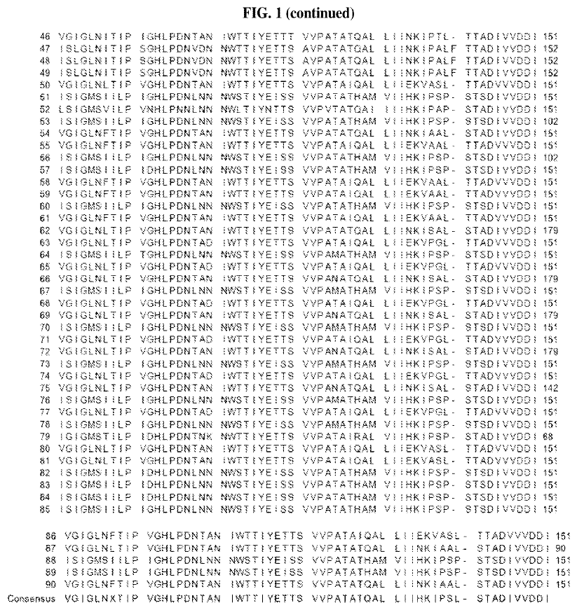

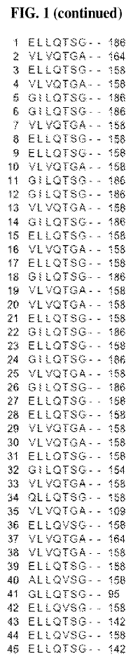

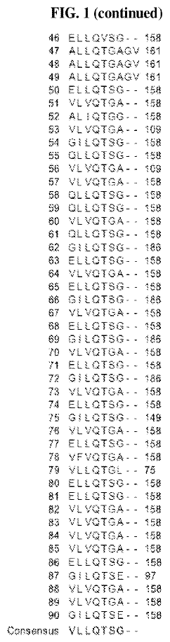

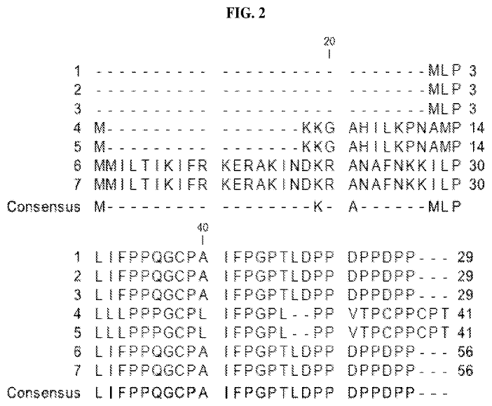

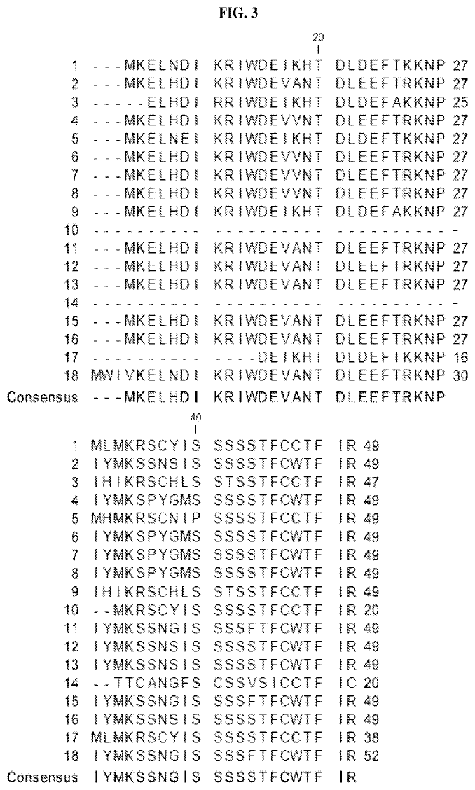

In contrast, the disclosure provides N-terminal targeting sequences and fusion proteins comprising the same that are capable of targeting fusion protein constructs to the exosporium of members of multiple genera (e.g., in Brevibacillus, Lysinibacillus, Viridibacillus, and/or Paenibacillus cells). The N-terminal signal sequence used to target the fusion protein to the exosporium may comprise a polypeptide having a sequence represented by any of the sequences disclosed herein, provided that such sequence is compatible with the selected bacterial genera (e.g., a sequence shown in Tables 1-4 or FIGS. 1-7, or a fragment or variant of any of these sequences which retains exosporium-targeting functionality in the selected bacterial genera). In select embodiments, this N-terminal signal sequence may comprise a fragment or variant of any of the sequences disclosed herein, sufficient to retain exosporium-targeting functionality in the same bacterial genus. These and other embodiments are described herein.

Throughout the disclosure, the term “comprise” or any derivative thereof (e.g., comprising, comprises) may be replaced with “consist essentially of”, “consist of”, or the applicable corresponding derivative thereof.

As used herein, “Brevibacillus” refers to endospore-producing bacteria classified in the Brevibacillus genus. This term encompasses, without limitation, various Brevibacillus family members including B. agri, B. aydinogluensis, B. borstelensis, B. brevis, B. centrosporus, B. choshinensis, B. fluminis, B. formosus, B. fulvus, B. ginsengisoli, B. invocatus, B. laterosporus, B. levickii, B. limnophilus, B. massiliensis, B. nitrificans, B. panacihumi, B. parabrevis, B. reuszeri, or B. thermoruber.

As used herein, “Lysinibacillus” refers to endospore-producing bacteria classified in the Lysinibacillus genus. This term encompasses, without limitation, various Lysinibacillus family members including Lysinibacillus sphaericus, Lysinibacillus boronitolerans, Lysinibacillus fusiformis, Lysinibacillus acetophenoni, Lysinibacillus alkaliphilus, Lysinibacillus chungkukjangi, Lysinibacillus composti, Lysinibacillus contaminans, Lysinibacillus cresolivorans, Lysinibacillus macroides, Lysinibacillus manganicus, Lysinibacillus mangiferihumi, Lysinibacillus massiliensis, Lysinibacillus meyeri, Lysinibacillus odysseyi, Lysinibacillus pakistanensis, Lysinibacillus parviboronicapiens, Lysinibacillus sinduriensis, Lysinibacillus tabacifolii, Lysinibacillus varians, Lysinibacillus xylanilyticus and Lysinibacillus halotolerans.

As used herein, “Viridibacillus” refers to endospore-producing bacteria classified in the Viridibacillus genus. This term encompasses, without limitation, various Viridibacillus family members including Viridibacillus arvi, Viridibacillus arenosi, and Viridibacillus neidei.

As used herein, “Paenibacillus” refers to endospore-producing bacteria classified in the Paenibacillus genus. This term encompasses, without limitation, various Paenibacillus family members including Paenibacillus sp. NRRL B-50972, Paenibacillus terrae, Paenibacillus polymyxa, and Paenibacillus peoriae.

In other aspects, the Paenibacillus species comprises: Paenibacillus abyssi, Paenibacillus aceti, Paenibacillus aestuarii, Paenibacillus agarexedens, Paenibacillus agaridevorans, Paenibacillus alginolyticus, Paenibacillus algorifonticola, Paenibacillus alkaliterrae, Paenibacillus alvei, Paenibacillus amylolyticus, Paenibacillus anaericanus, Paenibacillus antarcticus, Paenibacillus apiarius, Paenibacillus arachidis, Paenibacillus assamensis, Paenibacillus azoreducens, Paenibacillus azotofixans, Paenibacillus baekrokdamisoli, Paenibacillus barcinonensis, Paenibacillus barengoltzii, Paenibacillus borealis, Paenibacillus bovis, Paenibacillus brasilensis, Paenibacillus camelliae, Paenibacillus campinasensis, Paenibacillus castaneae, Paenibacillus catalpae, Paenibacillus cathormii, Paenibacillus cavernae, Paenibacillus cellulosilyticus, Paenibacillus cellulositrophicus, Paenibacillus chartarius, Paenibacillus chibensis, Paenibacillus chinjuensis, Paenibacillus chitinolyticus, Paenibacillus chondroitinus, Paenibacillus chungangensis, Paenibacillus cineris, Paenibacillus cisolokensis, Paenibacillus contaminans, Paenibacillus cookii, Paenibacillus cucumis, Paenibacillus curdlanolyticus, Paenibacillus daejeonensis, Paenibacillus darwinianus, Paenibacillus dauci, Paenibacillus dendritiformis, Paenibacillus dongdonensis, Paenibacillus doosanensis, Paenibacillus durus, Paenibacillus edaphicus, Paenibacillus ehimensis, Paenibacillus elgii, Paenibacillus endophyticus, Paenibacillus etheri, Paenibacillus faecis, Paenibacillus favisporus, Paenibacillus ferrarius, Paenibacillus filicis, Paenibacillus fonticola, Paenibacillus forsythiae, Paenibacillus frigoriresistens, Paenibacillus gansuensis, Paenibacillus gelatinilyticus, Paenibacillus ginsengarvi, Paenibacillus ginsengihumi, Paenibacillus ginsengisoli, Paenibacillus glacialis, Paenibacillus glucanolyticus, Paenibacillus glycanilyticus, Paenibacillus gordonae, Paenibacillus graminis, Paenibacillus granivorans, Paenibacillus guangzhouensis, Paenibacillus harenae, Paenibacillus hemerocallicola, Paenibacillus hispanicus, Paenibacillus hodogayensis, Paenibacillus hordei, Paenibacillus humicus, Paenibacillus hunanensis, Paenibacillus illinoisensis, Paenibacillus jamilae, Paenibacillus jilunlii, Paenibacillus kobensis, Paenibacillus koleovorans, Paenibacillus konsidensis, Paenibacillus koreensis, Paenibacillus kribbensis, Paenibacillus kyungheensis, Paenibacillus lactis, Paenibacillus larvae, Paenibacillus larvae, Paenibacillus larvae, Paenibacillus lautus, Paenibacillus lemnae, Paenibacillus lentimorbus, Paenibacillus lentus, Paenibacillus liaoningensis, Paenibacillus lupini, Paenibacillus macerans, Paenibacillus macquariensis, Paenibacillus macquariensis, Paenibacillus macquariensis, Paenibacillus marchantiophytorum, Paenibacillus marinisediminis, Paenibacillus massiliensis, Paenibacillus medicaginis, Paenibacillus mendelii, Paenibacillus methanolicus, Paenibacillus montaniterrae, Paenibacillus motobuensis, Paenibacillus mucilaginosus, Paenibacillus nanensis, Paenibacillus naphthalenovorans, Paenibacillus nasutitermitis, Paenibacillus nematophilus, Paenibacillus nicotianae, Paenibacillus oceanisediminis, Paenibacillus odorifer, Paenibacillus oenotherae, Paenibacillus oryzae, Paenibacillus pabuli, Paenibacillus panacisoli, Paenibacillus panaciterrae, Paenibacillus pasadenensis, Paenibacillus pectinilyticus, Paenibacillus periandrae, Paenibacillus phoenicis, Paenibacillus phyllosphaerae, Paenibacillus physcomitrellae, Paenibacillus pini, Paenibacillus pinihumi, Paenibacillus pinesoli, Paenibacillus pocheonensis, Paenibacillus popilliae, Paenibacillus populi, Paenibacillus prosopidis, Paenibacillus provencensis, Paenibacillus pueri, Paenibacillus puldeungensis, Paenibacillus pulvifaciens, Paenibacillus purispatii, Paenibacillus qingshengii, Paenibacillus quercus, Paenibacillus radicis, Paenibacillus relictisesami, Paenibacillus residui, Paenibacillus rhizoryzae, Paenibacillus rhizosphaerae, Paenibacillus rigui, Paenibacillus riograndensis, Paenibacillus ripae, Paenibacillus sabinae, Paenibacillus sacheonensis, Paenibacillus salinicaeni, Paenibacillus sanguinis, Paenibacillus sediminis, Paenibacillus segetis, Paenibacillus selenii, Paenibacillus selenitireducens, Paenibacillus senegalensis, Paenibacillus septentrionalis, Paenibacillus sepulcri, Paenibacillus shenyangensis, Paenibacillus shirakamiensis, Paenibacillus siamensis, Paenibacillus silagei, Paenibacillus sinopodophylli, Paenibacillus solani, Paenibacillus soli, Paenibacillus sonchi, Paenibacillus sophorae, Paenibacillus sputi, Paenibacillus stellifer, Paenibacillus susongensis, Paenibacillus swuensis, Paenibacillus taichungensis, Paenibacillus taiwanensis, Paenibacillus tarimensis, Paenibacillus telluris, Paenibacillus terreus, Paenibacillus terrigena, Paenibacillus thailandensis, Paenibacillus thermophilus, Paenibacillus thiaminolyticus, Paenibacillus tianmuensis, Paenibacillus tibetensis, Paenibacillus timonensis, Paenibacillus tundrae, Paenibacillus turicensis, Paenibacillus typhae, Paenibacillus uliginis, Paenibacillus urinalis, Paenibacillus validus, Paenibacillus vini, Paenibacillus vulneris, Paenibacillus wenxiniae, Paenibacillus wooponensis, Paenibacillus woosongensis, Paenibacillus wulumuqiensis, Paenibacillus wynnii, Paenibacillus xanthinilyticus, Paenibacillus xinjiangensis, Paenibacillus xylanexedens, Paenibacillus xylanilyticus, Paenibacillus xylanisolvens, Paenibacillus yonginensis, Paenibacillus yunnanensis, Paenibacillus zanthoxyli, or Paenibacillus zeae.

In certain aspects, the Brevibacillus member used to express the fusion protein is Brevibacillus brevis (formerly classified as Bacillus brevis), the Lysinibacillus member used to express the fusion protein is Lysinibacillus sphaericus (formerly classified as Bacillus sphaericus), the Viridibacillus member used to express the fusion protein is Viridibacillus arvi (formerly classified as Bacillus arvi), the Paenibacillus member used to express the fusion protein is Paenibacillus sp. NRRL B-50972. Each of these bacterial species is a Gram-positive, aerobic, and spore-forming bacterium commonly isolated from soils.

Characterization of a bacterium as a Bacillus brevis, Bacillus sphaericus, or Bacillus arvi was previously based solely on simple morphological features and a limited number of biochemical tests. However, recent genomics studies have revealed that several members of the Bacillus genus are quite distinct at the DNA level, resulting in reevaluation of the taxonomic position of multiple species, including the species now identified as B. brevis, L. sphaericus and V. arvi. Strikingly, both 16S rRNA and whole genome analysis of this genus reveals that it is a member of the Planococcaceae family rather than the Bacillaceae family, as it was originally classified. Previous studies have indicated that Brevibacillus, Lysinibacillus, and Viridibacillus spores possess an exosporium layer. However, unlike the exosporium in the B. cereus group, comparatively little is known about the composition and structure of the Brevibacillus, Lysinibacillus, Viridibacillus, or Paenibacillus exosporium. Given the general lack of knowledge about the basic composition or structure of the Brevibacillus, Lysinibacillus, Viridibacillus, or Paenibacillus exosporium, little is known about the process by which proteins are targeted to the exosporium of members of these genera during formation of this layer.

In certain aspects, the Brevibacillus, Lysinibacillus, Viridibacillus, or Paenibacillus member used to express the fusion protein is a bacterium that possesses a 16S rRNA gene that shares at least 97%, 98% or 99% identity with a 16S rRNA gene of B. brevis, L. sphaericus, V. arvi, Paenibacillus sp. NRRL B-50972, or any of the other exemplary Brevibacillus, Lysinibacillus, Viridibacillus, or Paenibacillus family members disclosed herein. Alternatively, the Brevibacillus, Lysinibacillus, Viridibacillus, or Paenibacillus member used to express the fusion protein is a bacterium that possesses a DNA-DNA hybridization value of at least 70% to that of B. brevis, L. sphaericus, V. arvi, Paenibacillus sp. NRRL B-50972, or any of the other exemplary Brevibacillus, Lysinibacillus, Viridibacillus, or Paenibacillus family members disclosed herein. In another instance, the Brevibacillus, Lysinibacillus, Viridibacillus, or Paenibacillus member used to express the fusion protein is a bacterium that possesses an average nucleotide identity of 95%, 96%, 97%, 98%, or 99% to that of B. brevis, L. sphaericus, V. arvi, Paenibacillus sp. NRRL B-50972, or any of the other exemplary Brevibacillus, Lysinibacillus, Viridibacillus, or Paenibacillus family members disclosed herein.

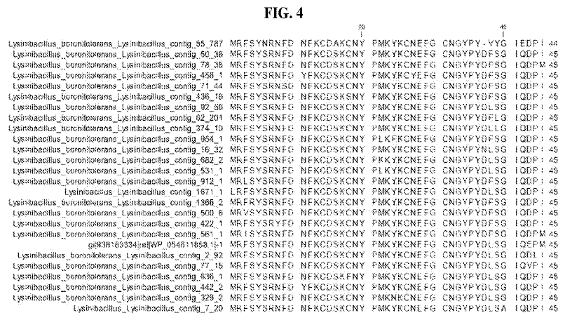

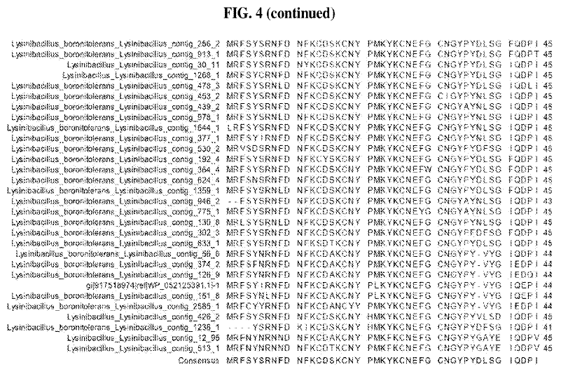

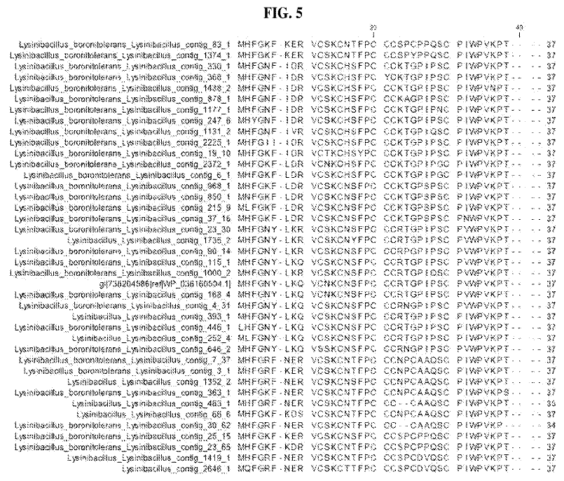

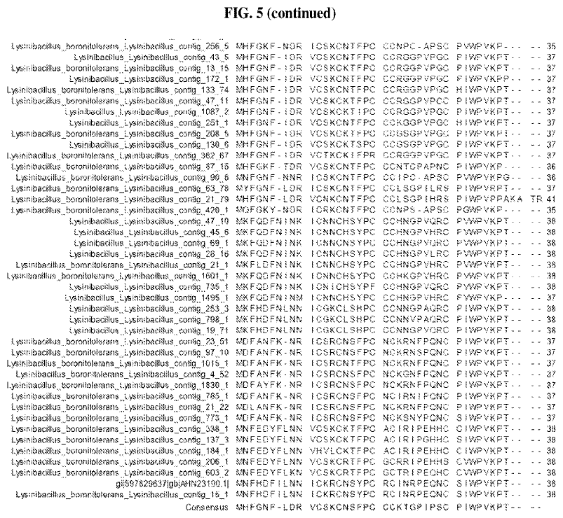

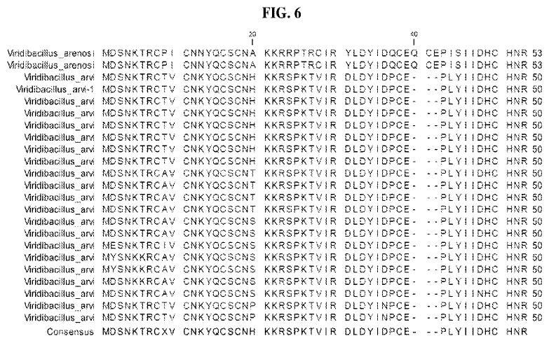

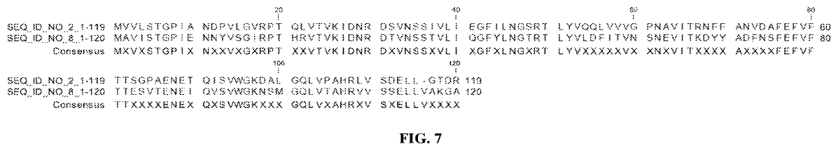

The term “N-terminal signal sequence” generally refers to a polypeptide sequence located at or proximal to the amino terminus of a polypeptide, which directs localization of the polypeptide to a subcellular compartment, or for secretion. It is recognized and understood that this term may be used interchangeably with the terms “N-terminal targeting sequence,” “targeting sequence,” “signal sequence,” and “signal peptide,” depending on context. The N-terminal signal sequence may be retained as part of the polypeptide sequence of a mature protein or alternatively cleaved during or after the localization process. This term may be used to specifically refer to a polypeptide sequence located at or proximal to the amino terminus of a polypeptide, which directs localization of the polypeptide to the exosporium of a Brevibacillus, Lysinibacillus, Viridibacillus, or Paenibacillus endospore. In the context of the present disclosure, it is understood that all N-terminal signal sequences must have the capability to target the polypeptide of which it is a part to the exosporium when expressed in members of at least two different genera selected from Brevibacillus, Lysinibacillus, Viridibacillus, and/or Paenibacillus. Exemplary N-terminal targeting sequences compatible with Brevibacillus are shown in Table 1 and FIGS. 1-3, exemplary N-terminal targeting sequences compatible with Lysinibacillus are shown in Table 2 and FIGS. 4-5, exemplary N-terminal targeting sequences compatible with Viridibacillus are shown in Table 3 and FIG. 6, and exemplary N-terminal targeting sequences compatible with Paenibacillus are shown in Table 4 and FIG. 7. As demonstrated by the experimental data disclosed herein (and summarized by Table 5), the N-terminal targeting sequences described herein may display exosporium-targeting functionality in two or more genera of bacteria, allowing for intergeneric use of such sequences and fusion proteins comprising the same.

A “plant” or “host plant,” includes any plant that possesses a rhizosphere or phyllosphere which Brevibacillus, Lysinibacillus, Viridibacillus, or Paenibacillus can colonize, as well as plants that can serve as a transient hosts for Brevibacillus, Lysinibacillus, Viridibacillus, or Paenibacillus bacteria. Colonization is not a requirement for the methods described herein and compositions to function, though it may be preferred in certain aspects of the disclosure.

As used herein, “biological control” is defined as control of a pathogen and/or insect and/or an acarid and/or a nematode by the use of a second organism or a biological molecule. Known mechanisms of biological control include bacteria that control root rot by out-competing fungi for space or nutrients on the surface of the root. Bacterial toxins, such as antibiotics, have been used to control pathogens. The toxin can be isolated and applied directly to the plant or the bacterial species may be administered so it produces the toxin in situ. Other means of exerting biological control include the application of certain fungi producing ingredients active against a target phytopathogen, insect, mite or nematode, or attacking the target pest/pathogen. “Biological control” may also encompass microorganisms having a beneficial effect on plant health, growth, vigor, stress response or yield. Application routes include spray application, soil application and seed treatment.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. Hybridization reactions can be performed under conditions of different “stringency”. In general, a low stringency hybridization reaction is carried out at about 40° C. in 10×SSC or a solution of equivalent ionic strength/temperature. A moderate stringency hybridization is typically performed at about 50° C. in 6×SSC, and a high stringency hybridization reaction is generally performed at about 60° C. in 1×SSC.

As used herein, the term “sequence identity” refers to the degree to which two polynucleotide or amino acid sequences are identical (i.e., on a nucleotide-by-nucleotide or residue-by-residue basis, respectively) over the window of comparison. The percentage of sequence identity is calculated by comparing two optimally aligned sequences over the window of comparison, determining the number of positions at which the identical nucleic acid base (e.g., A, T, C, G for a polynucleotide sequence) occurs in both sequences to yield the number of matched positions, dividing the number of matched positions by the total number of positions in the window of comparison (i.e., the window size), and multiplying the result by 100 to yield the percentage of sequence identity. An equivalent calculation can be performed by comparing two aligned amino acid sequences.

With respect to the comparison of amino acid sequences, in addition to the measurement of sequence identity, a comparison may also take into account whether residue changes constitute “conservative” substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

### N-Terminal Targeting Sequences

The disclosure provides N-terminal targeting sequences from Brevibacillus, Lysinibacillus, and Viridibacillus bacteria. Under stressful environmental conditions, Brevibacillus, Lysinibacillus, and Viridibacillus family bacteria undergo sporulation and form endospores that can stay dormant for extended periods of time. The outermost layer of Brevibacillus, Lysinibacillus, and Viridibacillus endospores is known as the exosporium and comprises a basal layer, and in some strains, external appendages/filaments/structures comprised of collagen-like protein.

Previously reported studies on the exosporium of bacteria from other genera have determined that the exosporium is predominantly formed by a collagen-like glycoprotein, e.g., BclA, in B. anthracis endospores. The basal layer is currently thought to be comprised of a number of different proteins. BclA, the major constituent of the B. anthracis surface nap, has been shown to be attached to the exosporium with its amino-terminus (N-terminus) positioned at the basal layer and its carboxy-terminus (C-terminus) extending outward from the spore. It was previously discovered that certain sequences from the N-terminal regions of BclA and BclB could be used to target a peptide or protein to the exosporium of a B. cereus family member endospore. See U.S. Patent Publication Nos. 2010/0233124 and 2011/0281316, and Thompson, et al., “Targeting of the BclA and BclB Proteins to the Bacillus anthracis Spore Surface,” Molecular Microbiology, 70(2):421-34 (2008), the entirety of each of which is hereby incorporated by reference.

Despite the growing body of literature available regarding B. cereus exosporium targeting sequences, there are no reported studies identifying homologous N-terminal targeting sequences in Brevibacillus, Lysinibacillus, Viridibacillus, or Paenibacillus. A bioinformatics analysis of the known exosporium-targeted collagen-like repeat proteins BclA, BclB or BetA fails to reveal any homologous N-terminal targeting sequences in Brevibacillus, Lysinibacillus, Viridibacillus, or Paenibacillus, suggesting that the exosporium targeting sequences of these proteins is highly specific to the B. cereus family. Given the limited characterization of proteins that form and localize to the exosporium of Brevibacillus, Lysinibacillus, Viridibacillus, or Paenibacillus, one cannot easily deduce the N-terminal signal sequences necessary to target proteins to the exosporium of Brevibacillus, Lysinibacillus, Viridibacillus, or Paenibacillus generally or in particular species within these genera (e.g., B. brevis, L. sphaericus, or V. arvi).

Despite this lack of guidance in the available literature, the inventors have identified N-terminal targeting sequences capable of directing endogenous and fusion proteins to the exosporium of Brevibacillus, Lysinibacillus, and Viridibacillus cells.

For ease of reference, the SEQ ID NOs. for the nucleotide and polypeptide sequences referred to herein are listed in Table 1 below.

TABLE 1
Exemplary Brevibacillus
N-Terminal Targeting Sequences

| Sequence | |
|---|---|
| Identifier | Sequence |
| SEQ ID NO: 1 | MPFSNKIVNGDFETGTLIPWSSXNVAITNLQ |
| | SHTGFFSALLFGNTANSLLFQAVPVTPGDSF |
| | EFFLSIAKLGNLVSPQVNIALLYLDVAATPV |
| | GIGLNXTIPVGHLPDNTANIWTTIYETTSVV |
| | PATATQALLIIHKIPSLSTADIVVDDIVLLQ |
| | TSG |
| SEQ ID NO: 2 | MVRTLSKIEDSTYTTLAPINPIMEVVIYLPF |
| | SNKVVNGDFETGTLFPWSFTNVSITNLQSHT |
| | GFFSALLFGDTANSLLFQAIPVTPGDSFELF |
| | LSIAKLGNLVSPQVNIALXYLDVATIPVGIG |
| | LNLTIPVGHLPDNTANIWTTIYETTSVVPAT |
| | ATQALLIINKIPALSTADIVVDDIGILQTSG |
| SEQ ID NO: 3 | MLPLIFPPQGCPAIFPGPTLDPPDPPDPP |
| SEQ ID NO: 4 | MMILTIKIFRKERAKINDKRANAFNKKILPL |
| | IFPPQGCPAIFPGPTLDPPDPPDPP |
| SEQ ID NO: 5 | MKELHDIKRIWDEVANTDLEEFTRKNPIYMK |
| | SSNGISSSSSTFCWTFIR |
| SEQ ID NO: 6 | MKELHDIKRIWDEVANTDLEEFTRKNPIYMK |
| | SSNGISSSSSTFCWTFIRGKT |
| SEQ ID NO: 7 | MVLALSKIGESTYNASVPINLIMEVVIYLPF |
| | SNIVENGNFESGTLFPWSFTNAAITDLQSHS |
| | GFFSALLFGGTANSLLFQTVPVTPGDSFELF |
| | LSIAKSGNLISPQVNIVLIYLDAANIPVGIG |
| | LNLTIPVGHLPDNTANIWTTIYETTAVVPAT |
| | AIEALLIISKVPAL-STADVILDDIELLQTS |
| | G |
| SEQ ID NO: 8 | MKAVIYLPFGNKIVNGNFETGSLVPWNFSNV |
| | TINNLQSHTGSFSALFFGNTTNSVLYQTVPV |
| | TSGEIFEFFLSIGKIGNLPSPQVNIALIYLN |
| | AASTPENIGINIILPVGKLPNNLNNNWSTIY |
| | EISSVVPAAATQAMVIIHKIPAPSTADIVVD |
| | DIVLVQTGA |
| SEQ ID NO: 9 | MPFGNKIVNGNFETGSLIPWSSSNVAISNLQ |
| | SHTGSYSALLFGNAANSFLFQAVPVTVGDSF |
| | EFFLSIAKIGNLPSPQVDIALIYLNAASTPI |
| | SNGMSIILPTGHLPDNLNNNWSTIYEISSVV |
| | PAMATHAMVIIHKIPSPSTSDIVVDDIVLVQ |
| | TGA |

| | |
|---|---|
| SEQ ID NO: 10 | NKVVNGDFETGTLFPWSFTNVSITNLQSHTG |
| | FFSALFFGDTANSLLFQAIPVTPGDSFELFL |
| | SIAKLGNLVSPQVNIALLYLDVATIPVGIGL |
| | NLTIPVGHLPDNTANIWTTIYETTSVVPATA |
| | TQALLIINKISALSTADIVVDDIGILQTSG |
| SEQ ID NO: 11 | MFQAVPVTVGDSFEFFLSIAKIGNLPSPEVN |
| | IALIYLNVASTPISIGMSIILPIGHLPDNLN |
| | NNWSTIYEISSVVPATATHAMVIIHKIPSP- |
| | STSDIVVDDIVLVQTGA |
| SEQ ID NO: 12 | MKAVIYLPLGNKIVNGNFETGSLVPWNFSNV |
| | AIYNLQSHTGSFSALFFGNTTNSVLYQTVPV |
| | TSGEIFEFFLSIGKIGNLPSPQVNIALIYLN |
| | AASTPENIGINIILPVGKLPNNLNNNWSTIY |
| | EISSVVPAAATQAMVIIHKIPAPSTADIVVD |
| | DIVLVQTGA |
| SEQ ID NO: 13 | MPFSNKIVNGDFETGTLIPWSFVNVAITNLQ |
| | SHTGFFSAQLFGTTANSLLFQAVPVTPGDSF |
| | EFFLSIAKLGNLLSPQVNIALLYLDVAATPV |
| | GIGLNITIPIGHLPDNAANIWTTIYETTTVV |
| | PATATQALLIINKIPTLTTADIVVDDIALLQ |
| | VSG |
| SEQ ID NO: 14 | MFLSIAKSGNVISPQVNIVLIYLDVANIPVG |
| | IGLNLTIPVGHLPDNTANIWTTIYETTSVVP |
| | ATAIEALLIINKVPALSTADVILDDIGLLQT |
| | SG |
| SEQ ID NO: 15 | LLPWSFTNAAITDLQSHSGFFSALLFGGTAN |
| | SLLFQTVPVTPGDSFELFLSIAKSGNLISPQ |
| | VNIVLIYLDAANIPVGIGLNLTIPVGHLPDN |
| | TANIWTTIYETTAVVPATAIEALLIISKVPA |
| | LSTADVILDDIELLQTSG |
| SEQ ID NO: 16 | MPFSNKIVNGDFETGTLIPWSFTNVAITNLQ |
| | SHSGFFSARLFGTTVNSLLFQSVPVTPGDSF |
| | EFFLSIAKLGTLLSPQVNIALLYLDVTATPV |
| | GIGLNITIPIGHLPDNTANIWTTIYETTTVV |
| | PATATQALLIINKIPTLTTADIVVDDIELLQ |
| | VSG |
| SEQ ID NO: 17 | MSFINKIVNGDFETGTLIPWSFVNVAINPLQ |
| | SHTGFFSARLFGNTANSLLFQAVPVTPGDSF |
| | EFFLSIAKIGNLTSPQVNIVLFYLDALANPI |
| | SLGLNITIPSGHLPDNVDNNWTTIYETTSAV |
| | PATATQALLIINKIPALFTTADIVVDDIALL |
| | QTGAGV |
| SEQ ID NO: 18 | MPFGNKIVNGNFETGSLIPWSSSNVVISNLQ |
| | SHTGSYSALLFGNEANSLMFQAVPVTVGDSF |
| | EFFLSIAKIGNLPSPQVNIALIYLNVASTPI |
| | SIGMSIILPIGHLPDNLNNNWSTIYEISSVV |
| | PATATHAMVIIHKIPSPSTSDIVVDDIVLVQ |
| | TGA |
| SEQ ID NO: 19 | MPVKNRAVNGDFETGSLVPWNSINVTISNQQ |
| | SHSGTFSALLFGTSANSLLFQTVPVITGDSF |
| | EFFLSIAKIGNLPSPQVNIAVIYVNATSTPL |
| | SIGMSVILPVNHLPNNLNNNWLTIYNTTSVV |
| | PVTATQAIIIIHKIPAPSTADIVVDDIALIQ |
| | TGG |
| SEQ ID NO: 20 | MPFSNKIVNGNFETGALIPWSSINVAITNLQ |
| | SHTGFFSARLFGNTTNSLLFQAVPVTPGDSF |
| | EFLLSIAKLGNLVSPQVNIALLYLDVTATPV |
| | GIGLNFTIPVGHLPDNTANIWTTIYETTSVV |
| | PATATQALLIINKIAALSTADIVVDDIGILQ |
| | TSG |
| SEQ ID NO: 21 | MPFSNKIVNGNFETGALIPWSSINVAITNLQ |
| | SHTGFFSARLFGNTTNSLLFQAVPVTPGDSF |
| | EFLLSIAKLGNLVSPQVNIALLYLDVTATPV |
| | GIGLNFTIPVGHLPDNTANIWTTIYETTSVV |
| | PATAIQALLIIEKVAALTTADVVVDDIQLLQ |
| | TSG |
| SEQ ID NO: 22 | MFQAVPVTVGDSFEFFLSIAKIGNLPSPEVN |
| | IALIYLNVASTPISIGMSIILPIGHLPDNLN |
| | NNWSTIYEISSVVPATATHAMVIIHKIPSPS |
| | TSDIVVDDIVLVQTGA |
| SEQ ID NO: 23 | MPFGNKIVNGNFETGSLIPWSSSNVVISNLQ |
| | SHTGSYSALLFGNAANSLMFQAVPVTVGDSF |
| | EFFLSIAKIGNLPSPQVNIALIYLNVASTPI |
| | SIGMSIILPIGHLPDNLNNNWSTIYEISSVV |
| | PATATHAMVIIHKIPSPSTSDIVVDDIVLVQ |
| | TGA |
| SEQ ID NO: 24 | MPFSNKIVNGNFETGALIPWSSINVAITNLQ |
| | SHTGFFSARLFGNTTNSLLFQAVPVTLGDSF |
| | EFLLSIAKLGNLVSPQVNIALLYLDVTATPV |
| | GIGLNFTIPVGHLPDNTANIWTTIYETTSVV |
| | PATAIQALLIIEKVAALTTADVVVDDIQLLQ |
| | TSG |
| SEQ ID NO: 25 | MVRTLSKIEDSTYTTLAPINPIMEVVIYLPF |
| | SNKVVNGDFETGTLFPWSFTNVSITNLQSHT |
| | GFFSALLFGDTANSLLFQAIPVTPGDSFELF |
| | LSIAKLGNLVSPQVNIALLYLDVATIPVGIG |
| | LNLTIPVGHLPDNTANIWTTIYETTSVVPAT |
| | ATQALLIINKISALSTADIVVDDIGILQTSG |
| SEQ ID NO: 26 | MPFGNKIVNGNFETGSLIPWSSSNVAISNLQ |
| | SHTGSYSALLFGNAANSFLFQAVPVTVGDSF |
| | EFFLSIAKIGNLPSPQVDIALIYLNAASTPI |
| | SIGMSIILPTGHLPDNLNNNWSTIYEISSVV |
| | PAMATHAMVIIHKIPSPSTSDIVVDDIVLVQ |
| | TGA |
| SEQ ID NO: 27 | MVRTLSKIEDSTYTTLAPINPIMEVVIYLPF |
| | SNKVVNGDFETGTLFPWSFTNVSITNLQSHT |
| | GFFSAILFGDTANSLLFQAIPVTPGDSFELF |
| | LSIAKLGNLVSPQVNIALLYLDVATIPVGIG |
| | LNLTIPVGHLPDNTANIWTTIYETTSVVPAN |
| | ATQALLIINKISALSTADIVVDDIGILQTSG |
| SEQ ID NO: 28 | GDFETGTLFPWSFTNVSITNLQSHTGFFSAI |
| | LFGDTANSLLFQAIPVTPGDSFELFLSIAKL |
| | GNLVSPQVNIALLYLDVATIPVGIGLNLTIP |
| | VGHLPDNTANIWTTIYETTSVVPANATQALL |
| | IINKISALSTADIVVDDIGILQTSG |
| SEQ ID NO: 29 | MPFGNKIVNGNFETGSLIPWSSSNVAISNLQ |
| | SHTGSYSALLFGNTANSLMFQAVPVTVGDSF |
| | EFFLSIAKIGNLPSAQVNIALIYLNVASTPI |
| | SIGMSIILPIGHLPDNLNNNWSTIYEISSVV |
| | PAMATHAMVIIHKIPSPSTSDIVVDDIVLVQ |
| | TGA |
| SEQ ID NO: 30 | MPFSNKIVNGDFETGTLIPWSSINVAITNLQ |
| | SHTGFFSARLFGNTANSLLFQAVPVTPGDSF |
| | EFLLSIAKLGNLVSPQVNIALLYLDVTATPV |
| | GIGLNLTIPVGHLPDNTADIWTTIYETTSVV |
| | PATAIQALLIIEKVPGLTTADVVVDDIELLQ |
| | TSG |
| SEQ ID NO: 31 | MPFGNKIVNGNFETGSLIPWSSSNVAISNLQ |
| | SHTGSYSALLFGNTANSLMFQAVPVTVGDSF |
| | EFFLSIAKIGNLPSAQVNIALIYLNVASTPI |
| | SIGMSIILPIGHLPDNLNNNWSTIYEISSVV |
| | PAMATHAMVIIHKIPSPSTSDIVVDDIVFVQ |
| | TGA |
| SEQ ID NO: 32 | VYLNVVATPIGIGMSTILPIDHLPDNTNKNW |
| | TTIYETTSVVPATAIRALVIIHKIPSPSTAD |
| | IVVDDIVLLQTGL |
| SEQ ID NO: 33 | MPFSNKIVNGDFETGTLIPWSSINVAITNLQ |
| | SHTGFFSARLFGNIANSLLFQAVPVTPGDSF |
| | EFLLSIAKLGNLVSPQVNIALLYLDVTATPV |
| | GIGLNLTIPVGHLPDNTANIWTTIYETTSVV |
| | PATAIQALLIIEKVASLTTADVVVDDIELLQ |
| | TSG |
| SEQ ID NO: 34 | MPFGNKIVNGNFETGSLIPWSSSNVVISNLQ |
| | SHTGSYSALLFGNAANSLMFQAVPVTVGDSF |
| | EFFLSIAKIGNLPSPQVNIALIYLNVASTPI |
| | SIGMSIILPIDHLPDNLNNNWSTIYEISSVV |
| | PATATHAMVIIHKIPSPSTSDIVVDDIVLVQ |
| | TGA |
| SEQ ID NO: 35 | MPFSNKIVNGDFETGTLIPWSSINVAITNLQ |
| | SHTGFFSARLFGNIANSLLFQAVPVTPGDSF |
| | EFLLSIAKLGNLVSPQVNIALLYLDVTATPV |
| | GIGLNFTIPVGHLPDNTANIWTTIYETTSVV |
| | PATAIQALLIIEKVASLTTADVVVDDIELLQ |
| | TSG |
| SEQ ID NO: 36 | FELFLSIAKLGNLVSPQVNIALLYLDVATIP |
| | VGIGLNLTIPVGHLPDNTANIWTTIYETTSV |
| | VPATATQALLIINKIAALSTADIVVDDIGIL |
| | QTSE |
| SEQ ID NO: 37 | MPFGNKIVNGNFETGSLIPWSSSNVVISNLQ |
| | SHTGSYSALLFGNATNSLMFQAVPVTVGDSF |
| | EFFLSIAKIGNLPSPQVNIALIYLNVASTPI |
| | SIGMSIILPIGHLPDNLNNNWSTIYEISSVV |
| | PATATHAMVIIHKIPSPSTSDIVVDDIVLVQ |
| | TGA |
| SEQ ID NO: 38 | MPFSNKIVNGDFETGTLIPWSSINVAITNLQ |
| | SHTGFFSARLFGNIANSLLFQAVPVTPGDSF |
| | EFLLSIAKLGNLVSPQVNIALLYLDVTATPV |
| | GIGLNFTIPVGHLPDNTANIWTTIYETTSVV |
| | PATATQALLIINKIAALSTADIVVDDIGILQ |
| | TSE |

TABLE 2
Exemplary Lysinibacillus N-Terminal Targeting Sequences

| Sequence | |
|---|---|
| Identifier | Sequence |
| SEQ ID NO: 39 | ATGCGCTTTAGCTATAGCCGCAACTTTGATAACTTTAAATGCGATAGCAAATGCAACTATCCGATGAAAT |
| | ATAAATGCAACGAATTTGGCTGCAACGGCTATCCGTATGATCTGAGCGGCATTCAGGATCCGATT |
| SEQ ID NO: 40 | ATGCATTTTGGCAACTTTAACCTGGATCGCGTGTGCAGCAAATGCAACAGCTTTCCGTGCTGCTGCAAAAC |
| | CGGCCCGATTCCGAGCTGCCCGATTTGGCCGGTGAAACCGACC |
| SEQ ID NO: 41 | MRFSYSRNFDNFKCDSKCNYPMKYKCNEFGCNGYPYDLSGIQDPI |
| SEQ ID NO: 42 | MHFGNFNLDRVCSKCNSFPCCCKTGPIPSCPIWPVKPT |
| SEQ ID NO: 43 | MRFSYNRNFDNFKCDAKCNYPMKYKCNEFGCNGYPYVYGIEDPI |
| SEQ ID NO: 44 | MRFSYSRNFDNFKCDSKCNYPMKYKCNEFGCNGYPYDFSGIQDPI |
| SEQ ID NO: 45 | MRFSYSRNFDNFKCDSKCNYPMKYKCNEFGCNGYPYDFSGIQDPM |
| SEQ ID NO: 46 | MRFSYSRNFDYFKCDSKCNYPMKYKCYEFGCNGYPYDFSGIQDPI |
| SEQ ID NO: 47 | MRFSYSRNSDNFKCDSKCNYPMKYKCNEFGCNGYPYDFSGIQDPI |
| SEQ ID NO: 48 | MRFSYSRNFDNFKCDSKSNYPMKYKCNEFGCNGYPYDFSGIQDPI |
| SEQ ID NO: 49 | MRFSYSRNFDNFKCDSKCNYPMKYKCNEFGCNGYPYDFLGIQDPI |
| SEQ ID NO: 50 | MRFSYSRNFDNFKCDSKCNYPMKYKCNEFGCNGYPYDLLGIQDPI |
| SEQ ID NO: 51 | MRFSYSRNFDNFKCDSKCNYPLKFKCNEFGCNGYPYDFSGIQDPI |
| SEQ ID NO: 52 | MRFSYSRNFDNFKCDSKCNYPMKYKCNEFGCNGYPYNLSGIQDPI |
| SEQ ID NO: 53 | MRFSYSRNFDNFKCDSKCNYPKKYKCNEFGCNGYPYDLSGIQDPI |
| SEQ ID NO: 54 | MRFSYSRNFDNFKCDSKCNYPLKYKCNEFGCNGYPYDLSGIQDPI |
| SEQ ID NO: 55 | MRLSYSRNFDNFKCDSKCNYPMKYKCNEFGCNGYPYDFSGIQDPI |
| SEQ ID NO: 56 | LRFSYSRNFDNFKCDSKCNYPMKYKCNEFGCNGYPYDLSGIQDPI |
| SEQ ID NO: 57 | MRFRYSRNFDNFKCDSKCNYPMKYKCNEFGCNGYPYDFSGIQDPI |
| SEQ ID NO: 58 | MRVSYSRNFDNFKCDSKCNYPMKYKCNEFGCNGYPYDFSGIQDPI |
| SEQ ID NO: 59 | MRFSYSRYFDNFKCDSKCNYPMKYKCNEFGCNGYPYDFSGIQDPI |
| SEQ ID NO: 60 | MRISYSRNFDNFKCDSKCNYPMKYKCNEFGCNGYPYDFSGIQDPM |
| SEQ ID NO: 61 | MRFSYSRNFDNFKCDSKCNYPMKYKCNEFGCNGYPYDLSGIQEPM |
| SEQ ID NO: 62 | MRFSYSRNFDNFKCDSKCNYPMKYKCNEFGCNGYPYDLSGIQDLI |
| SEQ ID NO: 63 | MRFSYSRNFDNFKCDSKCNYPMKYKCNEFGCNGYPYDLSGIQVPI |
| SEQ ID NO: 64 | MRFSYSRNFDNFKCDSKFNYPMKYKCNEFGCNGYPYDLSGIQDPI |
| SEQ ID NO: 65 | MRFSYSRNFDYFKCDSKCNYPMKYKCNEFGCNGYPYDLSGIQDPI |
| SEQ ID NO: 66 | MRFSYSRNFDNFKCDSKCNYPMKNKCNEFGCNGYPYDLSGIQDPI |
| SEQ ID NO: 67 | MRFSYSRNFDNFKCDSKCNYPMKYKCNEFGCNGYPYDLSAIQDPI |
| SEQ ID NO: 68 | MRFSYSRNFDNFKCDSKCNYPMKYKCNEFGCNGYPYDLSGFQDPI |
| SEQ ID NO: 69 | MRFSYSRNFDNFKCDSKCNYPMKYKCNEFGCNGYPYDLSGFQDPT |
| SEQ ID NO: 70 | MRFSYSRNFDNYKCDSKCNYPMKYKCNEFGCNGYPYDLSGIQDPI |
| SEQ ID NO: 71 | MRFSYCRNFDNFKCDSKCNYPMKYKCNEFGCNGYPYDLSGIQDPI |
| SEQ ID NO: 72 | MRFSYSRNLDNFKCDSKCNYPMKYKCNEFGCNGYPYDLSGIQDLI |
| SEQ ID NO: 73 | MRFSYSRNFDNFKCDSKCNYPMKYKCNEFGCIGYPYNLSGIQDPI |
| SEQ ID NO: 74 | MRFSYSRNFDNFKCDSKCNYPMKYKCNEFGCNGYAYNLSGIQDPI |
| SEQ ID NO: 75 | MRFSYSRNLDNFKCDSKCNYPMKYKCNEFGCNGYPYNLSGIQDPI |
| SEQ ID NO: 76 | LRFSYSRNFDNFKCDSKCNYPMKYKCNEFGCNGYPYNLSGIQDPI |
| SEQ ID NO: 77 | MRFSYIRNFDNFKCDSKCNYPMKYKCNEFGCNGYPYNLSGIQDPI |
| SEQ ID NO: 78 | MRVSDSRNFDNFKCDSKCNYPMKYKCNEFGCNGYPYDFSGIQDPI |
| SEQ ID NO: 79 | MRFSYSRNFDNFKCYSKCNYPMKYKCNEFGCNGYPYDLSGFQDPI |
| SEQ ID NO: 80 | MRFSYSRNFDNFKCDSKCNYPMKYKCNEFWCNGYPYDLSGFQDPI |
| SEQ ID NO: 81 | MRFSNSRNFDNFKCDSKCNYPMKYKCNEFGCNGYPYDLSGFQDPI |
| SEQ ID NO: 82 | MRFSYSRNLDNFKCDSKCNYPMKYKCNEFGCNGYPYDLSGFQDPI |
| SEQ ID NO: 83 | FSYSRNFDNFKCDSKCNYPMKYKCNEFGCNGYAYNLSGIQDPI |
| SEQ ID NO: 84 | MRFSYSRNFDNFKCDSKCNYPMKYKCNEYGCNGYAYNLSGIQDPI |
| SEQ ID NO: 85 | MRLSYSRNLDNFKCDSKCNYPMKYKCNEFGCNGYPYNLSGIQDPI |
| SEQ ID NO: 86 | MRFSYSRNFDNFKCDSKCNYPMKYKCNEFGCNGYPFDFSGFQDPI |
| SEQ ID NO: 87 | MRFSYSRNFDNFKSDTKCNYPMKYKCNEFGCNGYPYDLSGIQDPI |
| SEQ ID NO: 88 | MRFSYNRNFDNFKCDAKCNYPMKYKCNEFGCNGYPYVYGIQDPI |
| SEQ ID NO: 89 | MRFSFNRNFDNFKCDAKCNYPMKYKCNEFGCNGYPYVYGIEDPI |
| SEQ ID NO: 90 | MRFSYNRNFDNFKCDAKCNYPMKYKCNEFGCNGYPYVYGIEDQI |
| SEQ ID NO: 91 | MRFSYIRNFDNFKCDAKCNYPLKYKCNEFGCNGYPYVYGIQEPI |
| SEQ ID NO: 92 | MRFSYNLNFDNFKCDAKCNYPLKYKCNEFGCNGYPYVYGIQEPI |
| SEQ ID NO: 93 | MRFCYYRNFDNFKCDANCYYPMKYKCNEFGCNGYPYVYGIEDPI |
| SEQ ID NO: 94 | MRFSYSRNFDNFKCDSKCNYHMKYKCNEFGCNGYPYVLSDIQDPI |
| SEQ ID NO: 95 | YSRNFDKIKCDSKCNYHMKYKCNEFGCNGYPYDFSGIQDPI |
| SEQ ID NO: 96 | MRFNYNRNNDNFKCDAKCNYPMKFKCNEFGCNGYPYGAYEIQDPV |
| SEQ ID NO: 97 | MRFNYNRNNDNFKCDTKCNYPMKFKCNEFGCNGYPYGAYEIQDPV |
| SEQ ID NO: 98 | MHFGKFKERVCSKCNTFPCCCSPCPPQSCPIWPVKPT |
| SEQ ID NO: 99 | MHFGKFKERVCSKCNTFPCCCSPYPPQSCPIWPVKPT |
| SEQ ID NO: 100 | MHFGNFIDRVCSKCHSFPCCCKTGPIPSCPIWPVKPT |
| SEQ ID NO: 101 | MHFGNFIDRVCSKCHSFPCYCKTGPIPSCPIWPVKPT |
| SEQ ID NO: 102 | MHFGNFIDRVCSKCHSFPCCCKTGPIPSCPIWPVNPT |
| SEQ ID NO: 103 | MHFGNFIDRVCSKCHSFPCCCKAGPIPSCPIWPVKPT |
| SEQ ID NO: 104 | MHFGNFIDRVCSKCHSFPCCCKTGPIPNCPIWPVKPT |
| SEQ ID NO: 105 | MHYGNFIDRVCSKCHSFPCCCKTGPIPSCPIWPVKPT |
| SEQ ID NO: 106 | MHFGNFIVRVCSKCHSFPCCCKTGPIQSCPIWPVKPT |
| SEQ ID NO: 107 | MHFGIIIDRVCSKCHSFPCCCKTGPIPSCPIWPVKPT |
| SEQ ID NO: 108 | MHFGKFIDRVCTKCHSYPCCCKTGPIPSCPIWPVKPT |
| SEQ ID NO: 109 | MHFGKFLDRVCNKCHSFPCCCKTGPIPGCPIWPVKPT |
| SEQ ID NO: 110 | MHFGKFLDRVCSKCHSFPCCCKTGPIPGCPIWPVKPT |
| SEQ ID NO: 111 | MHFGKFLDRVCSKCNSFPCCCKTGPSPSCPIWPVKPT |
| SEQ ID NO: 112 | MNFGKFLDRVCSKCNSFPCCCKTGPSPSCPIWPVKPT |
| SEQ ID NO: 113 | MLFGKFLDRVCSKCNSFPCCCKTGPSPSCPIWPVKPT |
| SEQ ID NO: 114 | MHFGKFLDRVCSKCNSFPCCCKTGPSPSCPNWPVKPT |
| SEQ ID NO: 115 | MHFGNYLKRVCSKCNSFPCCCRTGPIPSCPVWPVKPT |
| SEQ ID NO: 116 | MHFGNYLKRVCSKCNYFPCCCRTGPIPSCPVWPVKPT |
| SEQ ID NO: 117 | MHFGNYLKRVCSKCNSFPCCCRPGPIPSCPVWPVKPT |
| SEQ ID NO: 118 | MHFGNYLKQVCSKCNSFPCCCRTGPIPSCPVWPVKPT |
| SEQ ID NO: 119 | MHFGNYLKRVCSKCNSFPCCCRTGPIQSCPVWPVKPT |
| SEQ ID NO: 120 | MHFGNYLKQVCNKCNSFPCCCRTGPIPSCPVWPVKPT |
| SEQ ID NO: 121 | MHFGNYLKQVCSKCNSFPCCCRNGPIPSCPVWPVKPT |
| SEQ ID NO: 122 | MHFGNYLKQVCSKCNSFPCCCRTGPIPSCPIWPVKPT |
| SEQ ID NO: 123 | LHFGNYLKQVCSKCNSFPCCCRTGPIPSCPIWPVKPT |
| SEQ ID NO: 124 | MLFGNYLKQVCSKCNSFPCCCRTGPIPSCPIWPVKPT |
| SEQ ID NO: 125 | MHFGNYLKQVSSKCNSFPCCCRNGPIPSCPVWPVKPT |
| SEQ ID NO: 126 | MHFGRFNERVCSKCNTFPCCCNPCAAQSCPIWPVKPT |
| SEQ ID NO: 127 | MHFGRFKERVCSKCNTFPCCCNPCAAQSCPIWPVKPT |
| SEQ ID NO: 128 | MHFGRFNERVCSKCNSFPCCCNPCAAQSCPIWPVKPT |
| SEQ ID NO: 129 | MHFGKFNERVCSKCNTFPCCCNPCAAQSCPIWPVKPS |
| SEQ ID NO: 130 | MHFGRFNERVCSKCNTFPCCCCAAQSCPIWPVKPT |
| SEQ ID NO: 131 | MHFGKFKDSVCSKCNTFPCCCNPCAAQSCPIWPVKPT |
| SEQ ID NO: 132 | MHFGRFNERVCSKCNTFPCCCCAAQSCPIWPVKP |
| SEQ ID NO: 133 | MHFGKFKERVCSKCNSFPCCCSPCPPQSCPIWPVKPT |
| SEQ ID NO: 134 | MHFGKFKDRVCSKCNTFPCCCSPCPPQSCPIWPVKPT |
| SEQ ID NO: 135 | MHFGRFNERVCSKCNTFPCCCSPCDVQSCPIWPVKPT |
| SEQ ID NO: 136 | MQFGRFNERVCSKCTTFPCCCSPCDVQSCPIWPVKPT |
| SEQ ID NO: 137 | MHFGKENDRICSKCNTFPCCCNPCAPSCPVWPVKP |
| SEQ ID NO: 138 | MHFGNFIDRVCSKCNTFPCCCRGGPVPGCPIWPVKPT |
| SEQ ID NO: 139 | MHFGNFIDRVCSKCKTFPCCCRGGPVPGCPIWPVKPT |
| SEQ ID NO: 140 | MHFGNFIDRVCSKCNTFPCCCRGGPVPGCPIWPVKPP |
| SEQ ID NO: 141 | MHFGNFIDRVCSKCNTFPCCCRGGPVPGCHIWPVKPT |
| SEQ ID NO: 142 | MHFGNFIDRVCSKCKTFPCCCRGGPVPCCPIWPVKPT |
| SEQ ID NO: 143 | MHFGNFIDRVCSKCKTIPCCCRGGPVPGCPIWPVKPT |
| SEQ ID NO: 144 | MHFGNFIDRVCSKCNTFPCCCKGGPVPGCHIWPVKPT |
| SEQ ID NO: 145 | MHFGNFIDRVCSKCKTFPCCCGSGPVPGCPIWPVKPT |
| SEQ ID NO: 146 | MHFGNFIDRVCSKCKTSPCCCGSGPVPGCPIWPVKPT |
| SEQ ID NO: 147 | MHFGNFIDRVCTKCKIFPRCCRGGPVPGCPIWPVKPT |
| SEQ ID NO: 148 | MHFGKFTDRVCSKCNTFPCCCNTCPAPNCPIWPVKP |
| SEQ ID NO: 149 | MNFGNFNNRICSKCNTFPCCCIPCAPSCPVWPVKPG |
| SEQ ID NO: 150 | MYFGNFLDRICSKCNTFPCCCLSGPILRSPIWPVRPT |
| SEQ ID NO: 151 | MHFGNFLDRVCNKCNTFPCCCLSGPIHRSPIWPVPPAKATR |
| SEQ ID NO: 152 | MQFGKYNDRICRKCNTFPCCCNPSAPSCPGWPVKP |
| SEQ ID NO: 153 | MKFQDFNINKICNNCHSYPCCCHNGPVQRCPVWPVKPT |
| SEQ ID NO: 154 | MKFQDFNINKICNNCHSYPCCCHNGPVHRCPVWPVKPT |
| SEQ ID NO: 155 | MKFQDFNINKICNNCNSYPCCCHNGPVQRCPVWPVKPT |
| SEQ ID NO: 156 | MKFQDFNINKICNNCHSYPCCCHNGPVLRCPVWPVKPT |
| SEQ ID NO: 157 | MKFLDFNINKICNNCHSYPCCCHNGPVHRCPVWPVKPT |
| SEQ ID NO: 158 | MKFQDFNINKICNNCHSYPCCCHKGPVHRCPVWPVKPT |
| SEQ ID NO: 159 | MKFQDFNINKICNICHSYPFCCHNGPVQRCPVWPVKPT |
| SEQ ID NO: 160 | MKFQDFNINMICNNCHSYPCCCHNGPVHRCPVWPVKP |
| SEQ ID NO: 161 | MKFHDFNLNNICGKCLSHPCCCNNGPAQRCPIWPVKPT |
| SEQ ID NO: 162 | MKFHDFNLNNICGKCLSHPCCCNNVPAQRCPIWPVKPT |
| SEQ ID NO: 163 | MKFHDFNLNNICGKCLSHPCCCNNGPVQRCPIWPVKPT |
| SEQ ID NO: 164 | MDFANFKNRICSRCNSFPCNCKRNFPQNCPIWPVKPT |
| SEQ ID NO: 165 | MDFANFKNRICSRCNSFPCNCKRNSPQNCPIWPVKPT |
| SEQ ID NO: 166 | MDFANFKNRICSRCNTFPCNCKRNFPQNCPIWPVKPT |
| SEQ ID NO: 167 | MDFANFKNRICSRCNSFPCNCKGNFPQNCPIWPVKPT |
| SEQ ID NO: 168 | MDFAYFKNRICSRCNSFPCNCKRNFPQNCPIWPVKPT |
| SEQ ID NO: 169 | MDFANFKNRICSRCNSFPCNCIRNIPQNCPIWPVKPT |
| SEQ ID NO: 170 | MDLANFKNRICSRCNSFPCNCKRNFPQNCPIWPVKPT |
| SEQ ID NO: 171 | MDFANFKNRICSRCNSFPCNCKSNYPQNCSIWPVKPT |
| SEQ ID NO: 172 | MNFEDYFLNNVCSKCKTFPCACIRIPEHHCCIWPVKPT |
| SEQ ID NO: 173 | MNFEDYFLNNVCSKCKTFPCACIRIPGHHCSIWPVKPT |
| SEQ ID NO: 174 | MNFEDYFLNNVHVLCKTFPCACIRIPEHHCCIWPVKPT |
| SEQ ID NO: 175 | MNFEDYFLNNVCSKCRTFPCGCRRIPEHHSCVWPVKPT |
| SEQ ID NO: 176 | MNFEDYFLKNVCSKCRTFPCGCTRIPEQHCCVWPVKPT |
| SEQ ID NO: 177 | MNFHDFILNNICKRCNSYPCRCINRPEQNCSIWPVKPT |

TABLE 3
Exemplary Viridibacillus N-Terminal Targeting Sequences

| Sequence | |
|---|---|
| Identifier | Sequence |
| SEQ ID NO: 178 | ATGGATTCAAATAAAACGAGGTGTGCTGTTTGTAATAAATATCAATGTTCTTGTAACTCCAAAAAAAGAAGTCCTAA |
| | AACAGTTATTAGAGATTTAGATTATATAGATCCATGTGAACCTCTTTATATAATAGATCATTGTCATAATAGA |
| SEQ ID NO: 179 | MDSNKTRCXVCNKYQCSCNHKKRSPKTVIRDLDYIDPCEPLYIIDHCHNR |
| SEQ ID NO: 180 | ATGGACTCAAATAAAACGAGGTGTCCTATTTGTAATAATTATCAATGTTCTTGTAACGCTAAAAAAAGAAGGCCTAC |
| | AAGATGTATTAGATATTTAGATTATATAGATCAATGTGAACAATGTGAACCTATTTCTATAATAGATCATTGTCATA |
| | ATAGA |
| SEQ ID NO: 181 | MDSNKTRCPICNNYQCSCNAKKRRPTRCIRYLDYIDQCEQCEPISIIDHCHNR |
| SEQ ID NO: 182 | ATGGACTCAAATAAAACGAGGTGTCCTATTTGTAATAATTATCAATGTTCTTGTAACGCTAAAAAAAGAAGGCCTAC |
| | AAGATGTATTAGATATTTAGATTATATAGATCAATGTGAACAATGTGAACCTATTTCTATAATAGATCATTGTCATA |
| | ATAGAGGTAAAACAGGACCGACCGGAGCAACTGGTGCAACCGGACCAACTGGAGCAACTGGAGCAACAGGACCAA |
| | CAGGACCAACTGGAGCAACAGGACCTACAGGAGCAACAGGACCAACAGGTGCAACTGGGGCGACAGGAATCACCG |
| | GGCCGACTGGAGCAACGGGAGCAACA |
| SEQ ID NO: 183 | MDSNKTRCPICNNYQCSCNAKKRRPTRCIRYLDYIDQCEQCEPISIIDHCHNRGKTGPTGATGATGPTGATGATGPTGPT |
| | GATGPTGATGPTGATGATGITGPTGATGAT |
| SEQ ID NO: 184 | ATGGAATCAAATAAAACGAGGTGTATTGTTTGTAATAAATATCAATGTTCTTGTAACTCCAAAAAAAGAAGTCCTAA |
| | AACAGTTATTAGAGATTTAGATTATATAGATCCATGTGAACCTCTTTATATAATAGATCATTGTCATAATAGA |
| SEQ ID NO: 185 | MESNKTRCIVCNKYQCSCNSKKRSPKTVIRDLDYIDPCEPLYIIDHCHNR |
| SEQ ID NO: 186 | ATGGAATCAAATAAAACGAGGTGTATTGTTTGTAATAAATATCAATGTTCTTGTAACTCCAAAAAAAGAAGTCCTAA |
| | AACAGTTATTAGAGATTTAGATTATATAGATCCATGTGAACCTCTTTATATAATAGATCATTGTCATAATAGAGGTA |
| | AAACAGGACCAACAGGACCAACGGGACCAACCGGAGTGACTGGAGCAACCGGACCAACAGGAGAAACAGGACCA |
| | ACGGGAGCAACCGGAGCAACTGGAGCAACAGGAGCAACAGGTCCAATGGGAGTAACTGGCTCGACCGGAGCAACT |
| | GGTGTAACGGGAGCAACAGGAGCAACAGGGTCAACCGGAGCAACAGGTGAAACCGGAGCGACTGGTGAAACCGG |
| | A |
| SEQ ID NO: 187 | MESNKTRCIVCNKYQCSCNSKKRSPKTVIRDLDYIDPCEPLYIIDHCHNRGKTGPTGPTGPTGVTGATGPTGETGPTGAT |
| | GATGATGATGPMGVTGSTGATGVTGATGATGSTGATGETGATGETG |
| SEQ ID NO: 188 | ATGTATTCAAATAAAAAGAGGTGTGCTGTTTGTAATAAATATCAATGTTCTTGTAACTCCAAAAAAAGAAGTCCTAA |
| | AACAGTTATTAGAGATTTAGATTATATAGATCCATGTGAACCTCTTTATATAATAGATCATTGTCATAATAGA |
| SEQ ID NO: 189 | MYSNKKRCAVCNKYQCSCNSKKRSPKTVIRDLDYIDPCEPLYIIDHCHNR |
| SEQ ID NO: 190 | ATGTATTCAAATAAAAAGAGGTGTGCTGTTTGTAATAAATATCAATGTTCTTGTAACTCCAAAAAAAGAAGTCCTAA |
| | AACAGTTATTAGAGATTTAGATTATATAGATCCATGTGAACCTCTTTATATAATAGATCATTGTCATAATAGAGGTA |
| | AAACAGGACCAACAGGAGCAACAGGTCATACAGGAACAACCGGAACAACAGGAGAGACTGGACCAACAGGAGCA |
| | ACAGGAGCAACAGGAATAACGGGAGCAACGGGAGAAACAGGAGCAACAGGACCAACGGGAACAACTGGTGTAAC |
| | CGGAGAAACCGGAGCAACAGGTCCAACTGGAGAAACTGGTGAAACAGGACCAACGGGAGCAACTGGGAACCGGA |
| | GCAACAGGAGAAACAGGAACAACTGGAGCAACAGGAAAAACGGGAACAACAGGAGAAACAGGAGAAACAGGAG |
| | CAACAGGAGAAACAGGAAAAACAGG |
| SEQ ID NO: 191 | MYSNKKRCAVCNKYQCSCNSKKRSPKTVIRDLDYIDPCEPLYIIDHCHNRGKTGPTGATGHTGTTGTTGETGPTGATGAT |
| | GITGATGETGATGPTGTTGVTGETGATGPTGETGETGPTGATGNRSNRRNRNNWSNRKNGNNRRNRRNRSNRRNRKNR |
| SEQ ID NO: 192 | ATGTATTCAAATAAAAAGAGGTGTGCTGTTTGTAATAAATATCAATGTTCTTGTAACTCCAAAAAAAGAAGTCCTAA |
| | AACAGTTATTAGAGATTTAGATTATATAGATCCATGTGAACCTCTTTATATAATAGATCATTGTCATAATAGA |
| SEQ ID NO: 193 | MYSNKKRCAVCNKYQCSCNSKKRSPKTVIRDLDYIDPCEPLYIIDHCHNR |
| SEQ ID NO: 194 | ATGTATTCAAATAAAAAGAGGTGTGCTGTTTGTAATAAATATCAATGTTCTTGTAACTCCAAAAAAAGAAGTCCTAA |
| | AACAGTTATTAGAGATTTAGATTATATAGATCCATGTGAACCTCTTTATATAATAGATCATTGTCATAATAGAGGTA |
| | AAACAGGACCAACAGGAGCAACAGGTCCTACAGGATCAACGGGACCAACAGGAGTGACTGGACCAACAGGAGCAA |
| | CAGGAACAACAGGATCAACAGGAACAACAGGAGCAACAGGAGCAACAGGAGAAACAGGAGCAACAGGACCAACA |
| | GGACCAACAGGAGAAACAGGAACAACA |
| SEQ ID NO: 195 | MYSNKKRCAVCNKYQCSCNSKKRSPKTVIRDLDYIDPCEPLYIIDHCHNRGKTGPTGATGPTGSTGPTGVTGPTGATGTT |
| | GSTGTTGATGATGETGATGPTGPTGETGTT |
| SEQ ID NO: 196 | ATGGATTCAAATAAAAAGAGGTGTGCTGTTTGTAATAAATATCAATGTTCTTGTAACTCCAAAAAAAGAAGTCCTAA |
| | AACAGTTATTAGAGATTTAGATTATATAGATCCATGTGAACCGCTTTATATAATAGATCATTGTCATAATAGA |
| SEQ ID NO: 197 | MDSNKKRCAVCNKYQCSCNSKKRSPKTVIRDLDYIDPCEPLYIIDHCHNR |
| SEQ ID NO: 198 | ATGGATTCAAATAAAAAGAGGTGTGCTGTTTGTAATAAATATCAATGTTCTTGTAACTCCAAAAAAAGAAGTCCTAA |
| | AACAGTTATTAGAGATTTAGATTATATAGATCCATGTGAACCGCTTTATATAATAGATCATTGTCATAATAGAGGTA |
| | AAACAGGACCAACAGGAGCAACAGGTCCTACAGGATCAATGGGACCAACCGGAGTGACTGGATCAACAGGAGCAA |
| | CCGGACCAACAGGGTCAACCGGAGTAACTGGGGAAACAGGATCAACCGGGACAACGGGACCAAGCGGATCAACTG |
| | GACCAACTGGTGCAACAGGGTCAACCGGAGAAACCGGAGCAACAGGGTCAACCGGAGCAACTGGAGAAACAGGTC |
| | CAACAGGACCAACGGGAACAACTGGTGTAACCGGAGAAACCGGAGCAACAGGTCCAACTGGAGCAACTGGAGCAA |
| | CTGGTGAAACAGGTTCAACAGGACCAACGGGAGCAACTGGAGAAACTGGTGAAACAGGTTCAACAGGACCAACGG |
| | GAGCAACGGGATCGACTGGAGCAACGGGACCAACGGGAGCAACGGGATCCACTGGAGCAACGGGATCGACTGGAG |
| | CAACGGGATCGACTGGAGCAACAGGACCAACAGGAGCAACAGGGTCAACCGGAGAAACAGGACCAACTGGTGAA |
| | ACAGGTTCAACGGGAGCAACTGGGTCGACCGGAGCAACAGGTGAAACAGGACCAACGGGAACAACTGGAGTAACC |
| | GGAGAAACCGGAGCAACAGGTCCAACAGGAGCAACTGGAGCAACTGGAGAAACTGGTGAAACAGGTTCAACAGGA |
| | CCAACGGGAGCAACTGGAGAAACTGGTGAAACAGGTTCAACAGGACCAACGGGAGCAACGGGATCGACTGGAGCA |
| | ACGGGACCAACTGGAGCAACGGGATCGACTGGAGCAACGGGATCGACTGGAGCAACGGGATCGACTGGAGCAACA |
| | GGACCAACAGGAGCAACAGGGTCAACCGGAGAAACAGGACCAACTGGTGAAACAGGTTCAACGGGAGCAACTGGG |
| | TCGACCGGAGCAACAGGTGAAACAGGACCAACGGGAGCAACTGGGTCGACCGGAGCAACTGGTGAAACAGGTGCA |
| | ACAGGACCAACAGGACCAACGGGAACAACTGGTGTAACCGGAGCAACAGGTCCAACTGGAGCAACTGGAGAAACA |
| | GGACCAACGGGAGCGACTGGAGAAGCAGGACCAACAGGGTCAACCGGAGCGACTGGACCAACCGGGGCAACGGG |
| | AATCACGGGAGCAACAGGACCAACAGGATCAACTGGTGCAACAGGGTCAACCGGAGAAACTGGATCAACTGGAGA |
| | AACCGGACCAACAGGAGCAACTGGTGTAACCGGAGCAACAGGTCCAACTGGAGAAACAGGTTCAACAGGACCAAT |
| | GGGAGCAACCGGAGAAACTGGTTCGACCGGATCAACTGGTGTAACGGGAGCAACAGGAGCAACGGGAGAAACCGG |
| | AGCAACAGGATCAACGGGAGCAACTGGCACGACCGGAGCAACAGGAGAAACAGGACCAACAGGACCAACTGGGT |
| | CCACCGGAGCAACAGGAGAAACAGGTCCAACAGGACCAACGGGAGCAACTGGAGTAACCGGAGCAACAGGAGCA |
| | ACGGGAGCAACTGGAGAAACAGGTTCAACAGGACCAATGGGAGCAACCGGAGAAACTGGTTCGACCGGATCAACT |
| | GGTGTAACGGGAGCAACAGGAGCGACTGGAGTAACAGGAGCAACTGGTGAAACAGGATCAACGGGAGCAACTGGG |
| | TCGACCGGAGCAACAGGTGAAACCGGACCAACAGGTCCAACAGGTCCAACAGGACCAACGGGAACAACTGGTGTA |
| | ACCGGAGTAACTGGAGAAACCGGACCAACAGGAGCGACTGGTGAAACAGGTTCAACAGGACCAACGGGAACAACT |
| | GGTGTAACCGGAGAAACCGGAGCAACAGGACCAACTGGTGCAACAGGGTCAACCGGAGAAACCGGAGCAACTGGA |
| | GAAACAGGTCGAACAGGACCAACGGGAACAACTGGTGTAACCGGAGAAACCGGAGCAACTGGAGAAACAGGACC |
| | AACAGGAGCAACGGGATCGACTGGAGCAACAGGACCAACGGGAGCAACTGGGGCGACCGGAGCAACAGGTGAAA |
| | CAGGACCAACGGGAGCGACTGGAGAAACAGGAGCAACGGGAGCAACTGGGTCAACCGGAGCAACAGGTCCAACA |
| | GGACCAACGGGAACAACTGGAGTAACCGGAGCAACAGGTCCAACTGGAGCAACTGGAGAAACAGGACCAACGGG |
| | AGCAACTGGAGAAGCAGGACCAACAGGGTCAACCGGATCGACTGGACCAACCGGGGCAACGGGAATCACGGGAGC |
| | AACAGGACCAACAGGAGCAACTGGTGCAACAGGGTCAACCGGAGAAACTGGATCAACTGGAGAAACCGGACCAAC |
| | AGGATCAACTGGT |
| SEQ ID NO: 199 | MDSNKKRCAVCNKYQCSCNSKKRSPKTVIRDLDYIDPCEPLYIIDHCHNRGKTGPTGATGPTGSMGPTGVTGSTGATGPT |
| | GSTGVTGETGSTGTTGPSGSTGPTGATGSTGETGATGSTGATGETGPTGPTGTTGVTGETGATGPTGATGATGETGSTGP |
| | TGATGETGETGSTGPTGATGSTGATGPTGATGSTGATGSTGATGSTGATGPTGATGSTGETGPTGETGSTGATGSTGATG |
| | ETGPTGTTGVTGETGATGPTGATGATGETGETGSTGPTGATGETGETGSTGPTGATGSTGATGPTGATGSTGATGSTGAT |
| | GSTGATGPTGATGSTGETGPTGETGSTGATGSTGATGETGPTGATGSTGATGETGATGPTGPTGTTGVTGATGPTGATGE |
| | TGPTGATGEAGPTGSTGATGPTGATGITGATGPTGSTGATGSTGETGSTGETGPTGATGVTGATGPTGETGSTGPMGATG |
| | ETGSTGSTGVTGATGATGETGATGSTGATGTTGATGETGPTGPTGSTGATGETGPTGPTGATGVTGATGATGATGETGS |
| | TGPMGATGETGSTGSTGVTGATGATGVTGATGETGSTGATGSTGATGETGPTGPTGPTGPTGTTGVTGVTGETGPTGAT |
| | GETGSTGPTGTTGVTGETGATGPTGATGSTGETGATGETGRTGPTGTTGVTGETGATGETGPTGATGSTGATGPTGATG |
| | ATGATGETGPTGATGETGATGATGSTGATGPTGPTGTTGVTGATGPTGATGETGPTGATGEAGPTGSTGSTGPTGATGIT |
| | GATGPTGATGATGSTGETGSTGETGPTGSTG |
| SEQ ID NO: 200 | ATGGATTCAAATAAAACGAGGTGTGCTGTTTGTAATAAATATCAATGTTCTTGTAACTCCAAAAAAAGAAGTCCTAA |
| | AACAGTTATTAGAGATTTAGATTATATAGATCCATGTGAACCTCTTTATATAATAAATCATTGTCATAATAGA |
| SEQ ID NO: 201 | MDSNKTRCAVCNKYQCSCNSKKRSPKTVIRDLDYIDPCEPLYIINHCHNR |
| SEQ ID NO: 202 | ATGGATTCAAATAAAACGAGGTGTGCTGTTTGTAATAAATATCAATGTTCTTGTAACTCCAAAAAAAGAAGTCCTAA |
| | AACAGTTATTAGAGATTTAGATTATATAGATCCATGTGAACCTCTTTATATAATAAATCATTGTCATAATAGAGGTA |
| | AAACTGGACCAACTGGACCAACCGGATCGACCGGACCAACAGGACCAACTGGTGAAACCGGACCAACTGGAGCAA |
| | CAGGACCAACAGGAGCTACTGGAGCAACTGGAGCAACTGGTGCAACTGGAGCAACGGGATCAACTGGAGCAACAG |
| | GATCAACTGGGGCAGCCGGAGTAACTGGTGAAACAGGACTAACAGGGTCAACCGGAGCGACAGGA |
| SEQ ID NO: 203 | MDSNKTRCAVCNKYQCSCNSKKRSPKTVIRDLDYIDPCEPLYIINHCHNRGKTGPTGPTGSTGPTGPTGETGPTGATGPT |
| | GATGATGATGATGATGSTGATGSTGAAGVTGETGLTGSTGATG |
| SEQ ID NO: 204 | ATGGATTCAAATAAAACGAGGTGTGCTGTTTGTAATAAATATCAATGTTCTTGTAATACCAAAAAAAGAAGTCCTAA |
| | AACAGTTATTAGAGATTTAGATTATATAGATCCATGTGAACCTCTTTATATAATAGATCATTGTCATAATAGA |
| SEQ ID NO: 205 | MDSNKTRCAVCNKYQCSCNTKKRSPKTVIRDLDYIDPCEPLYIIDHCHNR |
| SEQ ID NO: 206 | ATGGATTCAAATAAAACGAGGTGTGCTGTTTGTAATAAATATCAATGTTCTTGTAATACCAAAAAAAGAAGTCCTAA |
| | AACAGTTATTAGAGATTTAGATTATATAGATCCATGTGAACCTCTTTATATAATAGATCATTGTCATAATAGAGGTA |
| | AAACAGGACCAACAGGACCAACAGGAGCAACTGGACCAACCGGACCAACTGGAGCAACAGGGGCAACCGGAGCA |
| | ACAGGTCCAACAGGATCAACTGGCGCAACAGGAGCAACTGGACAAACGGGAGCAACTGGAGCAACGGGACCAACA |
| | GGAGCAACAGGAGCAACTGGAGAAACAGGAGCAACAGGAGCAACAGGAGCAACAGGAGCAACAGGACCAACGGG |
| | AGCAACTGGAGAAACAGGAGCA |
| SEQ ID NO: 207 | MDSNKTRCAVCNKYQCSCNTKKRSPKTVIRDLDYIDPCEPLYIIDHCHNRGKTGPTGPTGATGPTGPTGATGATGATGPT |
| | GSTGATGATGQTGATGATGPTGATGATGETGATGATGATGATGPTGATGETGA |
| SEQ ID NO: 208 | ATGGATTCAAATAAAACGAGGTGTGCTGTTTGTAATAAATATCAATGTTCTTGTAACTCCAAAAAAAGAAGTCCTAA |
| | AACAGTTATTAGAGATTTAGATTATATAGATCCATGTGAACCTCTTTATATAATAGATCATTGTCATAATAGA |
| SEQ ID NO: 209 | MDSNKTRCAVCNKYQCSCNSKKRSPKTVIRDLDYIDPCEPLYIIDHCHNR |
| SEQ ID NO: 210 | ATGGATTCAAATAAAACGAGGTGTGCTGTTTGTAATAAATATCAATGTTCTTGTAACTCCAAAAAAAGAAGTCCTAA |
| | AACAGTTATTAGAGATTTAGATTATATAGATCCATGTGAACCTCTTTATATAATAGATCATTGTCATAATAGAGGTA |
| | AAACAGGACCAACAGGAGCAACAGGTCCAACGGGACCAACCGGAGCAACGGGATCAACTGGTCCAACAGGATCAA |
| | CTGGAGCAACTGGAGCAACAGGGTCAACCGGAGCGACTGGAGAAACAGGACCAACTGGGGTAACGGGAATCACGG |
| | GACCGACTGGTGTAACTGGAGCAACAGGGTTCACAGGACTGACTGGAGCAACTGGAGCGACAGGAAATACAGGTC |
| | CAACAGGACCAACCGGAGCA |
| SEQ ID NO: 211 | MDSNKTRCAVCNKYQCSCNSKKRSPKTVIRDLDYIDPCEPLYIIDHCHNRGKTGPTGATGPTGPTGATGSTGPTGSTGAT |
| | GATGSTGATGETGPTGVTGITGPTGVTGATGFTGLTGATGATGNTGPTGPTGA |
| SEQ ID NO: 212 | ATGGATTCAAATAAAACGAGGTGTACTGTTTGTAATAAATATCAATGTTCATGTAACCACAAAAAAAGAAGTCCTA |
| | AAACAGTTATTAGAGATTTAGATTATATAGATCCATGTGAACCGCTTTATATAATAGATCATTGTCATAATAGA |
| SEQ ID NO: 213 | MDSNKTRCTVCNKYQCSCNHKKRSPKTVIRDLDYIDPCEPLYIIDHCHNR |
| SEQ ID NO: 214 | ATGGATTCAAATAAAACGAGGTGTACTGTTTGTAATAAATATCAATGTTCATGTAACCACAAAAAAAGAAGTCCTA |
| | AAACAGTTATTAGAGATTTAGATTATATAGATCCATGTGAACCGCTTTATATAATAGATCATTGTCATAATAGAGGT |
| | AAAACAGGTCCAACGGGGCCAACCGGAGCAACTGGAGCAACAGGTGCAACGGGATCAACTGGGCCGACAGGAGTA |
| | ACTGGTGCAAACTCATATACAAAACAAACACTGCCAACGACGTCAACAGAGAAGAAA |
| SEQ ID NO: 215 | MDSNKTRCTVCNKYQCSCNHKKRSPKTVIRDLDYIDPCEPLYIIDHCHNRGKTGPTGPTGATGATGATGSTGPTGVTGAN |
| | SYTKQTLPTTSTEKK |
| SEQ ID NO: 216 | ATGGATTCAAATAAAACGAGGTGTACTGTTTGTAATAAATATCAATGTTCATGTAACCCCAAAAAAAGAAGTCCTA |
| | AAACAGTTATTAGAGATTTAGATTATATAAATCCATGTGAACCGCTTTATATAATAGATCATTGTCATAATAGA |
| SEQ ID NO: 217 | MDSNKTRCTVCNKYQCSCNPKKRSPKTVIRDLDYINPCEPLYIIDHCHNR |
| SEQ ID NO: 218 | ATGGATTCAAATAAAACGAGGTGTACTGTTTGTAATAAATATCAATGTTCATGTAACCCCAAAAAAAGAAGTCCTA |
| | AAACAGTTATTAGAGATTTAGATTATATAAATCCATGTGAACCGCTTTATATAATAGATCATTGTCATAATAGAGGT |
| | AAAACAGGTCCAACGGGGCCAACCGGAGCAACCGGAGCAACGGGGCCAACCGGACCAACTGGAGCAACAGGGCC |
| | AACCGGAGCGACTGGAGCAACCGGACCAACGGGATCAACAGGTCCTACAGGATCAACCGGAGCAACAGGACCGAC |
| | TGGGGCTACAGGAGTAACTGGAGCAACGGGAGCAACTGGAGCAACGGGAGTAACTGGGGCAACAGGACCAACGGG |
| | ACCAACAGGGTTAACCGGAGCGACTGGAGCAACAGGAACGACTGGGGCTACAGGAGCAACTGGGGCAACGGGAGT |
| | AACTGGAGCAACAGGACCGACTGGGGCAACGGGAGTAACTGGAGCAACGGGAGTAACTGGGGCAACAGGACCAAC |
| | TGGAGCAACAGGATCGACTGGAGAAACTGGAACAACAGGAGTAACTGGAGCAACGGGAGTAACTGGAGCAACAGG |
| | TCCAACCGGGTCAATCGGATCAACGGGAGCAACTGGGGCAACCGGACCAACTGGAGCAACCGGGGCAACAGGAGC |
| | AACCGGACCAACGGGAGAAACCGGACCAACCGGACCAACAGGCCCAACGGGAATCACCGGGCCAACCGGAGCAAC |
| | TGGTTCAGGAGGCGTAATCCCATTTGCATCAGGAGGCACAGTAACAATAAATTCACTATTAACTGGGGTACCTAATA |
| | CCGGAGGTATTATTGCATTTGGCAATAGTACTACTAGTGTAGGGATTGGTAATGCTACAGTAAATCTTCAAAATATT |
| | AATAATGAAGCGTTTGTCGTTCCACATGCTGGAGCAATTACAAATGTTGCTGCATTCTTTAATATTACAGTTGCTCTT |
| | AATATAGCTGTTTTAGGTACAGCTACTATTACGGCACAAGTATGGAAATCACAAAATGCATCATCTAACGTATTTGC |
| | ACCTTTAGCATCGACTCTTATAAGCTTTACACCATTAGCTGGTGCCATAGCTGCATTTACTACATTAAATGGTGCTCT |
| | TGCAAATTTAAATGAACCAGTAAATGTTGGAGATCGTTTAATTATGGTATTCTCTGTGGCTACTACAGGGTTAAGTG |
| | TAATAACTGCTATTACAGGTACAGCAAGTGCGGGTATTACAATTAGTTGA |
| SEQ ID NO: 219 | MDSNKTRCTVCNKYQCSCNPKKRSPKTVIRDLDYINPCEPLYIIDHCHNRGKTGPTGPTGATGATGPTGPTGATGPTGAT |
| | GATGPTGSTGPTGSTGATGPTGATGVTGATGATGATGVTGATGPTGPTGLTGATGATGTTGATGATGATGVTGATGPT |
| | GATGVTGATGVTGATGPTGATGSTGETGTTGVTGATGVTGATGPTGSIGSTGATGATGPTGATGATGATGPTGETGPTG |
| | PTGPTGITGPTGATGSGGVIPFASGGTVTINSLLTGVPNTGGIIAFGNSTTSVGIGNATVNLQNINNEAFVVPHAGAITN |
| | VAAFFNITVALNIAVLGTATITAQVWKSQNASSNVFAPLASTLISFTPLAGALAAFTTLNGALANLNEPVNVGDRLIMVF |
| | SVATTGLSVITAITGTASAGITIS |

TABLE 4
Exemplary Paenibacillus N-Terminal Targeting Sequences

| Sequence | |
|---|---|
| Identifier | Sequence |
| SEQ ID NO: 226 | ATGGTAGTATTATCTACTGGACCTATTGCAAACGATCCTGTTCTAGGAGTCAGACCCACCCAACTGGTCACAGTAAA |
| | AATAGATAACCGAGATTCTGTAAATTCTTCTATCGTTTTGATCGAGGGTTTTATTTTAAACGGTAGCAGAACATTATA |
| | TGTACAACAATTAGTGGTAGTGGGACCAAATGCGGTTATAACGAGGAATTTCTTTGCAAATGTAGACGCATTTGAAT |
| | TCGTTTTTACCACTAGCGGACCAGCAGAGAATGAAACTCAAATTTCTGTTTGGGGTAAAGATGCATTGGGGCAATTA |
| | GTACCTGCCCATCGGTTAGTATCTGACGAACTTTTAGGAACCGATCGA |
| SEQ ID NO: 227 | MVVLSTGPIANDPVLGVRPTQLVTVKIDNRDSVNSSIVLIEGFILNGSRTLYVQQLVVVGPNAVITRNFFANVDAFEFVF |
| | TTSGPAENETQISVWGKDALGQLVPAHRLVSDELLGTDR |
| SEQ ID NO: 228 | ATGCCTGCCTTGGATGAATGGAGTAGTATACAACAAATCGATATGGAGGTGTTTGTATTGGGTCGTCCCGAATTGAA |
| | ACGAAAGAAAGGCCGTAAAAAAGACGTTTTTATCCGCTCTTGGTTTAGTAAAAAACGTCCGAAGAGAAAATGCCAT |
| | TCGAAACGAAAGTGCTTTTGCAAGGAAATCGTCGTCAGAAAGCAAATCGTCCGTGTAAATATACCTCAAAATGTTTT |
| | A |
| SEQ ID NO: 229 | MPALDEWSSIQQIDMEVFVLGRPELKRKKGRKKDVFIRSWFSKKRPKRKCHSKRKCFCKEIVVRKQIVRVNIPQNVL |
| SEQ ID NO: 230 | ATGAAACACAGAAAACCGTTCAGGTTCAGTGGTGCTTCAAAAAAAGACGAGGACTGCAAACCACCTAAAATTAGCA |
| | GAGAAACGGAAGAACTTCTCAAACTGATTAAGGAATTAGTCGCCATCATCCCGCTCGTTTTCGCAAACCCGTCTGTG |
| | GCTAATGTAACTTCATTGCAACAGATTTTACAGCGATTATTAGCTCTCGCAAATAAATTGAGACTTAGAGGCTCGGC |
| | TAAGACAGATTTATTAGCGGCGTTGGAACTGGCTATCGTGGCGTCGGAAGCCACTCTTTTCTCCCCGATCGGTGTTG |
| | GAACGACACTGCAACAACTGCTGGAAGTCTTATTGTCTATTATTTTGCAGGAACCCCTTGATCCTGCTCTTAAAGAC |
| | AGTTTGATCAGTGCAATCAGAAATGCCGAAACGGCTATCAGTATTGCGTTGGGT |
| SEQ ID NO: 231 | MKHRKPFRFSGASKKDEDCKPPKISRETEELLKLIKELVAIIPLVFANPSVANVTSLQQILQRLLALANKLRLRGSAKTD |
| | LLAALELAIVASEATLFSPIGVGTTLQQLLEVLLSIILQEPLDPALKDSLISAIRNAETAISIALG |
| SEQ ID NO: 232 | ATGGCGGTTATATCAACTGGACCCATAGAAAATAATTATGTCAGTGGTATTCGGCCTACTCATCGAGTTACCGTGAA |
| | AATTGATAATCGTGATACTGTGAATTCTTCTACGGTATTGATTCAGGGTTTTTATCTAAATGGTACAAGAACGTTATA |
| | TGTGCTTGATTTTATAACTGTAAATTCAAATGAAGTGATTACAAAAGATTATTATGCTGATTTTAATTCATTTGAGTT |
| | TGTTTTTACCACTGAAAGTGTTACAGAAAATGAGATTCAAGTTTCAGTCTGGGGTAAAAATTCAATGGGGCAGTTAG |
| | TGACAGCTCACCGTGTTGTATCTTCCGAATTGCTTGTAGCAAAAGGCGCG |
| SEQ ID NO: 233 | MAVISTGPIENNYVSGIRPTHRVTVKIDNRDTVNSSTVLIQGFYLNGTRTLYVLDFITVNSNEVITKDYYADFNSFEFVF |
| | TTESVTENEIQVSVWGKNSMGQLVTAHRVVSSELLVAKGA |
| SEQ ID NO: 234 | TTGGGAAATTTATTGTTGCGTAAAAGATATCGCTTGACCCAGGTGGCAAGGAAAAAAAAGAAGGAAAGAGATCAA |
| | AAGATGGGAGCGTTCCGTTTTATGCCCATTTATCGTACAGGAACGAGCTGCATTCGTAACAAAAAGGGAAATAAAC |
| | GTATTTATAGACAGGGTAGAAGAAGAGAGAGAATATGCGCTTATAGACATCATTTGCACGCTGAGCGGGTGCCCTC |
| | AGGTTTATCAAATAAAAAAATCTGTTTTATGAAATTCAAAGGTCAACGAAGACTGCGAGGCGGCGAACAGGAGCCT |
| | CAAGGCAATTCAGGAGGAGCAGTTCAA |
| SEQ ID NO: 235 | LGNLLLRKRYRLTQVARKKKKERDQKMGAFRFMPIYRTGTSCIRNKKGNKRIYRQGRRRERICAYRHHLHAERVPSGLS |
| | NKKICFMKFKGQRRLRGGEQEPQGNSGGAVQ |
| SEQ ID NO: 236 | MGNLLLRKRYRLTQVARKKKKERDQKMGAFRFMPIYRTGTSCIRNKKGNKRIYRQGRRRERICAYRHHLHAERVPSGLS |
| | NKKICFMKFKGQRRLRGGEQEPQGNSGGAVQ |
| SEQ ID NO: 237 | GAAACGGGAGTGGTGAAATCATTGATGCTCAGCGCATTGTTGCGGATGAGCAACTAGATTCTTGAAACACAACATA |
| | TGTACAGAGATAGAACCACAATCGTAACAAATGGTTGAGACATAAAATAGAGGGAACAGGATCTTGAGAAAGATC |
| | TCATTGTTCACAAAAAAGCTTGATTTTACTAGAAAGGAGGGAGTATCCA |
| SEQ ID NO: 238 | CTATATACATGCGCAAAAAACGGCTTCAAACTGCTTCATAATTACGGCACGTTTCTTCTGGCGCCTTCGGCTGTTCCT |
| | TGGTGTGAACCAAGGTAACAGCCGGGGGCGCTATTTTTATATAACTAGATGAATGTACCTGTACAAAGACCCATTTT |
| | TATCCAAAATTAGATCATTGCCTATCAACCACAGGACAGATGTCC |
| SEQ ID NO: 239 | AGCGTTACAAGTTGGAAGCCCGGTTTGGAAATACAGAAAATCGATATTAAAGCTTATGTACAAGCATCCAATAATA |
| | ATTCTTGTGTGGTGATTCACCCTTTTCGCTTCAGTAAATATATTGTTAATATCTGCGAAACGGGGCGATGATCCACCT |
| | GTCACCTCTACAGTAGGGAGAAATGTGAAGGAGGAGATATTTGAAC |
| SEQ ID NO: 240 | AGCGGTATTTTTTGTGCCCCACAAAAAAGGCTCCCTTATCAAAAGGGGTTTTTATCACATAGGAAATGTCCACACGT |
| | ATATATAGATGTTACATATTATATAAATCGTGAACATTCGAATCTCAATACTAGTTATAGAAGAGGTGGCATTAGTG |
| | ATAGGATTATAGCTTCGTTACTTTAGACAAAAGGAGAATCCAATAT |
| SEQ ID NO: 241 | ATTTATTTTTTTGAAAAATTACAGGGGATTCAGTCCCACTTTCAGTAAATTCAGAAAGAAAAATAATGTAACGGCGA |
| | AATGGAAGTGAGCATTAAAAATTTATTTTTTTGGAAAAAAATTTAAGGAGGTCATCTGTCCAATCAGGTTCGTTTAG |
| | ATTCCATAAGATAATGAAACTGTACTTAATTATGGAGGTGTCAGTA |
| SEQ ID NO: 242 | ATGGTAGTATTATCTACTGGACCTATTGCAAACGATCCTGTTCTAGGAGTCAGACCCACCCAACTGGTCACAGTAAA |
| | AATAGATAACCGAGATTCTGTAAATTCTTCTATCGTTTTGATCGAGGGTTTTATTTTAAACGGTAGCAGAACATTATA |
| | TGTACAACAATTAGTGGTAGTGGGACCAAATGCGGTTATAACGAGGAATTTCTTTGCAAATGTAGACGCATTTGAAT |
| | TCGTTTTTACCACTAGCGGACCAGCAGAGAATGAAACTCAAATTTCTGTTTGGGGTAAAGATGCATTGGGGCAATTA |
| | GTACCTGCCCATCGGTTAGTATCTGACGAACTTTTAGGAACCGATCGAGGAATCCAAGGACCTCAAGGAGTTCAGG |
| | GAGCCCAAGGCGACCAAGGTGACCAAGGACCTCAGGGTGTTCAAGGACCTCAAGGAGTTCAGGGAGCCCAAGGAG |
| | ACCAAGGAGTTCAAGGCGTACAAGGAGACCAAGGACCTCAAGGAGTCCAAGGCGACCAAGGTGACCAAGGACCTC |
| | AAGGAGTTCAAGGAGCGCAAGGTGACCAAGGCCCTCAAGGAGTTCAGGGAGCCCAAGGTGACCAAGGACCTCAAG |
| | GCGTTCAGGGAGCGCAAGGTGACCAAGGACCTCAAGGTGATCAAGGACCTCAGGGAGTTCAAGGAGACCAAGGCG |
| | ATCAAGGACCACAGGGAGTTCAAGGCGTACAAGGTGATCAAGGACCTCAGGGTGTTCAAGGAGACCAAGGCGACC |
| | AAGGACCTCAGGGTGTTCAAGGCGTACAAGGTGACCAAGGACCTCAGGGTGTTCAAGGCGTACAAGGTGACCAAG |
| | GACCTCAGGGAGTTCAAGGAGACCAAGGCGATCAAGGACCACAGGGAGTTCAAGGCGTACAAGGTGATCAAGGAC |
| | CTCAGGGTGTTCAAGGAGACCAAGGCGACCAAGGACCTCAGGGTGTTCAAGGCGTACAAGGTGACCAAGGACCTC |
| | AGGGTGTTCAAGGCGTACAAGGTGACCAAGGACCTCAGGGTGTTCAAGGCGTACAAGGTGACCAAGGACCTCAAG |
| | GAGTTCAGGGAGCCCAAGGTGACCAAGGACCACAGGGAGTTCAAGGCGACCAAGGACCTCAAGGACCTCAAGGAG |
| | TTCAAGGTGACCAAGGACCTCAGGGCGTTCAAGGATCCCAAGGTGATCAAGGACCTCAAGGAGTTCAAGGCGTACA |
| | AGGACCTCAAGGAGTTCAAGGCGTACAAGGCGACCAAGGACCTCAAGGTGTTCAGGGAGCCCAAGGCGACCAAGG |
| | CCCTCAAGGAGTTCAAGGAGTCCAAGGTGACCAAGGACCACAGGGAGTTCAAGGACCGCAAGGTGACCAAGGACC |
| | ACAGGGAGTTCAGGGAGTCCAAGGCGACCAAGGACCTCAAGGAGTCCAAGGCGACCAAGGTGACCAAGGACCTCA |
| | AGGAGTTCAAGGAGCGCAAGGTGACCAAGGCCCTCAAGGAGTTCAGGGAGCCCAAGGTGACCAAGGACCTCAAGG |
| | CGTTCAGGGAGCGCAAGGTGACCAAGGACCTCAAGGTGATCAAGGACCTCAGGGAGTTCAAGGAGACCAAGGCGA |
| | TCAAGGACCACAGGGAGTTCAAGGCGTACAAGGTGATCAAGGACCTCAGGGTGTTCAAGGAGACCAAGGCGACCA |
| | AGGACCTCAGGGTGTTCAAGGCGTACAAGGTGACCAAGGACCTCAGGGTGTTCAAGGCGTACAAGGTGACCAAGG |
| | ACCTCAGGGTGTTCAAGGCGTACAAGGTGACCAAGGACCTCAAGGAGTTCAGGGAGCCCAAGGTGACCAAGGACC |
| | ACAGGGAGTTCAAGGCGACCAAGGACCTCAAGGACCTCAAGGAGTTCAAGGTGACCAAGGACCTCAGGGCGTTCA |
| | AGGATCCCAAGGTGATCAAGGACCTCAAGGAGTTCAAGGCGTACAAGGACCTCAAGGAGTTCAAGGCGTACAAGG |
| | CGACCAAGGACCTCAAGGTGTTCAGGGAGCCCAAGGCGACCAAGGCCCTCAAGGAGTTCAAGGAGTCCAAGGTGA |
| | CCAAGGACCACAGGGAGTTCAAGGACCGCAAGGTGACCAAGGACCACAGGGAGTTCAGGGAGTCCAAGGCGACCA |
| | AGGACCTCAAGGTGACCAAGGACCTCAAGGTGACCAAGGACCTCAAGGTGTTCAAGGTGACCAAGGACCTCAAGG |
| | AGTTCAGGGAGCCCAAGGCGACCAAGGACCTCAAGGAGTTCAAGGACCGCAAGGTGACCAAGGACCTCAAGGAGT |
| | TCAAGGCGTACAAGGTGATCAAGGACCTCAAGGAGTTCAAGGCGTACAAGGTGACCAAGGACCACAGGGTGTTCA |
| | AGGCGTACAAGGTGACCAAGGACCTCAAGGTGTTCAAGGAGTCCAAGGTGATCAAGGACCTCAAGGAGTTCAGGG |
| | AGCCCAAGGCGACCAAGGACCTCAGGGAGTTCAGGGAGCCCAAGGCGACCAAGGACCTCAGGGAGTTCAGGGAGC |
| | CCAAGGTGACCAAGGACCTCAGGGCGTTCAAGGAGTACAAGGTGACCAAGGATCTCAAGGAGTTCAAGGACCGCA |
| | AGGTGACCAAGGACCTCAAGGAGTTCAAGGCGTACAAGGTGACCAAGGACCTCAAGGAGTTCAAGGAGTCCAAGG |
| | TGACCAAGGACCTCAAGGTGTTCAGGGAGCCCAAGGTGGCCAAGGACCTCAGGGCGTTCAAGGCGACCAAGGTGA |
| | CCAAGGACCACAGGGTGTTCAAGGATCTCAAGGTGACCAAGGACCACAAGGCGTTCAAGGAGCCCAAGGCGACCA |
| | AGGACCACAGGGTGTTCAAGGCGTACAAGGTGACCAAGGCCCTCAAGGAGTTCAAGGAGTTCAAGGTGACCAAGG |
| | ACCACAGGGAGTTCAAGGTGTTCAAGGACCGCAAGGTGACCAAGGACCACAGGGTGTTCAAGGAGCCCAAGGCGA |
| | CCAAGGACCACAGGGTGTTCAAGGAGTGCAAGGTGACCAAGGACCGCAAGGCGACCAAGGTGACCAAGGACCTCA |
| | AGGTGTTCAGGGAGTCCAAGGCGACCAAGGACCTCAAGGTGTTCAGGGAGTCCAAGGCGACCAAGGACCTCAAGG |
| | TGTTCAAGGTGACCAAGGACCACAGGGAGTTCAGGGAGCCCAAGGTGACCAAGGACCTCAGGGAGTTCAAGGTGA |
| | CCAAGGTGACCAAGGACCTCAAGGAGTTCAAGGTGTACAAGGTGACCAAGGACCTCAAGGTGTTCAGGGAGCCCA |
| | AGGTGACCAAGGACCTCAGGGCGTACAAGGCGACCAAGGTGACCAAGGACCACAGGGTGTTCAAGGCGTACAAGG |
| | TGATCAAGGACCTCAAGGAGTTCAAGGCGTACAAGGTGACCAAGGACCACAGGGTGTTCAAGGCGTACAAGGTGA |
| | CCAAGGACCACAGGGTGTTCAAGGCGACCAAGGTGACCAAGGACCTCAAGGCGTACAAGGCGATCAAGGACCTCA |
| | GGGTGTTCAAGGACCTCAGGGTGTTCAAGGACCTCAGGGTGTTCAAGGACCTCAAGGCGACCAAGGAGCTCAAGGT |
| | GTTCAAGGACCACAAGGTGACCAAGGACCGCAAGGCATTCTGTAA |
| SEQ ID NO: 243 | MVVLSTGPIANDPVLGVRPTQLVTVKIDNRDSVNSSIVLIEGFILNGSRTLYVQQLVVVGPNAVITRNFFANVDAFEFVF |
| | TTSGPAENETQISVWGKDALGQLVPAHRLVSDELLGTDRGIQGPQGVQGAQGDQGDQGPQGVQGPQGVQGAQGDQGVQG |
| | VQGDQGPQGVQGDQGDQGPQGVQGAQGDQGPQGVQGAQGDQGPQGVQGAQGDQGPQGDQGPQGVQGDQGDQGPQ |
| | GVQGVQGDQGPQGVQGDQGDQGPQGVQGVQGDQGPQGVQGVQGDQGPQGVQGDQGDQGPQGVQGVQGDQGPQGV |
| | QGDQGDQGPQGVQGVQGDQGPQGVQGVQGDQGPQGVQGVQGDQGPQGVQGAQGDQGPQGVQGDQGPQGPQGVQG |
| | DQGPQGVQGSQGDQGPQGVQGVQGPQGVQGVQGDQGPQGVQGAQGDQGPQGVQGVQGDQGPQGVQGPQGDQGPQ |
| | GVQGVQGDQGPQGVQGDQGDQGPQGVQGAQGDQGPQGVQGAQGDQGPQGVQGAQGDQGPQGDQGPQGVQGDQGD |
| | QGPQGVQGVQGDQGPQGVQGDQGDQGPQGVQGVQGDQGPQGVQGVQGDQGPQGVQGVQGDQGPQGVQGAQGDQG |
| | PQGVQGDQGPQGPQGVQGDQGPQGVQGSQGDQGPQGVQGVQGPQGVQGVQGDQGPQGVQGAQGDQGPQGVQGVQ |
| | GDQGPQGVQGPQGDQGPQGVQGVQGDQGPQGDQGPQGDQGPQGVQGDQGPQGVQGAQGDQGPQGVQGPQGDQGPQ |
| | GVQGVQGDQGPQGVQGVQGDQGPQGVQGVQGDQGPQGVQGVQGDQGPQGVQGAQGDQGPQGVQGAQGDQGPQGV |
| | QGAQGDQGPQGVQGVQGDQGSQGVQGPQGDQGPQGVQGVQGDQGPQGVQGVQGDQGPQGVQGAQGGQGPQGVQG |
| | DQGDQGPQGVQGSQGDQGPQGVQGAQGDQGPQGVQGVQGDQGPQGVQGVQGDQGPQGVQGVQGPQGDQGPQGVQ |
| | GAQGDQGPQGVQGVQGDQGPQGDQGDQGPQGVQGVQGDQGPQGVQGVQGDQGPQGVQGDQGPQGVQGAQGDQGP |
| | QGVQGDQGDQGPQGVQGVQGDQGPQGVQGAQGDQGPQGVQGDQGDQGPQGVQGVQGDQGPQGVQGVQGDQGPQG |
| | VQGVQGDQGPQGVQGDQGDQGPQGVQGDQGPQGVQGPQGVQGPQGVQGPQGDQGAQGVQGPQGDQGPQGIL |
| SEQ ID NO: 244 | MVVLSTGPIANDPVLGVRPTQLVTVKIDNRDSVNSSIVLIEGFILNGSRTLYVQQLVVVGPNAVITRNFFANVDAFEFVF |
| | TTSGPAENETQISVWGKDALGQLVPAHRLVSDELLGTDRGIQGPQGVQGAQGDQGDQGPQGVQGPQGVQGAQGDQGVQG |
| | VQGDQGPQGVQGDQGDQGPQGVQGAQGDQGPQGVQGAQGDQGPQGVQGAQGDQGPQGDQGPQGVQGDQGDQGPQ |
| | GVQGVQGDQGPQGVQGDQGDQGPQGVQGVQGDQGPQGVQGVQGDQGPQGVQGVQGDQGPQGVQGAQGDQGPQGV |
| | QGDQGPQGLQGVQGDQGPQGVQGSQGDQGPQGVQGVQGPQGGQGVQGDQGPQGVQGAQGDQGPQGVQGDQGDQG |
| | PQGVQGAQGDQGPQGVQGDQGDQGPQGVQGDQGTKELKEYKVTKELKEFKEPKVTKDLRAFKATKVTKDHRVFKEF |
| | KVTKDLKEFKEYKVTKDHRVFKAYKVTKDLKVFKATKVTKDLKAYKAIKDLRVFKDLRVFKDLRVFKDLKATKELKV |
| | FKDHKVTKDRKAFCKLKVKVYLDDSKVIITFGSFFVLS |
| SEQ ID NO: 245 | MVVLSTGPIANDPVLGVRPTQLVTVKIDNRDSVNSSIVLIEGFILNGSRTLYVQQLVVVGPNAVITRNFFANVDAFEFVF |
| | TTSGPAENETQISVWGKDALGQLVPAHRLVSDELLGTDRGIQGPQGVQGAQGDQGDQGPQGVQGPQGVQGAQGDQGVQG |
| | VQGDQGPQGVQGDQGDQGPQGVQGAQGDQGPQGVQGAQGDQGPQGVQGAQGDQGPQGDQGPQGVQGDQGDQGPQ |
| | GVQGVQGDQGPQGVQGDQGDQGPQGVQGVQGDQGPQGVQGVQGDQGPQGVQGVQGDQGPQGVQGAQGDQGPQGV |
| | QGDQGPQGPQGVQGDQGPQGVQGAQGDQGPQGVQGVQGPQGVQGVQGDQGPQGVQGAQGDQGPQGVQGVQGDQG |
| | PQGVQGAQGDQGPQGVQGVQGDQGPQGVQGDQGDQGPQGVQGAQGDQGPQGVQGAQGDQGPQGVQGAQG |
| SEQ ID NO: 246 | ATGCCTGCCTTGGATGAATGGAGTAGTATACAACAAATCGATATGGAGGTGTTTGTATTGGGTCGTCCCGAATTGAA |
| | ACGAAAGAAAGGCCGTAAAAAAGACGTTTTTATCCGCTCTTGGTTTAGTAAAAAACGTCCGAAGAGAAAATGCCAT |
| | TCGAAACGAAAGTGCTTTTGCAAGGAAATCGTCGTCAGAAAGCAAATCGTCCGTGTAAATATACCTCAAAATGTTTT |
| | AGGTATAACAGGCGCAACTGGAGCTATAGGTGTAGCAGGTAACGTAGGTGCAGCGGGCACTGTGGGTGCTGCTGGA |
| | GCCGTCGGAACTGCGGGAAATGTCGGGGCTGCCGGTAATGTGGGTACTGCGGGCACCGTTGGGACTGCCGGAAATG |
| | TAGGCGCAGCGGGGGCTGTGGGCACTGCGGGCGCTGTTGGAGCTGCGGGTGCGGTAGGACCAGTAGGTCCCGTAGG |
| | TCCTGCGGGCATTCCAGGGGCAGTCGGTCCAGCAGGTCCTGCGGGCGTTGCAGGGGCGGTCGGTCCTGTAGGTCCT |
| | GCGGGTGCGGTAGGTGCCACTGGGGCTACGGGTACCGCAGGAGCGACGGGGTCCACCGGGGCTACGGGAGCTACA |
| | GGAACCGCAGGTGGAATAGCTCAGTTTGGTTATATCTACAACTTAGGATCCCGAGTCGTTCCAATAGAAGCGGATGT |
| | CATTTTCGATACGAACGGTATACTTACACCTGGAATTACCCACGCTCCCGGCACTACGCAGATTGCAGTTACCGATG |
| | CGGGGAACTATGAAGTTAACTTTTCAGTATCGGGTGTAGAGCCAGGCCAATTTGCCATATTTATCAATGGCACTCTG |
| | GCAGCAGGAACCATATACGGCTCAGGAGCTGGTACGCAGCAAAACACAGGGCAGGCCATCCTCGCTCTAGCATCCG |
| | GTGATGTTCTTACCCTGCGAAATCATAGCTCTGCCGCTGCGGTTACCCTGCAAACCTTGGCTGGAGGTACCCAAGCC |
| | AACGTAAACGCTTCTGTCGTTATCAAAAAATTAAGTTAG |
| SEQ ID NO: 247 | MPALDEWSSIQQIDMEVFVLGRPELKRKKGRKKDVFIRSWFSKKRPKRKCHSKRKCFCKEIVVRKQIVRVNIPQNVLGIT |
| | GATGAIGVAGNVGAAGTVGAAGAVGTAGNVGAAGNVGTAGTVGTAGNVGAAGAVGTAGAVGAAGAVGPVGPVGPA |
| | GIPGAVGPAGPAGVAGAVGPVGPAGAVGATGATGTAGATGSTGATGATGTAGGIAQFGYIYNLGSRVVPIEADVIFDTN |
| | GILTPGITHAPGTTQLAVTDAGNYEVNFSVSGVEPGQFAIFINGTLAAGTIYGSGAGTQQNTGQAILALASGDVLTLRNH |
| | SSAAAVTLQTLAGGTQANVNASVVIKKLS |
| SEQ ID NO: 248 | ATGAAACACAGAAAACCGTTCAGGTTCAGTGGTGCTTCAAAAAAAGACGAGGACTGCAAACCACCTAAAATTAGCA |
| | GAGAAACGGAAGAACTTCTCAAACTGATTAAGGAATTAGTCGCCATCATCCCGCTCGTTTTCGCAAACCCGTCTGTG |
| | GCTAATGTAACTTCATTGCAACAGATTTTACAGCGATTATTAGCTCTCGCAAATAAATTGAGACTTAGAGGCTCGGC |
| | TAAGACAGATTTATTAGCGGCGTTGGAACTGGCTATCGTGGCGTCGGAAGCCACTCTTTTCTCCCCGATCGGTGTTG |
| | GAACGACACTGCAACAACTGCTGGAAGTCTTATTGTCTATTATTTTGCAGGAACCCCTTGATCCTGCTCTTAAAGAC |
| | AGTTTGATCAGTGCAATCAGAAATGCCGAAACGGCTATCAGTATTGCGTTGGGTGGCACGGCAGGAACCCCCGGTC |
| | CACAAGGGCCCGCTGGGCCTGCTGGTCCGGGCGGTGCTCCAGGACCTGTCGGTGGACCAGGGCCGGTGGGTGCGGC |
| | AGGACCAGCAGGTCCAGTTGGACCTGCTGGTCCTGTCGGACCTGTCGGGGCTGCCGGACCTGTTGGAGCCGCCGGA |
| | CCTGTTGGAGCCGCCGGACCTATCGGCGCCGCTGGGCCAGTAGGCGCCGCCGGGGCTGCTGGAGCCACCGGGGCTA |
| | CAGGAGCTACAGGCGCGGCAGGACCTGCCGGGGGGGCTACCGGGGCCACGGGCGCCGTTGGAGCCACAGGCGCTA |
| | CGGGCGCAGCGGGGGTCGCTGGGGCTACAGGAACTACGGGCACGGCGGGCGCTGTCGGAGCTACCGGGGCCACGG |
| | GCACGGCGGGGGCCATTGGAGCTACCGGGGCCACAGGCACGGCGGGGGCCGTCGGAGCTACCGGGGCCACAGGCA |
| | CGGCGGGCGCTGTCGGAGCTACCGGGGCCACGGGTACAGCAGGGGTTACTGGAGCCACCGGTTCGGGGGCAATCAT |
| | TCCATTTGCTTCGGGTGGACCAGCAATTTTGACAACCATTGTCGGCGGGCTGGTTGGAACCACAAGTTTGATCGGCT |
| | TTGGAAGCTCAGCAACAGGCATTAGCCTTGTGGGTGGAACCATTGACCTGACAGGCACACTTGCAGGGCCACTGAT |
| | TAACTTTGCTTTTTCTGTACCACGGGATGGCGTAATTACATCCATCGCTGGATATTTTAGTACAACAGCTGCGCTAAC |
| | TCTCGTTGGATCAACCGCGACGATTACTGCCCAGTTGTTTAGTTCGACTACACCTGATAACACCTTTACAGCGGTCCC |
| | TGGGGCTACCGTTACATTAGCTCCACCACTGACTGGCATCATTGCCTTGGGTACCATTTCCAATGGCATCACTACCG |
| | GATTGGCTATACCAGTAACCGCGCAGACTCGTCTGCTCCTTGTCTTCTCTGCAACAGCTACGGGACTCTCCCTCGTAA |
| | ACACCATCGTGGGTTATGCGAGCGCAGGCATTACCATCACCTGA |
| SEQ ID NO: 249 | MKHRKPFRFSGASKKDEDCKPPKISRETEELLKLIKELVAIIPLVFANPSVANVTSLQQILQRLLALANKLRLRGSAKTD |
| | LLAALELAIVASEATLFSPIGVGTTLQQLLEVLLSIILQEPLDPALKDSLISAIRNAETAISIALGGTAGTPGPQGPAGP |
| | AGPGGAPGPVGGPGPVGAAGPAGPVGPAGPVGPVGAAGPVGAAGPVGAAGPIGAAGPVGAAGAAGATGATGATGAAGPAG |
| | GATGATGAVGATGATGAAGVAGATGTTGTAGAVGATGATGTAGAIGATGATGTAGAVGATGATGTAGAVGATGATGTAG |
| | VTGATGSGAIIPFASGGPAILTTIVGGLVGTTSLIGFGSSATGISLVGGTIDLIGTLAGPLINFAFSVPRDGVITSIAGY |
| | FSTTAALTLVGSTATITAQLFSSTTPDNTFTAVPGATVTLAPPLTGIIALGTISNGITTGLAIPVTAQTRLLLVFSATAT |
| | GLSLVNTIVGYASAGITIT |
| SEQ ID NO: 250 | ATGGCGGTTATATCAACTGGACCCATAGAAAATAATTATGTCAGTGGTATTCGGCCTACTCATCGAGTTACCGTGAA |
| | AATTGATAATCGTGATACTGTGAATTCTTCTACGGTATTGATTCAGGGTTTTTATCTAAATGGTACAAGAACGTTATA |
| | TGTGCTTGATTTTATAACTGTAAATTCAAATGAAGTGATTACAAAAGATTATTATGCTGATTTTAATTCATTTGAGTT |
| | TGTTTTTACCACTGAAAGTGTTACAGAAAATGAGATTCAAGTTTCAGTCTGGGGTAAAAATTCAATGGGGCAGTTAG |
| | TGACAGCTCACCGTGTTGTATCTTCCGAATTGCTTGTAGCAAAAGGCGCGGGACCGACAGGGCTAACGGGAGCCAC |
| | TGGCGCTACCGGAGCTACTGGCGTCACGGGTGTTACCGGAGTCACTGGCGCTACCGGAACTACGGGCGTTATGGGT |
| | GATACCGGAGTCACTGGAGTTACCGGAGTTACTGGCGTTACCGGGGCTATCGGAGTCACTGGCGCTATCGGAGTCA |
| | CGGGGGCTACCGGAGCCACAGGAGTTACGGGGGCCACTGGAGTTACCGGGGCTATTGGAGTTACTGGCGCTATCGG |
| | AGTCACTGGCGCTACCGGAGCTACTGGCGTTACTGGGGCTACTGGCGCTACTGGAGTCACAGGAGTTACCGGGGCT |
| | ACTGGCGTTACCGGAGTTACCGGAGTTACTGGCATCACCGGGGCTATCGGAGCTACTGGCGTTACCGGAGCTACTG |
| | GCGTCACGGGTATTACCGGAGTCACTGGCGTTACCGGGGCTACTGGCGTTACTGGAGTTACTGGCATCACAGGCGTT |
| | ACCGGAGTTACTGGTGTTACTGGTGTTACTGGAGCTACTGGCGTTACCGGGGCTACTGGCGCTACCGGAGCCACTGG |
| | CGTTACTGGAGTTACTGGCGTTACTGGCGCTACTGGAGCTACTGGTGTTACCGGGGCTACCGGGGCTACCGGTGTCA |
| | CGGGTGATACCGGTGTCACTGGCGCTACCGGGGCTACCGGAGTTTCTGGCGCTACTGGGGCTACTGGTGTCACGGGT |
| | GATACCGGAGTTACCGGAGCTACTGGCGCTACAGGTGCTACCGGAGTTACTGGCGGAACAGGTGCAACCGGAGTTA |
| | CTGGAGTTACTGGCGTTACCGGGGCTATCGGAGTCACTGGCGCTACTGGAGCTACTGGAGCTGCTGGAATCACGGG |
| | TGTTACCGGAGTTACTGGCATCACCGGTGCTACCGGGGCTACGGGCGCTACCGGAGTTACTGGCATCACAGGAGTC |
| | ACTGGCGCTACCGGAGTTACTGGCGTAACAGGTGCAACCGGAGTTACTGGAGTTACCGGGGCTATCGGAGTTACTG |
| | GTGTCACCGGAGCTACTGGCGTCACGGGTGTTACCGGAGTCACTGGCGCTACCGGAGCTACTGGCGTTACGGGTGTT |
| | ACCGGAGTTACCGGAGTTACTGGCGTTACCGGAGCTACTGGCGTTACCGGAGTTACTGGAGTTACTGGAGTTATTGG |
| | AGTTACTGGAGTTACTGGAGTTACTGGAGTTACTGGAGTTACCGGAGTTACCGGAGTTACTGGAGTTACCGGGGCTA |
| | TCGGAGTCACTGGCGCTATCGGAGTCACGGGGGCTACCGGGGTCACTGGCGCTACCGGAGCTACTGGCGTAACAGG |
| | GGCTACTGGAGTTACCGGGGCTATCGGAGTCACTGGCGCTACTGGAGCTGCTGGAATCACGGGTGTTACCGGAGTC |
| | ACTGGTGTTACTGGAGTTACCGGAGCTACTGGCATCACGGGTGATACCGGAGTCACTGGCGCTACCGGAGCTACTG |
| | GCGTTACGGGTGTTACCGGAGTCACTGGGGCTACCGGAGCTACTGGCGTCACGGGTGATACCGGAGTTACTGGAGT |
| | CACTGGCGCTACCGGAGTTACTGGCGTAACAGGTGCAGCCGGAGTTACTGGCATCACGGGGGCTACCGGAGTTACT |
| | GGAGTTACCGGGGCTATTGGAGTCACTGGCGCTATCGGAGTCACGGGGGCTACCGGAGCCACAGGAGTTACGGGTA |
| | TTACCGGAGCTACTGGCGCTACTGGAGCCACAGGTGCTACCGGAGTTACTGGAGTTACTGGCGCTACCGGAGCTACT |
| | GGCGCTACTGGCGTCACGGGTTCTACTGGGGTCACTGGCGCTACTGGCGTTACCGGAGCTACTGGCGTCACGGGTTC |
| | TACTGGGGTCACTGGCGCTACTGGCGTTACCGGAGCTACTGGCGTCACGGGTATTACCGGAGTCACTGGCGTTACCG |
| | GAGTTACTGGTGCTACTGGAGCTACTGGCGTTACCGGGGCTACCGGAGTCACTGGGGCTACCGGAGCTACTGGCGT |
| | CACGGGTATTACCGGAGTCACTGGGGCTACCGGAGCTACTGGCGTCACGGGTGTTACCGGAGTCACCGGAGTCACT |
| | GGAGTTACTGGAGTTACTGGCGCTACCGGAGCTACTGGCGTTACCGGAGCTACTGGCGCTACTGGCGTCACGGGTG |
| | ATACCGGAGTCACTGGGGCTACCGGAGTTACCGGAGTCACTGGCGCTACTGGGGCTACTGGTGTCACGGGTGTTAC |
| | CGGAGTCACTGGCGCTACCGGGGCTACTGGTGTCACGGGTGTTACCGGGGCTACCGGAGCTACTGGCGACACGGGT |
| | GTTACCGGAGTCACTGGAGTCACTGGAGTTACCGGAGTTTCTGGCGCTACCGGAGTTACCGGAGTTTCTGGCGCTAC |
| | CGGAGTTACCGGAGCTACTGGCGTTACCGGGGCTGGGGCTACCGGAGCTACTGGCGCTACTGGAGTCACAGGTGTT |
| | ACCGGAGTCACTGGCGCTACCGGAGCTACTGGCGCTACTGGAGTCACGGGTGTTACCGGAGTCACTGGCGCTACCG |
| | GGGCTACTGGTGTCACGGGTGTTACCGGGGCTACCGGAGCTACTGGCGACACGGGTGTTACCGGAGTCACTGGAGT |
| | CACTGGAGTTACCGGAGTTTCTGGCGCTACCGGAGTTACCGGAGCTACTGGCGTTACCGGGGCTGGGGCTACCGGA |
| | GCTACTGGCGCTACTGGAGTCACAGGTGTTACCGGAGTCACTGGCGCTACCGGAGCTACTGGCGCTACTGGAGTCA |
| | CGGGTGTTACCGGAGTCACTGGCGCTACCGGGGCTACTGGCGCTACTGGAGTCACGGGTGTTACTGGCGTTACGGGT |
| | GTTACCGGAGTTTCTGGCATCACCGGTGCTACCGGGGCTATTGGACCTACTGGTGCCACAGGTGTTGGTATAACAGG |
| | TTCAACAGGTTCAACCGGCCCCACTGGCCCACCTCCTACGTTTATAGACGCATACTTTAACGGTAATATTCAACCTC |
| | AGACAATTGCTTCGGGATCAAACATTTTAAATATTACTCCAAACCAATCTACTGCACTTACTTATAACGCAGTAACA |
| | AGTGTTTTCACAATACAAAATGCGGGGTTGTATAACATTAGTGTTGTAATAAATCTTGCAACTGCCACACTACCAGA |
| | AGCAACAATTGGGTTATCACTAAATAATTCTACAGCATATCTCGCTCCTGCTGTAACCACGGCAACAAGTGGTCAAT |
| | TGGTTTTAGTTCAAATTGAGGCTCTTGCTGTCGGAGATACAATTCAATTTAGAAATATATCTGGGTTTCCTATTACCA |
| | TTGCTAATTCACCAGTAATAGCTAACAGCTCAGGTCATGTAGCTATTTCGAGATTCTCAGCTTTTTCATAA |
| SEQ ID NO: 251 | MAVISTGPIENNYVSGIRPTHRVTVKIDNRDTVNSSTVLIQGFYLNGTRTLYVLDFITVNSNEVITKDYYADFNSFEFVF |
| | TTESVTENEIQVSVWGKNSMGQLVTAHRVVSSELLVAKGAGPTGLTGATGATGATGVTGVTGVTGATGTTGVMGDTGVT |
| | GVTGVTGVTGAIGVTGAIGVTGATGATGVTGATGVTGAIGVTGAIGVTGATGATGVTGATGATGVTGVTGATGVTGVT |
| | GVTGITGAIGATGVTGATGVTGITGVTGVTGATGVTGVTGITGVTGVTGVTGVTGATGVTGATGATGATGVTGVTGVT |
| | GATGATGVTGATGATGVTGDTGVTGATGATGVSGATGATGVTGDTGVTGATGATGATGVTGGTGATGVTGVTGVTG |
| | AIGVTGATGATGAAGITGVTGVTGITGATGATGATGVTGITGVTGATGVTGVTGATGVTGVTGAIGVTGVTGATGVTGV |
| | TGVTGATGATGVTGVTGVTGVTGVTGATGVTGVTGVTGVIGVTGVTGVTGVTGVTGVTGVTGVTGAIGVTGAIGVTG |
| | ATGVTGATGATGVTGATGVTGAIGVTGATGAAGITGVTGVTGVTGVTGATGITGDTGVTGATGATGVTGVTGVTGATG |
| | ATGVTGDTGVTGVTGATGVTGVTGAAGVTGITGATGVTGVTGAIGVTGAIGVTGATGATGVTGITGATGATGATGATG |
| | VTGVTGATGATGATGVTGSTGVTGATGVTGATGVTGSTGVTGATGVTGATGVTGITGVTGVTGVTGATGATGVTGAT |
| | GVTGATGATGVTGITGVTGATGATGVTGVTGVTGVTGVTGVTGATGATGVTGATGATGVTGDTGVTGATGVTGVTGA |
| | TGATGVTGVTGVTGATGATGVTGVTGATGATGDTGVTGVTGVTGVTGVSGATGVTGVSGATGVTGATGVTGAGATG |
| | ATGATGVTGVTGVTGATGATGATGVTGVTGVTGATGATGVTGVTGATGATGDTGVTGVTGVTGVTGVSGATGVTGA |
| | TGVTGAGATGATGATGVTGVTGVTGATGATGATGVTGVTGVTGATGATGATGVTGVTGVTGVTGVSGITGATGAIGPT |
| | GATGVGITGSTGSTGPTGPPPTFIDAYFNGNIQPQTIASGSNILNITPNQSTALTYNAVTSVFTIQNAGLYNISVVINLA |
| | TATLPEATIGLSLNNSTAYLAPAVTTATSGQLVLVQIEALAVGDTIQFRNISGFPITIANSPVIANSSGHVAISRFSAFS |
| SEQ ID NO: 252 | TTGGGAAATTTATTGTTGCGTAAAAGATATCGCTTGACCCAGGTGGCAAGGAAAAAAAAGAAGGAAAGAGATCAA |
| | AAGATGGGAGCGTTCCGTTTTATGCCCATTTATCGTACAGGAACGAGCTGCATTCGTAACAAAAAGGGAAATAAAC |
| | GTATTTATAGACAGGGTAGAAGAAGAGAGAGAATATGCGCTTATAGACATCATTTGCACGCTGAGCGGGTGCCCTC |
| | AGGTTTATCAAATAAAAAAATCTGTTTTATGAAATTCAAAGGTCAACGAAGACTGCGAGGCGGCGAACAGGAGCCT |
| | CAAGGCAATTCAGGAGGAGCAGTTCAAGGGGTGCATGGATTAAGGGGGACCGATGGTAATGCTGGGCATCAAGGC |
| | ATACAAGGTCCGGCTGGGCCACAGGGCATTCCGGGTAGTGCCGGACCCCAGGGCCAGGCGGGCGCCATAGGCCCCC |
| | AAGGTGAACAGGGTCTTCAGGGGGTTCCAGGGATTCAAGGCTTGCAAGGAGAGGCTGGGCCACAGGGAGAGCAGG |
| | GACCACCGCTTAATTTGGATGGGATCACGGTTGTGCCTGAGGTACAGCGATATTTCTATTTTGCCGATTCAGATCTG |
| | ACGGGTACGGTTGAAATCCCTATTTCCCAGTTTACGAATGATGATGGACAGTTGGCAAGTCAGCTTCCAGAATTGGG |
| | TGCGAACAGCTACACGGATTTGTATATTAATGGGGTACTGCAGGAAAGCAGGTTGTACCAGATAAGTAGTACCACA |
| | TTGACTGTTGAATTGGAAGAAGCTCTTGTAATTGCGGGTACGCCGTTTATTTTCGAGGTTTTTCAATTTACATTAAGA |
| | ATGGCGAACTGA |
| SEQ ID NO: 253 | MGNLLLRKRYRLTQVARKKKKERDQKMGAFRFMPIYRTGTSCIRNKKGNKRIYRQGRRRERICAYRHHLHAERVPSGLS |
| | NKKICFMKFKGQRRLRGGEQEPQGNSGGAVQGVHGLRGTDGNAGHQGIQGPAGPQGIPGSAGPQGQAGAIGPQGEQGL |
| | QGVPGIQGLQGEAGPQGEQGPPLNLDGITVVPEVQRYFYFADSDLTGTVEIPISQFTNDDGQLASQLPELGANSYTDLYI |
| | NGVLQESRLYQISSTTLTVELEEALVIAGTPFIFEVFQFTLRMAN |
| SEQ ID NO: 254 | MXVXSTGPIXNXXVXGXRPTXXVTVKIDNRDXVNSSXVLIXGFXLNGXRTLYVXXXXXVX |
| SEQ ID NO: 255 | MXVXSTGPIXNXXVXGXRPTXXVTVKIDNRDXVNSSXVLIXGFXLNGXRTLYVXXXXXVX |
| | XNXVITXXXXXXXXFEFVFTTXXXXENEXQXSVWGKXXXGQLVXAHRXVSXELLVXXXX |
| SEQID NO: 256 | ATGGTAGTATTATCTACTGGACCTATTGCAAACGATCCTGTTCTAGGAGTCAGACCCACC |
| | CAACTGGTCACAGTAAAAATAGATAACCGAGATTCTGTAAATTCTTCTATCGTTTTGATC |
| | GAGGGTTTTATTTTAAACGGTAGCAGAACATTATATGTACAACAATTAGTGGTAGTGGGA |
| | CCAAATGCGGTTATAACGAGGAATTTCTTTGCAAATGTAGACGCATTTGAATTCGTTTTT |
| | ACCACTAGCGGACCAGCAGAGAATGAAACTCAAATTTCTGTTTGGGGTAAAGATGCATTG |
| SEQ ID NO: 257 | MVVLSTGPIANDPVLGVRPTQLVTVKIDNRDSVNSSIVLIEGFILNGSRTLYVQQLVVVGPNAVITRNFFANVDAFEFVF |
| | TTSGPAENETQISVWGKDAL |
| SEQ ID NO: 258 | ATGGTAGTATTATCTACTGGACCTATTGCAAACGATCCTGTTCTAGGAGTCAGACCCACCCAACTGGTCACAGTAAA |
| | AATAGATAACCGAGATTCTGTAAATTCTTCTATCGTTTTGATCGAGGGTTTTATTTTAAACGGTAGCAGAACATTATA |
| | TGTACAACAATTAGTGGTAGTGGGACCAAATGCGGTTATAACGAGGAATTTCTTTGCAAATGTAGACGCATTTGAAT |
| | TCGTTTTT |
| SEQ ID NO: 259 | MVVLSTGPIANDPVLGVRPTQLVTVKIDNRDSVNSSIVLIEGFILNGSRTLYVQQLVVVGPNAVITRNFFANVDAFEFVF |
| SEQ ID NO: 260 | ATGGTAGTATTATCTACTGGACCTATTGCAAACGATCCTGTTCTAGGAGTCAGACCCACCCAACTGGTCACAGTAAA |
| | AATAGATAACCGAGATTCTGTAAATTCTTCTATCGTTTTGATC |
| SEQ ID NO: 261 | MVVLSTGPIANDPVLGVRPTQLVTVKIDNRDSVNSSIVLI |
| SEQ ID NO: 262 | ATGTTTACCACTAGCGGACCAGCAGAGAATGAAACTCAAATTTCTGTTTGGGGTAAAGATGCATTG |
| SEQ ID NO: 263 | MFTTSGPAENETQISVWGKDAL |
| SEQ ID NO: 264 | ATGACTCAAATTTCTGTTTGGGGTAAAGATGCATTG |
| SEQ ID NO: 265 | MTQISVWGKDAL |
| SEQ ID NO: 266 | ATGACTCAAATTTCTGTTTGG |
| SEQ ID NO: 267 | MTQISVW |
| SEQ ID NO: 268 | ATGCAAATTTCTGTTTGGGGTAAAGAT |
| SEQ ID NO: 269 | MQISVWGKD |
| SEQ ID NO: 270 | ATGTGGGGTAAAGATGCATTG |
| SEQ ID NO: 271 | MWGKDAL |
| SEQ ID NO: 272 | MQISVWGKB |

Intergeneric N-terminal targeting sequences compatible with Brevibacillus, Lysinibacillus, Viridibacillus, and/or Paenibacillus are shown in Tables 1-4 and FIGS. 1-7. In addition to these sequences, the N-terminal targeting sequence may alternatively comprise a variant sharing at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to any of the sequences disclosed herein, so long as the sequence retains the capability to target the fusion protein to the exosporium of at least two of Brevibacillus, Lysinibacillus, Viridibacillus, and/or Paenibacillus. In some embodiments, a fragment of at least 10, 11, 12, 13, 14, 15, 16, 17, 18, 19, 20, 21, 22, 23, 24, 25, 26, 27, 28, 29, or 30 contiguous amino acids selected from any of the sequences disclosed in Tables 1-4 or FIGS. 1-7. This contiguous segment may include the N-terminal end or the C-terminal end of any of the sequences disclosed herein (e.g., an N-terminal targeting sequence may comprise the last 25 contiguous amino acids of any sequence disclosed herein). In some aspects, the only required functionality is that the sequence maintains the capability to target a fusion protein to the exosporium of at least two of Brevibacillus, Lysinibacillus, Viridibacillus, and/or Paenibacillus (i.e., intergeneric exosporium-targeting functionality must be maintained).

The N-terminal signal sequence used to target the fusion protein to the exosporium may comprise a polypeptide having a sequence as disclosed in Tables 1-4 or FIGS. 1-7. Alternatively, in select embodiments this N-terminal signal sequence may comprise a variant or fragment thereof that targets the fusion protein to an exosporium of at least two of Brevibacillus, Lysinibacillus, Viridibacillus, and/or Paenibacillus and a molecule of interest (e.g., peptide or polypeptide) sequence heterologous to the N-terminal signal sequence. In select embodiments, the N-terminal signal sequence comprises an amino acid sequence having at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity with the amino acid sequence of any of the sequences disclosed in Tables 1-4 or FIGS. 1-7. In select embodiments, the N-terminal signal sequence comprises a contiguous sequence of at least 10, 11, 12, 13, 14, 15, 16, 17, 18, 19, 20, 21, 22, 23, 24, or 25 amino acids that is identical to a contiguous sequence of the same number amino acids disclosed in Tables 1-4 or FIGS. 1-7.

As discussed herein, fusion protein constructs according to several aspects of the disclosure comprise an N-terminal signal sequence or a variant or fragment thereof that targets the fusion protein to the exosporium of a Brevibacillus, Lysinibacillus, Viridibacillus, or Paenibacillus endospore and a polypeptide sequence that is heterologous to the N-terminal signal sequence. However, in further aspects, any of the disclosed sequences, as well as the sequential variants and fragments thereof according to any of the disclosed aspects, may be used for other purposes. The disclosure's focus on aspects wherein these sequences function as N-terminal exosporium targeting sequences is not to be construed as a disclaimer of other functionalities.

In some embodiments, the N-terminal signal sequence comprises a polypeptide with an amino acid sequence represented by any of the sequences disclosed in Tables 1-4 or FIGS. 1-7. In alternative embodiments, the N-terminal signal sequence comprises a fragment of any of the sequences disclosed in Tables 1-4 or FIGS. 1-7, e.g., a polypeptide with an amino acid sequence comprising at least one contiguous subsequence found in any of the sequences disclosed in these tables or figures. In alternative embodiments, the N-terminal signal sequence comprises a variant of any of a sequence disclosed in Tables 1-4 or FIGS. 1-7, e.g., a polypeptide with an amino acid sequence that shares a minimum or exact degree of percentage identity with a sequence disclosed in these tables or figures. In select embodiments, the N-terminal signal sequence may qualify as both a fragment and as a variant, as defined above (e.g., an N-terminal signal sequence comprising a contiguous subsequence of a sequence disclosed herein, as well as a divergent sequence that falls within a disclosed sequence identity range).

In select embodiments, the N-terminal signal sequence comprises an amino acid sequence having at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity with an amino acid sequence disclosed in Tables 1-4 or FIGS. 1-7.

In select embodiments, the N-terminal signal sequence comprises a contiguous sequence of at least 10, 20 or 25 amino acids that is identical to a contiguous sequence of at least 10, 11, 12, 13, 14, 15, 16, 17, 18, 19, 20, 21, 22, 23, 24, or 25 amino acids of an amino acid sequence disclosed in Tables 1-4 or FIGS. 1-7.

In some embodiments, the N-terminal signal sequence comprises a polypeptide with an amino acid sequence encoded by any of the nucleotide sequences disclosed in Tables 1-4 or FIGS. 1-7. In alternative embodiments, the N-terminal signal sequence comprises a fragment of a polypeptide with an amino acid sequence encoded by any of the nucleotide sequences disclosed in Tables 1-4 or FIGS. 1-7, e.g., a polypeptide with an amino acid sequence comprising at least one contiguous subsequence found in a polypeptide with an amino acid sequence encoded by a nucleotide sequence disclosed in these tables or figures. In alternative embodiments, the N-terminal signal sequence comprises a variant of a polypeptide with an amino acid sequence encoded by a nucleotide sequence any of the nucleotide sequences disclosed in Tables 1-4 or FIGS. 1-7, e.g., a polypeptide with an amino acid sequence that shares a minimum or exact degree of percentage identity with a polypeptide with an amino acid sequence encoded by a nucleotide sequence disclosed in these tables or figures. In select embodiments, the N-terminal signal sequence may qualify as both a fragment and as a variant, as defined above, e.g., an N-terminal signal sequence comprising a contiguous subsequence found in a polypeptide with an amino acid sequence disclosed in Tables 1-4 or FIGS. 1-7, followed by a divergent sequence that falls within a minimum sequence identity range as described herein.

In select embodiments, the N-terminal signal sequence comprises a nucleotide sequence having at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity with any of the nucleotide sequences disclosed herein, e.g., any nucleotide sequences disclosed in Tables 1-4 or FIGS. 1-7.

In select embodiments, the N-terminal signal sequence comprises a nucleotide sequence that hybridizes to a nucleic acid probe complementary to a polynucleotide encoding any of the polypeptide sequences disclosed in Tables 1-4 or FIGS. 1-7, or a fragment thereof, under moderate or high stringency.

In select embodiments, the N-terminal signal sequence comprises a contiguous sequence of at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides that is identical to a contiguous sequence of the same number of nucleotides in any of the nucleotide sequences disclosed in Tables 1-4 or FIGS. 1-7.

With respect to any of the alternative N-terminal targeting sequences contemplated by this disclosure, such as the aforementioned embodiments, the minimum required functionality of such sequences in selected aspects is the capability to target a fusion protein to the exosporium of a Brevibacillus, Lysinibacillus, Viridibacillus, or Paenibacillus endospore.

### Fusion Proteins

The disclosure provides fusion proteins comprising an intergeneric N-terminal targeting sequence linked, directly or indirectly, to at least one molecule of interest (e.g., polypeptide sequence of a protein or peptide of interest, such as at least one plant growth stimulating protein or peptide). In selected embodiments, the indirect linkage may be an intervening spacer, linker or a regulatory sequence. The protein or peptide may comprise, but is not limited to, a peptide hormone, a non-hormone peptide, an enzyme involved in the production or activation of a plant growth stimulating compound or an enzyme that degrades or modifies a bacterial, fungal, or plant nutrient source. In general, any protein of interest capable of expression in a Brevibacillus, Lysinibacillus, Viridibacillus, or Paenibacillus endospore and heterologous to the selected N-terminal targeting sequence may be used. The targeting sequence can be any of the targeting sequences described above.

In some embodiments, the fusion proteins can comprise an N-terminal targeting sequence and at least one protein or peptide that protects a plant from a pathogen. The N-terminal targeting sequence can be any of the targeting sequences described above.

The fusion protein can be made using standard cloning and molecular biology methods known in the art. For example, a gene encoding a protein or peptide (e.g., a gene encoding a plant growth stimulating protein or peptide) can be amplified by polymerase chain reaction (PCR) and ligated to DNA coding for any of the above-described targeting sequences to form a DNA molecule that encodes the fusion protein. The DNA molecule encoding the fusion protein can be cloned into any suitable vector, for example a plasmid vector. The vector suitably comprises a multiple cloning site into which the DNA molecule encoding the fusion protein can be easily inserted. The vector also suitably contains a selectable marker, such as an antibiotic resistance gene, such that bacteria transformed, transfected, or mated with the vector can be readily identified and isolated. Where the vector is a plasmid, the plasmid suitably also comprises an origin of replication. The DNA encoding the fusion protein is suitably under the control of a sporulation promoter that will cause expression of the fusion protein on the exosporium of a Brevibacillus, Lysinibacillus, Viridibacillus, or Paenibacillus endospore (e.g., a native promoter from a Brevibacillus, Lysinibacillus, Viridibacillus, or Paenibacillus family member). Alternatively, DNA coding for the fusion protein, e.g., a sequence comprising any of the polynucleotide sequences disclosed in Tables 1-4 or FIGS. 1-7 can be integrated into the chromosomal DNA of a Brevibacillus, Lysinibacillus, Viridibacillus, or Paenibacillus cell.

The fusion protein can also comprise additional polypeptide sequences that are not part of the targeting sequence, or the linked protein of interest (e.g., the plant growth stimulating protein or peptide, the protein or peptide that protects a plant from a pathogen, the protein or peptide that enhances stress resistance in a plant, or the plant binding protein or peptide). For example, the fusion protein can include tags or markers to facilitate purification (e.g., a polyhistidine tag) or visualization (e.g., a fluorescent protein such as GFP or YFP) of the fusion protein itself or of the recombinant exosporium-producing Brevibacillus, Lysinibacillus, Viridibacillus, or Paenibacillus cells' spores expressing the fusion protein.

Expression of fusion proteins on the exosporium using the targeting sequences described herein is enhanced due to a lack of secondary structure in the amino-termini of these sequences, which allows for native folding of the fused proteins and retention of activity. Proper folding can be further enhanced by the inclusion of a short amino acid linker between the targeting sequence and the fusion partner protein.

Thus, any of the fusion proteins described herein can comprise an amino acid linker between the targeting sequence and the linked protein of interest (e.g., the plant growth stimulating protein or peptide, the protein or peptide that protects a plant from a pathogen, the protein or peptide that enhances stress resistance in a plant, or the plant binding protein or peptide).

The linker can comprise a polyalanine linker or a polyglycine linker. A linker comprising a mixture of both alanine and glycine residues can also be used. For example, where the targeting sequence comprises SEQ ID NO: 4, a fusion protein can have one of the following structures:
No linker: SEQ ID NO: 4—Fusion Partner Protein
Alanine Linker: SEQ ID NO: 4—Aₙ-Fusion Partner Protein
Glycine Linker: SEQ ID NO: 4—Gₙ-Fusion Partner Protein
Mixed Alanine and Glycine Linker: SEQ ID NO: 4—(A/G)ₙ-Fusion Partner Protein

where Aₙ, Gₙ, and (A/G), are any number of alanines, any number of glycines, or any number of a mixture of alanines and glycines, respectively.

For example, n can be any integer between 1 to 25, such as an integer between 6 to 10. Where the linker comprises a mixture of alanine and glycine residues, any combination of glycine and alanine residues can be used. The N-terminal targeting sequence represented by SEQ ID NO: 4 (for Brevibacillus) may be used, e.g., as shown above. However, any of the other N-terminal targeting sequences disclosed herein (including truncated and variant forms) may be substituted in place of SEQ ID NO: 4 in the exemplary configurations above, e.g., any of the sequences disclosed in Tables 1-4 or FIGS. 1-7. In the structures shown above, “Fusion Partner Protein” represents the linked protein of interest (e.g., a plant growth stimulating protein or peptide, the protein or peptide that protects a plant from a pathogen, the protein or peptide that enhances stress resistance in a plant, or the plant binding protein or peptide).

Alternatively, or in addition, the linker can comprise a protease recognition site. Inclusion of a protease recognition site allows for targeted removal, upon exposure to a protease that recognizes the protease recognition site, of the protein of interest (e.g., a plant growth stimulating protein or peptide, the protein or peptide that protects a plant from a pathogen, the protein or peptide that enhances stress resistance in a plant, or the plant binding protein or peptide).

In certain aspects, the fusion protein comprises an enzyme involved in the production or activation of a plant growth stimulating compound, such as an acetoin reductase, an indole-3-acetamide hydrolase, a tryptophan monooxygenase, an acetolactate synthetase, an α-acetolactate decarboxylase, a pyruvate decarboxylase, a diacetyl reductase, a butanediol dehydrogenase, an aminotransferase, a tryptophan decarboxylase, an amine oxidase, an indole-3-pyruvate decarboxylase, an indole-3-acetaldehyde dehydrogenase, a tryptophan side chain oxidase, a nitrile hydrolase, a nitrilase, a peptidase, a protease, an adenosine phosphate isopentenyltransferase, a phosphatase, an adenosine kinase, an adenine phosphoribosyltransferase, CYP735A, a 5′-ribonucleotide phosphohydrolase, an adenosine nucleosidase, a zeatin cis-trans isomerase, a zeatin O-glucosyltransferase, a β-glucosidase, a cis-hydroxylase, a CK cis-hydroxylase, a CK N-glucosyltransferase, a 2,5-ribonucleotide phosphohydrolase, an adenosine nucleosidase, a purine nucleoside phosphorylase, a zeatin reductase, a hydroxylamine reductase, a 2-oxoglutarate dioxygenase, a gibberellic 2B/3B hydrolase, a gibberellin 3-oxidase, a gibberellin 20-oxidase, a chitosanase, a chitinase, a β-1,3-glucanase, a β-1,4-glucanase, a β-1,6-glucanase, an aminocyclopropane-1-carboxylic acid deaminase, an enzyme involved in producing a nod factor, or any combination of the above.

In other aspects, the fusion protein comprises an enzyme that degrades or modifies a bacterial, fungal, or plant nutrient source, such as a cellulase, a lipase, a lignin oxidase, a protease, a glycoside hydrolase, a phosphatase, a nitrogenase, a nuclease, an amidase, a nitrate reductase, a nitrite reductase, an amylase, an ammonia oxidase, a ligninase, a glucosidase, a phospholipase, a phytase, a pectinase, a glucanase, a sulfatase, a urease, a xylanase, a siderophore, or any combination of the above.

In some embodiments, the fusion protein is expressed under the control of a sporulation promoter native to the targeting sequence, exosporium protein, or exosporium protein fragment of the fusion protein. The fusion protein may be expressed under the control of a high-expression sporulation promoter. In certain aspects, the high-expression sporulation promoter comprises a sigma-K sporulation-specific polymerase promoter sequence. In selected aspects, the fusion protein may be expressed under the control of a promoter that is native to the targeting sequence of the fusion protein. In some cases, the promoter that is native to the targeting sequence will be a high-expression sporulation promoter. In other cases, the promoter that is native to the targeting sequence will not be a high-expression sporulation promoter. In the latter cases, it may be advantageous to replace the native promoter with a high-expression sporulation promoter. Expression of the fusion protein under the control of a high-expression sporulation promoter provides for increased expression of the fusion protein on the exosporium of the Brevibacillus, Lysinibacillus, Viridibacillus, or Paenibacillus endospore. The high-expression sporulation promoter can comprise one or more sigma-K sporulation-specific promoter sequences.

As described above, the fusion proteins may comprise a targeting sequence and at least one heterologous protein that may comprise a growth stimulating protein or peptide. The plant growth stimulating protein or peptide can comprise, among other things, a peptide hormone, a non-hormone peptide, an enzyme involved in the production or activation of a plant growth-stimulating compound, or an enzyme that degrades or modifies a bacterial, fungal, or plant nutrient source. The plant growth stimulating protein or peptide can comprise an enzyme involved in the production or activation of a plant growth-stimulating compound. The enzyme involved in the production or activation of a plant growth stimulating compound can be any enzyme that catalyzes any step in a biological synthesis pathway for a compound that stimulates plant growth or alters plant structure, or any enzyme that catalyzes the conversion of an inactive or less active derivative of a compound that stimulates plant growth or alters plant structure into an active or more active form of the compound. Alternatively, the plant growth-stimulating compound can comprise a plant growth hormone, e.g., a cytokinin or a cytokinin derivative, ethylene, an auxin or an auxin derivative, a gibberellic acid or a gibberellic acid derivative, abscisic acid or an abscisic acid derivative, or a jasmonic acid or a jasmonic acid derivative.

Where the enzyme comprises a protease or peptidase, the protease or peptidase can be a protease or peptidase that cleaves proteins, peptides, proproteins, or preproproteins to create a bioactive peptide. The bioactive peptide can be any peptide that exerts a biological activity. The protease or peptidase that cleaves proteins, peptides, proproteins, or preproproteins to create a bioactive peptide can comprise subtilisin, an acid protease, an alkaline protease, a proteinase, an endopeptidase, an exopeptidase, thermolysin, papain, pepsin, trypsin, pronase, a carboxylase, a serine protease, a glutamic protease, an aspartate protease, a cysteine protease, a threonine protease, or a metalloprotease.

The plant growth stimulating protein can also comprise an enzyme that degrades or modifies a bacterial, fungal, or plant nutrient source. Such enzymes include cellulases, lipases, lignin oxidases, proteases, glycoside hydrolases, phosphatases, nitrogenases, nucleases, amidases, nitrate reductases, nitrite reductases, amylases, ammonia oxidases, ligninases, glucosidases, phospholipases, phytases, pectinases, glucanases, sulfatases, ureases, xylanases, and siderophores. When introduced into a plant growth medium or applied to a plant, seed, or an area surrounding a plant or a plant seed, fusion proteins comprising enzymes that degrade or modify a bacterial, fungal, or plant nutrient source can aid in the processing of nutrients in the vicinity of the plant and result in enhanced uptake of nutrients by the plant or by beneficial bacteria or fungi in the vicinity of the plant. The fusion proteins can comprise a targeting sequence and at least one protein or peptide that protects a plant from a pathogen. The protein or peptide can comprise a protein or peptide that stimulates a plant immune response. For example, the protein or peptide that stimulates a plant immune response can comprise a plant immune system enhancer protein or peptide. The plant immune system enhancer protein or peptide can be any protein or peptide that has a beneficial effect on the immune system of a plant. Alternatively, the protein or peptide that protects a plant from a pathogen can be a protein or peptide that has antibacterial activity, antifungal activity, or both antibacterial and antifungal activity. The protein or peptide that protects a plant from a pathogen can also be a protein or peptide that has insecticidal activity, helminthicidal activity, suppresses insect or worm predation, or a combination thereof. The protein that protects a plant from a pathogen can comprise an enzyme. Suitable enzymes include proteases and lactonases. The proteases and lactonases can be specific for a bacterial signaling molecule (e.g., a bacterial lactone homoserine signaling molecule). The enzyme can also be an enzyme that is specific for a cellular component of a bacterium or fungus.

The fusion proteins can comprise a targeting sequence and at least one protein or peptide that enhances stress resistance in a plant. For example, the protein or peptide that enhances stress resistance in a plant comprises an enzyme that degrades a stress-related compound. Stress-related compounds include, but are not limited to, aminocyclopropane-1-carboxylic acid (ACC), reactive oxygen species, nitric oxide, oxylipins, and phenolics. Specific reactive oxygen species include hydroxyl, hydrogen peroxide, oxygen, and superoxide. The enzyme that degrades a stress-related compound can comprise a superoxide dismutase, an oxidase, a catalase, an aminocyclopropane-1-carboxylic acid deaminase, a peroxidase, an antioxidant enzyme, or an antioxidant peptide.

The protein or peptide that enhances stress resistance in a plant can also comprise a protein or peptide that protects a plant from an environmental stress. The environmental stress can comprise, for example, drought, flood, heat, freezing, salt, heavy metals, low pH, high pH, or a combination thereof. For instance, the protein or peptide that protects a plant from an environmental stress can comprise an ice nucleation protein, a prolinase, a phenylalanine ammonia lyase, an isochorismate synthase, an isochorismate pyruvate lyase, or a choline dehydrogenase.

The fusion proteins can comprise a targeting sequence and at least plant binding protein or peptide. The plant binding protein or peptide can be any protein or peptide that is capable of specifically or non-specifically binding to any part of a plant (e.g., a plant root or an aerial portion of a plant such as a leaf, stem, flower, or fruit) or to plant matter. Thus, for example, the plant binding protein or peptide can be a root binding protein or peptide, or a leaf binding protein or peptide.

### Recombinant Brevibacillus, Lysinibacillus, Viridibacillus, and Paenibacillus Endospores and Cells Expressing Fusion Proteins

The fusion proteins described herein can be expressed by recombinant exosporium-producing Brevibacillus, Lysinibacillus, Viridibacillus, or Paenibacillus cells (e.g., B. brevis, L. sphaericus, V. arvi, or P. peoriae cells). The fusion protein can be any of the fusion proteins disclosed herein, provided that it retains intergeneric exosporium-targeting funcitonality, i.e., the fusion protein must comprise an N-terminal signal peptide shown in Tables 1-4 or FIGS. 1-7, or a fragment or variant of any one of these sequences, which retains exosporium-targeting functionality in at least two of Brevibacillus, Lysinibacillus, Viridibacillus, and/or Paenibacillus. Recombinant exosporium-producing Brevibacillus, Lysinibacillus, Viridibacillus, and/or Paenibacillus cells can co-express two or more of any of the fusion proteins disclosed herein. For example, the recombinant exosporium-producing Brevibacillus, Lysinibacillus, Viridibacillus, and/or Paenibacillus cells can co-express at least one fusion protein that comprises a plant binding protein or peptide, together with at least one fusion protein comprising a plant growth stimulating protein or peptide, at least one fusion protein comprising a protein or peptide that protects a plant from a pathogen, or at least one protein or peptide that enhances stress resistance in a plant.

The recombinant exosporium-producing Brevibacillus cells may comprise Brevibacillus cells, such as B. agri, B. aydinogluensis, B. borstelensis, B. brevis, B. centrosporus, B. choshinensis, B. fluminis, B. formosus, B. fulvus, B. ginsengisoli, B. invocatus, B. laterosporus, B. levickii, B. limnophilus, B. massiliensis, B. nitrificans, B. panacihumi, B. parabrevis, B. reuszeri, or B. thermoruber cells.

The recombinant exosporium-producing Lysinibacillus cells may comprise Lysinibacillus cells, such as Lysinibacillus sphaericus, Lysinibacillus boronitolerans, Lysinibacillus fusiformis, Lysinibacillus acetophenoni, Lysinibacillus alkaliphilus, Lysinibacillus chungkukjangi, Lysinibacillus composti, Lysinibacillus contaminans, Lysinibacillus cresolivorans, Lysinibacillus macroides, Lysinibacillus manganicus, Lysinibacillus mangiferihumi, Lysinibacillus massiliensis, Lysinibacillus meyeri, Lysinibacillus odysseyi, Lysinibacillus pakistanensis, Lysinibacillus parviboronicapiens, Lysinibacillus sinduriensis, Lysinibacillus tabacifolii, Lysinibacillus varians, Lysinibacillus xylanilyticus, or Lysinibacillus halotolerans cells.

The recombinant exosporium-producing Viridibacillus cells may comprise Viridibacillus cells, such as Viridibacillus arvi, Viridibacillus arenosi, or Viridibacillus neidei cells.

The recombinant exosporium-producing Paenibacillus cells may comprise Paenibacillus cells, such as Paenibacillus sp. NRRL B-50972, Paenibacillus terrae, Paenibacillus polymyxa, or Paenibacillus peoriae cells. In other aspects, the Paenibacillus cells may comprise: Paenibacillus abyssi, Paenibacillus aceti, Paenibacillus aestuarii, Paenibacillus agarexedens, Paenibacillus agaridevorans, Paenibacillus alginolyticus, Paenibacillus algorifonticola, Paenibacillus alkaliterrae, Paenibacillus alvei, Paenibacillus amylolyticus, Paenibacillus anaericanus, Paenibacillus antarcticus, Paenibacillus apiarius, Paenibacillus arachidis, Paenibacillus assamensis, Paenibacillus azoreducens, Paenibacillus azotofixans, Paenibacillus baekrokdamisoli, Paenibacillus barcinonensis, Paenibacillus barengoltzii, Paenibacillus borealis, Paenibacillus bovis, Paenibacillus brasilensis, Paenibacillus camelliae, Paenibacillus campinasensis, Paenibacillus castaneae, Paenibacillus catalpae, Paenibacillus cathormii, Paenibacillus cavernae, Paenibacillus cellulosilyticus, Paenibacillus cellulositrophicus, Paenibacillus chartarius, Paenibacillus chibensis, Paenibacillus chinjuensis, Paenibacillus chitinolyticus, Paenibacillus chondroitinus, Paenibacillus chungangensis, Paenibacillus cineris, Paenibacillus cisolokensis, Paenibacillus contaminans, Paenibacillus cookii, Paenibacillus cucumis, Paenibacillus curdlanolyticus, Paenibacillus daejeonensis, Paenibacillus darwinianus, Paenibacillus dauci, Paenibacillus dendritiformis, Paenibacillus dongdonensis, Paenibacillus doosanensis, Paenibacillus durus, Paenibacillus edaphicus, Paenibacillus ehimensis, Paenibacillus elgii, Paenibacillus endophyticus, Paenibacillus etheri, Paenibacillus faecis, Paenibacillus favisporus, Paenibacillus ferrarius, Paenibacillus filicis, Paenibacillus fonticola, Paenibacillus forsythiae, Paenibacillus frigoriresistens, Paenibacillus gansuensis, Paenibacillus gelatinilyticus, Paenibacillus ginsengarvi, Paenibacillus ginsengihumi, Paenibacillus ginsengisoli, Paenibacillus glacialis, Paenibacillus glucanolyticus, Paenibacillus glycanilyticus, Paenibacillus gordonae, Paenibacillus graminis, Paenibacillus granivorans, Paenibacillus guangzhouensis, Paenibacillus harenae, Paenibacillus hemerocallicola, Paenibacillus hispanicus, Paenibacillus hodogayensis, Paenibacillus hordei, Paenibacillus humicus, Paenibacillus hunanensis, Paenibacillus illinoisensis, Paenibacillus jamilae, Paenibacillus jilunlii, Paenibacillus kobensis, Paenibacillus koleovorans, Paenibacillus konsidensis, Paenibacillus koreensis, Paenibacillus kribbensis, Paenibacillus kyungheensis, Paenibacillus lactis, Paenibacillus larvae, Paenibacillus larvae, Paenibacillus larvae, Paenibacillus lautus, Paenibacillus lemnae, Paenibacillus lentimorbus, Paenibacillus lentus, Paenibacillus liaoningensis, Paenibacillus lupini, Paenibacillus macerans, Paenibacillus macquariensis, Paenibacillus macquariensis, Paenibacillus macquariensis, Paenibacillus marchantiophytorum, Paenibacillus marinisediminis, Paenibacillus massiliensis, Paenibacillus medicaginis, Paenibacillus mendelii, Paenibacillus methanolicus, Paenibacillus montaniterrae, Paenibacillus motobuensis, Paenibacillus mucilaginosus, Paenibacillus nanensis, Paenibacillus naphthalenovorans, Paenibacillus nasutitermitis, Paenibacillus nematophilus, Paenibacillus nicotianae, Paenibacillus oceanisediminis, Paenibacillus odorifer, Paenibacillus oenotherae, Paenibacillus oryzae, Paenibacillus pabuli, Paenibacillus panacisoli, Paenibacillus panaciterrae, Paenibacillus pasadenensis, Paenibacillus pectinilyticus, Paenibacillus periandrae, Paenibacillus phoenicis, Paenibacillus phyllosphaerae, Paenibacillus physcomitrellae, Paenibacillus pini, Paenibacillus pinihumi, Paenibacillus pinesoli, Paenibacillus pocheonensis, Paenibacillus popilliae, Paenibacillus populi, Paenibacillus prosopidis, Paenibacillus provencensis, Paenibacillus pueri, Paenibacillus puldeungensis, Paenibacillus pulvifaciens, Paenibacillus purispatii, Paenibacillus qingshengii, Paenibacillus quercus, Paenibacillus radicis, Paenibacillus relictisesami, Paenibacillus residui, Paenibacillus rhizoryzae, Paenibacillus rhizosphaerae, Paenibacillus rigui, Paenibacillus riograndensis, Paenibacillus ripae, Paenibacillus sabinae, Paenibacillus sacheonensis, Paenibacillus salinicaeni, Paenibacillus sanguinis, Paenibacillus sediminis, Paenibacillus segetis, Paenibacillus selenii, Paenibacillus selenitireducens, Paenibacillus senegalensis, Paenibacillus septentrionalis, Paenibacillus sepulcri, Paenibacillus shenyangensis, Paenibacillus shirakamiensis, Paenibacillus siamensis, Paenibacillus silagei, Paenibacillus sinopodophylli, Paenibacillus solani, Paenibacillus soli, Paenibacillus sonchi, Paenibacillus sophorae, Paenibacillus sputi, Paenibacillus stellifer, Paenibacillus susongensis, Paenibacillus swuensis, Paenibacillus taichungensis, Paenibacillus taiwanensis, Paenibacillus tarimensis, Paenibacillus telluris, Paenibacillus terreus, Paenibacillus terrigena, Paenibacillus thailandensis, Paenibacillus thermophilus, Paenibacillus thiaminolyticus, Paenibacillus tianmuensis, Paenibacillus tibetensis, Paenibacillus timonensis, Paenibacillus tundrae, Paenibacillus turicensis, Paenibacillus typhae, Paenibacillus uliginis, Paenibacillus urinalis, Paenibacillus validus, Paenibacillus vini, Paenibacillus vulneris, Paenibacillus wenxiniae, Paenibacillus wooponensis, Paenibacillus woosongensis, Paenibacillus wulumuqiensis, Paenibacillus wynnii, Paenibacillus xanthinilyticus, Paenibacillus xinjiangensis, Paenibacillus xylanexedens, Paenibacillus xylanilyticus, Paenibacillus xylanisolvens, Paenibacillus yonginensis, Paenibacillus yunnanensis, Paenibacillus zanthoxyli, or Paenibacillus zeae cells.

To generate recombinant exosporium-producing Brevibacillus, Lysinibacillus, Viridibacillus, or Paenibacillus cells expressing a fusion protein, any Brevibacillus, Lysinibacillus, Viridibacillus, or Paenibacillus bacterium may be transformed using standard methods known in the art (e.g., by electroporation, or by conjugation with a cell that has been transformed with a vector encoding the fusion protein). The bacteria can then be screened to identify transformants by any method known in the art. For example, where the vector includes an antibiotic resistance gene, the bacteria can be screened for antibiotic resistance. Alternatively, DNA encoding the fusion protein can be integrated into the chromosomal DNA of Brevibacillus, Lysinibacillus, Viridibacillus, and/or Paenibacillus cells. The recombinant exosporium-producing Brevibacillus, Lysinibacillus, Viridibacillus, and/or Paenibacillus cells can then exposed to conditions that will induce sporulation. Suitable conditions for inducing sporulation are known in the art. For example, the recombinant exosporium-producing Brevibacillus, Lysinibacillus, Viridibacillus, and/or Paenibacillus cells can be plated onto agar plates, and incubated at a temperature of about 30° C. for several days (e.g., 3 days), or alternatively cultured in Schaeffer Sporulation Medium.

Inactivated, non-toxic, or genetically manipulated strains of any of the species disclosed herein can also suitably be used. For example, a Brevibacillus, Lysinibacillus, Viridibacillus, and/or Paenibacillus strain that lacks the Bin toxins can be used. Alternatively or in addition, once a recombinant Brevibacillus, Lysinibacillus, Viridibacillus, and/or Paenibacillus species' spores expressing a fusion protein have been generated, they can be inactivated to prevent further germination. Any method for inactivating bacterial spores that is known in the art can be used. Suitable methods include, without limitation, heat treatment, gamma irradiation, x-ray irradiation, UV-A irradiation, UV-B irradiation, chemical treatment (e.g., treatment with gluteraldehyde, formaldehyde, hydrogen peroxide, acetic acid, bleach, or any combination thereof), or a combination thereof. Alternatively, spores derived from nontoxigenic strains, or genetically or physically inactivated strains, can be used.

Fusion protein constructs according to the present disclosure comprise: (1) an N-terminal signal sequence or a variant or fragment thereof that targets the fusion protein to the exosporium when expressed in multiple bacterial genera, particularly in at least two of Brevibacillus, Lysinibacillus, Viridibacillus, and/or Paenibacillus; and (2) a polypeptide sequence that is heterologous to the N-terminal signal sequence. In select embodiments, the N-terminal signal sequence and the polypeptide sequence that is heterologous to the N-terminal signal sequence are directly linked. In other aspects, an intervening linker or spacer sequence may be present. In further aspects, a cleavage sequence or other regulatory sequence may be positioned between the two regions. The polypeptide sequence that is heterologous to the N-terminal signal sequence may comprise one or more functional proteins. In aspects where multiple functional proteins are contained in the polypeptide sequence that is heterologous to the N-terminal signal sequence, at least one spacer, cleavage sequence or other regulatory element may be located between the two or more functional proteins.

The polypeptide sequence that is heterologous to the N-terminal signal sequence may be, for example: (a) a plant growth stimulating protein or peptide; (b) a protein or peptide that protects a plant from a pathogen; (c) a protein or peptide that enhances stress resistance of a plant; (d) a plant binding protein or peptide; (e) a plant immune system enhancer protein or peptide; or (f) a protein or peptide that enhances nutrient uptake. These fusion proteins are targeted to the exosporium layer of the endospore produced by members of at least two genera selected from Brevibacillus, Lysinibacillus, Viridibacillus, and/or Paenibacillus, and are physically oriented such that the protein or peptide is displayed on the outside of the endospore. In some aspects, the fusion proteins may be targeted to the exosporium of at least three, or all four, of these genera.

The presently disclosed Brevibacillus, Lysinibacillus, and Viridibacillus exosporium display systems can be used to deliver peptides, enzymes, and other proteins to plants (e.g., to plant foliage, fruits, flowers, stems, or roots) or to a plant growth medium such as soil. Peptides, enzymes, and proteins delivered to the soil or another plant growth medium in this manner persist and exhibit activity in the soil for extended periods of time. Introduction of recombinant exosporium-producing Brevibacillus, Lysinibacillus, Viridibacillus, and/or Paenibacillus cells expressing the fusion proteins described herein into soil or the rhizosphere of a plant may lead to a beneficial enhancement of plant growth in many different soil conditions. The use of a Brevibacillus, Lysinibacillus, Viridibacillus, or Paenibacillus exosporium display system to create these enzymes allows them to continue to exert their beneficial effects on the plant and the rhizosphere over the first months of a plants life, and in some aspects over longer period of time up to and including the life of the plant.

### Isolated and/or Purified Exosporia and Compositions Comprising the Same

The disclosure provides isolated and/or purified exosporia obtained from recombinant Brevibacillus, Lysinibacillus, Viridibacillus, or Paenibacillus endospores that have been modified to express fusion protein constructs as described herein, and compositions comprising the same. In selected aspects, these compositions comprise either the basal layer or the hair-like nap layer of a recombinant Brevibacillus, Lysinibacillus, Viridibacillus, and/or Paenibacillus exosporium. In others, the composition comprises both layers of a recombinant Brevibacillus, Lysinibacillus, Viridibacillus, and/or Paenibacillus exosporium. In selected aspects, the composition may comprise a specific fraction or extract of a recombinant Brevibacillus, Lysinibacillus, Viridibacillus, and/or Paenibacillus exosporium (e.g., an extract comprising exosporium components soluble in a particular solvent).

In other select aspects, the exosporium compositions may further comprise additional components (e.g., any of the plant growth-promoting compounds, pesticides, or other active agents disclosed herein). In additional aspects, the exosporium compositions may be treated to kill or render nonviable vegetative Brevibacillus, Lysinibacillus, Viridibacillus, and/or Paenibacillus cells or endospores in the composition. In select aspects, the exosporium composition contains no detectable amounts of Brevibacillus, Lysinibacillus, Viridibacillus, and/or Paenibacillus cells and/or endospores. In select aspects, the exosporium composition is processed to remove or reduce the level of bacterial toxins and/or immunogenic components in order to produce an exosporium composition that is less toxic or immunogenic, or otherwise more well-tolerated by a plant or animal that may be treated with or exposed to the exosporium composition.

In selected aspects, the exosporium composition comprises substantially intact exosporia collected from recombinant Brevibacillus, Lysinibacillus, Viridibacillus, and/or Paenibacillus endospores (e.g., using sonication). Alternatively, the composition may contain processed exosporia (e.g., ground up, suspended in a fluid, etc.) In alternative aspects, the exosporium composition may be dissolved in a solvent. In each case, the composition may be processed so that a particular subcomponent or compound is enriched. For example, exosporium compositions may be processed to enrich the concentration or amount of the recombinant fusion protein present in the enriched composition compared to the amount or composition in the crude exosporium collected from the recombinant Brevibacillus, Lysinibacillus, Viridibacillus, and/or Paenibacillus endospores.

In select aspects, the exosporium composition comprises an isolated and/or purified Brevibacillus, Lysinibacillus, Viridibacillus, and/or Paenibacillus exosporium containing a fusion protein according to any aspects described herein. For example, the fusion protein comprises any of the N-terminal targeting sequences disclosed herein, e.g., any of the sequences disclosed in Tables 1-4 or FIGS. 1-7.

The fusion proteins and/or exosporium compositions disclosed herein may be used to deliver a protein of interest to a plant. In select aspects, the fusion proteins or exosporium compositions according to any aspect described herein may be applied directly to a plant (e.g., as a powder, suspension or solution, to a seed, or to a field). In select aspects, the fusion protein or exosporium composition is applied to a field prior to or after seeding, or alternatively prior to or after sprouting (e.g., pre- or post-planting, or pre- or post-emergence).

In alternative aspects, the fusion proteins and/or exosporium compositions disclosed herein may be delivered to a plant, seed, and/or field indirectly by applying recombinant Brevibacillus, Lysinibacillus, Viridibacillus, and/or Paenibacillus cells to the plant, seed, or field. In these aspects, the fusion protein and/or exosporium composition may be expressed or generated by the recombinant Brevibacillus, Lysinibacillus, Viridibacillus, and/or Paenibacillus cells (e.g., in the field), resulting in delivery of the fusion protein to the plant, seed, or field.

### Recombinant Exosporium-Producing Brevibacillus, Lysinibacillus, and Viridibacillus Cells Having Plant-Growth Promoting Effects and/or Other Beneficial Attributes

Some Brevibacillus, Lysinibacillus, Viridibacillus, and Paenibacillus bacteria are known to have inherent beneficial attributes. For example, some strains have plant-growth promoting effects. Any of the fusion proteins described herein can be expressed in such strains.

For example, the recombinant exosporium-producing Brevibacillus, Lysinibacillus, Viridibacillus, and/or Paenibacillus cells may comprise a plant-growth promoting species or strain of Brevibacillus, Lysinibacillus, Viridibacillus, and/or Paenibacillus. This plant-growth promoting species or strain of bacteria can comprise a strain of bacteria that produces an insecticidal toxin (e.g., a Bin toxin), produces a fungicidal compound (e.g., a β-1,3-glucanase, a chitosinase, a lyticase, or a combination thereof), produces a nematocidal compound (e.g., a Cry toxin), produces a bacteriocidal compound, is resistant to one or more antibiotics, comprises one or more freely replicating plasmids, binds to plant roots, colonizes plant roots, forms biofilms, solubilizes nutrients, secretes organic acids, or any combination thereof.

### Biological Control Agents

Compositions provided by the disclosure may further include biological control agents. Biological control agents can include, in particular, bacteria, fungi or yeasts, protozoa, viruses, entomopathogenic nematodes, inoculants and botanicals and/or mutants of them having all identifying characteristics of the respective strain, and/or at least one metabolite produced by the respective strain that exhibits activity against insects, mites, nematodes and/or phytopathogens. The disclosure provides combinations of the above-described recombinant Brevibacillus, Lysinibacillus, Viridibacillus, and/or Paenibacillus endospores with the particular biological control agents described herein and/or to mutants of specific strains of microorganisms described herein, where the mutants have all the identifying characteristics of the respective strain, and/or at least one metabolite produced by the respective strain that exhibits activity against insects, mites, nematodes and/or phytopathogens or promotes plant growth and/or enhances plant health. According to the disclosure, the biological control agents described herein may be employed or used in any physiologic state such as active or dormant.

### Selected Compositions According to the Present Disclosure

In selected aspects, the disclosure provides compositions comprising (a) recombinant exosporium-producing Brevibacillus, Lysinibacillus, Viridibacillus, and/or Paenibacillus cells that expresses a fusion protein comprising: a targeting sequence that localizes the fusion protein, which comprises a heterologous protein of interest, to the exosporium of at least two of a Brevibacillus, Lysinibacillus, Viridibacillus, and/or Paenibacillus family member; and (b) at least one further and different particular biological control agent disclosed herein and/or a mutant of a specific species of a microorganism disclosed herein having all identifying characteristics of the respective species, and/or at least one metabolite produced by the respective species that exhibits activity against insects, mites, nematodes and/or phytopathogens in a synergistically effective amount. In alternative aspects, the composition comprises at least one additional fungicide and/or at least one insecticide, with the proviso that the recombinant exosporium-producing Brevibacillus, Lysinibacillus, Viridibacillus, and/or Paenibacillus cells, the insecticide and the fungicide are not identical. In another aspect, composition is used for reducing overall damage of plants and plant parts, as well as, losses in harvested fruits or vegetables caused by insects, mites, nematodes and/or phytopathogens. In another aspect, the composition increases the overall plant health.

The term “plant health” generally comprises various sorts of improvements of plants that are not connected to the control of pests. For example, advantageous properties that may be mentioned are improved crop characteristics including: emergence, crop yields, protein content, oil content, starch content, more developed root system, improved root growth, improved root size maintenance, improved root effectiveness, improved stress tolerance (e.g., against drought, heat, salt, UV, water, cold), reduced ethylene (reduced production and/or inhibition of reception), tillering increase, increase in plant height, bigger leaf blade, less dead basal leaves, stronger tillers, greener leaf color, pigment content, photosynthetic activity, less input needed (such as fertilizers or water), less seeds needed, more productive tillers, earlier flowering, early grain maturity, less plant verse (lodging), increased shoot growth, enhanced plant vigor, increased plant stand and early and better germination.

Compositions provided by the disclosure may be screened to identify potential benefits to plant growth, health, or other positive attributes by comparing plants which are grown under the same environmental conditions, whereby a part of said plants is treated with a composition according to the present disclosure and another part of said plants is not treated with a composition according to the present disclosure. Instead, said other part is not treated at all or is treated with a suitable control (i.e., an application without a composition according to the disclosure such as an application without all active ingredients), an application without the recombinant exosporium-producing Brevibacillus, Lysinibacillus, Viridibacillus, and/or Paenibacillus cells as described herein, or an application without a further particular biological control agent disclosed herein.

The composition according to the present disclosure may be applied in any desired manner, such as in the form of a seed coating, soil drench, and/or directly in-furrow and/or as a foliar spray and applied either pre-emergence, post-emergence or both. In other words, the composition can be applied to the seed, the plant or to harvested fruits and vegetables or to the soil wherein the plant is growing or wherein it is desired to grow (plant's locus of growth).

Reducing the overall damage of plants and plant parts often results in healthier plants and/or in an increase in plant vigor and yield. Preferably, the composition according to the present disclosure is used for treating conventional or transgenic plants or seed thereof. Methods of Using Recombinant Brevibacillus, Lysinibacillus, and Viridibacillus Constructs and Compositions

The present disclosure also relates to methods for stimulating plant growth using any of the compositions described above comprising recombinant exosporium-producing Brevibacillus, Lysinibacillus, Viridibacillus, and/or Paenibacillus cells that express a fusion protein and at least one of the further particular biological control agents described herein. The method for stimulating plant growth comprises applying to a plant, a seed, a plant part, to the locus surrounding the plant or in which the plant will be planted (e.g., soil or other growth medium) a composition comprising recombinant exosporium-producing Brevibacillus, Lysinibacillus, Viridibacillus, and/or Paenibacillus cells that express a fusion protein comprising: (i) a heterologous protein (e.g., at least one plant growth stimulating protein); and (ii) a targeting sequence; and at least one further particular biological control agent disclosed herein and/or a mutant of a specific species of a microorganism disclosed herein having all identifying characteristics of the respective species, and/or at least one metabolite produced by the respective species that exhibits activity against insects, mites, nematodes and/or phytopathogens in a synergistically effective amount.

In another aspect of the present disclosure a method for reducing overall damage of plants and plant parts as well as losses in harvested fruits or vegetables caused by insects, mites, nematodes and/or phytopathogens is provided comprising the step of simultaneously or sequentially applying the recombinant exosporium-producing Brevibacillus, Lysinibacillus, Viridibacillus, and/or Paenibacillus cells, and at least one further particular biological control agent described herein in a synergistically effective amount.

In one embodiment of the present method the composition further comprises at least one fungicide. In one aspect, the at least one fungicide is a synthetic fungicide. In another embodiment, the composition comprises at least one insecticide in addition to the fungicide or in place of the fungicide, provided that the insecticide, the fungicide, the recombinant exosporium-producing Brevibacillus, Lysinibacillus, Viridibacillus, and/or Paenibacillus cells and the particular biological control agent disclosed herein are not identical.

The method of the present disclosure includes the following application methods, namely both of the recombinant exosporium-producing Brevibacillus, Lysinibacillus, Viridibacillus, and/or Paenibacillus cells and the at least one further particular biological control agent disclosed herein may be formulated into a single, stable composition with an agriculturally acceptable shelf life (so called “solo-formulation”), or being combined before or at the time of use (so called “combined-formulations”).

If not mentioned otherwise, the expression “combination” stands for the various combinations of the recombinant exosporium-producing Brevibacillus, Lysinibacillus, Viridibacillus, and/or Paenibacillus cells and at least one further particular biological control agent disclosed herein, and optionally at least one fungicide and/or at least one insecticide, in a solo-formulation, in a single “ready-mix” form, in a combined spray mixture composed from solo-formulations, such as a “tank-mix”, and especially in a combined use of the single active ingredients when applied in a sequential manner, i.e., one after the other within a reasonably short period, such as a few hours or days, e.g., 2 hours to 7 days. The order of applying the composition according to the present disclosure is not essential for working the present disclosure. Accordingly, the term “combination” also encompasses the presence of the recombinant exosporium-producing Brevibacillus, Lysinibacillus, Viridibacillus, and/or Paenibacillus cells and the at least one further particular biological control agent disclosed herein, and optionally at least one fungicide and/or insecticide on or in a plant to be treated or its surrounding, habitat or storage space, e.g., after simultaneously or consecutively applying the recombinant exosporium-producing Brevibacillus, Lysinibacillus, Viridibacillus, and/or Paenibacillus cells and the at least one further particular biological control agent disclosed herein, and optionally at least one fungicide and/or at least one insecticide to a plant or its surrounding, habitat or storage space.

If the recombinant exosporium-producing Brevibacillus, Lysinibacillus, Viridibacillus, and/or Paenibacillus cells and the at least one further particular biological control agent described herein, and optionally at least one fungicide and/or at least one insecticide are employed or used in a sequential manner, it is preferred to treat the plants or plant parts (which includes seeds and plants emerging from the seed), harvested fruits and vegetables according to the following method: firstly applying at least one fungicide and/or at least one insecticide on the plant or plant parts, and secondly applying the further particular biological control agent described herein and the recombinant exosporium-producing Brevibacillus, Lysinibacillus, Viridibacillus, and/or Paenibacillus cells to the same plant or plant parts. By this application manner the amount of residues of insecticides/fungicides on the plant upon harvesting is as low as possible. The time periods between the first and the second application within a (crop) growing cycle may vary and depend on the effect to be achieved. For example, the first application is done to prevent an infestation of the plant or plant parts with insects, mites, nematodes and/or phytopathogens (this is particularly the case when treating seeds) or to combat the infestation with insects, mites, nematodes and/or phytopathogens (this is particularly the case when treating plants and plant parts) and the second application is done to prevent or control the infestation with insects, mites, nematodes and/or phytopathogens and/or to promote plant growth. Control in this context means that the composition comprising the recombinant exosporium-producing Brevibacillus, Lysinibacillus, Viridibacillus, and/or Paenibacillus cells and the particular biological control agent disclosed herein are not able to fully exterminate the pests or phytopathogenic fungi but are able to keep the infestation on an acceptable level.

The present disclosure also provides methods of enhancing the killing, inhibiting, preventative and/or repelling activity of the compositions of the present disclosure by multiple applications. In some other embodiments, the compositions of the present disclosure are applied to a plant and/or plant part for two times, during any desired development stages or under any predetermined pest pressure, at an interval of about 1 hour, about 5 hours, about 10 hours, about 24 hours, about two days, about 3 days, about 4 days, about 5 days, about 1 week, about 10 days, about two weeks, about three weeks, about 1 month or more. Still in some embodiments, the compositions of the present disclosure are applied to a plant and/or plant part for more than two times, for example, 3 times, 4 times, 5 times, 6 times, 7 times, 8 times, 9 times, 10 times, or more, during any desired development stages or under any predetermined pest pressure, at an interval of about 1 hour, about 5 hours, about 10 hours, about 24 hours, about two days, about 3 days, about 4 days, about 5 days, about 1 week, about 10 days, about two weeks, about three weeks, about 1 month or more. The intervals between each application can vary if it is desired. One skilled in the art will be able to determine the application times and length of interval depending on plant species, plant pest species, and other factors.

By following the before mentioned steps, a very low level of residues of the at least one fungicide and/or at least one insecticide on the treated plant, plant parts, and the harvested fruits and vegetables can be achieved.

If not mentioned otherwise the treatment of plants or plant parts (which includes seeds and plants emerging from the seed), harvested fruits and vegetables with the composition according to the disclosure is carried out directly or by action on their surroundings, habitat or storage space using customary treatment methods, for example dipping, spraying, atomizing, irrigating, evaporating, dusting, fogging, broadcasting, foaming, painting, spreading-on, watering (drenching), drip irrigating. It is furthermore possible to apply the recombinant exosporium-producing Brevibacillus, Lysinibacillus, Viridibacillus, and/or Paenibacillus cells, the at least one further particular biological control agent described herein, and optionally the at least one fungicide and/or the at least one insecticide as solo-formulation or combined-formulations by the ultra-low volume method, or to inject the composition according to the present disclosure as a composition or as sole-formulations into the soil (in-furrow).

The term “plant to be treated” encompasses every part of a plant including its root system and the material—e.g., soil or nutrition medium—which is in a radius of at least 10 cm, 20 cm, 30 cm around the caulis or bole of a plant to be treated or which is at least 10 cm, 20 cm, 30 cm around the root system of said plant to be treated, respectively.

The amount of the recombinant exosporium-producing Brevibacillus, Lysinibacillus, Viridibacillus, and/or Paenibacillus cells, which is used or employed in combination with at least one further particular biological control agent described herein, optionally in the presence of at least one fungicide and/or the at least one insecticide, depends on the final formulation as well as size or type of the plant, plant parts, seeds, harvested fruits and vegetables to be treated. Usually, the recombinant exosporium-producing Brevibacillus, Lysinibacillus, Viridibacillus, and/or Paenibacillus cells to be employed or used according to the disclosure is present in about 1% to about 80% (w/w), preferably in about 1% to about 60% (w/w), more preferably about 10% to about 50% (w/w) of its solo-formulation or combined-formulation with the at least one further particular biological control agent described herein, and optionally the fungicide and/or the at least one insecticide.

Also the amount of the at least one further particular biological control agent disclosed herein which is used or employed in combination with the recombinant exosporium-producing Brevibacillus, Lysinibacillus, Viridibacillus, and/or Paenibacillus cells, optionally in the presence of at least one fungicide and/or the at least one insecticide, depends on the final formulation as well as size or type of the plant, plant parts, seeds, harvested fruit or vegetable to be treated. Usually, the further particular biological control agent described herein to be employed or used according to the disclosure is present in about 0.1% to about 80% (w/w), preferably 1% to about 60% (w/w), more preferably about 10% to about 50% (w/w) of its solo-formulation or combined-formulation with the recombinant exosporium-producing Brevibacillus, Lysinibacillus, Viridibacillus, and/or Paenibacillus cells, and optionally the at least one fungicide and/or the at least one insecticide.

Application of the recombinant exosporium-producing Brevibacillus, Lysinibacillus, Viridibacillus, and/or Paenibacillus cells may be effected as a foliar spray, as a soil treatment, and/or as a seed treatment/dressing. When used as a foliar treatment, in one embodiment, about 1/16 to about 5 gallons of whole broth are applied per acre. When used as a soil treatment, in one embodiment, about 1 to about 5 gallons of whole broth are applied per acre. When used for seed treatment about 1/32 to about ¼ gallons of whole broth are applied per acre. For seed treatment, the end-use formulation contains at least 1×10⁴, at least 1×10⁵, at least 1×10⁶, 1×10⁷, at least 1×10⁸, at least 1×10⁹, at least 1×10¹¹ colony forming units per gram.

The ratio can be calculated based on the amount of the at least one further particular biological control agent disclosed herein, at the time point of applying said component of a combination according to the disclosure to a plant or plant part and the amount of the recombinant exosporium-producing Brevibacillus, Lysinibacillus, Viridibacillus, and/or Paenibacillus cells shortly prior (e.g., 48 h, 24 h, 12 h, 6 h, 2 h, 1 h) or at the time point of applying said component of a combination according to the disclosure to a plant or plant part.

The application of the recombinant exosporium-producing Brevibacillus, Lysinibacillus, Viridibacillus, and/or Paenibacillus cells and the at least one further particular biological control agent disclosed herein to a plant or a plant part can take place simultaneously or at different times as long as both components are present on or in the plant after the application(s). In cases where the recombinant exosporium-producing Brevibacillus, Lysinibacillus, Viridibacillus, and/or Paenibacillus cells and further particular biological control agent disclosed herein are applied at different times and the further particular biological control agent disclosed herein is applied prior to the recombinant exosporium-producing Brevibacillus, Lysinibacillus, Viridibacillus, and/or Paenibacillus cells, the skilled person can determine the concentration of further particular biological control agent disclosed herein on/in a plant by chemical analysis known in the art, at the time point or shortly before the time point of applying the recombinant exosporium-producing Brevibacillus, Lysinibacillus, Viridibacillus, and/or Paenibacillus cells. Similarly, when the recombinant exosporium-producing Brevibacillus, Lysinibacillus, Viridibacillus, and/or Paenibacillus cells are applied to a plant first, the concentration of the recombinant exosporium-producing Brevibacillus, Lysinibacillus, Viridibacillus, and/or Paenibacillus cells can be determined using tests which are also known in the art, at the time point or shortly before the time point of applying the further particular biological control agent disclosed herein.

In another aspect of the present disclosure a seed treated with the composition as described above is provided. The control of insects, mites, nematodes and/or phytopathogens by treating the seed of plants has been known for a long time and is a subject of continual improvements. Nevertheless, the treatment of seed entails a series of problems which cannot always be solved in a satisfactory manner. Thus, it is desirable to develop methods for protecting the seed and the germinating plant that remove the need for, or at least significantly reduce, the additional delivery of crop protection compositions in the course of storage, after sowing or after the emergence of the plants. It is desirable, furthermore, to optimize the amount of active ingredient employed in such a way as to provide the best-possible protection to the seed and the germinating plant from attack by insects, mites, nematodes and/or phytopathogens, but without causing damage to the plant itself by the active ingredient employed. In particular, methods for treating seed ought also to take into consideration the intrinsic insecticidal and/or nematicidal properties of pest-resistant or pest-tolerant transgenic plants, in order to achieve optimum protection of the seed and of the germinating plant with a minimal use of crop protection compositions.

The present disclosure therefore also relates in particular to a method for protecting seed and germinating plants from attack by pests, by treating the seed with the recombinant exosporium-producing Brevibacillus, Lysinibacillus, Viridibacillus, and/or Paenibacillus cells as defined above and at least one further biological control agent selected from particular microorganisms disclosed herein and/or a mutant of a specific strain of microorganism disclosed herein having all identifying characteristics of the respective strain, and/or at least one metabolite produced by the respective strain that exhibits activity against insects, mites, nematodes and/or phytopathogens and optionally at least one fungicide and/or optionally at least one insecticide of the disclosure. The method of the disclosure for protecting seed and germinating plants from attack by pests encompasses a method in which the seed is treated simultaneously in one operation with the recombinant exosporium-producing Brevibacillus, Lysinibacillus, Viridibacillus, and/or Paenibacillus cells and the at least one further particular biological control agent described herein, and optionally the at least one fungicide and/or the at least one insecticide. It also encompasses a method in which the seed is treated at different times with the recombinant exosporium-producing Brevibacillus, Lysinibacillus, Viridibacillus, and/or Paenibacillus cells and the at least one further particular biological control agent disclosed herein, and optionally the at least one fungicide and/or the at least one insecticide.

The disclosure further provides methods of treating seeds for the purpose of protecting the seed and the resultant plant against insects, mites, nematodes and/or phytopathogens. The disclosure also relates to seed which at the same time has been treated with a the recombinant exosporium-producing Brevibacillus, Lysinibacillus, Viridibacillus, and/or Paenibacillus cells and at least one further particular biological control agent described herein, and optionally at least one fungicide and/or the at least one insecticide. The disclosure further relates to seed which has been treated at different times with the recombinant exosporium-producing Brevibacillus, Lysinibacillus, Viridibacillus, and/or Paenibacillus cells and the at least one further particular biological control agent disclosed herein and optionally the at least one fungicide and/or the at least one insecticide. In the case of seed which has been treated at different times with the recombinant exosporium-producing Brevibacillus, Lysinibacillus, Viridibacillus, and/or Paenibacillus cells and the at least one further particular biological control agent disclosed herein, and optionally the at least one fungicide and/or the at least one insecticide, the individual active ingredients in the composition of the disclosure may be present in different layers on the seed.

Furthermore, the disclosure relates to seed which, following treatment with the composition of the disclosure, is subjected to a film-coating process in order to prevent dust abrasion of the seed.

One of the advantages of the present disclosure is that, owing to the particular systemic properties of the compositions of the disclosure, the treatment of the seed with these compositions provides protection from insects, mites, nematodes and/or phytopathogens not only to the seed itself but also to the plants originating from the seed, after they have emerged. In this way, it may not be necessary to treat the crop directly at the time of sowing or shortly thereafter. A further advantage is to be seen in the fact that, through the treatment of the seed with composition of the disclosure, germination and emergence of the treated seed may be promoted.

The compositions of the disclosure are suitable for protecting seed of any variety of plant which is used in agriculture, in greenhouses, in forestry or in horticulture. More particularly, the seed in question is that of cereals (e.g., wheat, barley, rye, oats and millet), maize, cotton, soybeans, rice, potatoes, sunflower, coffee, tobacco, canola, oilseed rape, beets (e.g., sugar beet and fodder beet), peanuts, vegetables (e.g., tomato, cucumber, bean, brassicas, onions and lettuce), fruit plants, lawns and ornamentals. Particularly important is the treatment of the seed of cereals (such as wheat, barley, rye and oats) maize, soybeans, cotton, canola, oilseed rape and rice.

For the purposes of the present disclosure, the composition of the disclosure is applied alone or in a suitable formulation to the seed. The seed is preferably treated in a condition in which its stability is such that no damage occurs in the course of the treatment. Generally speaking, the seed may be treated at any point in time between harvesting and sowing. Typically, seed is used which has been separated from the plant and has had cobs, hulls, stems, husks, hair or pulp removed. Thus, for example, seed may be used that has been harvested, cleaned and dried to a moisture content of less than 15% by weight. Alternatively, seed can also be used that after drying has been treated with water, for example, and then dried again.

When treating seed it is necessary, generally speaking, to ensure that the amount of the composition of the disclosure, and/or of other additives, that is applied to the seed is selected such that the germination of the seed is not adversely affected, and/or that the plant which emerges from the seed is not damaged. This is the case in particular with active ingredients which may exhibit phytotoxic effects at certain application rates.

The compositions of the disclosure can be applied directly, in other words without comprising further components and without having been diluted. As a general rule, it is preferable to apply the compositions in the form of a suitable formulation to the seed. Suitable formulations and methods for seed treatment are known to the skilled person and are described in, for example, the following documents: U.S. Pat. Nos. 4,272,417 A; 4,245,432 A; 4,808,430 A; 5,876,739 A; U.S. Patent Application Publication No. 2003/0176428 A1; WO 2002/080675 A1; WO 2002/028186 A2, the contents of each of which being incorporated herein by reference.

The combinations which can be used in accordance with the disclosure may be converted into the customary seed-dressing formulations, such as solutions, emulsions, suspensions, powders, foams, slurries or other coating compositions for seed, and also ULV formulations. These formulations are prepared in a known manner, by mixing composition with customary adjuvants, such as, for example, customary extenders and also solvents or diluents, colorants, wetters, dispersants, emulsifiers, antifoams, preservatives, secondary thickeners, stickers, gibberellins, and also water. Colorants which may be present in the seed-dressing formulations which can be used in accordance with the invention include all colorants which are customary for such purposes. In this context it is possible to use not only pigments, which are of low solubility in water, but also water-soluble dyes. Examples include the colorants known under designations Rhodamin B, C.I. Pigment Red 112, and C.I. Solvent Red 1.

Depending on the plant species or plant cultivars, their location and growth conditions (soils, climate, vegetation period, diet), using or employing the composition according to the present disclosure the treatment according to the disclosure may also result in super-additive (“synergistic”) effects. Thus, for example, by using or employing inventive composition in the treatment according to the disclosure, reduced application rates and/or a widening of the activity spectrum and/or an increase in the activity better plant growth, increased tolerance to high or low temperatures, increased tolerance to drought or to water or soil salt content, increased flowering performance, easier harvesting, accelerated maturation, higher harvest yields, bigger fruits, larger plant height, greener leaf color, earlier flowering, higher quality and/or a higher nutritional value of the harvested products, higher sugar concentration within the fruits, better storage stability and/or processability of the harvested products are possible, which exceed the effects which were actually to be expected.

At certain application rates of the inventive composition in the treatment according to the disclosure may also have a strengthening effect in plants. The defense system of the plant against attack by unwanted phytopathogenic fungi and/or microorganisms and/or viruses is mobilized. Plant-strengthening (resistance-inducing) substances are to be understood as meaning, in the present context, those substances or combinations of substances which are capable of stimulating the defense system of plants in such a way that, when subsequently inoculated with unwanted phytopathogenic fungi and/or microorganisms and/or viruses, the treated plants display a substantial degree of resistance to these phytopathogenic fungi and/or microorganisms and/or viruses. Thus, by using or employing composition according to the present disclosure in the treatment according to the disclosure, plants can be protected against attack by the abovementioned pathogens within a certain period of time after the treatment. The period of time within which protection is effected generally extends from 1 to 10 days, preferably 1 to 7 days, after the treatment of the plants with the active compounds.

Any of the compositions disclosed herein may include one or more agrochemicals. Similarly, the methods of applying compositions according to the disclosure may further comprise introducing at least one agrochemical into the plant growth medium or applying at least one agrochemical to plants or seeds.

The agrochemical can comprise a fertilizer (e.g., a liquid fertilizer), a micronutrient fertilizer material (e.g., boric acid, a borate, a boron frit, copper sulfate, a copper frit, a copper chelate, a sodium tetraborate decahydrate, an iron sulfate, an iron oxide, iron ammonium sulfate, an iron frit, an iron chelate, a manganese sulfate, a manganese oxide, a manganese chelate, a manganese chloride, a manganese frit, a sodium molybdate, molybdic acid, a zinc sulfate, a zinc oxide, a zinc carbonate, a zinc frit, zinc phosphate, a zinc chelate, or a combination thereof), an insecticide (e.g., an organophosphate, a carbamate, a pyrethroid, an acaricide, an alkyl phthalate, boric acid, a borate, a fluoride, sulfur, a haloaromatic substituted urea, a hydrocarbon ester, a biologically-based insecticide, or a combination thereof), an herbicide (e.g., a chlorophenoxy compound, a nitrophenolic compound, a nitrocresolic compound, a dipyridyl compound, an acetamide, an aliphatic acid, an anilide, a benzamide, a benzoic acid, a benzoic acid derivative, anisic acid, an anisic acid derivative, a benzonitrile, benzothiadiazinone dioxide, a thiocarbamate, a carbamate, a carbanilate, chloropyridinyl, a cyclohexenone derivative, a dinitroaminobenzene derivative, a fluorodinitrotoluidine compound, isoxazolidinone, nicotinic acid, isopropylamine, an isopropylamine derivatives, oxadiazolinone, a phosphate, a phthalate, a picolinic acid compound, a triazine, a triazole, a uracil, a urea derivative, endothall, sodium chlorate, or a combination thereof), a fungicide (e.g., a substituted benzene, a thiocarbamate, an ethylene bis dithiocarbamate, a thiophthalidamide, a copper compound, an organomercury compound, an organotin compound, a cadmium compound, anilazine, benomyl, cyclohexamide, dodine, etridiazole, iprodione, metlaxyl, thiamimefon, triforine, or a combination thereof), a molluscicide, an algicide, a plant growth amendment, a bacterial inoculant (e.g., a bacterial inoculant of the genus Rhizobium, a bacterial inoculant of the genus Bradyrhizobium, a bacterial inoculant of the genus Mesorhizobium, a bacterial inoculant of the genus Azorhizobium, a bacterial inoculant of the genus Allorhizobium, a bacterial inoculant of the genus Sinorhizobium, a bacterial inoculant of the genus Kluyvera, a bacterial inoculant of the genus Azotobacter, a bacterial inoculant of the genus Pseudomonas, a bacterial inoculant of the genus Azospirillium, a bacterial inoculant of the genus Bacillus, a bacterial inoculant of the genus Streptomyces, a bacterial inoculant of the genus Paenibacillus, a bacterial inoculant of the genus Paracoccus, a bacterial inoculant of the genus Enterobacter, a bacterial inoculant of the genus Alcaligenes, a bacterial inoculant of the genus Mycobacterium, a bacterial inoculant of the genus Trichoderma, a bacterial inoculant of the genus Gliocladium, a bacterial inoculant of the genus Glomus, a bacterial inoculant of the genus Klebsiella, or a combination thereof), a fungal inoculant (e.g., a fungal inoculant of the family Glomeraceae, a fungal inoculant of the family Claroidoglomeraceae, a fungal inoculant of the family Gigasporaceae, a fungal inoculant of the family Acaulosporaceae, a fungal inoculant of the family Sacculosporaceae, a fungal inoculant of the family Entrophosporaceae, a fungal inoculant of the family Pacidsporaceae, a fungal inoculant of the family Diversisporaceae, a fungal inoculant of the family Paraglomeraceae, a fungal inoculant of the family Archaeosporaceae, a fungal inoculant of the family Geosiphonaceae, a fungal inoculant of the family Ambisporaceae, a fungal inoculant of the family Scutellosporaceae, a fungal inoculant of the family Dentiscultataceae, a fungal inoculant of the family Racocetraceae, a fungal inoculant of the phylum Basidiomycota, a fungal inoculant of the phylum Ascomycota, a fungal inoculant of the phylum Zygomycota, or a combination thereof), or a combination thereof.

The fertilizer can comprise ammonium sulfate, ammonium nitrate, ammonium sulfate nitrate, ammonium chloride, ammonium bisulfate, ammonium polysulfide, ammonium thiosulfate, aqueous ammonia, anhydrous ammonia, ammonium polyphosphate, aluminum sulfate, calcium nitrate, calcium ammonium nitrate, calcium sulfate, calcined magnesite, calcitic limestone, calcium oxide, calcium nitrate, dolomitic limestone, hydrated lime, calcium carbonate, diammonium phosphate, monoammonium phosphate, magnesium nitrate, magnesium sulfate, potassium nitrate, potassium chloride, potassium magnesium sulfate, potassium sulfate, sodium nitrates, magnesian limestone, magnesia, urea, urea-formaldehydes, urea ammonium nitrate, sulfur-coated urea, polymer-coated urea, isobutylidene diurea, K₂SO₄—(MgSO₄)₂, kainite, sylvinite, kieserite, Epsom salts, elemental sulfur, marl, ground oyster shells, fish meal, oil cakes, fish manure, blood meal, rock phosphate, super phosphates, slag, bone meal, wood ash, manure, bat guano, peat moss, compost, green sand, cottonseed meal, feather meal, crab meal, fish emulsion, humic acid, or a combination thereof. The agrochemical can comprise any fungicide, bacterial inoculant, or herbicide, as described herein. The spore-forming bacterium, alone or in combination with the insecticide, can further comprise an effective amount of at least one fungicide.

In general, a “fungicide” is a substance to increase mortality or inhibit the growth rate of fungi. The term “fungus” or “fungi” includes a wide variety of nucleated spore-bearing organisms that are devoid of chlorophyll. Examples of fungi include yeasts, molds, mildews, rusts, and mushrooms. Typical fungicidal ingredients also include captan, fludioxonil, iprodione, tebuconazole, thiabendazole, azoxystrobin, prochloraz, and oxadixyl. Select compositions, plant seeds, or inoculums according to the disclosure may comprise any natural or synthetic fungicide, such as: aldimorph, ampropylfos, ampropylfos potassium, andoprim, anilazine, azaconazole, azoxystrobin, benalaxyl, benodanil, benomyl, benzamacril, benzamacryl-isobutyl, bialaphos, binapacryl, biphenyl, bitertanol, blasticidin-S, boscalid, bromuconazole, bupirimate, buthiobate, calcium polysulphide, capsimycin, captafol, captan, carbendazim, carvon, quinomethionate, chlobenthiazone, chlorfenazole, chloroneb, chloropicrin, chlorothalonil, chlozolinate, clozylacon, cufraneb, cymoxanil, cyproconazole, cyprodinil, cyprofuram, debacarb, dichlorophen, diclobutrazole, diclofluanid, diclomezine, dicloran, diethofencarb, dimethirimol, dimethomorph, dimoxystrobin, diniconazole, diniconazole-M, dinocap, diphenylamine, dipyrithione, ditalimfos, dithianon, dodemorph, dodine, drazoxolon, edifenphos, epoxiconazole, etaconazole, ethirimol, etridiazole, famoxadon, fenapanil, fenarimol, fenbuconazole, fenfuram, fenitropan, fenpiclonil, fenpropidin, fenpropimorph, fentin acetate, fentin hydroxide, ferbam, ferimzone, fluazinam, flumetover, fluopyram, fluoromide, fluquinconazole, flurprimidol, flusilazole, flusulfamide, flutolanil, flutriafol, folpet, fosetyl-aluminium, fosetyl-sodium, fthalide, fuberidazole, furalaxyl, furametpyr, furcarbonil, furconazole, furconazole-cis, furmecyclox, guazatine, hexachlorobenzene, hexaconazole, hymexazole, imazalil,imibenconazole, iminoctadine, iminoctadine albesilate, iminoctadine triacetate, iodocarb, iprobenfos (IBP), iprodione, irumamycin, isoprothiolane, isovaledione, kasugamycin, kresoxim-methyl, copper preparations, such as: copper hydroxide, copper naphthenate, copper oxychloride, copper sulphate, copper oxide, oxine-copper and Bordeaux mixture, mancopper, mancozeb, maneb, meferimzone, mepanipyrim, mepronil, metalaxyl, metconazole, methasulfocarb, methfuroxam, metiram, metomeclam, metsulfovax, mildiomycin, myclobutanil, myclozolin, nickel dimethyldithiocarbamate, nitrothal-isopropyl, nuarimol, ofurace, oxadixyl, oxamocarb, oxolinic acid, oxycarboxim, oxyfenthiin, paclobutrazole, pefurazoate, penconazole, pencycuron, phosdiphen, pimaricin, piperalin, polyoxin, polyoxorim, probenazole, prochloraz, procymidone, propamocarb, propanosine-sodium, propiconazole, propineb, prothiocinazole, pyrazophos, pyrifenox, pyrimethanil, pyroquilon, pyroxyfur, quinconazole, quintozene (PCNB), sulphur and sulphur preparations, tebuconazole, tecloftalam, tecnazene, tetcyclasis, tetraconazole, thiabendazole, thicyofen, thifluzamide, thiophanate-methyl, tioxymid, tolclofos-methyl, tolylfluanid, triadimefon, triadimenol, triazbutil, triazoxide, trichlamide, tricyclazole, tridemorph, trifloxystrobin, triflumizole, triforine, uniconazole, validamycin A, vinclozolin, viniconazole, zarilamide, zineb, ziramor, or a combination thereof. The fungicide can also comprise a substituted benzene, a thiocarbamate, an ethylene bis dithiocarbamate, a thiophthalidamide, a copper compound, an organomercury compound, an organotin compound, a cadmium compound, anilazine, benomyl, cyclohexamide, dodine, etridiazole, iprodione, metlaxyl, thiamimefon, triforine, or a combination thereof. One of ordinary skill in the art will readily appreciate that other known synthetic or naturally-occurring fungicides used for agricultural purposes may also be selected for inclusion in a composition, plant seed or inoculum according to the disclosure.

If a composition, plant seed, or inoculum comprises a fungicide, the fungicide can be a foliar fungicide. Foliar fungicides include copper, mancozeb, penthiopyrad, triazoles, cyproconazole, metconazole, propiconazole, prothioconazole, tebuconazole, azoxystrobin, pyraclastobin, fluoxastrobin, picoxystrobin, trifloxystrobin, sulfur, boscalid, thiophanate methyl, chlorothanonil, penthiopyrad, difenconazole, flutriafol, cyprodinil, fluzinam, iprodione, penflufen, cyazofamid, flutolanil, cymoxanil, dimethomorph, pyrimethanil, zoxamide, mandipropamid, metrinam, propamocarb, fenamidone, tetraconazole, chloronab, hymexazol, tolclofos, and fenbuconazole. One of ordinary skill in the art will readily appreciate that other known synthetic or naturally-occurring foliar fungicides used for agricultural purposes may also be selected for inclusion in a composition, plant seed or inoculum according to the disclosure.

Compositions, seeds, and inoculants according to the disclosure comprising an insecticide, possess the ability to increase mortality or inhibit growth rate of insects. As used herein, the term “insects” includes all organisms in the class “Insecta”. The term “pre-adult” insects refers to any form of an organism prior to the adult stage, including, for example, eggs, larvae, and nymphs. As used herein, the terms “insecticide” and “insecticidal” also encompass “nematicide” and “nematicidal” and “acaricide” and “acaricidal.” “Nematicides” and “nematicidal” refers to the ability of a substance to increase mortality or inhibit the growth rate of nematodes. In general, the term “nematode” comprises eggs, larvae, juvenile and mature forms of said organism. “Acaricide” and “acaricidal” refers to the ability of a substance to increase mortality or inhibit growth rate of ectoparasites belonging to the class Arachnida, sub-class Acari.

According to one aspect of the present disclosure, the at least one insecticide comprises: (1) Acetylcholinesterase (AChE) inhibitors, such as, for example, carbamates, for example alanycarb, bendiocarb, benfuracarb, butocarboxim, butoxycarboxim, carbofuran, carbosulfan, ethiofencarb, furathiocarb, isoprocarb, metolcarb, oxamyl, pirimicarb, propoxur, thiofanox, triazamate, trimethacarb, XMC and xylylcarb; or organophosphates, for example acephate, azamethiphos, azinphos-ethyl, azinphos-methyl, cadusafos, chlorethoxyfos, chlorfenvinphos, chlormephos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, famphur, fenitrothion, fosthiazate, heptenophos, imicyafos, isofenphos, isopropyl O-(methoxyaminothiophosphoryl) salicylate, isoxathion, malathion, mecarbam, methidathion, mevinphos, monocrotophos, naled, omethoate, parathion-methyl, phenthoate, phorate, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, and triclorfon. (2) GABA-gated chloride channel antagonists, such as, for example, cyclodiene-organochlorines, for example chlordane and/or phenylpyrazoles. (3) Sodium channel modulators/voltage-gated sodium channel blockers such as, for example, pyrethroids, e.g., acrinathrin, allethrin, d-cis-trans allethrin, d-trans allethrin, bifenthrin, bioallethrin, bioallethrin s-cyclopentenyl isomer, bioresmethrin, cycloprothrin, cyhalothrin, lambda-cyhalothrin, gamma-cyhalothrin, empenthrin [(EZ)-(IR)-isomer], esfenvalerate, etofenprox, fenpropathrin, fenvalerate, flucythrinate, flumethrin, tau-fluvalinate, halfenprox, imiprothrin, kadethrin, permethrin, phenothrin [(IR)-trans-isomer], prallethrin, pyrethrins (pyrethrum), resmethrin, tefluthrin, tetramethrin, tetramethrin [(1R)-isomer)], and transfluthrin or DDT or methoxychlor. (4) Nicotinergic acetylcholine receptor (nAChR) agonists, such as, for example, neonicotinoids, e.g., dinotefuran, nitenpyram, and thiamethoxam or nicotine or sulfoxaflor. (5) Allosteric activators of the nicotinergic acetylcholine receptor (nAChR) such as, for example, spinosyns, e.g., spinetoram and spinosad. (6) Chloride channel activators, such as, for example, avermectins/milbemycins, for example abamectin, emamectin benzoate, lepimectin and milbemectin. (7) Juvenile hormone imitators such as, for example, juvenile hormone analogues, e.g., hydroprene, kinoprene and methoprene or fenoxycarb or pyriproxyfen. (8) Active compounds with unknown or nonspecific mechanisms of action such as, for example, alkyl halides, e.g., methyl bromide and other alkyl halides; or chloropicrine or sulphuryl fluoride or borax or tartar emetic. (9) Selective antifeedants, for example pymetrozine or flonicamid. (10) Mite growth inhibitors, for example clofentezine, hexythiazox and diflovidazin or etoxazole. (11) Microbial disrupters of the insect gut membrane, for example Bacillus thuringiensis subspecies israelensis, Lysinibacillus sphaericus, Bacillus thuringiensis subspecies aizawai, Bacillus thuringiensis subspecies kurstaki, Bacillus thuringiensis subspecies tenebrionis, and Bt plant proteins: Cry1Ab, Cry1Ac, Cry1Fa, Cry2Ab, mCry3A, Cry3Ab, Cry3Bb, Cry34/35Abl. (12) Oxidative phosphorylation inhibitors, ATP disrupters such as, for example, diafenthiuron or organotin compounds, for example azocyclotin, cyhexatin and fenbutatin oxide or propargite or tetradifon. (13) Oxidative phosphorylation decouplers acting by interrupting the H proton gradient such as, for example, chlorfenapyr, DNOC and sulfluramid. (14) Nicotinergic acetylcholine receptor antagonists such as, for example, bensultap, cartap hydrochloride, thiocylam, and thiosultap-sodium. (15) Chitin biosynthesis inhibitors, type 0, such as, for example, bistrifluron, chlorfluazuron, diflubenzuron, flucycloxuron, flufenoxuron, hexaflumuron, lufenuron, novaluron, noviflumuron, and teflubenzuron. (16) Chitin biosynthesis inhibitors, type 1, for example buprofezin. (17) Moulting inhibitors (in particular for Diptera, i.e., dipterans) such as, for example, cyromazine. (18) Ecdysone receptor agonists such as, for example, chromafenozide, halofenozide, methoxyfenozide and tebufenozide. (19) Octopaminergic agonists. (20) Complex-Ill electron transport inhibitors such as, for example, hydramethylnone or acequinocyl or fluacrypyrim. (21) Complex-I electron transport inhibitors, for example from the group of the METI acaricides, e.g., fenazaquin, fenpyroximate, pyrimidifen, pyridaben, tebufenpyrad and tolfenpyrad or rotenone (Derris). (22) Voltage-gated sodium channel blockers, for example indoxacarb or metaflumizone. (23) Inhibitors of acetyl-CoA carboxylase. (24) Complex-IV electron transport inhibitors such as, for example, phosphines, e.g., aluminium phosphide, calcium phosphide, phosphine and zinc phosphide or cyanide. (25) Complex II electron transport inhibitors, such as, for example, cyenopyrafen and cyflumetofen. (26) Ryanodine receptor effectors, such as, for example, diamides, e.g., chlorantraniliprole, which is also known by the trade name RYNAXYPYR™, and cyantraniliprole, or any combination of one or more of the compounds or classes of compounds identified above.

One of ordinary skill in the art will readily appreciate that other known synthetic or naturally-occurring insecticides used for agricultural purposes may also be selected for inclusion in a composition, plant seed or inoculum according to the disclosure.

### Screening Methods

The fusion protein constructs and recombinant Brevibacillus, Lysinibacillus, Viridibacillus, and/or Paenibacillus cells disclosed herein may be used as a platform for high-throughput screening of heterologous proteins that generate new and/or modified plant attributes, as discussed throughout the disclosure. Such attributes may include commercially significant improvements in plant yields and other plant characteristics, such as: altered plant protein or oil content/composition, altered plant carbohydrate content/composition; altered seed carbohydrate content/composition, altered seed oil or protein composition; increased tolerance to environmental or chemical stresses (e.g., resistance to cold or heat, drought, insecticides or herbicides); delayed senescence or disease resistance; growth improvement, health enhancement; herbivore resistance; improved nitrogen fixation or nitrogen utilization; improved root architecture or length; improved water use efficiency; increased biomass; increased seed weight; increased shoot length; increased yield; modified kernel mass or moisture content; metal tolerance; pathogen or pest resistance; photosynthetic capability improvement; salinity tolerance; vigor improvement; increased dry and/or fresh weight of mature seeds, increased number of mature seeds per plant; increased chlorophyll content; a detectable modulation in the level of a metabolite or in the metabolome relative to a reference plant/seed; a detectable modulation in the level of a transcript or in the transcriptome relative to a reference plant/seed; a detectable modulation in the level of a protein or in the proteome relative to a reference plant; and combinations of any of the traits or attributes above. Moreover, the preceding list is intended as a non-limiting set of examples. One of ordinary skill will appreciate that the high-throughput delivery platform disclosed herein is suitable for screening for various other plant traits and attributes discussed elsewhere in the disclosure or otherwise known in the art.

Endospores produced by recombinant Brevibacillus, Lysinibacillus, Viridibacillus, and/or Paenibacillus cells modified to express a fusion protein according to the disclosure may be applied to plant cells grown in vitro, a host plant seed, seedling, or to a vegetative or otherwise mature plant. The heterologous protein may in turn modify or confer a trait or attribute to the plant cells grown in vitro, host plant seed, seedling or mature plant. In select embodiments, the Brevibacillus, Lysinibacillus, Viridibacillus, and/or Paenibacillus endospores may be used to inoculate a seed and the resulting new or modified trait or attribute may be immediately apparent, whereas on other embodiments it may not become apparent until a later stage of development of the host plant.

In some embodiments, the Brevibacillus, Lysinibacillus, Viridibacillus, and/or Paenibacillus bacterium used to deliver the fusion protein is exogenous to the host plant species. In others, the selected Brevibacillus, Lysinibacillus, Viridibacillus, and/or Paenibacillus bacterium is an endogenous endophyte known to colonize the host plant species. The host plant may be any suitable plant disclosed here (a monocot, dicot, conifer, etc.)

The recombinant Brevibacillus, Lysinibacillus, Viridibacillus, and/or Paenibacillus bacterium used to deliver the fusion protein may be used to inoculate a host plant seed, seedling, vegetative or otherwise mature plant specimen by way of a coating or spray, or any other method of applying endospores to a host plant known in the art. When applied as a liquid, for example, as a solution or suspension, the Brevibacillus, Lysinibacillus, Viridibacillus, and/or Paenibacillus endospores may be mixed or suspended in aqueous solutions. Suitable liquid diluents or carriers include aqueous solutions, petroleum distillates, or other liquid carriers. Solid compositions can be prepared by dispersing the Brevibacillus, Lysinibacillus, Viridibacillus, and/or Paenibacillus endospores in and on an appropriately divided solid carrier, such as peat, wheat, bran, vermiculite, clay, talc, bentonite, diatomaceous earth, fuller's earth, pasteurized soil, and the like. When such formulations comprise wettable powders, dispersing agents such as non-ionic, anionic, amphoteric, or cationic dispersing and emulsifying agents can be used.

Brevibacillus, Lysinibacillus, Viridibacillus, and/or Paenibacillus endospores may be applied directly to the surface of host plant seeds or to the leaves and stem of a vegetative plant directly, or as part of a composition comprising additional components. The additional components may include one or more compounds that enhance the rate of colonization, compounds that enhance plant growth or health, pesticides or herbicides, or any other compounds disclosed herein as suitable for promoting cultivation and growth of plants. Moreover, the composition may include additional Brevibacillus, Lysinibacillus, Viridibacillus, and/or Paenibacillus endospores that have been modified to express fusion proteins comprising different amino acid sequences. For example, a composition may comprise a first Brevibacillus, Lysinibacillus, Viridibacillus, and/or Paenibacillus endospore that expresses a fusion protein comprising a plant growth promoting factor as well as a second Brevibacillus, Lysinibacillus, Viridibacillus, ad/or Paenibacillus endospore that expresses a fusion protein that comprises a protein that enhances pesticide-resistance.

In select embodiments, the recombinant Brevibacillus, Lysinibacillus, Viridibacillus, and/or Paenibacillus endospore which is coated onto the seed of a host plant is capable, upon germination of the seed into a vegetative state, of localizing to a different tissue of the plant. For example, the recombinant Brevibacillus, Lysinibacillus, Viridibacillus, and/or Paenibacillus cells can be capable of localizing to any one of the tissues in the plant, including: the root, adventitious root, seminal root, root hair, shoot, leaf, flower, bud, tassel, meristem, pollen, pistil, ovaries, stamen, fruit, stolon, rhizome, nodule, tuber, trichome, guard cells, hydathode, petal, sepal, glume, rachis, vascular cambium, phloem, and xylem. In other embodiments, the recombinant Brevibacillus, Lysinibacillus, Viridibacillus, and/or Paenibacillus cells may be capable of localizing to the root and/or the root hair of the plant. In alternative embodiments, the recombinant Brevibacillus, Lysinibacillus, Viridibacillus, and/or Paenibacillus cells may be capable of localizing to the photosynthetic tissues, for example, leaves and shoots of the plant; or to the vascular tissues of the plant, for example, in the xylem and phloem.

In other embodiments, the recombinant Brevibacillus, Lysinibacillus, Viridibacillus, and/or Paenibacillus cells are capable of localizing to the reproductive tissues (flower, pollen, pistil, ovaries, stamen, fruit) of the plant. In still another embodiment, the recombinant Brevibacillus, Lysinibacillus, Viridibacillus, and/or Paenibacillus cells colonize a fruit or seed tissue of the plant. In still another embodiment, the recombinant Brevibacillus, Lysinibacillus, Viridibacillus, and/or Paenibacillus cells are able to colonize the plant such that it is present on the surface of the plant (e.g., the plant exterior or the phyllosphere of the plant). In still other embodiments, the recombinant Brevibacillus, Lysinibacillus, Viridibacillus, and/or Paenibacillus cells are capable of localizing to substantially all, or all, tissues of the plant.

Compositions comprising the recombinant Brevibacillus, Lysinibacillus, Viridibacillus, and/or Paenibacillus endospores designed for application to a host plant may comprise a seed coating composition, a root treatment, or a foliar application composition. The seed coating composition, or the root treatment, or the foliar application composition may comprise a fungicide, an antibacterial agent, an herbicide, a nematicide, an insecticide, a plant growth regulator, a nutrient, or combinations thereof. The seed coating composition, or the root treatment, or the foliar application composition can further comprise an agriculturally acceptable carrier, a tackifier, a microbial stabilizer, or a combination thereof. In select embodiments, the seed coating composition, or the root treatment, or the foliar application composition can contain a second bacteria, including but not limited to a rhizobial bacterial preparation. The compositions may also contain a surfactant. In one embodiment, the surfactant is present at a concentration of between 0.01% v/v to 10% v/v. In another embodiment, the surfactant is present at a concentration of between 0.1% v/v to 1% v/v. In some embodiments, the composition may include a microbial stabilizer (e.g., a stabilizer).

Upon inoculation, a treated host plant (e.g., a treated seed, seedling, vegetative or otherwise mature plant) may be screened for the existence of new or modified attributes or traits. Screening can occur at any time point following treatment. In select embodiments, a seed may be treated and screening may not occur until the seed has sprouted or reached a more advanced stage of development. In other embodiments, a seed, seedling or vegetative plant may be treated and screening may not occur until the treated plant has produced a harvested end product which may comprise the sample to be screened for a new or modified trait or attribute.

During screening, various tests may be performed both in vitro and in vivo to determine what benefits, if any, are conferred upon the treated host plant. In vivo screening assays include tests that measure phenotypic traits or attributes of a plant or seed (e.g., assays measuring plant growth rate or height; crop yield; resistance to an environmental stress such as heat, cold, or salinity; resistance to biological pathogens or insect pests; resistance to chemical treatments such as insecticides or herbicides). In vitro screening assays include, but are not limited to, tests that measure the composition or properties of plant extracts, tissue samples, cell samples, and the like. In some embodiments, in vitro screening may comprise purifying and measuring the amount or activity of a given protein, enzyme, gene transcript, metabolite or other compound found in the cells or tissue of the treated host plant. In other embodiments, screening may comprise visual inspection of the structure of cells or tissue of the treated host plant, whether by the naked eye or via microscopy.

In alternative embodiments, screening may comprise assays of recombinant Brevibacillus, Lysinibacillus, Viridibacillus, and/or Paenibacillus endospores or vegetative cells modified to express a fusion protein according to the present disclosure, as opposed to assays directed to treated host plants. In these embodiments, the Brevibacillus, Lysinibacillus, Viridibacillus, and/or Paenibacillus family member cells or endospores may be subject to in vitro assays of one or more activities, such as but not limited to the ability to liberate complexed phosphates or complexed iron (e.g., through secretion of siderophores); production of phytohormones; production of antibacterial, antifungal, or insecticidal, or nematicidal compounds; production and/or secretion of ACC deaminase, acetoin, pectinase, cellulase, or RNase. Screening methods directed to the Brevibacillus, Lysinibacillus, Viridibacillus, and/or Paenibacillus family member cells or endospores, rather than vegetative plants, are particularly advantageous in that such methods may allow detection of useful heterologous proteins sooner than methods directed to treated host plants.

### DEPOSIT INFORMATION

Samples of the Brevibacillus, Lysinibacillus, and Viridibacillus strains of the invention have been deposited with the Agricultural Research Service Culture Collection located at the National Center for Agricultural Utilization Research, Agricultural Research Service, U.S. Department of Agriculture (NRRL), 1815 North University Street, Peoria, IL 61604, U.S.A., under the Budapest Treaty. The Brevibacillus and Lysinibacillus strains NRRL B-67865, and NRRL B-67864, respectively, both were deposited on Oct. 10, 2019. The Viridibacillus strain NRRL B-67869 was deposited on Oct. 17, 2019. The Brevibacillus, Lysinibacillus, and Viridibacillus strains have been deposited under conditions that assure that access to the culture will be available during the pendency of this patent application to one determined by the Commissioner of Patents and Trademarks to be entitled thereto under 37 C.F.R. § 1.14 and 35 U.S.C. § 122. However, it should be understood that the availability of a deposit does not constitute a license to practice the subject invention in derogation of patent rights granted by governmental action. All other Brevibacillus, Lysinibacillus, Viridibacillus, and Paenibacillus species referenced herein are believed to be commercially available and/or available to the public from recognized cell/culture repositories (e.g., the NRRL or the ATCC).

The following non-limiting examples are provided to further illustrate the present disclosure.

### EXAMPLES

### Example 1: General Protocol for Preparing Recombinant Brevibacillus, Lysinibacillus, Viridibacillus, or Paenibacillus Endospores

To create a fusion protein construct, a polynucleotide encoding one or more heterologous proteins may be fused to a polynucleotide encoding the amino acids of any N-terminal targeting sequence disclosed herein (e.g., any of the amino acid sequences disclosed in Tables 1-4 or FIGS. 1-7, or a fragment or variant thereof) that retains exosporium-targeting functionality when expressed in at least two of Brevibacillus, Lysinibacillus, Viridibacillus, and/or Paenibacillus. The fusion construct may be under the control of the native promoter of the disclosed N-terminal targeting sequence. Such constructs may be generated using the splicing by overlapping extension (SOE) technique or Gibson assembly resulting in a linear amplicon. At this stage, the protocol may also be modified to fuse the N-terminal targeting sequence to an in-frame coding sequence for a tag (e.g., GFP) or to insert a linker or protease recognition sequence between the N-terminal targeting sequence and the heterologous protein. Regardless of whether the optional tag, linker, and/or protease recognition sequence is included, correct amplicons may then be selected and cloned into a suitable shuttle vector (e.g., pHP13 for E. coli/Brevibacillus), and correct vector constructs screened by DNA sequencing. This vector construct may be electroporated into untransformed Brevibacillus, Lysinibacillus, Viridibacillus, and/or Paenibacillus cells. Correct transformants may then be grown in a suitable sporulation medium (e.g., Schaeffer's Sporulation Medium broth overnight at 30° C.) until sporulation has occurred (typically 2-3 days). Spores expressing the fusion construct may be harvested and then be subjected to one or more in vitro screening assays, applied directly to a host plant or seed, or applied to a host plant or seed which is then later subjected to one or more in vitro or in vivo screening assays.

### Example 2: Use of Recombinant Brevibacillus, Lysinibacillus, Viridibacillus, or Paenibacillus Endospores for Delivery of a Fusion Protein Involved in the Production of a Plant Growth Promoting Compound to a Seed, Seedling, Plant, or Plant Part

Enzymes responsible for the production of plant growth promoting compounds can be delivered to plants using the Brevibacillus, Lysinibacillus, and/or Viridibacillus endospore delivery systems disclosed herein. For example, butanediol dehydrogenase converts acetoin to 2,3-butanediol. 2,3-butanediol is a plant growth promoting compound. Brevibacillus, Lysinibacillus, Viridibacillus, and/or Paenibacillus endospores expressing this enzyme can be applied as a seed treatment or seed coating or delivered to the area surrounding a seed, seedling, plant, or plant part by drip or spray.

### Example 3: Use of Recombinant Brevibacillus, Lysinibacillus, Viridibacillus, or Paenibacillus Endospores for Delivery of More than One Fusion Protein on a Single Brevibacillus, Lysinibacillus, Viridibacillus, or Paenibacillus Endospore to a Seed, Seedling, Plant, or Plant Part

A single recombinant Brevibacillus, Lysinibacillus, Viridibacillus, and/or Paenibacillus endospore can be used to display more than one heterologous fusion protein. This is accomplished by constructing two (or more) separate fusion proteins. The coding sequence for each heterologous protein to be displayed on the Brevibacillus, Lysinibacillus, Viridibacillus, and/or Paenibacillus endospore surface is fused separately to an N-terminal targeting sequence under control of its native promoters. The fusion protein constructs can be cloned either into the same plasmid vector or different plasmid vectors and introduced into Brevibacillus, Lysinibacillus, Viridibacillus, and/or Paenibacillus cells by electroporation. The resulting Brevibacillus, Lysinibacillus, Viridibacillus, and/or Paenibacillus endospores will then express a mixture of both heterologous proteins on the spore surface. This is particularly useful for stacking multiple proteinaceous invertebrate toxins to mitigate pest resistance.

### Example 4: Use of More than One Recombinant Brevibacillus, Lysinibacillus, Viridibacillus, or Paenibacillus Endospores in Combination, Each Displaying One or More Different Fusion Proteins to a Seed, Seedling, Plant, or Plant Part

In certain cases, delivery of more than one Brevibacillus, Lysinibacillus, Viridibacillus, and/or Paenibacillus endospore in combination each expressing one or more different heterologous proteins (as described above) are provided. For example, the delivery of nitrogen fixation enzymes to the area surrounding the roots of a plant reduces the need for chemical nitrogen fertilizers. Nitrogen fixation in bacteria may require, at minimum, eight or nine different enzymes and potentially upwards of twenty different enzymes depending on the species. Here, delivery of a combination of Brevibacillus, Lysinibacillus, Viridibacillus, and/or Paenibacillus endospores each expressing different enzyme components of the nitrogen fixation pathway may useful. For example, Brevibacillus, Lysinibacillus, Viridibacillus, and/or Paenibacillus endospores heterologously displaying NifH, NifD, and NifK may be combined in a mixture with Brevibacillus, Lysinibacillus, Viridibacillus, and/or Paenibacillus endospores heterologously displaying NifE, NifN, and NifD and delivered to the area surrounding the roots.

### Example 5: Use of Recombinant Brevibacillus, Lysinibacillus, Viridibacillus, or Paenibacillus Endospores for Delivery of an Invertebrate Toxin that Kills Invertebrate Plant Pests to the Area Surrounding a Seed, Seedling, Plant, or Plant Part or as a Seed Treatment

Proteinaceous toxins antagonistic towards invertebrates including but not limited to insects or nematodes can be delivered using the Brevibacillus, Lysinibacillus, Viridibacillus, and/or Paenibacillus endospore systems disclosed herein. For example, Cry toxins including but not limited to Cry5B and Cry21A which are both insecticidal and nematicidal may be fused to the N-terminal targeting sequence for expression in Brevibacillus, Lysinibacillus, Viridibacillus, and/or Paenibacillus endospores. Brevibacillus, Lysinibacillus, Viridibacillus, and/or Paenibacillus endospores expressing Cry toxins or other proteinaceous invertebrate toxins can be applied as a seed treatment or seed coating or delivered to the area surrounding a seed, seedling, plant, or plant part by drip or spray for protection against invertebrate plant pathogens.

### Example 6: Use of Brevibacillus, Lysinibacillus, Viridibacillus, or Paenibacillus Endospores for Delivery of a Peptide, Protein, or Enzyme that is Antagonistic Towards Bacterial Plant Pests to the Area Surrounding a Seed, Seedling, Plant, or Plant Part or as a Seed Treatment

Bacteriocins are small peptides produced by bacteria with antagonistic activity towards other bacteria. Due to the fact that bacteriocins are ribosomally synthesized as opposed to other antimicrobial molecules (e.g., bacitracin), which are synthesized by large non-ribosomal peptide synthetases, bacteriocins are especially well suited for delivery using the Brevibacillus endospore system. The coding sequence for one or more bacteriocins may be fused to the N-terminal targeting sequence for expression in Brevibacillus, Lysinibacillus, Viridibacillus, and/or Paenibacillus endospores. Brevibacillus, Lysinibacillus, Viridibacillus, and/or Paenibacillus endospores expressing bacteriocins can be applied as a seed treatment or seed coating or delivered to the area surrounding a seed, seedling, plant, or plant part by drip or spray for protection against bacterial plant pathogens.

### Example 7: Use of Brevibacillus, Lysinibacillus, Viridibacillus, or Paenibacillus Endospores for Delivery of a Peptide, Protein, or Enzyme that is Antagonistic Towards Fungal Plant Pests to the Area Surrounding a Seed, Seedling, Plant, or Plant Part or as a Seed Treatment

The primary cell wall component of fungi is chitin. Chitinase is an enzyme that degrades chitin and can be expressed on the surface of Brevibacillus, Lysinibacillus, Viridibacillus, and/or Paenibacillus endospores to protect against fungal plant pathogens by destroying their cell walls. Brevibacillus, Lysinibacillus, Viridibacillus, and/or Paenibacillus endospores expressing chitinase can be applied as a seed treatment or seed coating or delivered to the area surrounding a seed, seedling, plant, or plant part by drip or spray.

### Example 8: Use of Brevibacillus, Lysinibacillus, Viridibacillus, or Paenibacillus Endospores for Delivery of an Enzyme that Degrades or Modifies a Bacterial, Fungal, or Plant Nutrient Source to the Area Surrounding a Seed, Seedling, Plant, or Plant Part or as a Seed Treatment

Enzymes responsible for the degradation or modification of a bacterial, fungal, or plant nutrient source can be delivered to plants using recombinant Brevibacillus, Lysinibacillus, Viridibacillus, and/or Paenibacillus endospores. For example, a glycoside hydrolase which breaks down complex polysaccharides can be used to make available simple sugars for beneficial rhizobacteria by treating a plant or seed with recombinant Brevibacillus, Lysinibacillus, Viridibacillus, and/or Paenibacillus endospores expressing this (or another) enzyme of interest.

### Example 9: Use of Brevibacillus, Lysinibacillus, Viridibacillus, or Paenibacillus Endospores for Assessing Responses to Plant Growth Promoting Biocontrol Agents by Screening of Genomic DNA Libraries Derived from Plant Growth Promoting Biocontrol Agents

Many of the biocontrol strains used today are recalcitrant to exogenous DNA uptake rendering researchers unable to generate targeted genetic modifications of said strains. Due to this challenge, elucidating the mechanism of action of the plant growth promoting effects of these biocontrol strains is incredibly difficult. Brevibacillus, Lysinibacillus, Viridibacillus, and/or Paenibacillus endospores present a novel approach for identifying specific genes responsible for the underlying plant growth promoting effects of biocontrol strains. First, the N-terminal targeting sequence and native promoter are cloned into a suitable shuttle vector (e.g., pHP13 for Brevibacillus), resulting in a vector suitable for heterologous protein expression on Brevibacillus, Lysinibacillus, Viridibacillus, and/or Paenibacillus endospores. All cloning steps and plasmid propagation are performed in E. coli. Next, total gDNA is extracted from a target plant growth promoting biocontrol strain. The gDNA is sheared into fragments (enzymatically or sonically) and ligated into the above described vector for expression of heterologous proteins in Brevibacillus, Lysinibacillus, Viridibacillus, and/or Paenibacillus endospores to generate a gDNA library comprised of all the genetic material originating from the biocontrol strain of interest. The resulting vector library is introduced into a Brevibacillus, Lysinibacillus, Viridibacillus, and/or Paenibacillus member by electroporation and the bacteria are plated onto agar plates containing an appropriate antibiotic selection agent to select for successful transformants. Individual transformants, each expressing a different fragment of the target biocontrol strain's gDNA, are assessed for plant growth promoting effects. These effects can include but are not limited to enhanced greening, improved germination, increased plant vigor, increased root length, increased root mass, increased plant height, increased leaf area, or resistance to pests. The vector in Brevibacillus, Lysinibacillus, Viridibacillus, and/or Paenibacillus endospore transformants found to modulate the above mentioned plant health parameters can be sequenced to identify the genetic determinants originating from the biocontrol strain responsible for the observed plant growth promoting effects.

### Example 10: Use of Brevibacillus, Lysinibacillus, Viridibacillus, or Paenibacillus Endospores for Identifying Novel or Uncharacterized Toxins Antagonistic Against Plant Invertebrate, Bacterial, and Fungal Plant Pathogens

Many of the biocontrol strains in use today are recalcitrant to exogenous DNA uptake rendering researchers unable to generate targeted genetic modifications of said strains. Due to this challenge, elucidating the mechanism of action by which biocontrol strains are toxic towards invertebrate, bacterial, and fungal plant pathogens is incredibly difficult. Brevibacillus, Lysinibacillus, Viridibacillus, and/or Paenibacillus endospores present a novel approach for identifying specific genes responsible for the underlying plant protective effects of biocontrol strains. First, the N-terminal targeting sequence and native promoter are cloned into a suitable shuttle vector (e.g., pHP13 for Brevibacillus) resulting in a vector suitable for heterologous protein expression on Brevibacillus, Lysinibacillus, Viridibacillus, and/or Paenibacillus endospores. All cloning steps and plasmid propagation are performed in E. coli, total gDNA is extracted from a target plant growth promoting biocontrol strain. The gDNA is sheared into fragments (enzymatically or sonically) and ligated into the above described vector for expression of heterologous proteins on Brevibacillus, Lysinibacillus, Viridibacillus, and/or Paenibacillus endospores to generate a gDNA library comprised of all the genetic material originating from the biocontrol strain of interest. The resulting vector library is introduced into a Brevibacillus, Lysinibacillus, Viridibacillus, and/or Paenibacillus member by electroporation and the bacteria are plated onto agar plates containing an appropriate antibiotic selection agent to select for successful transformants. Individual transformants, each expressing a different fragment of the target biocontrol strain's gDNA, are assessed for antagonist activity towards invertebrate, bacterial, and fungal plant pathogens. The vector in Brevibacillus, Lysinibacillus, Viridibacillus, and/or Paenibacillus transformants that are found to be antagonistic towards the above plant pathogens can be sequenced to identify the genetic determinants originating from the biocontrol strain responsible for the observed plant protective effects.

### Example 11: Use of Purified Exosporiums from Brevibacillus, Lysinibacillus, Viridibacillus, or Paenibacillus Endospores as a Treatment to a Seed, Seedling, Plant, or Plant Part to Improve Plant Health

There may be a need to deliver plant health promoting proteins/enzymes using the Brevibacillus, Lysinibacillus, Viridibacillus, and/or Paenibacillus endospore systems disclosed herein without viable Brevibacillus, Lysinibacillus, Viridibacillus, and/or Paenibacillus endospores. To that end, the exosporium from a Brevibacillus, Lysinibacillus, Viridibacillus, and/or Paenibacillus endospore (e.g., produced by a cell modified to product a heterologous protein using the N-terminal targeting sequences disclosed herein) can be stripped away from the endospore via sufficient agitation through sonication. The stripped exosporiums are then further purified through filtration. The resulting purified exosporiums can be applied as a seed treatment or seed coating or delivered to the area surrounding a seed, seedling, plant, or plant part by drip or spray.

### Example 12: Use of Non-Viable Brevibacillus, Lysinibacillus, Viridibacillus, or Paenibacillus Endospores as a Treatment to a Seed, Seedling, Plant, or Plant Part for the Purposes of Protecting Plants from Pathogens or Improving Plant Health

There may be a need to deliver plant health promoting proteins/enzymes or plant protection proteins/enzymes using the Brevibacillus, Lysinibacillus, Viridibacillus, and/or Paenibacillus endospore delivery systems disclosed herein, with non-viable (dead) Brevibacillus endospores. Brevibacillus, Lysinibacillus, Viridibacillus, and/or Paenibacillus endospores can be inactivated and rendered non-viable via sufficient heat treatment, UV light, gamma irradiation, or high-pressure processing. The resulting non-viable Brevibacillus, Lysinibacillus, Viridibacillus, and/or Paenibacillus endospores can be applied as a seed treatment or seed coating or delivered to the area surrounding a seed, seedling, plant, or plant part by drip or spray.

### Example 13: A General Protocol for Preparing Recombinant Brevibacillus Endospores Displaying Tandem-Dimer Tomato (tdTomato)

The complete genome of Brevibacillus sp. NRRL B-67865 was searched for ORFs containing collagen-like GXX repeats by bioinformatics analysis. Sequences from ORFs containing collagen-like repeats were compiled and trimmed to yield only N-terminal amino acids upstream from collagen-like repeats. This general protocol was used to identify endogenous Brevibacillus sp. NRRL B-67865 proteins having the N-terminal targeting sequences disclosed in Table 1 and FIGS. 1-3. For example, this approach was used to identify the N-terminal targeting sequence represented by SEQ ID NO: 3, which was found to be directly upstream of a CLR repeat domain (SEQ ID NO: 220) and under the control of a native promoter encoded by SEQ ID NO: 221.

TABLE 5
CLR and Promoter Sequences Associated With an Exemplary Brevibacillus
Exosporium-Targeted Protein

| Sequence | |
|---|---|
| Identifier | Sequence |
| SEQ ID NO: 220 | GVTGATGATGPTGATGATGATGPTGVTGATGAAGGDTGATGATGQTGET |
| | GVAGATGATGQTGETGVAGATGATGQTGETGVAGATGATGQTGETGVA |
| | GATGATGQTGETGVAGATGATGQTGETGVAGATGATGQTGETGVAGAT |
| | GATGQTGETGVAGATGATGQTGETGVAGATGATGQTGETGKAGATGAT |
| | GQTGETGVTGATGATGQTGETGEAGATGAAGQTGETGVTGATGATGQT |
| | GETGVAGATGATGQTGETGVAGATGATGATGQTGETGVVGATGATGQT |
| | GETGVVGATGATGQTGETGVAGATGATGATGQTGETGVAGATGATGQT |
| | GETGVAGATGATGQTGETGVAGATGATGQTGETGVAGATGATGQTGEA |
| | GVAGVTGATG |
| SEQ ID NO: 221 | TGAAAAGATGGGGAATTCTCCATCTTTTTTCTTTTGTCTGGAGTGGTGA |
| | GATATACGACATACAACGAATGAGCATAACATGAATAGTTTATAAAAT |
| | CGCAAAAAATTTTTCGGAAGGAGAGAGCGAAAATTAACAATAAGCGG |
| | GCTAACGCATTCAATAAGAAG |

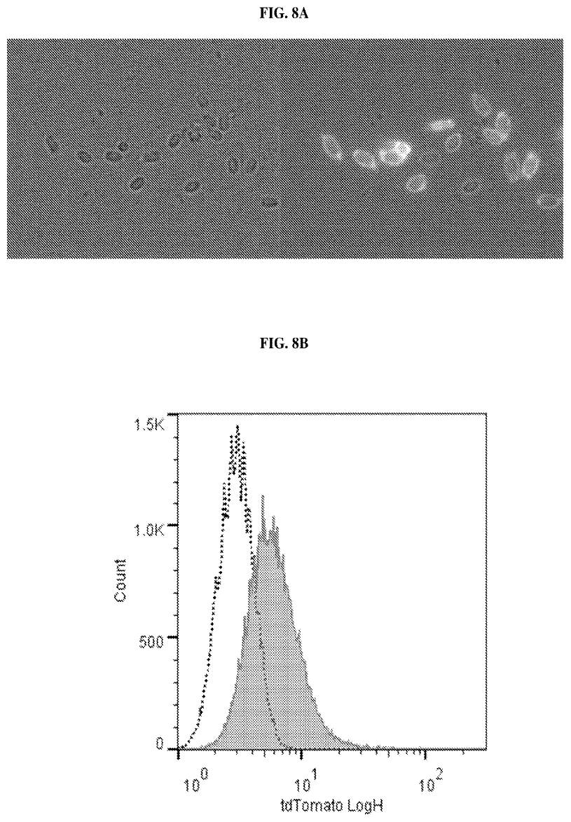

To create fusion constructs, the gene coding for tdTomato was fused to a DNA segment encoding the amino acids of the disclosed N-terminal targeting sequence (SEQ ID NO: 3) of Brevibacillus sp. NRRL B-67865 under control of the native promoter of the disclosed N-terminal targeting sequences by gene synthesis and cloned into an E. coli/Brevibacillus shuttle vector, pAP13. The resulting vector construct was introduced into Brevibacillus sp. NRRL B-67865 by electroporation similar to that described by Huang et al. (2010), “Production of an In Vitro-Derived Deletion Mutation of Brevibacillus laterosporus by Constructing a Homology-Driven Integration Vector,” Current Microbiology, 61:401-406, doi:10.1007/s00284-010-9627-0. Correct transformants were then grown in a glucose-based broth medium at 30° C. until sporulation. Brevibacillus sp. NRRL B-67865 spores expressing the fusion construct were then examined by epifluorescent microscopy. TdTomato is visible on spores expressing the fusion construct (FIG. 8A). Brevibacillus sp. NRRL B-67865 spores were also examined by flow cytometry. Spores expressing the fusion construct are significantly more fluorescent than wild-type spores (FIG. 8B).

### Example 14: A General Protocol for Preparing Recombinant Lysinibacillus Endospores Displaying Tandem-Dimer Tomato (tdTomato)

The complete genome of Lysinibacillus sp. NRRL B-67864 was searched for ORFs containing collagen-like GXX repeats by bioinformatics analysis. Sequences from ORFs containing collagen-like repeats were compiled and trimmed to yield only N-terminal amino acids upstream from collagen-like repeats. This general protocol was used to identify endogenous Lysinibacillus sp. NRRL B-67864 proteins having the N-terminal targeting sequences disclosed in Table 2 and FIGS. 4-5. For example, this approach was used to identify the N-terminal targeting sequence represented by SEQ ID NO: 43, which was found to be directly upstream of a CLR repeat domain (SEQ ID NO: 222 and under the control of a native promoter encoded by SEQ ID NO: 223.

TABLE 6
CLR and Promoter Sequences Associated With an Exemplary Lysinibacillus
Exosporium-Targeted Protein

| Sequence | |
|---|---|
| Identifier | Sequence |
| SEQ ID NO: 222 | GVTGATGATGATGAQGPPGAPGPQGVAGAMGPRGEIGPQGVAGATGAT |
| | GPQGEMGLQGVAGVTGASGPQGEIGPQGVAGVTGAPGPQGEIGPQGVAG |
| | ATGVPGPQGEIGPQGVAGETGATGPQGEMGLQGVAGVTGATGPQGEMG |
| | PQGVAGVTGATGPQGEIGPQGVAGVTGAPGPQGEIGPQGVAGVTGAPGP |
| | QGEMGLQGVAGVTGATGPQGIIGATGLRGLAGITGATGERGLAGITGATG |
| | VQGVAGSTGATGSQGITGATGVQGVTGSTGATGSQGITGATGVQGVTGS |
| | TGATGSQGITGATGVQGVTGSTGATGSQGITGATGVQGVAGSTGATGSQ |
| | GITGATGVQGVTGSTGATGARGTTGVTGTTGPSGSGLMEVFLSTDQSVG |
| | NNDFLGTGNSSASFVRSSIVIPENATIRSLTLNIRDHALSAGQTASAQIFVST |
| | NCGFTSVATGIIATVTGPNSSTTPNCCAKVIANYPVSSCTLLSVQVSTTGG |
| | AFSNGVSATVLFNTV |
| SEQ ID NO: 223 | TGTTGAAACATATTATTGAGCATTGGGTTTTATATGACCTAATGTTCTT |
| | TTTATTTTAAGATACATATTTAAATCATTCTAATATTCTAGGTGCATTA |
| | ATTATCTCAATAAGTAGACTTTTGAATATGATAAAAAGGGACAGGAGG |
| | TGAAGTCGCA |

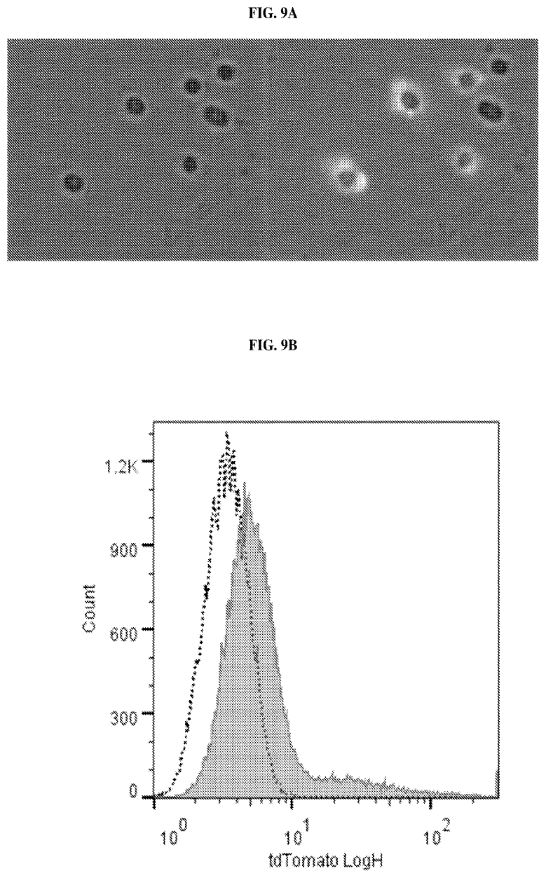

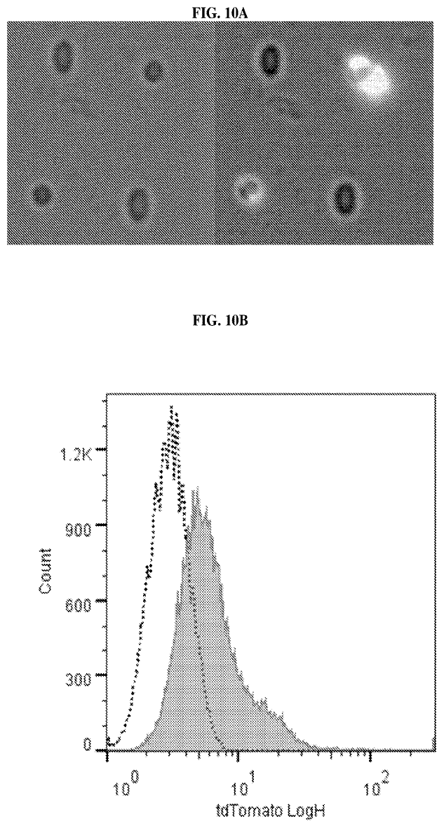

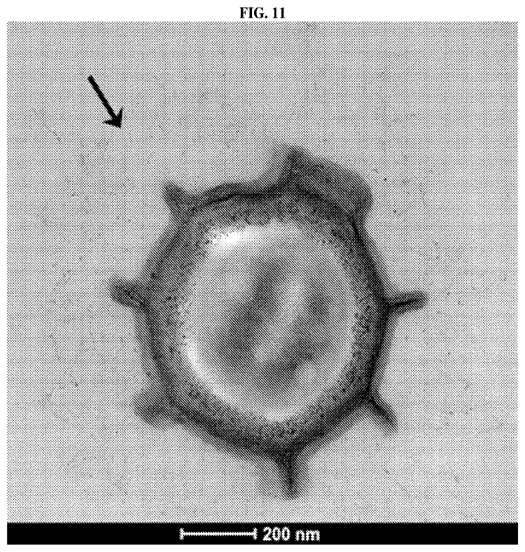

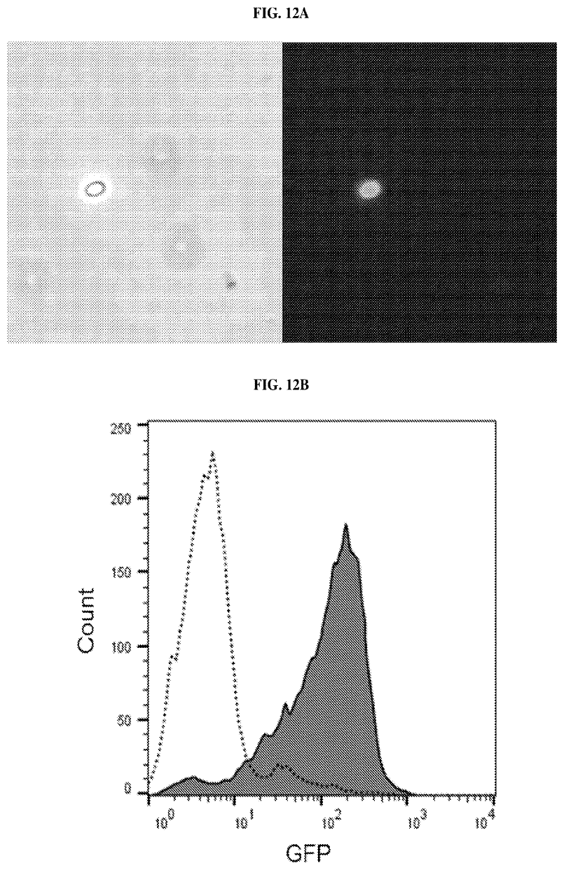

To create fusion constructs, the gene coding for tdTomato was fused to a DNA segment encoding the amino acids of the disclosed N-terminal targeting sequence (SEQ ID NO: 43) of Lysinibacillus sp. NRRL B-67864 under control of the native promoter of the disclosed N-terminal targeting sequences by gene synthesis and cloned into an E. coli/Lysinibacillus shuttle vector, pAP13. The resulting vector construct was introduced into Lysinibacillus sp. NRRL B-67864 by electroporation similar to that described by Taylor and Burke (1990), “Transformation of an entomopathic strain of Bacillus sphaericus by high voltage electroporation,” FEMS Microbiology Letters, 66:125-128, doi.org/10.1111/j.1574-6968.1990.tb03983.x. Correct transformants were then grown in a glucose-based broth medium at 30° C. until sporulation. Lysinibacillus sp. NRRL B-67864 spores expressing the fusion construct were then examined by epifluorescent microscopy. TdTomato is visible on spores expressing the fusion construct (FIG. 9A). Lysinibacillus sp. NRRL B-67864 spores were also examined by flow cytometry. Spores expressing the fusion construct are significantly more fluorescent than wild-type spores (FIG. 9B).

### Example 15: A General Protocol for Preparing Recombinant Viridibacillus Endospores Displaying Tandem-Dimer Tomato (tdTomato)

The approach described in Examples 13 and 14 was used to identify N-terminal targeting sequences in Viridibacillus. This approach led to the identification of the N-terminal targeting sequence represented by SEQ ID NO: 197, which was found to be directly upstream of a CLR repeat domain (SEQ ID NO: 224 and under the control of a native promoter encoded by SEQ ID NO: 225).

TABLE 7
CLR and Promoter Sequences Associated With an Exemplary Viridibacillus
Exosporium-Targeted Protein

| Sequence | |
|---|---|
| Identifier | Sequence |
| SEQ ID NO: 224 | GKTGPTGATGPTGSMGPTGVTGSTGATGPTGSTGVTGETGSTGTTGPSGS |
| | TGPTGATGSTGETGATGSTGATGETGPTGPTGTTGVTGETGATGPTGATG |
| | ATGETGSTGPTGATGETGETGSTGPTGATGSTGATGPTGATGSTGATGST |
| | GATGSTGATGPTGATGSTGETGPTGETGSTGATGSTGATGETGPTGTTGV |
| | TGETGATGPTGATGATGETGETGSTGPTGATGETGETGSTGPTGATGSTG |
| | ATGPTGATGSTGATGSTGATGSTGATGPTGATGSTGETGPTGETGSTGAT |
| | GSTGATGETGPTGATGSTGATGETGATGPTGPTGTTGVTGATGPTGATGE |
| | TGPTGATGEAGPTGSTGATGPTGATGITGATGPTGSTGATGSTGETGSTGE |
| | TGPTGATGVTGATGPTGETGSTGPMGATGETGSTGSTGVTGATGATGET |
| | GATGSTGATGTTGATGETGPTGPTGSTGATGETGPTGPTGATGVTGATGA |
| | TGATGETGSTGPMGATGETGSTGSTGVTGATGATGVTGATGETGSTGAT |
| | GSTGATGETGPTGPTGPTGPTGTTGVTGVTGETGPTGATGETGSTGPTGT |
| | TGVTGETGATGPTGATGSTGETGATGETGRTGPTGTTGVTGETGATGETG |
| | PTGATGSTGATGPTGATGATGATGETGPTGATGETGATGATGSTGATGPT |
| | GPTGTTGVTGATGPTGATGETGPTGATGEAGPTGSTGSTGPTGATGITGA |
| | TGPTGATGATGSTGETGSTGETGPTGSTG |
| SEQ ID NO: 225 | ATATTTTAGAAAGTGAAAAATACAGATAAAAGCCTTTGCTGATTCATT |
| | TTTCTGGATTTATTTAAAAGAACGATTGGAGAGTATTCTTCAGCCGTAT |
| | CTTTTTTTTGTTGCTAAAAATATTAATAATTTTACTTGAGGATTTTAAT |
| | AGATTAAGAAATTTTTAATTCTGATACCTATATTGTATTACAGGAATAT |
| | TTTAGAAATAAGCATATCCCATTTTCATTTTACAGTTTAATAGCATGCT |
| | AGAGAAATACATAATTATCAGACTACCATGAATGAGATGATTATCCGA |
| | TTTTTTTCTGTCACATTACAATTGTCCAATATCAATCTATTTCACGAGA |
| | ATAGTATGTATATGATGGGAGGTGATAATA |

To create fusion constructs, the gene coding for tdTomato was fused to a DNA segment encoding the amino acids of the disclosed N-terminal targeting sequence (SEQ ID NO: 197) of Viridibacillus sp. NRRL B-67869 under control of the native promoter of the disclosed N-terminal targeting sequences by gene synthesis and cloned into an E. coli/Viridibacillus shuttle vector, pAP13. The resulting vector construct was introduced into Viridibacillus sp. NRRL B3-67869 by electroporation using LBSP media, ampicillin treatment, and washing with TSMMKK buffer similar to that described by Zhang et al. (2015), “Development of an Efficient Electroporation Method for Iturin A-Producing Bacillus subtilis ZK,” International Journal of Molecular Sciences, 16:7334-7351, doi:10.3390/ijms16047334. Correct transformants were then grown in a glucose-based broth medium at 30° C. until sporulation. Viridibacillus sp. NRRL B-67869 spores expressing the fusion construct were then examined by epifluorescent microscopy. TdTomato is visible on spores expressing the fusion construct (FIG. 10A). Viridibacillus sp. NRRL B-67869 spores were also examined by flow cytometry. Spores expressing the fusion construct are significantly more fluorescent than wild-type spores (FIG. 10B).

### Example 16. General Protocol for Preparing Recombinant Paenibacillus Endospores Displaying Beta-Galactosidase (B-Gal) from Escherichia coli

To create fusion constructs, the gene coding for 3-gal was fused to a DNA segment encoding the amino acids of the disclosed N-terminal targeting sequence (SEQ ID NO: 226) of Paenibacillus sp. NRRL B-50972 under control of the native promoter of the disclosed N-terminal targeting sequences by gene synthesis and cloned into an E. coli/Paenibacillus shuttle vector derived from the pMiniMad vector described in Patrick, J E and Kearns, D B. 2008. MinJ (YvjD) is a Topological Determinant of Cell Division in Bacillus subtilis. Molecular Microbiology. 70: 1166-1179. The resulting vector construct was introduced into a Paenibacillus polymyxa strain (Strain 1) by electroporation similar to that described by Kim and Timmusk (2013), “A Simplified Method for Gene Knockout and Direct Screening of Recombinant Clones for Application in Paenibacillus polymyxa,” PLoS ONE, 8(6): e68092. A control was also prepared that contained the shuttle vector without the targeting sequence. Correct transformants were then grown in Schaeffer's Sporulation Medium broth at 30° C. until sporulation. The resulting culture was centrifuged to separate supernatant from spores. Paenibacillus polymyxa spores expressing the fusion construct or containing the empty shuttle vector only and corresponding supernatant were then examined by in vitro assay. β-gal is functional on spores expressing the fusion construct based on hydrolysis of 5-bromo-4-chloro-3-indolyl-β-D-galacto-pyranoside (X-Gal). Results are shown below in Table 8.

TABLE 8
Beta-galactosidase Activity of Supernatants and Spores

| | X-Gal |
|---|---|
| Sample | Hydrolysis^{a} |
| P. polymyxa empy shuttle vector supernatant | − |
| P. polymyxa N-terminal targeting sequence- | − |
| β-galactosidase supernatant | |
| P. polymyxa empy shuttle vector spores | − |
| P. polymyxa N-terminal targeting sequence- | + |
| β-galactosidase-pAP13 spores | |

^{a}X-gal hydrolysis was scored as (−) for no color or (+) for blue color denoting hydrolysis of X-gal by β-galactosidase.

### Example 17. General Protocol for Preparing Recombinant Paenibacillus Endospores Displaying Vegetative Insecticidal Protein 3 (Vip3) from Bacillus thuringiensis

To create fusion constructs, the gene coding for vip3 (SEQ ID NO: 242) was fused to a DNA segment encoding the amino acids of the disclosed N-terminal targeting sequence (SEQ ID NO: 226) of Paenibacillus sp. NRRL B-50972 by Gibson Assembly into the E. coli/Paenibacillus shuttle vector described in Example 16. Expression of the fusion is under control of the native promoter of the disclosed N-terminal targeting sequence. The resulting vector construct was introduced into a Paenibacillus polymyxa strain (Strain 1) by electroporation, as described above. Correct transformants were then grown in Schaeffer's Sporulation Medium broth at 30° C. until sporulation.

### Example 18. Activity of the Paenibacillus polymyxa Strain Expressing Vip3 Against Spodoptera exigua

The insecticidal activity of the Paenibacillus polymyxa strain expressing Vip3, from Example 17, was evaluated against Spodotera exigua (beet armyworm). A 96-well plate assay was performed to test the insecticidal activity of each Paenibacillus polymyxa strain including an empty vector control and an active cargo (SEQ ID NO: 227-Vip3). Spores of the strains were produced by growing the strains in Schaeffer's Sporulation Medium broth until sporulation and centrifuging the resulting whole broth culture to separate spores from supernatant. The spore samples from the strains were then applied to 96-well microplates containing an agar substrate similar to that described in Marrone et al., (1985), “Improvements in Laboratory Rearing of the Southern Corn Rootworm, Diabrotica undecimpuncta howardi Barber (Coleoptera: Chrysomelidae), on an Artificial Diet and Corn,” J. Econ. Entomol., 78: 290-293. The spore samples were then diluted in water and applied at concentrations of 100%, 33%, 11%, 3.7%, and 1.2% to the plates.

After the treatments had been allowed to dry, about 20 eggs from Spodotera exigua (beet armyworm) were added to each well. Several days later, the insecticidal activity was determined by evaluating the stunting scores and mortality scores of the treated larvae. Insect stunting scores were rated according to the following scale: 1=severely stunted; 2=highly stunted, minimal growth; 3=slightly smaller than untreated control; 4=same size as untreated control. The insect mortality score is based on the following scale: 4=0-25% mortality, 3=26-50% mortality, 2=51-79% mortality, 1=80-100% mortality.

Spodotera exigua larvae treated with 11% Paenibacillus spores expressing targeted Vip3 (i.e., SEQ ID NO: 227-Vip3) experienced 2-fold greater stunting that those treated with the same concentration of Paenibacillus spores expressing the empty vector (see Table 9). Similarly, larvae treated with 11% Paenibacillus spores expressing the targeted Vip3 experienced 1.5-fold greater mortality than those treated with the same concentration of Paenibacillus spores expressing the empty vector (see Table 9).

TABLE 9
Stunting Ratings of Treated Spodotera exigua (Beet Armyworm)

| | Stunting Score | |
|---|---|---|
| Application | SEQ ID NO: 2-Vip3 | Empty Vector |

| Rate | Mean | Std Err | Mean | Std Err |
|---|---|---|---|---|
| 1 | 1 | 0 | 1 | 0 |
| 0.33 | 1 | 0 | 1.7 | 0.7 |
| 0.11 | 1.5 | 0.5 | 3 | 1 |
| 0.037 | 3.3 | 0.7 | 4 | 0 |
| 0.012 | 4 | 0 | 4 | 0 |

### Example 19. A Demonstration of Intergeneric Display Using Exosporium-Targeted Fusion Proteins According to the Present Disclosure

This experiment evaluated the expression of several N-terminal targeting sequences according to the present disclosure, in Brevibacillus, Lysinibacillus, Viridibacillus, and Paenibacillus, using the constructs and promoters described in Table 10 below.

An N-terminal targeting sequence from Brevibacillus sp. NRRL B-67865 (SEQ ID NO: 3) was utilized to evaluate intergeneric display by fusing an N-terminal targeting sequence of a collagen-like repeat protein to that of tandem-dimer Tomato (tdTomato) fluorescent protein. Expression of the N-terminal targeting sequence-tdTomato fusion is driven by the native promoter upstream from the targeting sequence (SEQ ID NO: 221). The targeting sequence-tdTomato fusion gene was constructed by gene synthesis and subcloned into an E. coli/gram positive shuttle vector, pAP13. The resulting vector construct was introduced into the following strains: Brevibacillus sp. NRRL B-67865, B. brevis strain, and B. laterosporus strain by electroporation similar to that described by Huang et al. (2010), “Production of an In Vitro-Derived Deletion Mutation of Brevibacillus laterosporus by Constructing a Homology-Driven Integration Vector,” Current Microbiology, 61:401-406; Lysinibacillus sp. NRRL B-67864 by electroporation similar to that described by Taylor and Burke (1990), “Transformation of an entomopathic strain of Bacillus sphaericus by high voltage electroporation,” FEMS Microbiology Letters, 66:125-128; Paenibacillus peoriae strain or P. chitinolyticus strain by electroporation similar to that described by Kim and Timmusk (2013), “A Simplified Method for Gene Knockout and Direct Screening of Recombinant Clones for Application in Paenibacillus polymyxa,” PLoS ONE, 8(6): e68092; and Viridibacillus sp. NRRL B-67869 by electroporation using LBSP media, ampicillin treatment, and washing with TSMMKK buffer similar to that described by Zhang et al. (2015), “Development of an Efficient Electroporation Method for Iturin A-Producing Bacillus subtilis ZK,” International Journal of Molecular Sciences, 16:7334-7351. Correct transformants were then grown in a glucose-based broth medium at 30° C. until sporulation. Brevibacillus sp. NRRL B-67865, B. brevis, B. laterosporus, Lysinibacillus sp. NRRL B-67864, P. peoriae, P. chitinolyticus, and Viridibacillus sp. NRRL B-67869 spores expressing the fusion construct were then examined by epifluorescence microscopy. As summarized by Table 11, the tested Brevibacillus N-terminal targeting sequence was able to target tdTomato to the exosporia of endospores produced by members of several different genera of bacteria, demonstrating the intergeneric use of N-terminal targeting sequences disclosed herein.

An N-terminal targeting sequence from Lysinibacillus sp. NRRL B-67864 (SEQ ID NO: 43) was utilized to evaluate intergeneric display by fusing an N-terminal targeting sequence of a collagen-like repeat protein to that of tandem-dimer Tomato (tdTomato) fluorescent protein. Expression of the N-terminal targeting sequence-tdTomato fusion is driven by the native promoter upstream from the targeting sequence (SEQ ID NO: 223). The targeting sequence-tdTomato fusion was constructed by gene synthesis and subcloned into an E. coli gram positive shuttle vector, pAP13. The resulting vector construct was introduced into the following strains: Brevibacillus sp. NRRL B-67865, B. brevis strain, and B. laterosporus strain by electroporation similar to that described by Huang et al. (2010), “Production of an In Vitro-Derived Deletion Mutation of Brevibacillus laterosporus by Constructing a Homology-Driven Integration Vector,” Current Microbiology, 61:401-406; Lysinibacillus sp. NRRL B-67864 by electroporation similar to that described by Taylor and Burke (1990), “Transformation of an entomopathic strain of Bacillus sphaericus by high voltage electroporation,” FEMS Microbiology Letters, 66:125-128; Paenibacillus peoriae strain or P. chitinolyticus strain by electroporation similar to that described by Kim and Timmusk (2013), “A Simplified Method for Gene Knockout and Direct Screening of Recombinant Clones for Application in Paenibacillus polymyxa,” PLoS ONE, 8(6): e68092; and Viridibacillus sp. NRRL B-67869 by electroporation using LBSP media, ampicillin treatment, and washing with TSMMKK buffer similar to that described by Zhang et al. (2015), “Development of an Efficient Electroporation Method for Iturin A-Producing Bacillus subtilis ZK,” International Journal of Molecular Sciences, 16:7334-7351. Correct transformants were then grown in a glucose-based broth medium at 30° C. until sporulation. Brevibacillus sp. NRRL B-67865, B. brevis, B. laterosporus, Lysinibacillus sp. NRRL B-67864, P. peoriae, P. chitinolyticus, and Viridibacillus sp. NRRL B-67869 spores expressing the fusion construct were then examined by epifluorescence microscopy. As summarized by Table 11, the tested Lysinibacillus N-terminal targeting sequence was able to target tdTomato to the exosporia of endospores produced by members of several different genera of bacteria, demonstrating the intergeneric use of N-terminal targeting sequences disclosed herein.

An N-terminal targeting sequence from Paenibacillus sp. NRRL B-50972 (SEQ ID NO: 227) was utilized to evaluate intergeneric display by fusing an N-terminal targeting sequence of a collagen-like repeat protein to that of tandem-dimer Tomato (tdTomato) fluorescent protein. Expression of the N-terminal targeting sequence-tdTomato fusion is driven by the native promoter upstream from the targeting sequence (SEQ ID NO: 237). The targeting sequence-tdTomato fusion was constructed by gene synthesis and subcloned into an E. coli gram positive shuttle vector, pAP13. The resulting vector construct was introduced into the following strains: Brevibacillus sp. NRRL B-67865, B. brevis strain, and B. laterosporus strain by electroporation similar to that described by Huang et al. (2010), “Production of an In Vitro-Derived Deletion Mutation of Brevibacillus laterosporus by Constructing a Homology-Driven Integration Vector,” Current Microbiology, 61:401-406; Lysinibacillus sp. NRRL B-67864 by electroporation similar to that described by Taylor and Burke (1990), “Transformation of an entomopathic strain of Bacillus sphaericus by high voltage electroporation,” FEMS Microbiology Letters, 66:125-128; Paenibacillus peoriae strain or P. chitinolyticus strain by electroporation similar to that described by Kim and Timmusk (2013), “A Simplified Method for Gene Knockout and Direct Screening of Recombinant Clones for Application in Paenibacillus polymyxa,” PLoS ONE, 8(6): e68092; and Viridibacillus sp. NRRL B-67869 by electroporation using LBSP media, ampicillin treatment, and washing with TSMMKK buffer similar to that described by Zhang et al. (2015), “Development of an Efficient Electroporation Method for Iturin A-Producing Bacillus subtilis ZK,” International Journal of Molecular Sciences, 16:7334-7351. Correct transformants were then grown in a glucose-based broth medium at 30° C. until sporulation. Brevibacillus sp. NRRL B-67865, B. brevis, B. laterosporus, Lysinibacillus sp. NRRL B-67864, P. peoriae, P. chitinolyticus, and Viridibacillus sp. NRRL B-67869 spores expressing the fusion construct were then examined by epifluorescence microscopy. As summarized by Table 11, the tested Paenibacillus N-terminal targeting sequence was able to target tdTomato to the exosporia of endospores produced by members of several different genera of bacteria, demonstrating the intergeneric use of N-terminal targeting sequences disclosed herein.

An N-terminal targeting sequence from Viridibacillus sp. NRRL B-67869 (SEQ ID NO: 197) was utilized to evaluate intergeneric display by fusing an N-terminal targeting sequence of a collagen-like repeat protein to that of tandem-dimer Tomato (tdTomato) fluorescent protein. Expression of the N-terminal targeting sequence-tdTomato fusion is driven by the native promoter upstream from the targeting sequence (SEQ ID NO: 225). The targeting sequence-tdTomato fusion was constructed by gene synthesis and subcloned into an E. coli/gram positive shuttle vector, pAP13. The resulting vector construct was introduced into the following strains: Brevibacillus sp. NRRL B-67865, B. brevis strain, and B. laterosporus strain by electroporation similar to that described by Huang et al. (2010), “Production of an In Vitro-Derived Deletion Mutation of Brevibacillus laterosporus by Constructing a Homology-Driven Integration Vector,” Current Microbiology, 61:401-406; Lysinibacillus sp. NRRL B-67864 by electroporation similar to that described by Taylor and Burke (1990), “Transformation of an entomopathic strain of Bacillus sphaericus by high voltage electroporation,” FEMS Microbiology Letters, 66:125-128; Paenibacillus peoriae strain or P. chitinolyticus strain by electroporation similar to that described by Kim and Timmusk (2013), “A Simplified Method for Gene Knockout and Direct Screening of Recombinant Clones for Application in Paenibacillus polymyxa,” PLoS ONE, 8(6): e68092; and Viridibacillus sp. NRRL B-67869 by electroporation using LBSP media, ampicillin treatment, and washing with TSMMKK buffer similar to that described by Zhang et al. (2015), “Development of an Efficient Electroporation Method for Iturin A-Producing Bacillus subtilis ZK,” International Journal of Molecular Sciences, 16:7334-7351. Correct transformants were then grown in a glucose-based broth medium at 30° C. until sporulation. Brevibacillus sp. NRRL B-67865, B. brevis, B. laterosporus, Lysinibacillus sp. NRRL B-67864, P. peoriae, P. chitinolyticus, and Viridibacillus sp. NRRL B-67869 spores expressing the fusion construct were then examined by epifluorescence microscopy. As summarized by Table 11, the tested Viridibacillus N-terminal targeting sequence was able to target tdTomato to the exosporia of endospores produced by members of several different genera of bacteria, demonstrating the intergeneric use of N-terminal targeting sequences disclosed herein. Correct transformants could also be subjected to spore purification methods, such as centrifugation or use of a density gradient to obtain a spore-only or substantially spore-only sample which could be subjected to various analytical methods, such as microscopy, whole cell fluorescence, whole cell surface plasmon resonance, whole cell immunoassay, or other whole cell assay, such as flow cytometry.

This example tested several representative N-terminal targeting sequences according to the present disclosure and confirmed that each of the tested N-terminal targeting sequences maintained functionality in at least two different genera (e.g., in Brevibacillus, Lysinibacillus, Viridibacillus and/or Paenibacillus). Extrapolating from these results, it is expected that all of the N-terminal targeting sequences disclosed herein will display intergeneric exosporium-targeting functionality with respect to at least two, three, or all four of the above-identified genera.

Specifically, Table 11 illustrates the broad capability and utility of the targeting sequence from Paenibacillus. The plasmid using the Paenibacillus targeting sequence produced positive results, namely, host spores displaying visible tdTomato, in every strain of bacteria tested with the exception of Brevibacillus brevis, in which no plasmid tested was able to produce positive results. Furthermore, the plasmid with the Paenibacillus targeting sequence was the only plasmid to produce positive results in Paenibacillus peoriae. These results indicate that the Paenibacillus targeting sequence can serve as a powerful tool for screening putative host bacteria, such as non-Bacillus bacterial strains, for strains that are capable of displaying heterologous proteins with this method; bacteria that produced negative results with the Paenibacillus plasmid did not produce positive results with any other targeting sequence either, and every bacterial strain that produced positive results with any targeting sequence also produced positive results from the Paenibacillus plasmid. The use of the plasmid with the Brevibacillus targeting sequence, for example, produced a negative result in Paenibacillus peoriae that could cause this species to be discarded as a putative host, whereas P. peoriae is shown to be capable of displaying the tdTomato by the plasmid with the Paneibacillus targeting sequence. Thus, the Paenibacillus targeting sequence can be used as a reliable indicator for whether a bacterial strain is capable of displaying heterologous proteins on its exosporia.

Exemplary non-Bacillus bacterial host strains include at least endospore-forming bacteria, such as those set forth herein.

TABLE 10
Sequences Used to Construct and Express the Intergeneric Constructs Tested in
Experiment 19

| Sequence | |
|---|---|
| Identifier | Sequence |
| SEQ ID NO: 3 | MLPLIFPPQGCPAIFPGPTLDPPDPPDPP |
| SEQ ID NO: 221 | TGAAAAGATGGGGAATTCTCCATCTTTTTTCTTTTGTCTGGAGTGGTGA |
| | GATATACGACATACAACGAATGAGCATAACATGAATAGTTTATAAAAT |
| | CGCAAAAAATTTTTCGGAAGGAGAGAGCGAAAATTAACAATAAGCGG |
| | GCTAACGCATTCAATAAGAAG |
| SEQ ID NO: 43 | MRFSYNRNFDNFKCDAKCNYPMKYKCNEFGCNGYPYVYGIEDPI |
| SEQ ID NO: 223 | TGTTGAAACATATTATTGAGCATTGGGTTTTATATGACCTAATGTTCTT |
| | TTTATTTTAAGATACATATTTAAATCATTCTAATATTCTAGGTGCATTA |
| | ATTATCTCAATAAGTAGACTTTTGAATATGATAAAAAGGGACAGGAGG |
| | TGAAGTCGCA |
| SEQ ID NO: 227 | MVVLSTGPIANDPVLGVRPTQLVTVKIDNRDSVNSSIVLIEGFILNGSRTLY |
| | VQQLVVVGPNAVITRNFFANVDAFEFVFTTSGPAENETQISVWGKDALGQ |
| | LVPAHRLVSDELLGTDR |
| SEQ ID NO: 237 | GAAACGGGAGTGGTGAAATCATTGATGCTCAGCGCATTGTTGCGGATG |
| | AGCAACTAGATTCTTGAAACACAACATATGTACAGAGATAGAACCACA |
| | ATCGTAACAAATGGTTGAGACATAAAATAGAGGGAACAGGATCTTGA |
| | GAAAGATCTCATTGTTCACAAAAAAGCTTGATTTTACTAGAAAGGAGG |
| | GAGTATCCA |
| SEQ ID NO: 197 | MDSNKKRCAVCNKYQCSCNSKKRSPKTVIRDLDYIDPCEPLYIIDHCHNR |
| SEQ ID NO: 225 | ATATTTTAGAAAGTGAAAAATACAGATAAAAGCCTTTGCTGATTCATT |
| | TTTCTGGATTTATTTAAAAGAACGATTGGAGAGTATTCTTCAGCCGTAT |
| | CTTTTTTTTGTTGCTAAAAATATTAATAATTTTACTTGAGGATTTTAATA |
| | GATTAAGAAATTTTTAATTCTGATACCTATATTGTATTACAGGAATATT |
| | TTAGAAATAAGCATATCCCATTTTCATTTTACAGTTTAATAGCATGCTA |
| | GAGAAATACATAATTATCAGACTACCATGAATGAGATGATTATCCGAT |
| | TTTTTTCTGTCACATTACAATTGTCCAATATCAATCTATTTCACGAGAA |
| | TAGTATGTATATGATGGGAGGTGATAATA |

TABLE 11
Summary of the Results of Experiment 19

| | Plasmid Used to | | | |
|---|---|---|---|---|
| | Express Fusion Protein Constructs | | | |
| Host | pPaeni | pBrevi | pLysini | pViridi |
| Paenibacillus peoriae | + | − | − | − |
| Paenibacillus | + | + | + | + |
| chitinolyticus #1 | | | | |
| Paenibacillus | + | + | + | + |
| chitinolyticus #2 | | | | |
| Brevibacillus reuszeri | + | + | + | + |
| Brevibacillus brevis | − | − | − | − |
| Brevibacillus | + | + | + | + |
| laterosporus | | | | |
| Lysinibacillus | + | + | + | + |
| sphaericus | | | | |
| Viridibacillus arvi | + | + | + | + |

A “+” symbol denotes host spores which displayed visible tdTomato fused to targeting sequences from each genera.
A “−” symbol denotes host spores which did not display visible tdTomato fused when fused to targeting sequence.

We claim:

1. A nucleic acid molecule encoding a fusion protein, comprising (a) a first polynucleotide sequence encoding an N-terminal signal peptide, operably linked to (b) a second polynucleotide sequence encoding a polypeptide heterologous to the N-terminal signal peptide, wherein the first polynucleotide sequence comprises:
(i) a polynucleotide sequence having at least 60%, 70%, 80%, 90%, 95%, or 100% sequence identity with a polynucleotide sequence encoding a polypeptide sequence shown in Tables 1-4; or
(ii) a polynucleotide sequence comprising a fragment of at least 15, 30, 45, 60, 75, 90, 105, 120, 150, 210, 270, 330, 390 or 450 consecutive nucleotides of a polynucleotide sequence encoding a polypeptide sequence shown in Tables 1-4; and
wherein the N-terminal signal peptide is capable of targeting the fusion protein to an exosporium when expressed in members of at least two different bacterial genera selected from Brevibacillus, Lysinibacillus, Viridibacillus, and/or Paenibacillus.

2. The nucleic acid molecule of claim 1, wherein the fragment includes:
a) the first nucleotide of a polynucleotide sequence encoding any amino acid sequence shown in Tables 1-4; or
b) the last nucleotide of a polynucleotide sequence encoding any amino acid sequence shown in Tables 1-4.

3. The nucleic acid molecule of claim 1 or 2, wherein the first polynucleotide sequence comprises a polynucleotide sequence having at least 60%, 70%, 80%, 90%, or 95% sequence identity with a polynucleotide sequence encoding an amino acid sequence shown in Tables 1-4.

4. The nucleic acid molecule of any one of the preceding claims, wherein the fragment encodes amino acids 1-5, 1-10, 1-15, 1-20, 1-25, 1-30, 1-35, 1-40 or 1-45, 1-50, 1-75, 1-100, 1-125 or 1-150 of any one the amino acid sequences shown in Tables 1-4.

5. The nucleic acid molecule of any one of the preceding claims, wherein the polypeptide heterologous to the N-terminal signal peptide comprises:
(a) a plant growth-stimulating protein;
(b) an enzyme;
(c) a protein;
(d) a polypeptide heterologous to Brevibacillus, Lysinibacillus, Viridibacillus, or Paenibacillus;
(e) a therapeutic protein; or
(f) a plant immune-stimulating protein.

6. The nucleic acid molecule of any one of the preceding claims, further comprising a third polynucleotide sequence, encoding:
(a) a polypeptide comprising one or more protease cleavage sites, wherein the polypeptide is positioned between the N-terminal signal peptide and the polypeptide heterologous to the N-terminal signal peptide;
(b) a polypeptide comprising a selectable marker;
(c) a polypeptide comprising a visualization marker;
(d) a polypeptide comprising a protein recognition/purification domain; or
(e) a polypeptide comprising a flexible linker element, which connects the N-terminal signal peptide and the polypeptide heterologous to the N-terminal signal peptide.

7. The nucleic acid molecule of any one of the preceding claims, wherein:
a) the Brevibacillus endospore is an endospore formed by a Brevibacillus species, comprising: B. agri, B. aydinogluensis, B. borstelensis, B. brevis, B. centrosporus, B. choshinensis, B. fluminis, B. formosus, B. fulvus, B. ginsengisoli, B. invocatus, B. laterosporus, B. levickii, B. limnophilus, B. massiliensis, B. nitrificans, B. panacihumi, B. parabrevis, B. reuszeri, or B. thermorube; or an endospore formed by a bacterium that possesses a 16S rRNA gene that shares at least 97%, 98% or 99% identity with a 16S rRNA gene of a Brevibacillus species;
b) the Lysinibacillus endospore is an endospore formed by a Lysinibacillus species, comprising: Lysinibacillus sphaericus, Lysinibacillus boronitolerans, Lysinibacillus fusiformis, Lysinibacillus acetophenoni, Lysinibacillus alkaliphilus, Lysinibacillus chungkukjangi, Lysinibacillus composti, Lysinibacillus contaminans, Lysinibacillus cresolivorans, Lysinibacillus macroides, Lysinibacillus manganicus, Lysinibacillus mangiferihumi, Lysinibacillus massiliensis, Lysinibacillus meyeri, Lysinibacillus odysseyi, Lysinibacillus pakistanensis, Lysinibacillus parviboronicapiens, Lysinibacillus sinduriensis, Lysinibacillus tabacifolii, Lysinibacillus varians, Lysinibacillus xylanilyticus or Lysinibacillus halotolerans; or an endospore formed by a bacterium that possesses a 16S rRNA gene that shares at least 97%, 98% or 99% identity with a 16S rRNA gene of a Lysinibacillus species;
c) the Viridibacillus endospore is an endospore formed by a Viridibacillus species, comprising: Viridibacillus arvi, Viridibacillus arenosi, or Viridibacillus neidei; or an endospore formed by a bacterium that possesses a 16S rRNA gene that shares at least 97%, 98% or 99% identity with a 16S rRNA gene of a Viridibacillus species; or
d) the Paenibacillus endospore is an endospore formed by a Paenibacillus species, comprising: Paenibacillus sp. NRRL B-50972, Paenibacillus terrae, Paenibacillus polymyxa, or Paenibacillus peoriae; or an endospore formed by a bacterium that possesses a 16S rRNA gene that shares at least 97%, 98% or 99% identity with a 16S rRNA gene of a Paenibacillus species.

8. The nucleic acid molecule of any one of the preceding claims, operatively linked to a promoter element that is heterologous to at least one of: (i) the second polynucleotide sequence; or a (ii) Brevibacillus, Lysinibacillus, Viridibacillus, or Paenibacillus cell.

9. The nucleic acid molecule of any one of the preceding claims, wherein the first polynucleotide sequence comprises:
a codon-optimized polynucleotide sequence having at least 60%, 70%, 80% or 90% sequence identity with a polynucleotide sequence encoding an amino acid sequence shown in Tables 1-4, which is expressed at a higher rate or level in the Brevibacillus, Lysinibacillus, Viridibacillus, or Paenibacillus endospore compared to the polynucleotide sequence encoding an amino acid sequence shown in Tables 1-4, under identical conditions.

10. The nucleic acid molecule of any of the preceding claims, wherein the first polynucleotide sequence comprises:
(i) a polynucleotide sequence having at least 90% sequence identity with a polynucleotide sequence encoding a polypeptide sequence of any one of SEQ ID NOs: 1-11, 15, 41-43, 56, 60, 78, 83, 95, 98, 107, 112, 136, 153, 164, 179, 185, 189, 197, 227, 257, 259, 261, 263, 265, 269, and 272.

11. The nucleic acid molecule of any of the preceding claims, wherein the first polynuceotide sequence comprises a polynucleotide sequence having at least 95% sequence identity with a polynucleotide sequence encoding a polypeptide sequence of any one of SEQ ID NOs: 3, 43, 197, 227, and 269.

12. A fusion protein comprising an N-terminal signal peptide operably linked to a polypeptide heterologous to the N-terminal signal peptide, wherein the N-terminal signal peptide comprises:
(a) a polypeptide sequence having at least 60%, 70%, 80%, 90%, 95%, or 100% sequence identity with any one of the polypeptide sequences shown in Tables 1-4; or
(b) a polypeptide sequence comprising a fragment of at least 5, 10, 15, 20, 25, 30, 35, 40, 45, 50, 75, 100, 125 or 150 consecutive amino acids selected from any of the polypeptide sequences shown in Tables 1-4;
wherein the N-terminal signal peptide is capable of targeting the fusion protein to an exosporium when expressed in members of at least two different bacterial genera selected from Brevibacillus, Lysinibacillus, Viridibacillus, and/or Paenibacillus.

13. The fusion protein of claim 12, wherein the fragment includes:
a) the first amino acid of any polypeptide sequence shown in Tables 1-4; or
b) the last amino acid of any polypeptide sequence shown in Tables 1-4.

14. The fusion protein of claim 12 or 13, wherein the polypeptide sequence comprises a sequence having at least 60%, 70%, 80%, 90%, 95% or 100% sequence identity with any polypeptide sequence shown in Tables 1-4.

15. The fusion protein of any one of claims 12 to 14, wherein the fragment comprises amino acids 1-5, 1-10, 1-15, 1-20, 1-25, 1-30, 1-35, 1-40, 1-45, 1-50, 1-75, 1-100, 1-125 or 1-150 of any polypeptide sequence shown in Tables 1-4.

16. The fusion protein of any one of claims 12 to 15, wherein the polypeptide heterologous to the N-terminal signal peptide comprises:
(a) a plant growth-stimulating protein;
(b) an enzyme;
(c) a protein;
(d) a polypeptide heterologous to a Brevibacillus, Lysinibacillus, Viridibacillus, or Paenibacillus cell;
(e) a therapeutic protein; or
(f) a plant immune-stimulating protein.

17. The fusion protein of any one of claims 12 to 16, wherein the fusion protein further comprises:
(a) a polypeptide containing one or more protease cleavage sites, positioned between the N-terminal signal peptide and the polypeptide heterologous to the N-terminal signal peptide;
(b) a polypeptide comprising a selectable marker;
(c) a polypeptide comprising a visualization marker;
(d) a polypeptide comprising at least one protein recognition/purification domain; or
(e) a polypeptide comprising a flexible linker element, connecting the signal peptide and the polypeptide heterologous to the N-terminal signal peptide.

18. The fusion protein of any one of claims 12 to 17, wherein:
a) the Brevibacillus endospore is an endospore formed by a Brevibacillus species, comprising: B. agri, B. aydinogluensis, B. borstelensis, B. brevis, B. centrosporus, B. choshinensis, B. fluminis, B. formosus, B. fulvus, B. ginsengisoli, B. invocatus, B. laterosporus, B. levickii, B. limnophilus, B. massiliensis, B. nitrificans, B. panacihumi, B. parabrevis, B. reuszeri, or B. thermorube; or an endospore formed by a bacterium that possesses a 16S rRNA gene that shares at least 97%, 98% or 99% identity with a 16S rRNA gene of a Brevibacillus species;
b) the Lysinibacillus endospore is an endospore formed by a Lysinibacillus species, comprising: Lysinibacillus sphaericus, Lysinibacillus boronitolerans, Lysinibacillus fusiformis, Lysinibacillus acetophenoni, Lysinibacillus alkaliphilus, Lysinibacillus chungkukjangi, Lysinibacillus composti, Lysinibacillus contaminans, Lysinibacillus cresolivorans, Lysinibacillus macroides, Lysinibacillus manganicus, Lysinibacillus mangiferihumi, Lysinibacillus massiliensis, Lysinibacillus meyeri, Lysinibacillus odysseyi, Lysinibacillus pakistanensis, Lysinibacillus parviboronicapiens, Lysinibacillus sinduriensis, Lysinibacillus tabacifolii, Lysinibacillus varians, Lysinibacillus xylanilyticus or Lysinibacillus halotolerans; or an endospore formed by a bacterium that possesses a 16S rRNA gene that shares at least 97%, 98% or 99% identity with a 16S rRNA gene of a Lysinibacillus species;
c) the Viridibacillus endospore is an endospore formed by a Viridibacillus species, comprising: Viridibacillus arvi, Viridibacillus arenosi, or Viridibacillus neidei; or an endospore formed by a bacterium that possesses a 16S rRNA gene that shares at least 97%, 98% or 99% identity with a 16S rRNA gene of a Viridibacillus species; or
d) the Paenibacillus endospore is an endospore formed by a Paenibacillus species, comprising: Paenibacillus sp. NRRL B-50972, Paenibacillus terrae, Paenibacillus polymyxa, or Paenibacillus peoriae; or an endospore formed by a bacterium that possesses a 16S rRNA gene that shares at least 97, 98 or 99% identity with a 16S rRNA gene of a Paenibacillus species.

19. The fusion protein comprising an N-terminal signal peptide operably linked to a polypeptide heterologous to the N-terminal signal peptide, wherein the N-terminal signal peptide comprises a polypeptide sequence having at least 90% sequence identity with any one of the polypeptide sequences of SEQ ID NOs: 1-11, 15, 41-43, 56, 60, 78, 83, 95, 98, 107, 112, 136, 153, 164, 179, 185, 189, 197, 227, 257, 259, 261, 263, 265, 269, and 272.

20. The fusion protein comprising an N-terminal signal peptide operably linked to a polypeptide heterologous to the N-terminal signal peptide, wherein the N-terminal signal peptide comprises a polypeptide sequence having at least 95% sequence identity with any one of the polypeptide sequences of SEQ ID NOs: SEQ ID NOs: 3, 43, 197, 227, and 269.

21. A recombinant Brevibacillus, Lysinibacillus, Viridibacillus, or Paenibacillus cell comprising a bacterial chromosome comprising the nucleic acid molecule of any one of claims 1-11.

22. A vector comprising the nucleic acid molecule of any one of claims 1-11, wherein the vector comprises a plasmid, an artificial chromosome, or a viral vector.

23. The vector of claim 20, further comprising at least one of the following:
(a) an origin of replication that provides stable maintenance in a Brevibacillus, Lysinibacillus, Viridibacillus, or Paenibacillus cell;
(b) an origin of replication that provides selectively non-stable maintenance in a Brevibacillus, Lysinibacillus, Viridibacillus, or Paenibacillus cell;
(c) a temperature-sensitive origin of replication that provides selectively non-stable maintenance in a Brevibacillus, Lysinibacillus, Viridibacillus, or Paenibacillus cell;
(d) a polynucleotide encoding a selection marker, operably linked to an expression control sequence; or
(e) a polynucleotide encoding a plant growth stimulating protein, operably linked to an expression control sequence.

24. A recombinant Brevibacillus, Lysinibacillus, Viridibacillus, or Paenibacillus cell transformed with a vector comprising the nucleic acid molecule of any one of claims 1-11.

25. The recombinant Brevibacillus, Lysinibacillus, Viridibacillus, or Paenibacillus cell of claim 22, wherein:
a) the Brevibacillus cell is a Brevibacillus species, comprising: B. agri, B. aydinogluensis, B. borstelensis, B. brevis, B. centrosporus, B. choshinensis, B. fluminis, B. formosus, B. fulvus, B. ginsengisoli, B. invocatus, B. laterosporus, B. levickii, B. limnophilus, B. massiliensis, B. nitrificans, B. panacihumi, B. parabrevis, B. reuszeri, or B. thermoruber; or a bacterium that possesses a 16S rRNA gene that shares at least 97%, 98% or 99% identity with a 16S rRNA gene of a Brevibacillus species;
b) the Lysinibacillus endospore is an endospore formed by a Lysinibacillus species, comprising: Lysinibacillus sphaericus, Lysinibacillus boronitolerans, Lysinibacillus fusiformis, Lysinibacillus acetophenoni, Lysinibacillus alkaliphilus, Lysinibacillus chungkukjangi, Lysinibacillus composti, Lysinibacillus contaminans, Lysinibacillus cresolivorans, Lysinibacillus macroides, Lysinibacillus manganicus, Lysinibacillus mangiferihumi, Lysinibacillus massiliensis, Lysinibacillus meyeri, Lysinibacillus odysseyi, Lysinibacillus pakistanensis, Lysinibacillus parviboronicapiens, Lysinibacillus sinduriensis, Lysinibacillus tabacifolii, Lysinibacillus varians, Lysinibacillus xylanilyticus or Lysinibacillus halotolerans; or an endospore formed by a bacterium that possesses a 16S rRNA gene that shares at least 97%, 98% or 99% identity with a 16S rRNA gene of a Lysinibacillus species;
c) the Viridibacillus endospore is an endospore formed by a Viridibacillus species, comprising: Viridibacillus arvi, Viridibacillus arenosi, or Viridibacillus neidei; or an endospore formed by a bacterium that possesses a 16S rRNA gene that shares at least 97%, 98% or 99% identity with a 16S rRNA gene of a Viridibacillus species; or
d) the Paenibacillus endospore is an endospore formed by a Paenibacillus species, comprising: Paenibacillus sp. NRRL B-50972, Paenibacillus terrae, Paenibacillus polymyxa, or Paenibacillus peoriae; or an endospore formed by a bacterium that possesses a 16S rRNA gene that shares at least 97%, 98% or 99% identity with a 16S rRNA gene of a Paenibacillus species.

26. A method of displaying a heterologous fusion protein on an exosporium of a Brevibacillus, Lysinibacillus, Viridibacillus, or Paenibacillus endospore, the method comprising:
a) transforming a Brevibacillus, Lysinibacillus, Viridibacillus, or Paenibacillus cell capable of sporulation with a recombinant vector comprising the nucleic acid molecule of any one of claims 1-11; and
b) expressing the fusion protein encoded by the nucleic acid molecule of any one of claims 1-11 under sporulation conditions such that the fusion protein is targeted to the exosporium of the Brevibacillus, Lysinibacillus, Viridibacillus, or Paenibacillus endospore resulting from the sporulation;
wherein the N-terminal signal peptide comprises: (i) a polypeptide sequence having at least 60%, 70%, 80%, 90%, 95% or 100% sequence identity with any polypeptide sequence shown in Tables 1-4; or (ii) a fragment of at least 5, 10, 15, 20, 25, 30, 35, 40, 45, 50, 75, 100, 125 or 150 consecutive amino acids of any polypeptide sequence shown in Tables 1-4, and
wherein the N-terminal signal peptide is capable of targeting the fusion protein to an exosporium when expressed in members of at least two different bacterial genera selected from Brevibacillus, Lysinibacillus, Viridibacillus, and/or Paenibacillus.

27. A composition comprising:
a) one or more recombinant exosporium-producing Brevibacillus, Lysinibacillus, Viridibacillus, or Paenibacillus cells that express the fusion protein of any one of claims 12-20, wherein the polypeptide heterologous to the N-terminal signal peptide comprises a plant growth or immune stimulating protein; and
b) at least one biological control agent; optionally,
in a synergistically effective amount.

28. A seed treated with the nucleic acid of any one of claims 1-11, the fusion protein of any one of claims 12-20, the recombinant bacterial cell of claim 24 or 25, or the composition of claim 27.

29. A method of treating a plant, a seed, a plant part, or the soil surrounding the plant to enhance plant growth and/or promote plant health comprising the step of simultaneously or sequentially applying:
a) recombinant exosporium-producing Brevibacillus, Lysinibacillus, Viridibacillus, or Paenibacillus endospores that express the fusion protein of any one of claims 12-20, wherein the polypeptide heterologous to the N-terminal signal peptide comprises a plant growth or immune stimulating protein; and
b) at least one biological control agent; optionally,
in a synergistically effective amount.

30. A method of screening a host plant treated with a recombinant Brevibacillus, Lysinibacillus, Viridibacillus, or Paenibacillus endospore, comprising the following steps:
a) applying a composition comprising a Brevibacillus, Lysinibacillus, Viridibacillus, or Paenibacillus endospore modified to express a fusion protein according to any one of claims 12-20, to a seed, a seedling, or a vegetative plant capable of being permanently or transiently colonized by Brevibacillus, Lysinibacillus, Viridibacillus, or Paenibacillus, to produce a treated seed, seedling, or vegetative plant; and
b) screening the treated seed, seedling, or vegetative plant by detecting and optionally measuring a trait, component, or attribute of the treated seed, seedling, or vegetative plant.

31. The method of claim 30, wherein the screening step comprises one or more of the following:
a) at least one in vitro assay comprising detecting and optionally quantifying the presence, level, change in level, activity, or localization of one or more compounds contained in an extract prepared from a cell or tissue sample obtained from the treated seed, seedling, or vegetative plant; and/or
b) at least one in vivo assay comprising detecting and optionally quantifying a trait, component, or attribute of the treated seed, seedling, or vegetative plant.

32. A method of screening heterologous proteins or peptides expressed in a Brevibacillus cell for agriculturally-significant properties, comprising:
a) modifying a Brevibacillus, Lysinibacillus, Viridibacillus, or Paenibacillus cell to express a fusion protein according to any one of claims 12-20 to produce a recombinant Brevibacillus, Lysinibacillus, Viridibacillus, or Paenibacillus cell; and
b) screening the recombinant Brevibacillus, Lysinibacillus, Viridibacillus, or Paenibacillus cell by detecting, and optionally quantifying, a level or activity of a compound produced by the recombinant Brevibacillus, Lysinibacillus, Viridibacillus, or Paenibacillus cell.

33. A method of treating a plant, a seed, a human, or an animal, comprising:
administering to the plant, seed, human, or animal a composition comprising an exosporium isolated from an endospore produced by a recombinant Brevibacillus, Lysinibacillus, Viridibacillus, or Paenibacillus cell;
wherein the recombinant cell expresses the fusion protein of any one of claims 12-20.

34. The method of claim 29, wherein the composition has been heat-inactivated or sterilized such that no viable Brevibacillus, Lysinibacillus, Viridibacillus, or Paenibacillus cells remain.

35. A composition comprising an isolated and/or purified fusion protein according to any one of claims 12-20.

36. A composition comprising an isolated and/or purified exosporium produced by a recombinant Brevibacillus, Lysinibacillus, Viridibacillus, or Paenibacillus endospore, which has been modified to express a fusion protein according to any one of claims 12-20.

37. A composition comprising an exosporium produced by a recombinant Brevibacillus, Lysinibacillus, Viridibacillus, or Paenibacillus endospore, which has been modified to express a fusion protein according to any one of claims 12-20.

38. The composition of claim 37, wherein the exosporium produced by a recombinant Brevibacillus, Lysinibacillus, Viridibacillus, or Paenibacillus endospore comprises:
a) a basal layer of an exosporium;
b) a hair-like layer of an exosporium;
c) a mixture of both a) and b);
d) a fraction or extract of a crude exosporium obtained from a Brevibacillus, Lysinibacillus, Viridibacillus, or Paenibacillus endospore; and/or
e) a fraction or extract of a crude exosporium obtained from a Brevibacillus, Lysinibacillus, Viridibacillus, or Paenibacillus endospore that is enriched in an amount or concentration of the fusion protein compared to a same amount of the crude exosporium.

39. A method of delivering a protein of interest to a plant, seed or field, comprising:
applying a composition comprising an exosporium obtained from a recombinant Brevibacillus, Lysinibacillus, Viridibacillus, or Paenibacillus endospore to a plant, seed, or field;
wherein the recombinant endospore has been modified to express a fusion protein according to any one of claims 12-20.

40. The method of claim 39, wherein the composition is applied to a field:
a) pre- or post-planting;
b) pre- or post-emergence;
c) as a powder, suspension or solution; and/or
d) wherein the composition further comprises one or more additional compounds that stimulate plant growth or protect plants from pests.

41. The nucleic acid molecule of any of the preceding claims, wherein the N-terminal signal peptide is capable of targeting the fusion protein to an exosporium in members of at least three genera selected from Brevibacillus, Lysinibacillus, Viridibacillus, and/or Paenibacillus.

42. The nucleic acid molecule of any of the preceding claims, wherein the N-terminal signal peptide is capable of targeting the fusion protein to an exosporium in members of the genera Brevibacillus, Lysinibacillus, Viridibacillus, and Paenibacillus.

43. The fusion protein of any of the preceding claims, wherein the N-terminal signal peptide is capable of targeting the fusion protein to an exosporium in members of at least three genera selected from Brevibacillus, Lysinibacillus, Viridibacillus, and/or Paenibacillus.

44. The fusion protein of any of the preceding claims, wherein the N-terminal signal peptide is capable of targeting the fusion protein to an exosporium in members of the genera Brevibacillus, Lysinibacillus, Viridibacillus, and Paenibacillus.

45. The nucleic acid molecule of any of the preceding claims, wherein the N-terminal signal peptide is capable of targeting the fusion protein to an exosporium of:
a) two or more bacterial species selected from P. peoriae, P. chitinolyticus, B. reuszeri, B. laterosporus, L. sphaericus, and/or V. arvi;
b) three or more bacterial species selected from P. peoriae, P. chitinolyticus, B. reuszeri, B. laterosporus, L. sphaericus, and/or V. arvi;
c) four or more bacterial species selected from P. peoriae, P. chitinolyticus, B. reuszeri, B. laterosporus, L. sphaericus, and/or V. arvi;
d) five or more bacterial species selected from P. peoriae, P. chitinolyticus, B. reuszeri, B. laterosporus, L. sphaericus, and/or V. arvi; or
e) P. peoriae, P. chitinolyticus, B. reuszeri, B. laterosporus, L. sphaericus, and V. arvi.

46. The fusion protein of any of the preceding claims, wherein the N-terminal signal peptide is capable of targeting the fusion protein to an exosporium of:
a) two or more bacterial species selected from P. peoriae, P. chitinolyticus, B. reuszeri, B. laterosporus, L. sphaericus, and/or V. arvi;
b) three or more bacterial species selected from P. peoriae, P. chitinolyticus, B. reuszeri, B. laterosporus, L. sphaericus, and/or V. arvi;
c) four or more bacterial species selected from P. peoriae, P. chitinolyticus, B. reuszeri, B. laterosporus, L. sphaericus, and/or V. arvi;
d) five or more bacterial species selected from P. peoriae, P. chitinolyticus, B. reuszeri, B. laterosporus, L. sphaericus, and/or V. arvi; or
e) P. peoriae, P. chitinolyticus, B. reuszeri, B. laterosporus, L. sphaericus, and V. arvi.

47. A method of screening non-Bacillus bacterial strains to identify non-Bacillus bacterial strains that are capable of exosporial display of heterologous proteins, comprising:
a) selecting a pool of non-Bacillus bacterial strains;
b) expressing in each of said non-Bacillus bacterial strains a first nucleic acid construct encoding a first fusion protein, wherein the first nucleic acid construct comprises
(i) a first polynucleotide sequence encoding an N-terminal signal peptide, and
(ii) a second polynucleotide sequence encoding a polypeptide that is is detectable when displayed on spores of the non-Bacillus bacterial strains,
wherein the first polynucleotide sequence comprises a polynucleotide sequence having at least 90%, at least 95%, at least 96%, at least 97%, at least 98%, or at least 99% sequence identity with a polynucleotide sequence encoding the polypeptide sequence of SEQ ID NO: 227, and
wherein the first and second polynucleotide sequences are operably linked;
c) screening said pool of non-Bacillus bacterial strains expressing the nucleic acid construct of part b) for successful exosporial display of the first fusion protein;
d) selecting from said pool one or more non-Bacillus bacterial strains for which successful exosporial display of the fusion protein was detected.

48. The method of claim 47, further comprising:
e) expressing in one of the one or more non-Bacillus bacterial strains selected in part d) a second nucleic acid construct encoding a second fusion protein, wherein the second nucleic acid construct comprises
(i) a first polynucleotide sequence encoding an N-terminal signal peptide, and
(ii) a second polynucleotide sequence encoding a polypeptide that is heterologous to the N-terminal signal peptide that is endogenous to the non-Bacillus bacterial strains,
wherein the first polynucleotide sequence comprises a polynucleotide sequence having at least 90%, at least 95%, at least 96%, at least 97%, at least 98%, or at least 99% sequence identity with a polynucleotide sequence encoding an N-terminal signal peptide that is endogenous to the non-Bacillus bacterial strains, and
wherein the first and second polynucleotide sequences are operably linked.

49. The method of claim 48, wherein the second polynucleotide sequence encodes a polypeptide that is detectable when displayed on spores of the non-Bacillus bacterial strains.

50. The method of any of claims 47-48, wherein the non-Bacillus bacterial strains are spore-forming.

51. The method of claim 50, wherein the the non-Bacillus bacterial strains are selected from the group consisting of Brevibacillus, Lysinibacillus, Viridibacillus, and Paenibacillus.

52. The method of any of claims 48-51, wherein the N-terminal signal peptide that is endogenous to the non-Bacillus bacterial strains is an N-terminal signal peptide from an endogenous collagen-like glycoprotein.

53. The method of any of claims 47-52, wherein display of the fusion proteins is screened in step c) using microscopy, enzyme activity assays, antibody binding assays, colorimetric assays, whole cell fluorescence, whole cell surface plasmon resonance, whole cell immunoassay, or other whole cell assay.

54. The method of any of claims 47 and 49-53, wherein display of either of the fusion proteins is detectable by microscopy.

55. The method of any of claims 47 and 49-53, wherein display of either of the fusion proteins is detectable by flow cytometry.

56. The method of any of claims 47 and 49-55, wherein the polypeptide that is detectable when displayed is selected from: a fluorescent protein, a chemiluminescent-assay enzyme, or a chromogenic-assay enzyme.

57. The method of claim 56, wherein the fluorescent protein is tdTomato.

58. The method of any of claims 47-57, wherein the polynucleotide sequence encoding the N-terminal signal peptide of the first nucleic acid construct comprises a polynucleotide sequence encoding the polypeptide sequence of SEQ ID NO: 227.

59. A nucleic acid molecule encoding a fusion protein, comprising:
(i) a first polynucleotide sequence encoding an N-terminal signal peptide, and
(ii) a second polynucleotide sequence encoding a polypeptide that is is detectable when displayed on spores of a non-Bacillus bacterial strain that is not Paenibacillus spp.,
wherein the first polynucleotide sequence comprises a polynucleotide sequence having at least 90%, at least 95%, at least 96%, at least 97%, at least 98%, or at least 99% sequence identity with a polynucleotide sequence encoding the polypeptide sequence of SEQ ID NO: 227, and
wherein the first and second polynucleotide sequences are operably linked.

60. The nucleic acid molecule of claim 59, wherein the first polynucleotide sequence comprises the polynucleotide sequence of SEQ ID NO: 227.

61. The nucleic acid molecule of any of claims 59-60, wherein display of the fusion protein is detectable using microscopy, whole cell fluorescence, whole cell surface plasmon resonance, whole cell immunoassay, or other whole cell assay.

62. The nucleic acid molecule of any of claims 59-61, wherein display of the fusion protein is detectable by microscopy.

63. The nucleic acid molecule of any of claims 59-62, wherein the polypeptide that is detectable when displayed is selected from: a fluorescent protein, a chemiluminescent-assay enzyme, or a chromogenic-assay enzyme.

64. The nucleic acid molecule of claim 63, wherein the fluorescent protein is tdTomato.
